(19) United States
(12) Patent Application Publication
(10) Pub. No.: US 2023/0146665 A1
(43) Pub. Date: May 11, 2023

(54) IL-18-FC FUSION PROTEINS

(71) Applicant: Xencor, Inc., Monrovia, CA (US)

(72) Inventors: Alex Nisthal, Monrovia, CA (US); John Desjarlais, Pasadena, CA (US); Gregory Moore, Azusa, CA (US); Sung-Hyung Lee, San Gabriel, CA (US)

(21) Appl. No.: 17/815,194

(22) Filed: Jul. 26, 2022

Related U.S. Application Data
(60) Provisional application No. 63/226,093, filed on Jul. 27, 2021.
(60) Provisional application No. 63/310,519, filed on Feb. 15, 2022.
(60) Provisional application No. 63/327,705, filed on Apr. 5, 2022.

(51) Int. Cl. C07K 14/54

(52) U.S. Cl. CPC C07K 14/54; C07K 2319/30

(57) ABSTRACT

Provided herein are IL18-Fc fusion proteins that include an empty-Fc domain and an IL18 protein connected to another Fc domain. Also provided herein are additional are IL18-Fc fusion proteins such as IL18 x Fab-Fc fusion proteins that include a first monomer containing an IL18 protein connected to a first Fc domain, a second monomer containing a variable heavy chain connected to a second Fc domain, and a variable light chain, such that the variable heavy and light chains form an empty Fab. Various variant IL18 proteins with modifications to reduce heterogeneity and/or reduce affinity/potency and/or improve stability and/or improve production yield are also described.

### PRIORITY CLAIM

This application claims priority to U.S. Provisional Patent Application No. 63/226,093, filed Jul. 27, 2021, U.S. Provisional Patent Application No. 63/310,519, filed Feb. 15, 2022 and U.S. Provisional Patent Application No. 63/327,705, filed Apr. 5, 2022, which are hereby incorporated by reference in their entireties.

### SEQUENCE LISTING

The instant application contains a Sequence Listing which has been submitted electronically in ST.26 XML format and is hereby incorporated by reference in its entirety. Said ST.26 XML format file was created on Jul. 25, 2022, is named 067461-5291-WO SL.txt and is 2,108,595 bytes in size

### BACKGROUND

In order for the immune system to mount an effective anti-tumor response, two things must occur. T cells in the tumor environment must first engage antigenic tumor peptides presented by major histocompatibility complexes (MHC) on tumor cells. Next, the T cells must be induced by cytokines such as IL-15 and IL-2 to produce costimulatory cytokines such as IFNy. Recognition of tumor peptides alone in the absence of cytokine induction leads to T cells becoming anergic, thereby leading to tolerance. Accordingly, a very promising approach in cancer immunotherapy is cytokine-based treatments. For example, IL-2 has been approved for use in patients with metastatic renal-cell carcinoma and malignant melanoma.

IL18 is proinflammatory cytokine that exerts cell signaling upon binding to the IL18 receptor IL18R1 and the IL18 receptor accessory protein (IL18RAP) to form a ternary signaling complex which activates NF-kappa-B, and in turn activates synthesis of inflammatory mediators. The activity of IL18 can be suppressed by the IL18 binding protein (IL18BP) which binds to IL18 and prevents it from binding to the IL18 receptor. Recombinant IL18 is a promising cytokine-treatment due to its broad effect in activating the immune system as IL-18 signaling contributes to cytokine production and immune response by Th1 and Th2 lymphocytes. Thus, there remains a need for novel IL18 based compositions for the treatment of cancers.

### BRIEF SUMMARY

In some aspects, provided herein is a composition comprising a variant human IL18 protein, wherein the variant IL18 protein comprises a modification at one or more amino acid positions selected from the group consisting of Y1, E6, S7, K8, S10, V11, N14, L15, D17, Q18, D23, R27, P28, L29, E31, M33, T34, D35, S36, D37, C38, R39, D40, N41, R44, I46, I49, S50, M51, K53, D54, S55, Q56, P57, M60, A61, V62, T63, S65, K67, C68, E69, I71, C76, E77, I80, I81, N87, P88, D90, K93, T95, K96, S97, Q103, H109, D110, N111, M113, S119, A126, C127, D132, L136, L138, K139, E141, L144, D146, R147, I149, M150, N155, E156, and D157, as compared to wildtype human IL18.

In some embodiments, the variant human IL18 protein of the composition comprises an additional 1, 2, 3, 4, 5, 6, 7, 8, 9, or 10 amino acid modifications.

In some embodiments, the variant human IL18 protein of the composition comprising one or more amino acid substitutions selected from the group consisting of Y1F, Y1H, E6A, E6Q, S7C, S7P, K8E, K8Q, K8Y, S10C, V11I, N14C, N14W, L15C, D17N, Q18L, D23N, D23S, R27Q, P28C, L29V, E31Q, M33C, T34P, D35N, D35E, S36D, S36N, D37N, C38S, C38Q, C38R, C38E, C38L, C38I, C38V, C38K, C38D, R39S, R39T, D40N, N41Q, R44Q, I46V, I49C, S50C, S50Y, M51I, M51K, M51Q, M51R, M51L, M51H, M51F, M51Y, K53A, K53D, K53E, K53G, K53H, K53I, K53L, K53M, K53N, K53Q, K53R, K53S, K53T, K53V, K53Y, K53F, D54C, S55N, S55Q, S55D, S55E, S55T, Q56I, Q56L, P57A, P57E, P57T, P57V, P57Q, P57D, P57Y, P57N, M60I, M60L, M60K, M60Y, M60F, A61C, V62C, T63C, S65C, K67Q, C68S, C68I, C68F, C68Y, C68D, C68N, C68E, C68Q, C68K, E69K, I71M, C76S, C76E, C76K, E77K, 180T, I81L, I81V, N87S, P88C, D90E, K93D, K93N, T95E, K96G, K96Q, S97N, Q103C, Q103E, Q103I, Q103L, H109W, H109Y, D110N, D110Q, D110R, N111D, N111Q, N111S, N111T, N111E, M113I, S119L, A126C, C127S, C127W, C127Y, C127F, C127D, C127E, C127K, D132Q, D132E, L136C, L138C, K139C, E141K, E141Q, L144N, D146F, D146L, D146Y, R147C, R147K, I149V, M150F, M150T, N155C, E156Q, D157A, D157S, D157N, and D157del, as compared to wildtype human IL18. In some embodiments, the amino acid substitution can include 4CS, 4CS/D193S, 4CS/D193A, 4CS/delD193, 4CS/S38E, 4CS/S68E, 4CS/S76E, 4CS/S127E, 4CS/S38K, 4CS/S68K, 4CS/S76K, 4CS/S127K, 4CS/S38D, 4CS/Y1F, 4CS/Y1H, 4CS/E6A, 4CS/E6Q, 4CS/D17N, 4CS/E31Q, 4CS/D35N, 4CS/D37N, 4CS/D40N, 4CS/N41Q, 4CS/K53R, 4CS/K53H, 4CS/K53M, 4CS/K53E, 4CS/K53Q, 4CS/K53A, 4CS/Q103E, 4CS/D110N, 4CS/N111Q, 4CS/E6A/K53A, 4CS/N14C/E31Q/S127C, 4CS/E31Q/K53A, 4CS/E31Q/D35N/K53A, 4C S/E31Q/N41Q/K53A, 4CS/E31Q/D35N/N41Q/K53A, 4CS/E31Q/D35N, 4CS/E31Q/N41Q, 4CS/E31Q/D35N/N41Q, 4CS/E31Q/D37N, 4CS/E31Q/D37N/K53A, 4C S/E31Q/M33C/S38C, 4CS/E31Q/S76C/L138C, 4CS/E31Q/S68I, 4CS/E31Q/S68F, 4CS/E31Q/S127W, 4C S/E31Q/S127Y, 4CS/E31Q/S127F, 4CS/S10C/E31Q/I49C, 4C S/L15C/E31Q/R147C, 4C S/P28C/E31Q/L136C, 4CS/E31Q/S50C/P88C, 4CS/E31Q/T63C/P88C, 4C S/E31Q/V62C/Q103C, 4CS/S10C/E31Q/N155C, 4CS/E31Q/S65C/P88C, 4CS/S7C/E31Q/S50C, 4CS/E31Q/D54C/A61C, 4C S/E31Q/A126C/K139C, 4CS/N14W/E31Q, 4CS/E31Q/D146Y, 4CS/E31Q/D146L, 4C S/E31Q/D146F, 4C S/E31Q/Q103L, 4CS/E31Q/Q103I, 4CS/E31Q/M150F, 4CS/Q18L/E31Q, 4CS/E31Q/S68Y, 4CS/E31Q/S38Q, 4C S/E31Q/S38R, 4CS/E31Q/S68D, 4CS/S7P/E31Q, 4CS/V11I/E31Q, 4CS/D23N/E31Q, 4CS/D23S/E31Q, 4CS/R27Q/E31Q, 4CS/L29V/E31Q, 4CS/E31Q/T34P, 4CS/E31Q/R39T, 4CS/E31Q/R39S, 4CS/E31Q/R44Q, 4CS/E31Q/I46V, 4CS/E31Q/S50Y, 4CS/E31Q/Q56L, 4CS/E31Q/Q56L/P57T, 4C S/E31Q/P57T, 4CS/E31Q/P57V, 4CS/E31Q/M60L, 4CS/E31Q/K67Q, 4CS/E31Q/E69K, 4CS/E31Q/I71M, 4CS/E31Q/E77K, 4CS/E31Q/I80T, 4CS/E31Q/I81V, 4CS/E31Q/I81L, 4C S/E31Q/N87S, 4CS/E31Q/D90E, 4CS/E31Q/K93D/T95E, 4CS/E31Q/K93N/T95E, 4CS/E31Q/T95E, 4CS/E31Q/K96G, 4CS/E31Q/S97N, 4CS/E31Q/N111D, 4CS/E31Q/M113I, 4CS/E31Q/S119L, 4CS/E31Q/L144N, 4CS/E31Q/R147K, 4CS/E31Q/I149V, 4CS/E31Q/M150T, 4CS/E31Q/E156Q/D157N, 4CS/K53S, 4CS/K53G, 4CS/K53T, 4CS/K53I, 4CS/K53L, 4CS/K53N, 4CS/K53D, 4CS/M51K, 4CS/M51Q, 4CS/M51I, 4CS/S55N, 4CS/S55Q, 4CS/Q56L, 4CS/Q56I, 4CS/P57A, 4CS/P57E, 4CS/M60L, 4CS/M60I, 4CS/K8Y, 4CS/K8Q, 4CS/K8E, 4CS/H109W, 4CS/H109Y, 4CS/E31Q/S38E, 4CS/E31Q/S38L, 4CS/E31Q/S38I, 4C S/E31Q/S38V, 4C S/E31Q/S68N, 4CS/E31Q/S68E, 4CS/E31Q/S68Q, 4C S/E31Q/S76C, 4CS/E31Q/S127D, 4CS/E31Q/S127E, 4CS/D23N/E31Q/R27Q, 4CS/E31Q/Q56L/T95E, 4CS/E31Q/K96Q/S119L, 4CS/E31Q/E141K/I149V, 4CS/E31Q/E141Q/I149V, 4CS/S7P/E31Q/S50Y, 4C S/E31Q/I80T/I81L/delD193, 4C S/E31Q/P57A/S119L/delD193, 4CS/E31Q/P57A/180T/181L/S119L/delD193, 4C S/E31Q/P57A/K93D/T95E/S119L/delD193, 4C S/E31Q/I80T/S119L/delD193, 4C S/E31Q/I80T/I81L/K93D/T95E/delD193, 4CS/E31Q/P57A/180T/181L/K93D/T95E/S119L/delD193, 4CS/S7C/E31Q/S50C/delD193, 4CS/S7C/E31Q/S50C/P57A/delD193, 4CS/S7C/E31Q/S50C/S119L/delD193, 4CS/S7C/E31Q/S50C/I80T/delD193, 4CS/S7C/E31Q/S50C/I80T/S119L/delD193, 4C S/S7C/E31Q/S50C/P57A/I80T/S119L/delD193, 4C S/S10C/E31Q/N155C/delD193, 4C S/S10C/E31Q/P57A/N155C/delD193, 4C S/S10C/E31Q/S119L/N155C/delD193, 4C S/S10C/E31Q/I80T/N155C/delD193, 4C S/S10C/E31Q/I80T/S119L/N155C/delD193, 4C S/S10C/E31Q/P57A/I80T/S119L/N155C/delD193, 4C S/S10C/E31Q/I49C/delD193, 4C S/L15C/E31Q/R147C/delD193, 4C S/E31Q/T63C/P88C/delD193, 4CS/N14C/E31Q/S127C/delD193, 4CS/E31Q/S38R/S127W/delD193, 4CS/S10C/D35E/N155C, 4CS/S10C/S36D/N155C, 4CS/S10C/S36N/N155C, 4CS/S10C/K53V/N155C, 4CS/S10C/K53Y/N155C, 4CS/S10C/K53F/N155C, 4CS/S10C/M51R/N155C, 4CS/S10C/M51L/N155C, 4CS/S10C/M51H/N155C, 4CS/S10C/M51F/N155C, 4CS/S10C/M51Y/N155C, 4CS/S10C/S55D/N155C, 4CS/S10C/S55E/N155C, 4CS/S10C/S55T/N155C, 4CS/S10C/P57Q/N155C, 4CS/S10C/P57D/N155C, 4CS/S10C/P57Y/N155C, 4CS/S10C/P57N/N155C, 4CS/S10C/M60Y/N155C, 4CS/S10C/M60F/N155C, 4CS/S10C/D110Q/N155C, 4CS/S10C/D110R/N155C, 4CS/S10C/N111D/N155C, 4CS/S10C/N111S/N155C, 4CS/S10C/N111T/N155C, 4CS/S10C/N111E/N155C, 4CS/S10C/D132Q/N155C, 4CS/S10C/D132EN155C, 4CS/E6Q/S10C/K53D/N155C, 4CS/E6Q/S10C/M51K/K53D/N155C, 4CS/S10C/E31Q/D35N/N41Q/K53A/N155C, 4CS/S10C/E31Q/N41Q/K53A/N155C, 4CS/S10C/E31Q/K53A/N155C, 4CS/S10C/K53T/N155C, 4CS/S10C/P57A/N155C, 4CS/S10C/N155C, 4CS/S10C/S76G/N155C, 4CS/S10C/S76A/N155C, 4CS/S10C/M51K/K53D/N155C, 4CS/S10C/M51K/K53E/N155C, 4CS/E6Q/S10C/K53E/N155C, 4CS/E6Q/S10C/M51K/K53E/N155C, 4CS/E6Q/S10C/M51K/P57E/N155C, 4CS/S10C/M51K/P57E/N155C, 4CS/E6Q/S10C/P57E/N155C, 4CS/S10C/E31Q/K53T/N155C, 4CS/S10C/K53G/P57E/N155C, 4CS/S10C/K53T/P57E/N155C, 4CS/S10C/K53A/P57E/N155C, 4CS/S10C/P57E/N155C, 4CS/S10C/K53D/N155C, 4CS/S10C/E31Q/N41Q/N155C, 4CS/S10C/K53A/N155C, 4CS/S10C/K53G/N155C, 4CS/S10C/K53E/N155C, 4CS/S10C/K53S/N155C, 4CS/S10C/M51L/K53D/N155C, 4CS/S10C/K53D/D110R/N155C, 4CS/S10C/K53D/N111T/N155C, 4CS/S10C/K53D/S55T/N155C, 4CS/S10C/K53D/S55T/D110R/N155C, 4CS/S10C/M51L/K53D/S55T/D110R/N111T/N155C, 4CS/S10C/M51L/K53D/S55T/D110R/N155C, 4CS/S10C/K53D/S55T/D110R/N111T/N155C, 4CS/S10C/K53D/S55T/N111T/N155C, 4CS/S10C/E31Q/D35N/N155C, 4CS/S10C/N41Q/N155C, 4CS/S10C/D35N/N155C, 4CS/S10C/D37N/N155C, 4CS/S10C/E31Q/D37N/N155C, 4CS/S10C/D35N/D37N/N155C, 4CS/E6Q/S10C/M51L/K53D/S55T/D110R/N111T/N155C, 4CS/S10C/K53D/H109Y/N155C, 4CS/S10C/D37N/K53D/N155C, 4CS/S10C/D35N/K53D/N155C, 4CS/K8E/S10C/K53D/N155C, 4CS/S10C/E31Q/K53D/N155C, 4CS/S10C/N41Q/K53D/N155C, 4CS/S10C/K53D/P57V/N155C, 4CS/S10C/K53D/P57T/N155C, 4CS/E6Q/S10C/K53D/N111T/N155C, E6A/K53A, D35N/K53A, N41Q/K53A, D35N/N41Q/K53A, D35N/N41Q, D37N/K53A, E6Q/K53D, E6Q/M51K/K53D, M51K/K53D, M51K/K53E, E6Q/K53E, E6Q/M51K/K53E, E6Q/M51K/P57E, M51K/P57E, E6Q/P57E, K53G/P57E, K53T/P57E, K53A/P57E, M51L/K53D, K53D/D110R, K53D/N111T, K53D/S55T, K53D/S55T/D110R, M51L/K53D/S55T/D110R/N111T, M51L/K53D/S55T/D110R, K53D/S55T/D110R/N111T, K53D/S55T/N111T, D35N/D37N, E6Q/M51L/K53D/S55T/D110R/N111T, K53D/H109Y, D37N/K53D, D35N/K53D, K8E/K53D, N41Q/K53D, K53D/P57V, K53D/P57T, E6Q/K53D/N111T, Q56L/P57T, K93D/T95E, K93N/T95E, E156Q/D157N, D23N/R27Q, Q56L/T95E, K96Q/S119L, E141K/I149V, E141Q/I149V, S7P/S50Y, I80T/I81L, P57A/S119L, P57A/I80T/I81L/S119L, P57A/K93D/T95E/S119L, I80T/S119L, I80T/I81L/K93D/T95E, P57A/I80T/I81L/K93D/T95E/S119L, P57A/I80T/S119L, N14C/S127C, M33C/S38C, S76C/L138C, S10C/I49C, L15C/R147C, P28C/L136C, S50C/P88C, T63C/P88C, V62C/Q103C, S10C/N155C, S65C/P88C, S7C/S50C, D54C/A61C, A126C/K139C, C38R/C127W, E31Q/K53A, E31Q/D35N/K53A, E31Q/N41Q/K53A, E31Q/D35N/N41Q/K53A, E31Q/D35N, E31Q/N41Q, E31Q/D35N/N41Q, E31Q/D37N, E31Q/D37N/K53A, S10C/E31Q/I49C, L15C/E31Q/R147C, P28C/E31Q/L136C, E31Q/S50C/P88C, E31Q/T63C/P88C, E31Q/V62C/Q103C, S10C/E31Q/N155C, E31Q/S65C/P88C, S7C/E31Q/S50C, E31Q/D54C/A61C, E31Q/A126C/K139C, N14W/E31Q, E31Q/D146Y, E31Q/D146L, E31Q/D146F, E31Q/Q103L, E31Q/Q103I, E31Q/M150F, Ql8L/E31Q, S7P/E31Q, V11I/E31Q, D23N/E31Q, D23 S/E31Q, R27Q/E31Q, L29V/E31Q, E31Q/T34P, E31Q/R39T, E31Q/R39S, E31Q/R44Q, E31Q/I46V, E31Q/S50Y, E31Q/Q56L, E31Q/Q56L/P57T, E31Q/P57T, E31Q/P57V, E31Q/M60L, E31Q/K67Q, E31Q/E69K, E31Q/I71M, E31Q/E77K, E31Q/I80T, E31Q/I81V, E31Q/I81L, E31Q/N87S, E31Q/D90E, E31Q/K93D/T95E, E31Q/K93N/T95E, E31Q/T95E, E31Q/K96G, E31Q/S97N, E31Q/N111D, E31QN1113I, E31Q/S119L, E31Q/L144N, E31Q/R147K, E31Q/I149V, E31Q/M150T, E31Q/E156Q/D157N, D23N/E31Q/R27Q, E31Q/Q56L/T95E, E31Q/K96Q/S119L, E31Q/E141K/I149V, E31Q/E141Q/I149V, S7P/E31Q/S50Y, E31Q/180T/181L/delD193, E31Q/P57A/S119L/delD193, E31Q/P57A/I80T/I81L/S119L/delD193, E31Q/P57A/K93D/T95E/S119L/delD193, E31Q/I80T/S119L/delD193, E31Q/I80T/I81L/K93D/T95E/delD193, E31Q/P57A/180T/181L/K93D/T95E/S119L/delD193, S7C/E31Q/S50C/delD193, S7C/E31Q/S50C/P57A/delD193, S7C/E31Q/S50C/S119L/delD193, S7C/E31Q/S50C/I80T/delD193, S7C/E31Q/S50C/I80T/S119L/delD193, S7C/E31Q/S50C/P57A/I80T/S119L/delD193, S10C/E31Q/N155C/delD193, S10C/E31Q/P57A/N155C/delD193, S10C/E31Q/S119L/N155C/delD193, S10C/E31Q/I80T/N155C/delD193, S10C/E31Q/I80T/S119L/N155C/delD193, S10C/E31Q/P57A/I80T/S119L/N155C/delD193, S10C/E31Q/I49C/delD193, L15C/E31Q/R147C/delD193, E31Q/T63C/P88C/delD193, S10C/D35E/N155C, S10C/S36D/N155C, S10C/S36N/N155C, S10C/K53V/N155C, S10C/K53Y/N155C, S10C/K53F/N155C, S10C/M51R/N155C, S10C/M51L/N155C, S10C/M51H/N155C, S10C/M51F/N155C, S10C/M51Y/N155C, S10C/S55D/N155C, S10C/S55E/N155C, S10C/S55T/N155C, S10C/P57Q/N155C, S10C/P57D/N155C, S10C/P57Y/N155C, S10C/P57N/N155C, S10C/M60Y/N155C, S10C/M60F/N155C, S10C/D110Q/N155C, S10C/D110R/N155C, S10C/N111D/N155C, S10C/N111S/N155C, S10C/N111T/N155C, S10C/N111E/N155C, S10C/D132Q/N155C, S10C/D132E/N155C, E6Q/S10C/K53D/N155C, E6Q/S10C/M51K/K53D/N155C, S10C/E31Q/D35N/N41Q/K53A/N155C, S10C/E31Q/N41Q/K53A/N155C, S10C/E31Q/K53A/N155C, S10C/K53T/N155C, S10C/P57A/N155C, S10C/M51K/K53D/N155C, S10C/M51K/K53E/N155C, E6Q/S10C/K53E/N155C, E6Q/S10C/M51K/K53E/N155C, E6Q/S10C/M51K/P57E/N155C, S10C/M51K/P57E/N155C, E6Q/S10C/P57E/N155C, S10C/E31Q/K53T/N155C, S10C/K53G/P57E/N155C, S10C/K53T/P57E/N155C, S10C/K53A/P57E/N155C, S10C/P57E/N155C, S10C/K53D/N155C, S10C/E31Q/N41Q/N155C, S10C/K53A/N155C, S10C/K53G/N155C, S10C/K53E/N155C, S10C/K53S/N155C, S10C/M51L/K53D/N155C, S10C/K53D/D110R/N155C, S10C/K53D/N111TN155C, S10C/K53D/S55T/N155C, S10C/K53D/S55T/D110R/N155C, S10C/M51L/K53D/S55T/D110R/N111T/N155C, S10C/M51L/K53D/S55T/D110R/N155C, S10C/K53D/S55T/D110R/N111T/N155C, S10C/K53D/S55T/N111T/N155C, S10C/E31Q/D35N/N155C, S10C/N41Q/N155C, S10C/D35N/N155C, S10C/D37N/N155C, S10C/E31Q/D37N/N155C, S10C/D35N/D37N/N155C, E6Q/S10C/M51L/K53D/S55T/D110R/N111TN155C, S10C/K53D/H109Y/N155C, S10C/D37N/K53D/N155C, S10C/D35N/K53D/N155C, K8E/S10C/K53D/N155C, S10C/E31Q/K53D/N155C, S10C/N41Q/K53DN155C, S10C/K53D/P57V/N155C, S10C/K53D/P57T/N155C, or E6Q/S10C/K53D/N111T/N155C.

In some embodiments, the variant human IL18 protein exhibits reduced binding affinity to the IL18 receptor 1 (IL18R1), IL18 receptor accessory protein (IL18RAP), IL18R1:IL18RAP complex and/or the IL18 binding protein (IL18BP), compared to wildtype human IL18. In some embodiments, the variant human IL18 protein exhibits reduced heterogeneity compared to wildtype human IL18. In some embodiments, the variant human IL18 protein exhibits improved production yield compared to wildtype human IL18. In some embodiments, the variant human IL18 protein exhibits improved stability compared to wildtype human IL18. In some embodiments, the variant human IL18 protein exhibits modulated potency, compared to wildtype human IL18. In some embodiments, the variant human IL18 protein exhibits reduced IL18BP sink compared to wildtype human IL18.

Provided herein is a nucleic acid encoding any one of the variant human IL18 proteins described herein. Provided herein is an expression vector comprising any one of the nucleic acids described. Provided herein is a host cell comprising any one of the nucleic acids described or any one of the expression vectors described. In some embodiments, provided herein is a method of making a variant human IL18 protein comprising culturing any one of the host cells described herein and recovering the variant human IL18.

In some aspects, provided herein is a monovalent Fc fusion protein comprising: (a) a first monomer comprising from N-terminus to C-terminus: a wildtype or variant IL-18 protein and a first Fc domain; and (b) a second monomer comprising a second Fc domain.

In some embodiments of the monovalent Fc fusion protein, the first Fc domain further comprises a set of amino acid substitutions Q295E/N384D/Q418E/N421D, according to EU numbering. In some embodiments, first and/or second Fc domains further comprise an amino acid modification of K447del, according to EU numbering. In some embodiments, the first and/or second Fc domains further comprise a set of amino acid modifications selected from the group consisting of C219S, C220S, S228P, G236R/L328R, E233P/L234V/L235A/G236del/S239K, E233P/L234V/L235A/G236del/S239K/A327G, E233P/L234V/L235A/G236del/S267K/A327G, E233P/L234V/L235A/G236de1, E233P/L234V/L235A/G236del/S267K, and C220S/ E233P/L234V/L235A/G236del/S267K, according to EU numbering. In some embodiments, the first and second Fc domains each further comprises amino acid modifications C220S/E233P/L234V/L235A/G236del/S267K, according to EU numbering. In some embodiments, the first Fc domain and the second Fc domain have a set of amino acid substitutions selected from the group consisting of: (i) S267K/L368D/K370S: S267K/S364K/E357Q; (ii) S364K/E357Q: L368D/K370S; (iii) L368D/K370S: S364K; (iv) L368E/K370S: S364K; (v) T411E/K360E/Q362E: D401K; (vi) L368D/K370S: S364K/E357Q, and (vii) K370S: S364K/E357Q, according to EU numbering. In some embodiments, the first and second Fc domains further comprise amino acid substitutions M428L/N434S, according to EU numbering.

In some embodiments, the wildtype or variant IL-18 protein is covalently attached to the N-terminus of the first Fc domain. In some embodiments, the wildtype or variant IL-18 protein is covalently attached to a domain linker which is covalently attached to the N-terminus of the first Fc domain. In some embodiments, the domain linker is selected from any one of the domain linkers in FIG. 8.

In some embodiments, the wildtype IL18 protein has an amino acid sequence selected from the group consisting of SEQ ID NO:1 (human precursor IL18) and SEQ ID NO:2 (human mature IL18).

In some embodiments, the variant IL18 protein has at least 90% sequence identity to an amino acid sequence selected from the group consisting of SEQ ID NO:1 (human precursor IL18) and SEQ ID NO:2 (human mature IL18).

In some embodiments, the variant IL18 protein comprises a modification at one or more amino acid positions selected from the group consisting of Y1, E6, S7, K8, S10, V11, N14, L15, D17, Q18, D23, R27, P28, L29, E31, M33, T34, D35, S36, D37, C38, R39, D40, N41, R44, I46, I49, S50, M51, K53, D54, S55, Q56, P57, M60, A61, V62, T63, S65, K67, C68, E69, I71, C76, E77, I80, I81, N87, P88, D90, K93, T95, K96, S97, Q103, H109, D110, N111, M113, S119, A126, C127, D132, L136, L138, K139, E141, L144, D146, R147, I149, M150, N155, E156, and D157, as compared to wildtype human IL18.

In some embodiments, the variant IL18 protein comprises one or more amino acid substitutions selected from the group consisting of Y1F, Y1H, E6A, E6Q, S7C, S7P, K8E, K8Q, K8Y, S10C, V11I, N14C, N14W, L15C, D17N, Q18L, D23N, D23S, R27Q, P28C, L29V, E31Q, M33C, T34P, D35N, D35E, S36D, S36N, D37N, C38S, C38K, C38D, C38Q, C38R, C38E, C38L, C381, C38V, R39S, R39T, D40N, N41Q, R44Q, I46V, I49C, S50C, S50Y, M51I, M51K, M51Q, M51R, M51L, M51H, M51F, M51Y, K53A, K53D, K53E, K53G, K53H, K531, K53L, K53M, K53N, K53Q, K53R, K53S, K531, K53V, K53Y, K53F, D54C, S55N, S55Q, S55D, S55E, S551, Q561, Q56L, P57A, P57E, P571, P57V, P57Q, P57D, P57Y, P57N, M60I, M60L, M60K, M60Y, M60F, A61C, V62C, T63C, S65C, K67Q, C68S, C681, C68F, C68Y, C68D, C68N, C68E, C68Q, C68K, E69K, I71M, C76S, C76E, C76K, E77K, 180T, I81L, I81V, N87S, P88C, D90E, K93D, K93N, T95E, K96G, K96Q, S97N, Q103C, Q103E, Q103I, Q103L, H109W, H109Y, D110N, D110Q, D110R, N111D, N111Q, N111S, N111T, N111E, M113I, S119L, A126C, C127S, C127W, C127Y, C127F, C127D, C127E, C127K, D132Q, D132E, L136C, L138C, K139C, E141K, E141Q, L144N, D146F, D146L, D146Y, R147C, R147K, I149V, M150F, M150T, N155C, E156Q, D157A, D157S, D157N, and D157del, as compared to wildtype human IL18. In some embodiments, the amino acid substitution can include 4CS, 4CS/D193S, 4CS/D193A, 4CS/delD193, 4CS/S38E, 4CS/S68E, 4CS/S76E, 4CS/S127E, 4CS/S38K, 4CS/S68K, 4CS/S76K, 4CS/S127K, 4CS/S38D, 4CS/Y1F, 4CS/Y1H, 4CS/E6A, 4CS/E6Q, 4CS/D17N, 4CS/E31Q, 4CS/D35N, 4CS/D37N, 4CS/D40N, 4CS/N41Q, 4CS/K53R, 4CS/K53H, 4CS/K53M, 4CS/K53E, 4CS/K53Q, 4CS/K53A, 4CS/Q103E, 4CS/D110N, 4CS/N111Q, 4CS/E6A/K53A, 4CS/N14C/E31Q/S127C, 4CS/E31Q/K53A, 4CS/E31Q/D35N/K53A, 4CS/E31Q/N41Q/K53A, 4CS/E31Q/D35N/N41Q/K53A, 4CS/E31Q/D35N, 4CS/E31Q/N41Q, 4C S/E31Q/D35N/N41Q, 4C S/E31Q/D37N, 4CS/E31Q/D37N/K53A, 4CS/E31Q/M33C/S38C, 4C S/E31Q/S76C/L138C, 4CS/E31Q/S68I, 4CS/E31Q/S68F, 4CS/E31Q/S127W, 4CS/E31Q/S127Y, 4CS/E31Q/S127F, 4CS/S10C/E31Q/149C, 4C S/L15C/E31Q/R147C, 4C S/P28C/E31Q/L136C, 4CS/E31Q/S50C/P88C, 4CS/E31Q/163C/P88C, 4C S/E31Q/V62C/Q103C, 4CS/S10C/E31Q/N155C, 4CS/E31Q/S65C/P88C, 4CS/S7C/E31Q/S50C, 4CS/E31Q/D54C/A61C, 4C S/E31Q/A126C/K139C, 4CS/N14W/E31Q, 4CS/E31Q/D146Y, 4CS/E31Q/D146L, 4C S/E31Q/D146F, 4C S/E31Q/Q103L, 4CS/E31Q/Q103I, 4CS/E31Q/M150F, 4CS/Q18L/E31Q, 4CS/E31Q/S68Y, 4CS/E31Q/S38Q, 4C S/E31Q/S38R, 4CS/E31Q/S68D, 4CS/S7P/E31Q, 4CS/V11I/E31Q, 4CS/D23N/E31Q, 4CS/D23S/E31Q, 4CS/R27Q/E31Q, 4CS/L29V/E31Q, 4CS/E31Q/T34P, 4CS/E31Q/R39T, 4CS/E31Q/R39S, 4CS/E31Q/R44Q, 4CS/E31Q/I46V, 4CS/E31Q/S50Y, 4CS/E31Q/Q56L, 4CS/E31Q/Q56L/P57T, 4C S/E31Q/P57T, 4CS/E31Q/P57V, 4CS/E31Q/M60L, 4CS/E31Q/K67Q, 4CS/E31Q/E69K, 4CS/E31Q/I71M, 4CS/E31Q/E77K, 4CS/E31Q/I80T, 4CS/E31Q/I81V, 4CS/E31Q/I81L, 4C S/E31Q/N87S, 4CS/E31Q/D90E, 4CS/E31Q/K93D/T95E, 4CS/E31Q/K93N/T95E, 4CS/E31Q/T95E, 4CS/E31Q/K96G, 4CS/E31Q/S97N, 4CS/E31Q/N111D, 4CS/E31QN1113I, 4CS/E31Q/S119L, 4CS/E31Q/L144N, 4CS/E31Q/R147K, 4CS/E31Q/I149V, 4CS/E31Q/M150T, 4CS/E31Q/E156Q/D157N, 4CS/K53S, 4CS/K53G, 4CS/K53T, 4CS/K53I, 4CS/K53L, 4CS/K53N, 4CS/K53D, 4CS/M51K, 4CS/M51Q, 4CS/M51I, 4CS/S55N, 4CS/S55Q, 4CS/Q56L, 4CS/Q56I, 4CS/P57A, 4CS/P57E, 4CS/M60L, 4CS/M60I, 4CS/K8Y, 4CS/K8Q, 4CS/K8E, 4CS/H109W, 4CS/H109Y, 4CS/E31Q/S38E, 4CS/E31Q/S38L, 4CS/E31Q/S38I, 4C S/E31Q/S38V, 4C S/E31Q/S68N, 4CS/E31Q/S68E, 4CS/E31Q/S68Q, 4C S/E31Q/S76C, 4C S/E31Q/S127D, 4CS/E31Q/S127E, 4CS/D23N/E31Q/R27Q, 4CS/E31Q/Q56L/T95E, 4CS/E31Q/K96Q/S119L, 4CS/E31Q/E141K/I149V, 4CS/E31Q/E141Q/I149V, 4CS/S7P/E31Q/S50Y, 4C S/E31Q/I80T/I81L/delD193, 4C S/E31Q/P57A/S119L/delD193, 4CS/E31Q/P57A/180T/181L/S119L/delD193, 4CS/E31Q/P57A/K93D/T95E/S119L/delD193, 4C S/E31Q/I80T/S119L/delD193, 4C S/E31Q/I80T/I81L/K93D/T95E/delD193, 4CS/E31Q/P57A/180T/181L/K93D/T95E/S119L/delD193, 4CS/S7C/E31Q/S50C/delD193, 4CS/S7C/E31Q/S50C/P57A/delD193, 4C S/S7C/E31Q/S50C/S119L/delD193, 4CS/S7C/E31Q/S50C/I80T/delD193, 4CS/S7C/E31Q/S50C/I80T/S119L/delD193, 4CS/S7C/E31Q/S50C/P57A/I80T/S119L/delD193, 4CS/S10C/E31Q/N155C/delD193, 4CS/S10C/E31Q/P57A/N155C/delD193, 4CS/S10C/E31Q/S119L/N155C/delD193, 4CS/S10C/E31Q/I80T/N155C/delD193, 4C S/S10C/E31Q/I80T/S119L/N155C/delD193, 4CS/S10C/E31Q/P57A/I80T/S119L/N155C/delD193, 4C S/S10C/E31Q/I49C/delD193, 4CS/L15C/E31Q/R147C/delD193, 4CS/E31Q/T63C/P88C/delD193, 4CS/N14C/E31Q/S127C/delD193, 4CS/E31Q/S38R/S127W/delD193, 4CS/S10C/D35E/N155C, 4CS/S10C/S36D/N155C, 4CS/S10C/S36N/N155C, 4CS/S10C/K53V/N155C, 4CS/S10C/K53Y/N155C, 4CS/S10C/K53F/N155C, 4CS/S10C/M51R/N155C, 4CS/S10C/M51L/N155C, 4CS/S10C/M51H/N155C, 4CS/S10C/M51F/N155C, 4CS/S10C/M51Y/N155C, 4CS/S10C/S55D/N155C, 4CS/S10C/S55E/N155C, 4CS/S10C/S55T/N155C, 4CS/S10C/P57Q/N155C, 4CS/S10C/P57D/N155C, 4CS/S10C/P57Y/N155C, 4CS/S10C/P57N/N155C, 4CS/S10C/M60Y/N155C, 4CS/S10C/M60F/N155C, 4CS/S10C/D110Q/N155C, 4CS/S10C/D110R/N155C, 4CS/S10C/N111D/N155C, 4CS/S10C/N111S/N155C, 4CS/S10C/N111T/N155C, 4CS/S10C/N111E/N155C, 4CS/S10C/D132Q/N155C, 4CS/S10C/D132EN155C, 4CS/E6Q/S10C/K53D/N155C, 4CS/E6Q/S10C/M51K/K53D/N155C, 4CS/S10C/E31Q/D35N/N41Q/K53A/N155C, 4CS/S10C/E31Q/N41Q/K53A/N155C, 4CS/S10C/E31Q/K53A/N155C, 4CS/S10C/K53T/N155C, 4CS/S10C/P57A/N155C, 4CS/S10C/N155C, 4CS/S10C/S76G/N155C, 4CS/S10C/S76A/N155C, 4CS/S10C/M51K/K53D/N155C, 4CS/S10C/M51K/K53E/N155C, 4CS/E6Q/S10C/K53E/N155C, 4CS/E6Q/S10C/M51K/K53E/N155C, 4CS/E6Q/S10C/M51K/P57E/N155C, 4CS/S10C/M51K/P57E/N155C, 4CS/E6Q/S10C/P57E/N155C, 4CS/S10C/E31Q/K53T/N155C, 4CS/S10C/K53G/P57E/N155C, 4CS/S10C/K53T/P57E/N155C, 4CS/S10C/K53A/P57E/N155C, 4CS/S10C/P57E/N155C, 4CS/S10C/K53D/N155C, 4CS/S10C/E31Q/N41Q/N155C, 4CS/S10C/K53A/N155C, 4CS/S10C/K53G/N155C, 4CS/S10C/K53E/N155C, 4CS/S10C/K53S/N155C, 4CS/S10C/M51L/K53D/N155C, 4CS/S10C/K53D/D110R/N155C, 4CS/S10C/K53D/N111T/N155C, 4CS/S10C/K53D/S55T/N155C, 4CS/S10C/K53D/S55T/D110R/N155C, 4CS/S10C/M51L/K53D/S55T/D110R/N111T/N155C, 4CS/S10C/M51L/K53D/S55T/D110R/N155C, 4CS/S10C/K53D/S55T/D110R/N111T/N155C, 4CS/S10C/K53D/S55T/N111T/N155C, 4CS/S10C/E31Q/D35N/N155C, 4CS/S10C/N41Q/N155C, 4CS/S10C/D35N/N155C, 4CS/S10C/D37N/N155C, 4CS/S10C/E31Q/D37N/N155C, 4CS/S10C/D35N/D37N/N155C, 4CS/E6Q/S10C/M51L/K53D/S55T/D110R/N111T/N155C, 4CS/S10C/K53D/H109Y/N155C, 4CS/S10C/D37N/K53D/N155C, 4CS/S10C/D35N/K53D/N155C, 4CS/K8E/S10C/K53D/N155C, 4CS/S10C/E31Q/K53D/N155C, 4CS/S10C/N41Q/K53D/N155C, 4CS/S10C/K53D/P57V/N155C, 4CS/S10C/K53D/P57T/N155C, 4CS/E6Q/S10C/K53D/N111T/N155C, E6A/K53A, D35N/K53A, N41Q/K53A, D35N/N41Q/K53A, D35N/N41Q, D37N/K53A, E6Q/K53D, E6Q/M51K/K53D, M51K/K53D, M51K/K53E, E6Q/K53E, E6Q/M51K/K53E, E6Q/M51K/P57E, M51K/P57E, E6Q/P57E, K53G/P57E, K53T/P57E, K53A/P57E, M51L/K53D, K53D/D110R, K53D/N111T, K53D/S55T, K53D/S55T/D110R, M51L/K53D/S55T/D110R/N111T, M51L/K53D/S55T/D110R, K53D/S55T/D110R/N111T, K53D/S55T/N111T, D35N/D37N, E6Q/M51L/K53D/S55T/D110R/N111T, K53D/H109Y, D37N/K53D, D35N/K53D, K8E/K53D, N41Q/K53D, K53D/P57V, K53D/P57T, E6Q/K53D/N111T, Q56L/P57T, K93D/T95E, K93N/T95E, E156Q/D157N, D23N/R27Q, Q56L/T95E, K96Q/S119L, E141K/I149V, E141Q/I149V, S7P/S50Y, I80T/I81L, P57A/S119L, P57A/I80T/I81L/S119L, P57A/K93D/T95E/S119L, I80T/S119L, I80T/I81L/K93D/T95E, P57A/I80T/I81L/K93D/T95E/S119L, P57A/I80T/S119L, N14C/S127C, M33C/S38C, S76C/L138C, S10C/I49C, L15C/R147C, P28C/L136C, S50C/P88C, T63C/P88C, V62C/Q103C, S10C/N155C, S65C/P88C, S7C/S50C, D54C/A61C, A126C/K139C, C38R/C127W, E31Q/K53A, E31Q/D35N/K53A, E31Q/N41Q/K53A, E31Q/D35N/N41Q/K53A, E31Q/D35N, E31Q/N41Q, E31Q/D35N/N41Q, E31Q/D37N, E31Q/D37N/K53A, S10C/E31Q/I49C, L15C/E31Q/R147C, P28C/E31Q/L136C, E31Q/S50C/P88C, E31Q/T63C/P88C, E31Q/V62C/Q103C, S10C/E31Q/N155C, E31Q/S65C/P88C, S7C/E31Q/S50C, E31Q/D54C/A61C, E31Q/A126C/K139C, N14W/E31Q, E31Q/D146Y, E31Q/D146L, E31Q/D146F, E31Q/Q103L, E31Q/Q103I, E31Q/M150F, Ql8L/E31Q, S7P/E31Q, V11I/E31Q, D23N/E31Q, D23 S/E31Q, R27Q/E31Q, L29V/E31Q, E31Q/T34P, E31Q/R39T, E31Q/R39S, E31Q/R44Q, E31Q/I46V, E31Q/S50Y, E31Q/Q56L, E31Q/Q56L/P57T, E31Q/P57T, E31Q/P57V, E31Q/M60L, E31Q/K67Q, E31Q/E69K, E31Q/I71M, E31Q/E77K, E31Q/I80T, E31Q/I81V, E31Q/I81L, E31Q/N87S, E31Q/D90E, E31Q/K93D/T95E, E31Q/K93N/T95E, E31Q/T95E, E31Q/K96G, E31Q/S97N, E31Q/N111D, E31Q/M113I, E31Q/S119L, E31Q/L144N, E31Q/R147K, E31Q/I149V, E31Q/M150T, E31Q/E156Q/D157N, D23N/E31Q/R27Q, E31Q/Q56L/T95E, E31Q/K96Q/S119L, E31Q/E141K/I149V, E31Q/E141Q/I149V, S7P/E31Q/S50Y, E31Q/I80T/I81L/delD193, E31Q/P57A/S119L/delD193, E31Q/P57A/I80T/I8 1L/S 11 9L/delD193, E31Q/P57A/K93D/T95E/S119L/delD193, E31Q/I80T/S119L/delD193, E31Q/I80T/I81L/K93D/T95E/delD193, E31Q/P57A/180T/181L/K93D/T95E/S119L/delD193, S7C/E31Q/S50C/delD193, S7C/E31Q/S50C/P57A/delD193, S7C/E31Q/S50C/S119L/delD193, S7C/E31Q/S50C/I80T/delD193, S7C/E31Q/S50C/I80T/S119L/delD193, S7C/E31Q/S50C/P57A/I80T/S119L/delD193, S10C/E31Q/N155C/delD193, S10C/E31Q/P57A/N155C/delD193, S10C/E31Q/S119L/N155C/delD193, S10C/E31Q/I80T/N155C/delD193, S10C/E31Q/I80T/S119L/N155C/delD193, S10C/E31Q/P57A/I80T/S119L/N155C/delD193, S10C/E31Q/I49C/delD193, L15C/E31Q/R147C/delD193, E31Q/T63C/P88C/delD193, S10C/D35E/N155C, S10C/S36D/N155C, S10C/S36N/N155C, S10C/K53V/N155C, S10C/K53Y/N155C, S10C/K53F/N155C, S10C/M51R/N155C, S10C/M51L/N155C, S10C/M51H/N155C, S10C/M51F/N155C, S10C/M51Y/N155C, S10C/S55D/N155C, S10C/S55E/N155C, S10C/S55T/N155C, S10C/P57Q/N155C, S10C/P57D/N155C, S10C/P57Y/N155C, S10C/P57N/N155C, S10C/M60Y/N155C, S10C/M60F/N155C, S10C/D110Q/N155C, S10C/D110R/N155C, S10C/N111D/N155C, S10C/N111S/N155C, S10C/N111T/N155C, S10C/N111E/N155C, S10C/D132Q/N155C, S10C/D132E/N155C, E6Q/S10C/K53D/N155C, E6Q/S10C/M51K/K53D/N155C, S10C/E31Q/D35N/N41Q/K53A/N155C, S10C/E31Q/N41Q/K53A/N155C, S10C/E31Q/K53A/N155C, S10C/K53T/N155C, S10C/P57A/N155C, S10C/M51K/K53D/N155C, S10C/M51K/K53E/N155C, E6Q/S10C/K53E/N155C, E6Q/S10C/M51K/K53E/N155C, E6Q/S10C/M51K/P57E/N155C, S10C/M51K/P57E/N155C, E6Q/S10C/P57E/N155C, S10C/E31Q/K53T/N155C, S10C/K53G/P57E/N155C, S10C/K53T/P57E/N155C, S10C/K53A/P57E/N155C, S10C/P57E/N155C, S10C/K53D/N155C, S10C/E31Q/N41Q/N155C, S10C/K53A/N155C, S10C/K53G/N155C, S10C/K53E/N155C, S10C/K53S/N155C, S10C/M51L/K53D/N155C, S10C/K53D/D110R/N155C, S10C/K53D/N111T/N155C, S10C/K53D/S55T/N155C, S10C/K53D/S55T/D110R/N155C, S10C/M51L/K53D/S55T/D110R/N111T/N155C, S10C/M51L/K53D/S55T/D110R/N155C, S10C/K53D/S55T/D110R/N111T/N155C, S10C/K53D/S55T/N111T/N155C, S10C/E31Q/D35N/N155C, S10C/N41Q/N155C, S10C/D35N/N155C, S10C/D37N/N155C, S10C/E31Q/D37N/N155C, S10C/D35N/D37N/N155C, E6Q/S10C/M51L/K53D/S55T/D110R/N111T/N155C, S10C/K53D/H109Y/N155C, S10C/D37N/K53D/N155C, S10C/D35N/K53D/N155C, K8E/S10C/K53D/N155C, S10C/E31Q/K53D/N155C, S10C/N41Q/K53D/N155C, S10C/K53D/P57V/N155C, S10C/K53D/P57T/N155C, or E6Q/S10C/K53D/N111T/N155C.

In some embodiments, the first and second monomer each comprises an amino acid sequence selected from the group consisting of: SEQ ID NOS:239-292 and 960-1039 (XENP30792, XENP31296, XENP31812-XENP31814, XENP34106-XENP34114, XENP34281, XENP37825, XENP37826, XENP38869, XENP39138-XENP39142, XENP39149-XENP39152, XENP39672, XENP39804, XENP39805, XENP40024, XENP40025, XENP40685, XENP40962-XENP40968, XENP41756-41770, XENP41974-41975, XENP42006-XENP42012, and XENP42141-42148 in FIGS. 21A-21M).

In some aspects, provided herein is a monovalent Fc fusion protein comprising: (a) a first monomer comprising from N-terminus to C-terminus: a variant IL-18 protein and a first Fc domain, wherein the variant human IL-18 protein comprises amino acid substitutions 4CS/E6Q/S10C/K53D/N111T/N155C, and wherein the variant IL-18 protein is covalently attached to the N-terminus of the first Fc domain; and (b) a second monomer comprising a second Fc domain.

Provided herein is one or more nucleic acids encoding any one of the monovalent Fc fusion proteins described. Provided herein is an expression vector comprising any of the one or more nucleic acids described herein. Provided herein is a host cell comprising any of the one of more nucleic acids described or any of the expression vectors described herein. In some embodiments, provided is a method of making a monovalent Fc fusion protein comprising culturing any of the host cells described and recovering the monovalent Fc fusion protein from the cell culture.

In some aspect, provided herein is a Fab-Fc fusion protein comprising: (a) a first monomer comprising from N-terminus to C-terminus: a variable heavy (VH) chain and a first Fc domain; (b) a second monomer comprising from N-terminus to C-terminus: a wildtype or variant IL-18 protein and a second Fc domain; and (c) a third monomer comprising a variable light (VL) chain, wherein the VH and VL form an antigen binding fragment (Fab).

In some embodiments of the Fab-Fc fusion protein, the second Fc domain further comprises a set of amino acid substitutions Q295E/N384D/Q418E/N421D, according to EU numbering. In some embodiments, the first and/or second Fc domains further comprise a modification of K447del, according to EU numbering. In some embodiments, the first and/or second Fc domains further comprise a set of amino acid substitutions selected from the group consisting of C219S, C220S, S228P, G236R/L328R, E233P/L234V/L235A/G236del/S239K, E233P/L234V/L235A/G236del/S239K/A327G, E233P/L234V/L235A/G236del/S267K/A327G, E233P/L234V/L235A/G236del, E233P/L234V/L235A/G236del/S267K, and C220S/E233P/L234V/L235A/G236del/S267K, according to EU numbering. In some embodiments, the first and second Fc domains each further comprises modifications C220S/E233P/L234V/L235A/G236del/S267K, according to EU numbering. In some embodiments, the first Fc domain and the second Fc domain have a set of amino acid substitutions selected from the group consisting of: (i) S267K/L368D/K370S: S267K/S364K/E357Q; (ii) S364K/E357Q: L368D/K370S; (iii) L368D/K370S: S364K; (iv) L368E/K370S: S364K; (v) T411E/K360E/Q362E: D401K; (vi) L368D/K370S: S364K/E357Q, and (vii) K370S: S364K/E357Q, according to EU numbering. In some embodiments, the first and second Fc domains further comprise amino acid substitutions M428L/N434S, according to EU numbering.

In some embodiments of the Fab-Fc fusion protein, the wildtype or variant IL-18 protein is covalently attached to the N-terminus of the first Fc domain. In some embodiments, the wildtype or variant IL-18 protein is covalently attached to a domain linker which is covalently attached to the N-terminus of the first Fc domain. In some embodiments, the domain linker is selected from any one of the domain linkers in FIG. 8.

In some embodiments, the wildtype IL18 protein has an amino acid sequence selected from the group consisting of SEQ ID NO:1 (human precursor IL18) and SEQ ID NO:2 (human mature IL18).

In some embodiments, the variant IL18 protein has at least 90% sequence identity to an amino acid sequence selected from the group consisting of SEQ ID NO:1 (human precursor IL18) and SEQ ID NO:2 (human mature IL18).

In some embodiments, the variant IL18 protein comprising a modification at one or more amino acid positions selected from the group consisting of Y1, E6, S7, K8, S10, V11, N14, L15, D17, Q18, D23, R27, P28, L29, E31, M33, T34, D35, S36, D37, C38, R39, D40, N41, R44, I46, 149, S50, M51, K53, D54, S55, Q56, P57, M60, A61, V62, T63, S65, K67, C68, E69, I71, C76, E77, I80, I81, N87, P88, D90, K93, T95, K96, S97, Q103, H109, D110, N111, M113, S119, A126, C127, D132, L136, L138, K139, E141, L144, D146, R147, I149, M150, N155, E156, and D157, as compared to wildtype human IL18.

In some embodiments, the variant IL18 protein comprising one or more amino acid substitutions selected from the group consisting of Y1F, Y1H, E6A, E6Q, S7C, S7P, K8E, K8Q, K8Y, S10C, V11I, N14C, N14W, L15C, D17N, Q18L, D23N, D23S, R27Q, P28C, L29V, E31Q, M33C, T34P, D35N, D35E, S36D, S36N, D37N, C38S, C38Q, C38R, C38E, C38L, C381, C38V, C38K, C38D, R39S, R39T, D40N, N41Q, R44Q, I46V, I49C, S50C, S50Y, M51I, M51K, M51Q, M51R, M51L, M51H, M51F, M51Y, K53A, K53D, K53E, K53G, K53H, K531, K53L, K53M, K53N, K53Q, K53R, K53S, K531, K53V, K53Y, K53F, D54C, S55N, S55Q, S55D, S55E, S55T,Q56I, Q56L, P57A, P57E, P571, P57V, P57Q, P57D, P57Y, P57N, M60I, M60L, M60K, M60Y, M60F, A61C, V62C, T63C, S65C, K67Q, C68S, C68I, C68F, C68Y, C68D, C68N, C68E, C68Q, C68K, E69K, I71M, C76S, C76E, C76K, E77K, 180T, I81L, I81V, N87S, P88C, D90E, K93D, K93N, T95E, K96G, K96Q, S97N, Q103C, Q103E, Q103I, Q103L, H109W, H109Y, D110N, D110Q, D110R, N111D, N111Q, N111S, N111T, N111E, M113I, S119L, A126C, C127S, C127W, C127Y, C127F, C127D, C127E, C127K, D132Q, D132E, L136C, L138C, K139C, E141K, E141Q, L144N, D146F, D146L, D146Y, R147C, R147K, I149V, M150F, M150T, N155C, E156Q, D157A, D157S, D157N, and D157del, as compared to wildtype human IL18. In some embodiments, the amino acid substitution can include 4CS, 4CS/D193S, 4CS/D193A, 4CS/delD193, 4CS/S38E, 4CS/S68E, 4CS/S76E, 4CS/S127E, 4CS/S38K, 4CS/S68K, 4CS/S76K, 4CS/S127K, 4CS/S38D, 4CS/Y1F, 4CS/Y1H, 4CS/E6A, 4CS/E6Q, 4CS/D17N, 4CS/E31Q, 4CS/D35N, 4CS/D37N, 4CS/D40N, 4CS/N41Q, 4CS/K53R, 4CS/K53H, 4CS/K53M, 4CS/K53E, 4CS/K53Q, 4CS/K53A, 4CS/Q103E, 4CS/D110N, 4CS/N111Q, 4CS/E6A/K53A, 4CS/N14C/E31Q/S127C, 4CS/E31Q/K53A, 4CS/E31Q/D35N/K53A, 4CS/E31Q/N41Q/K53A, 4CS/E31Q/D35N/N41Q/K53A, 4CS/E31Q/D35N, 4CS/E31Q/N41Q, 4C S/E31Q/D35N/N41Q, 4C S/E31Q/D37N, 4CS/E31Q/D37N/K53A, 4CS/E31Q/M33C/S38C, 4C S/E31Q/S76C/L138C, 4CS/E31Q/S68I, 4CS/E31Q/S68F, 4CS/E31Q/S127W, 4CS/E31Q/S127Y, 4CS/E31Q/S127F, 4CS/S10C/E31Q/149C, 4C S/L15C/E31Q/R147C, 4C S/P28C/E31Q/L136C, 4CS/E31Q/S50C/P88C, 4CS/E31Q/163C/P88C, 4C S/E31Q/V62C/Q103C, 4CS/S10C/E31Q/N155C, 4CS/E31Q/S65C/P88C, 4CS/S7C/E31Q/S50C, 4CS/E31Q/D54C/A61C, 4C S/E31Q/A126C/K139C, 4CS/N14W/E31Q, 4CS/E31Q/D146Y, 4CS/E31Q/D146L, 4C S/E31Q/D146F, 4C S/E31Q/Q103L, 4CS/E31Q/Q103I, 4C S/E31Q/M150F, 4CS/Q18L/E31Q, 4CS/E31Q/S68Y, 4CS/E31Q/S38Q, 4C S/E31Q/S38R, 4CS/E31Q/S68D, 4CS/S7P/E31Q, 4CS/V11I/E31Q, 4CS/D23N/E31Q, 4CS/D23S/E31Q, 4CS/R27Q/E31Q, 4CS/L29V/E31Q, 4CS/E31Q/T34P, 4CS/E31Q/R39T, 4CS/E31Q/R39S, 4CS/E31Q/R44Q, 4CS/E31Q/I46V, 4CS/E31Q/S50Y, 4CS/E31Q/Q56L, 4CS/E31Q/Q56L/P57T, 4C S/E31Q/P57T, 4CS/E31Q/P57V, 4CS/E31Q/M60L, 4CS/E31Q/K67Q, 4CS/E31Q/E69K, 4CS/E31Q/I71M, 4CS/E31Q/E77K, 4CS/E31Q/I80T, 4CS/E31Q/I81V, 4CS/E31Q/I81L, 4C S/E31Q/N87S, 4CS/E31Q/D90E, 4CS/E31Q/K93D/T95E, 4CS/E31Q/K93N/T95E, 4CS/E31Q/T95E, 4CS/E31Q/K96G, 4CS/E31Q/S97N, 4CS/E31Q/N111D, 4CS/E31QN1113I, 4CS/E31Q/S119L, 4CS/E31Q/L144N, 4CS/E31Q/R147K, 4CS/E31Q/I149V, 4CS/E31Q/M150T, 4CS/E31Q/E156Q/D157N, 4CS/K53S, 4CS/K53G, 4CS/K53T, 4CS/K53I, 4CS/K53L, 4CS/K53N, 4CS/K53D, 4CS/M51K, 4CS/M51Q, 4CS/M51I, 4CS/S55N, 4CS/S55Q, 4CS/Q56L, 4CS/Q56I, 4CS/P57A, 4CS/P57E, 4CS/M60L, 4CS/M601, 4CS/K8Y, 4CS/K8Q, 4CS/K8E, 4CS/H109W, 4CS/H109Y, 4CS/E31Q/S38E, 4CS/E31Q/S38L, 4CS/E31Q/S38I, 4C S/E31Q/S38V, 4C S/E31Q/S68N, 4CS/E31Q/S68E, 4CS/E31Q/S68Q, 4C S/E31Q/S76C, 4C S/E31Q/S127D, 4CS/E31Q/S127E, 4CS/D23N/E31Q/R27Q, 4CS/E31Q/Q56L/T95E, 4CS/E31Q/K96Q/S119L, 4CS/E31Q/E141K/I149V, 4CS/E31Q/E141Q/I149V, 4CS/S7P/E31Q/S50Y, 4C S/E31Q/I80T/I81L/delD193, 4C S/E31Q/P57A/S119L/delD193, 4CS/E31Q/P57A/180T/181L/S119L/delD193, 4CS/E31Q/P57A/K93D/T95E/S119L/delD193, 4C S/E31Q/I80T/S119L/delD193, 4C S/E31Q/I80T/I81L/K93D/T95E/delD193, 4CS/E31Q/P57A/180T/181L/K93D/T95E/S119L/delD193, 4CS/S7C/E31Q/S50C/delD193, 4CS/S7C/E31Q/S50C/P57A/delD193, 4C S/S7C/E31Q/S50C/S119L/delD193, 4CS/S7C/E31Q/S50C/I80T/delD193, 4CS/S7C/E31Q/S50C/I80T/S119L/delD193, 4CS/S7C/E31Q/S50C/P57A/I80T/S119L/delD193, 4CS/S10C/E31Q/N155C/delD193, 4CS/S10C/E31Q/P57A/N155C/delD193, 4CS/S10C/E31Q/S119L/N155C/delD193, 4CS/S10C/E31Q/I80T/N155C/delD193, 4C S/S10C/E31Q/I80T/S119L/N155C/delD193, 4CS/S10C/E31Q/P57A/I80T/S119L/N155C/delD193, 4C S/S10C/E31Q/I49C/delD193, 4CS/L15C/E31Q/R147C/delD193, 4CS/E31Q/T63C/P88C/delD193, 4CS/N14C/E31Q/S127C/delD193, 4CS/E31Q/S38R/S127W/delD193, 4CS/S10C/D35E/N155C, 4CS/S10C/S36D/N155C, 4CS/S10C/S36N/N155C, 4CS/S10C/K53V/N155C, 4CS/S10C/K53Y/N155C, 4CS/S10C/K53F/N155C, 4CS/S10C/M51R/N155C, 4CS/S10C/M51L/N155C, 4CS/S10C/M51H/N155C, 4CS/S10C/M51F/N155C, 4CS/S10C/M51Y/N155C, 4CS/S10C/S55D/N155C, 4CS/S10C/S55E/N155C, 4CS/S10C/S55T/N155C, 4CS/S10C/P57Q/N155C, 4CS/S10C/P57D/N155C, 4CS/S10C/P57Y/N155C, 4CS/S10C/P57N/N155C, 4CS/S10C/M60Y/N155C, 4CS/S10C/M60F/N155C, 4CS/S10C/D110Q/N155C, 4CS/S10C/D110R/N155C, 4CS/S10C/N111D/N155C, 4CS/S10C/N111S/N155C, 4CS/S10C/N111T/N155C, 4CS/S10C/N111E/N155C, 4CS/S10C/D132Q/N155C, 4CS/S10C/D132E/N155C, 4CS/E6Q/S10C/K53D/N155C, 4CS/E6Q/S10C/M51K/K53D/N155C, 4CS/S10C/E31Q/D35N/N41Q/K53A/N155C, 4CS/S10C/E31Q/N41Q/K53A/N155C, 4CS/S10C/E31Q/K53A/N155C, 4CS/S10C/K53T/N155C, 4CS/S10C/P57A/N155C, 4CS/S10C/N155C, 4CS/S10C/S76G/N155C, 4CS/S10C/S76A/N155C, 4CS/S10C/M51K/K53D/N155C, 4CS/S10C/M51K/K53E/N155C, 4CS/E6Q/S10C/K53E/N155C, 4CS/E6Q/S10C/M51K/K53E/N155C, 4CS/E6Q/S10C/M51K/P57E/N155C, 4CS/S10C/M51K/P57E/N155C, 4CS/E6Q/S10C/P57E/N155C, 4CS/S10C/E31Q/K53T/N155C, 4CS/S10C/K53G/P57E/N155C, 4CS/S10C/K53T/P57E/N155C, 4CS/S10C/K53A/P57E/N155C, 4CS/S10C/P57E/N155C, 4CS/S10C/K53D/N155C, 4CS/S10C/E31Q/N41Q/N155C, 4CS/S10C/K53A/N155C, 4CS/S10C/K53G/N155C, 4CS/S10C/K53E/N155C, 4CS/S10C/K53S/N155C, 4CS/S10C/M51L/K53D/N155C, 4CS/S10C/K53D/D110R/N155C, 4CS/S10C/K53D/N111T/N155C, 4CS/S10C/K53D/S55T/N155C, 4CS/S10C/K53D/S55T/D110R/N155C, 4CS/S10C/M51L/K53D/S55T/D110R/N111T/N155C, 4CS/S10C/M51L/K53D/S55T/D110R/N155C, 4CS/S10C/K53D/S55T/D110R/N111T/N155C, 4CS/S10C/K53D/S55T/N111T/N155C, 4CS/S10C/E31Q/D35N/N155C, 4CS/S10C/N41Q/N155C, 4CS/S10C/D35N/N155C, 4CS/S10C/D37N/N155C, 4CS/S10C/E31Q/D37N/N155C, 4CS/S10C/D35N/D37N/N155C, 4CS/E6Q/S10C/M51L/K53D/S55T/D110R/N111T/N155C, 4CS/S10C/K53D/H109Y/N155C, 4CS/S10C/D37N/K53D/N155C, 4CS/S10C/D35N/K53D/N155C, 4CS/K8E/S10C/K53D/N155C, 4CS/S10C/E31Q/K53D/N155C, 4CS/S10C/N41Q/K53D/N155C, 4CS/S10C/K53D/P57V/N155C, 4CS/S10C/K53D/P57T/N155C, 4CS/E6Q/S10C/K53D/N111T/N155C, E6A/K53A, D35N/K53A, N41Q/K53A, D35N/N41Q/K53A, D35N/N41Q, D37N/K53A, E6Q/K53D, E6Q/M51K/K53D, M51K/K53D, M51K/K53E, E6Q/K53E, E6Q/M51K/K53E, E6Q/M51K/P57E, M51K/P57E, E6Q/P57E, K53G/P57E, K53T/P57E, K53A/P57E, M51L/K53D, K53D/D110R, K53D/N111T, K53D/S55T, K53D/S55T/D110R, M51L/K53D/S55T/D110R/N111T, M51L/K53D/S55T/D110R, K53D/S55T/D110R/N111T, K53D/S55T/N111T, D35N/D37N, E6Q/M51L/K53D/S55T/D110R/N111T, K53D/H109Y, D37N/K53D, D35N/K53D, K8E/K53D, N41Q/K53D, K53D/P57V, K53D/P57T, E6Q/K53D/N111T, Q56L/P57T, K93D/T95E, K93N/T95E, E156Q/D157N, D23N/R27Q, Q56L/T95E, K96Q/S119L, E141K/I149V, E141Q/I149V, S7P/S50Y, I80T/I81L, P57A/S119L, P57A/I80T/I81L/S119L, P57A/K93D/T95E/S119L, I80T/S119L, I80T/I81L/K93D/T95E, P57A/I80T/I81L/K93D/T95E/S119L, P57A/I80T/S119L, N14C/S127C, M33C/S38C, S76C/L138C, S10C/I49C, L15C/R147C, P28C/L136C, S50C/P88C, T63C/P88C, V62C/Q103C, S10C/N155C, S65C/P88C, S7C/S50C, D54C/A61C, A126C/K139C, C38R/C127W, E31Q/K53A, E31Q/D35N/K53A, E31Q/N41Q/K53A, E31Q/D35N/N41Q/K53A, E31Q/D35N, E31Q/N41Q, E31Q/D35N/N41Q, E31Q/D37N, E31Q/D37N/K53A, S10C/E31Q/I49C, L15C/E31Q/R147C, P28C/E31Q/L136C, E31Q/S50C/P88C, E31Q/T63C/P88C, E31Q/V62C/Q103C, S10C/E31Q/N155C, E31Q/S65C/P88C, S7C/E31Q/S50C, E31Q/D54C/A61C, E31Q/A126C/K139C, N14W/E31Q, E31Q/D146Y, E31Q/D146L, E31Q/D146F, E31Q/Q103L, E31Q/Q103I, E31Q/M150F, Ql8L/E31Q, S7P/E31Q, V11I/E31Q, D23N/E31Q, D23 S/E31Q, R27Q/E31Q, L29V/E31Q, E31Q/T34P, E31Q/R39T, E31Q/R39S, E31Q/R44Q, E31Q/I46V, E31Q/S50Y, E31Q/Q56L, E31Q/Q56L/P57T, E31Q/P57T, E31Q/P57V, E31Q/M60L, E31Q/K67Q, E31Q/E69K, E31Q/I71M, E31Q/E77K, E31Q/I80T, E31Q/I81V, E31Q/I81L, E31Q/N87S, E31Q/D90E, E31Q/K93D/T95E, E31Q/K93N/T95E, E31Q/T95E, E31Q/K96G, E31Q/S97N, E31Q/N111D, E31Q/M113I, E31Q/S119L, E31Q/L144N, E31Q/R147K, E31Q/I149V, E31Q/M150T, E31Q/E156Q/D157N, D23N/E31Q/R27Q, E31Q/Q56L/T95E, E31Q/K96Q/S119L, E31Q/E141K/I149V, E31Q/E141Q/I149V, S7P/E31Q/S50Y, E31Q/I80T/I81L/delD193, E31Q/P57A/S119L/delD193, E31Q/P57A/I80T/I8 1L/S 11 9L/delD193, E31Q/P57A/K93D/T95E/S119L/delD193, E31Q/I80T/S119L/delD193, E31Q/I80T/I81L/K93D/T95E/delD193, E31Q/P57A/I80T/I81L/K93D/T95E/S119L/delD193, S7C/E31Q/S50C/delD193, S7C/E31Q/S50C/P57A/delD193, S7C/E31Q/S50C/S119L/delD193, S7C/E31Q/S50C/I80T/delD193, S7C/E31Q/S50C/I80T/S119L/delD193, S7C/E31Q/S50C/P57A/I80T/S119L/delD193, S10C/E31Q/N155C/delD193, S10C/E31Q/P57A/N155C/delD193, S10C/E31Q/S119L/N155C/delD193, S10C/E31Q/I80T/N155C/delD193, S10C/E31Q/I80T/S119L/N155C/delD193, S10C/E31Q/P57A/I80T/S119L/N155C/delD193, S10C/E31Q/I49C/delD193, L15C/E31Q/R147C/delD193, E31Q/T63C/P88C/delD193, S10C/D35E/N155C, S10C/S36D/N155C, S10C/S36N/N155C, S10C/K53V/N155C, S10C/K53Y/N155C, S10C/K53F/N155C, S10C/M51R/N155C, S10C/M51L/N155C, S10C/M51H/N155C, S10C/M51F/N155C, S10C/M51Y/N155C, S10C/S55D/N155C, S10C/S55E/N155C, S10C/S55T/N155C, S10C/P57Q/N155C, S10C/P57D/N155C, S10C/P57Y/N155C, S10C/P57N/N155C, S10C/M60Y/N155C, S10C/M60F/N155C, S10C/D110Q/N155C, S10C/D110R/N155C, S10C/N111D/N155C, S10C/N111S/N155C, S10C/N111T/N155C, S10C/N111E/N155C, S10C/D132Q/N155C, S10C/D132E/N155C, E6Q/S10C/K53D/N155C, E6Q/S10C/M51K/K53D/N155C, S10C/E31Q/D35N/N41Q/K53A/N155C, S10C/E31Q/N41Q/K53A/N155C, S10C/E31Q/K53A/N155C, S10C/K53T/N155C, S10C/P57A/N155C, S10C/M51K/K53D/N155C, S10C/M51K/K53E/N155C, E6Q/S10C/K53E/N155C, E6Q/S10C/M51K/K53E/N155C, E6Q/S10C/M51K/P57E/N155C, S10C/M51K/P57E/N155C, E6Q/S10C/P57E/N155C, S10C/E31Q/K53T/N155C, S10C/K53G/P57E/N155C, S10C/K53T/P57E/N155C, S10C/K53A/P57E/N155C, S10C/P57E/N155C, S10C/K53D/N155C, S10C/E31Q/N41Q/N155C, S10C/K53A/N155C, S10C/K53G/N155C, S10C/K53E/N155C, S10C/K53S/N155C, S10C/M51L/K53D/N155C, S10C/K53D/D110R/N155C, S10C/K53D/N111T/N155C, S10C/K53D/S55T/N155C, S10C/K53D/S55T/D110R/N155C, S10C/M51L/K53D/S55T/D110R/N111T/N155C, S10C/M51L/K53D/S55T/D110R/N155C, S10C/K53D/S55T/D110R/N111T/N155C, S10C/K53D/S55T/N111T/N155C, S10C/E31Q/D35N/N155C, S10C/N41Q/N155C, S10C/D35N/N155C, S10C/D37N/N155C, S10C/E31Q/D37N/N155C, S10C/D35N/D37N/N155C, E6Q/S10C/M51L/K53D/S55T/D110R/N111T/N155C, S10C/K53D/H109Y/N155C, S10C/D37N/K53D/N155C, S10C/D35N/K53D/N155C, K8E/S10C/K53D/N155C, S10C/E31Q/K53D/N155C, S10C/N41Q/K53D/N155C, S10C/K53D/P57V/N155C, S10C/K53D/P57T/N155C, or E6Q/S10C/K53D/N111T/N155C.

In some embodiments, the first second and third monomers each comprises an amino acid sequence selected from the group consisting of: SEQ ID NOS:294-774 and 1040-1264 (XENP38850-XENP38868, XENP38952-XENP38954, XENP38868, XENP38956, XENP38957, XENP39601-XENP39604, XENP40027, XENP40046-XENP40054, XENP40175-XENP40244, XENP40246-XENP40269, XENP40617-XENP40632, XENP40657-XENP40663, XENP40686, XENP40934-XENP40961, XENP41076-XENP41096, XENP41353, XENP41416-XENP41431, XENP41440, and XENP41513-XENP41520 in FIGS. 22A-22CZ).

In some aspects, provided herein is a Fab-Fc fusion protein comprising: (a) a first monomer comprising from N-terminus to C-terminus: a variable heavy (VH) chain and a first Fc domain; (b) a second monomer comprising from N-terminus to C-terminus: a variant IL-18 protein and a second Fc domain, wherein the variant human IL-18 protein comprises amino acid substitutions 4CS/E6Q/S10C/K53D/N111T/N155C; and (c) a third monomer comprising a variable light (VL) chain, wherein the VH and VL form an antigen binding fragment (Fab), wherein the first Fc domain comprises amino acid substitutions C220S/PVA /S267K/L368D/K370S/M428L/N434S, and the second Fc domain comprises amino acid substitutions PVA /S267K/S364K/E357Q/M428L/N434S, according to EU numbering.

Provided herein is one or more nucleic acids encoding any one of the Fab-Fc fusion proteins described herein. Provided herein is an expression vector comprising any of the one or more nucleic acids described herein. Provided herein is a host cell comprising any of the one or more nucleic acids described herein or any of the expression vectors described herein. In some aspects, provided is a method of making a Fab-Fc fusion protein comprising culturing any of the host cells described herein and recovering the Fab-Fc fusion protein from the cell culture.

In some aspect, provided is a method of reducing a tumor comprising contacting the tumor with a composition comprising a composition comprising any one of the variant human IL-18 proteins described, any one of the monovalent Fc fusion proteins described, and any one of the Fab-Fc fusion proteins described. In some embodiments, the subject is a human subject.

In some aspect, provided is a method of reducing a tumor in a subject in need thereof comprising administering to the subject a composition comprising any one of the variant human IL-18 proteins described, any one of the monovalent Fc fusion proteins described, and any one of the Fab-Fc fusion proteins described. In some embodiments, the subject is a human subject.

In some aspect, provided is a method of treating a subject having a cancer, comprising administering to the subject a composition any one of the variant human IL-18 proteins described, any one of the monovalent Fc fusion proteins described, and any one of the Fab-Fc fusion proteins described. In some embodiments, the subject is a human subject.

### BRIEF DESCRIPTION OF THE DRAWINGS

FIGS. 1A-1B depict the sequences for human IL18, its receptors, and IL18 binding protein.

FIGS. 2A-2B depict the sequences for mouse IL18 and its receptors to facilitate investigation of IL18 fusion proteins of the invention in preclinical studies.

FIGS. 3A-3B depict the sequences for cynomolgus IL18 and its receptors to facilitate investigation of IL18 fusion proteins of the invention in preclinical studies.

FIGS. 4A-4E depict useful pairs of Fc heterodimerization variant sets (including skew and pI variants). Variants without a corresponding “monomer 2” are pI variants which can be used alone on either monomer

FIG. 5 depict a list of isosteric variant antibody constant regions and their respective substitutions. pI_(−) indicates lower pI variants, while pI (+) indicates higher pI variants. These can be optionally and independently combined with other heterodimerization variants of the inventions (and other variant types as well, as outlined herein.)

FIG. 6 depict useful ablation variants that ablate FcγR binding (sometimes referred to as “knock outs” or “KO” variants). Generally, ablation variants are found on both monomers, although in some cases they may be on only one monomer.

FIG. 7 shows particularly useful embodiments of “non-cytokine”/“non-Fv” components of the IL18 fusions of the invention.

FIG. 8 depicts a number of exemplary domain linkers. In some embodiments, these linkers find use linking an IL18 monomer to an Fc chain. In other embodiments, these linkers find use linking a variable heavy region to an Fc chain (optionally via a CH1 region as depicted in FIG. 10). While the “hinge” based domain linkers in this Figure are based on IgG1 hinge, hinge regions from other IgG isotypes may also be used. In the case of IgG2, the hinge sequence may include C219S and/or C220S substitutions. Additionally, each of these domain linkers may be used in multiples (e.g. EAAAKEAAAK; SEQ ID NO:775) or in combination (e.g. EAAAKEPKSSDKTHTCPPCP; SEQ ID NO:776).

FIGS. 9A-9E show the sequences of several useful heterodimeric IL18 fusion backbones based on human IgG, without the cytokine, Fv sequences, or domain linkers. Heterodimeric Fc backbone 1 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants . Heterodimeric Fc backbone 2 is based on human IgG1 (356E/358M allotype), and includes the L368D/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants . Heterodimeric Fc backbone 3 is based on human IgG1 (356E/358M allotype), and includes the L368E/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants . Heterodimeric Fc backbone 4 is based on human IgG1 (356E/358M allotype), and includes the K360E/Q362E/T411E skew variants and the Q295E/N384D/Q418E/N421D pI variants on a first heterodimeric Fc chain, the D401K skew variant on a second heterodimeric Fc chain, and the E233P/L234V/L235A/G236del/S267K ablation variants. Heterodimeric Fc backbone 5 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants. Heterodimeric Fc backbone 6 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants, N297A variant that removes glycosylation, . Heterodimeric Fc backbone 7 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants, N297S variant that removes glycosylation, . Heterodimeric Fc backbone 8 is based on human IgG4, and includes the L368D/K370S skew variants and the Q295E/N384D/Q418E/N421D pI variants on a first heterodimeric Fc chain, the S364K/E357Q skew variants on a second heterodimeric Fc chain, and the S228P (according to EU numbering, S241P in Kabat) variant that ablates Fab arm exchange (as is known in the art) on both chains. Heterodimeric Fc backbone 9 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain. Heterodimeric Fc backbone 10 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain, and the S267K ablation variant on both chains. Heterodimeric Fc backbone 11 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants, M428L/N434S Xtend variants, . Heterodimeric Fc backbone 12 is based on human IgG1 (356E/358M allotype), and includes the L368D/K370S skew variants on a first heterodimeric Fc chain, the S364K/E357Q skew variants and P217R/P229R/N276K pI variants on a second heterodimeric Fc chain, and the E233P/L234V/L235A/G236del/S267K ablation variants. Heterodimeric Fc backbone 13 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains. Heterodimeric Fc backbone 14 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains. Heterodimeric Fc backbone 15 is based on human IgG1 (356E/358M allotype), and includes the L368D/K370S skew variants, the Q295E/N384D/Q418E/N421D pI variants, and H435R/Y436 rapid purification variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 16 is based on human IgG1 (356E/358M allotype), and includes the L368D/K370S skew variants and the Q295E/N384D/Q418E/N421D pI variants on a first heterodimeric Fc chain, the S364K/E357Q skew variants and H435R/Y436 rapid purification variants on a second heterodimeric Fc chain, and the E233P/L234V/L235A/G236del/S267K ablation variants on both chains. Heterodimeric Fc backbone 17 is based on human IgG1 (356E/358M allotype), and includes the L368D/K370S skew variants, the Q295E/N384D/Q418E/N421D pI variants, and H435R/Y436 rapid purification variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 18 is based on human IgG1 (356E/358M allotype), and includes the L368D/K370S skew variants and the Q295E/N384D/Q418E/N421D pI variants on a first heterodimeric Fc chain, the S364K/E357Q skew variants and H435R/Y436 rapid purification variants on a second heterodimeric Fc chain, and the E233P/L234V/L235A/G236del/S267K ablation variants and M428L/N434S Xtend variants on both chains.

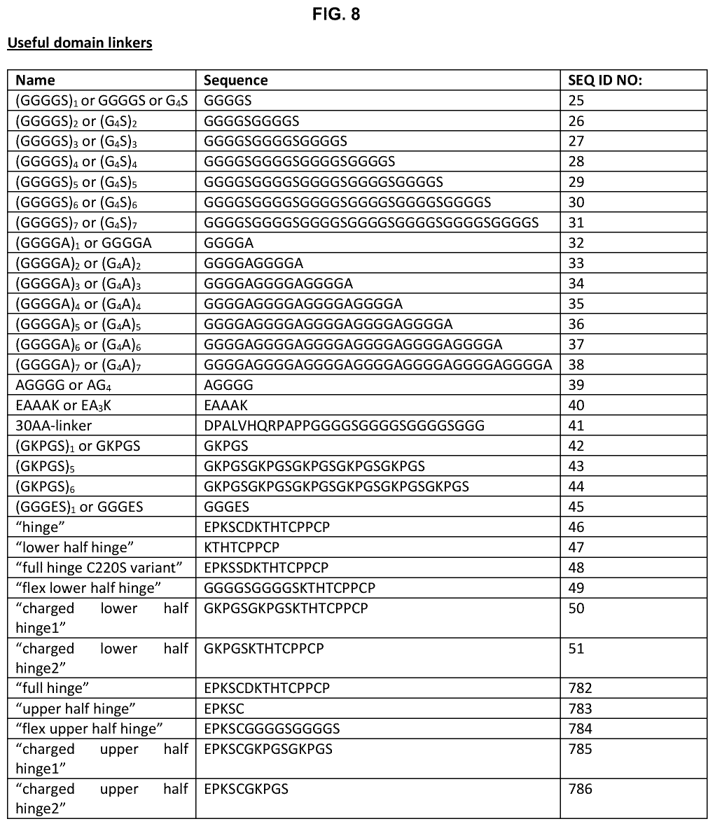

As will be appreciated by those in the art and outlined below, these sequences can be used with any IL18 fusion formats requiring a heterodimeric Fc region. It should be noted that the backbones may further comprise deletion of K447 (i.e. K447_ or K447del) on one or both chains. These sequences can also be used with any of the IL18 x Fab-Fc fusions of the invention. In targeted IL-10 fusion formats which include a variable heavy domain covalently linked to the Fc, the variable heavy domain may be covalently linked to the Fc domain by a corresponding CH1 domain (as depicted in FIG. 10) and domain linkers (as depicted in FIG. 8). Additionally, each of these backbone sequences may include the H435R/Y436F variants on monomer 1 or monomer 2 to ablate Protein A binding.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition or as an alternative to the skew, pI and ablation variants contained within the backbones of this Figure.

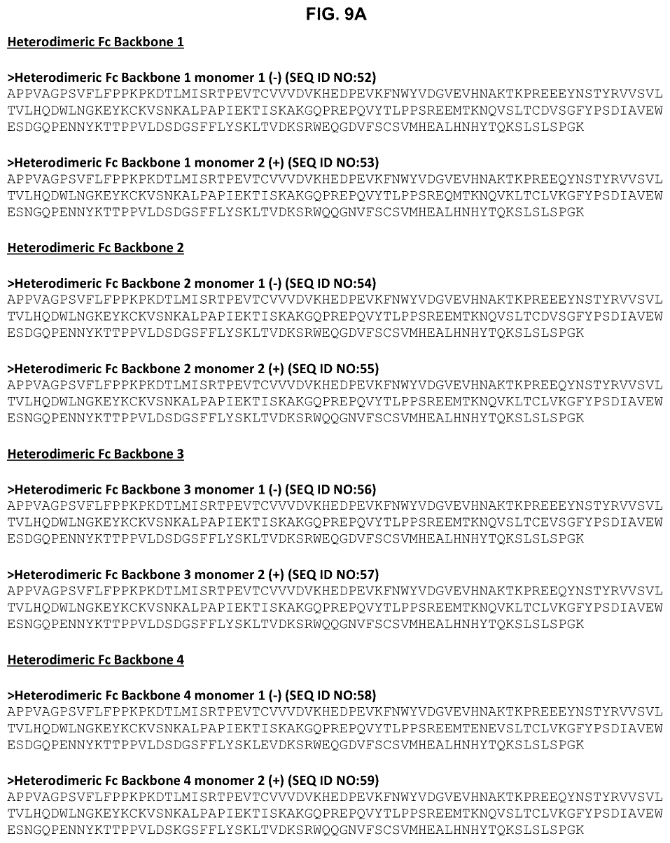

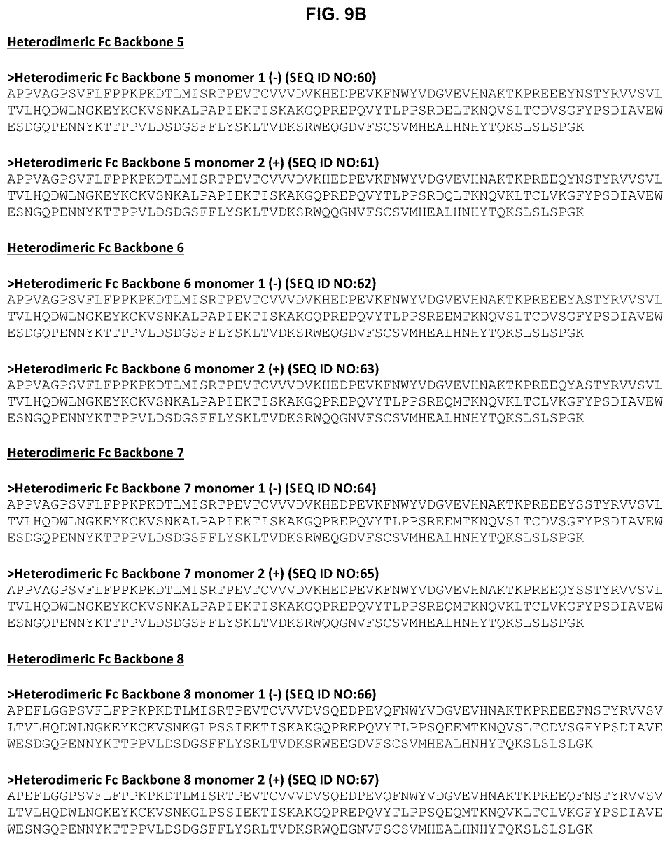

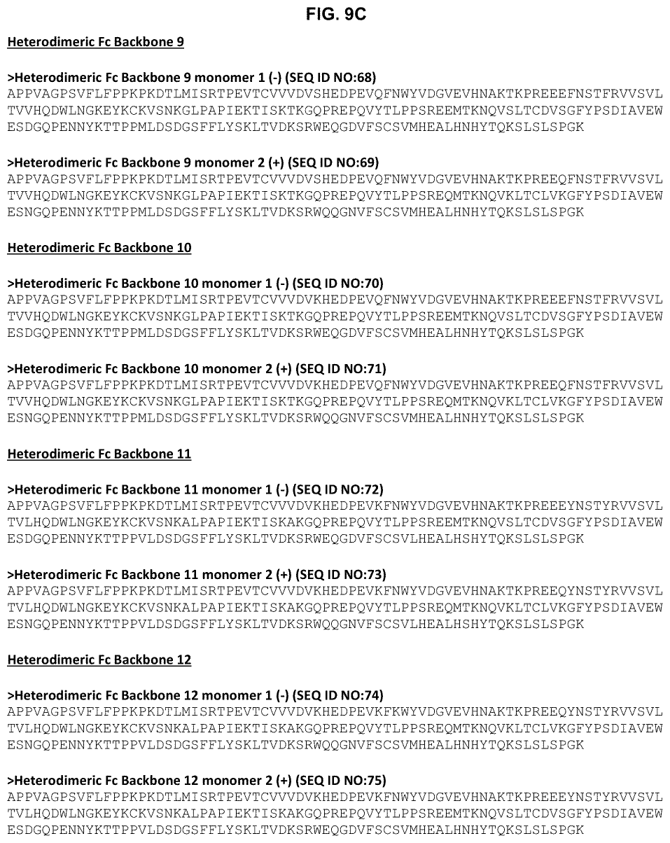

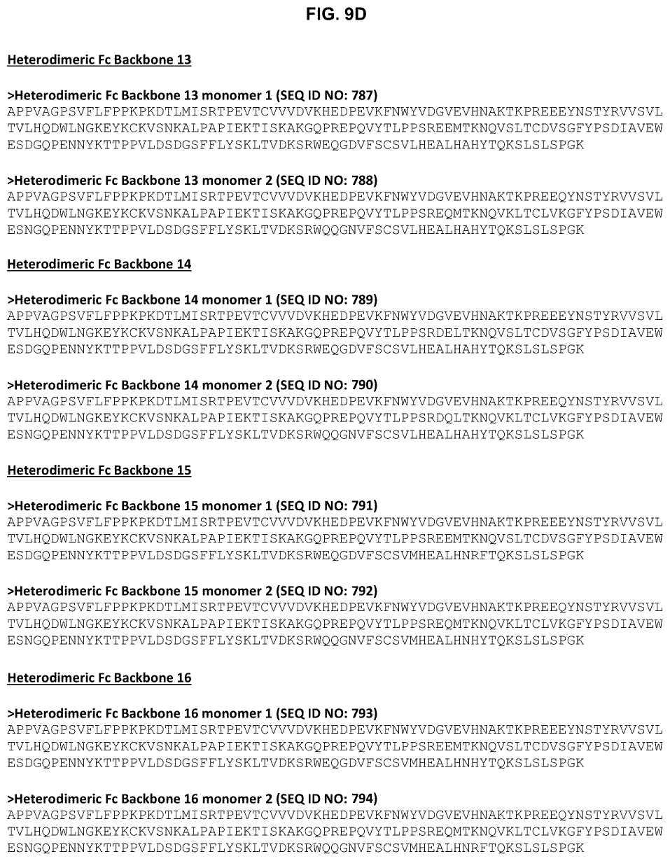

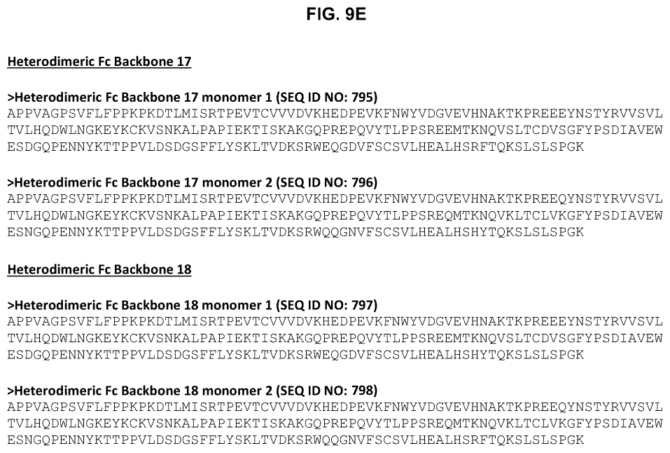

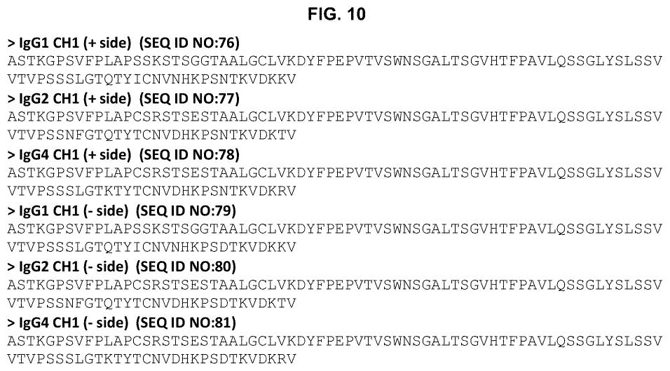

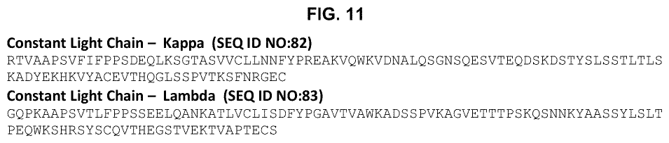

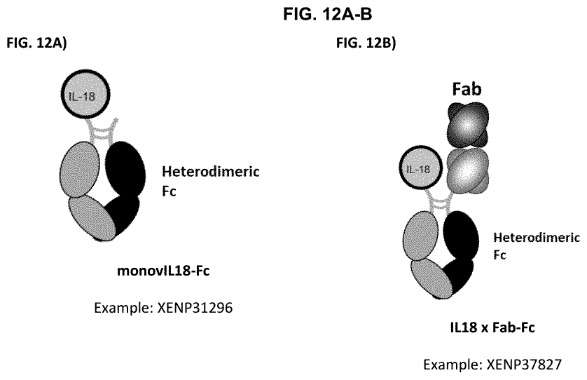

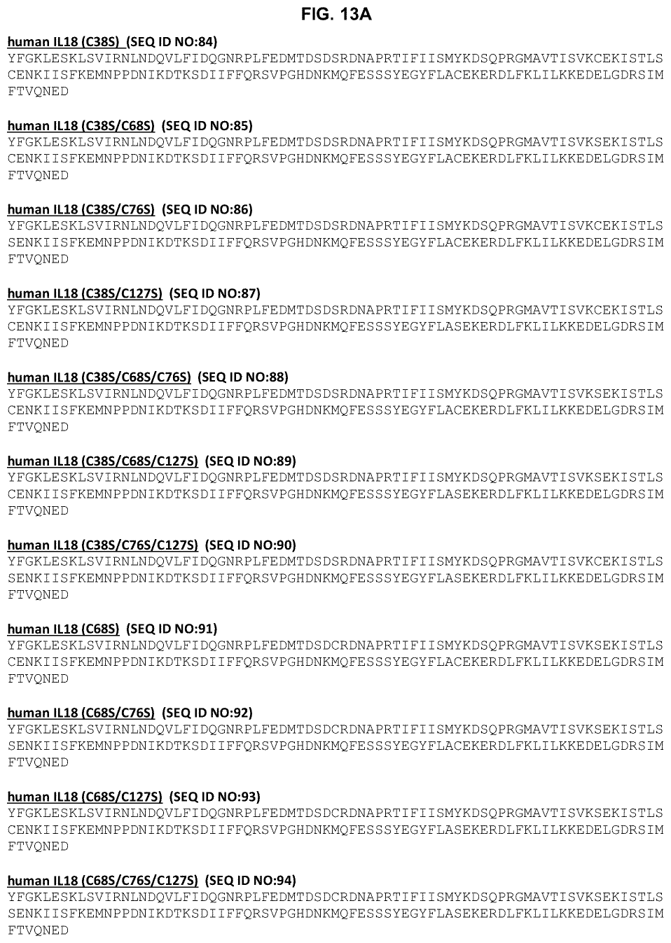

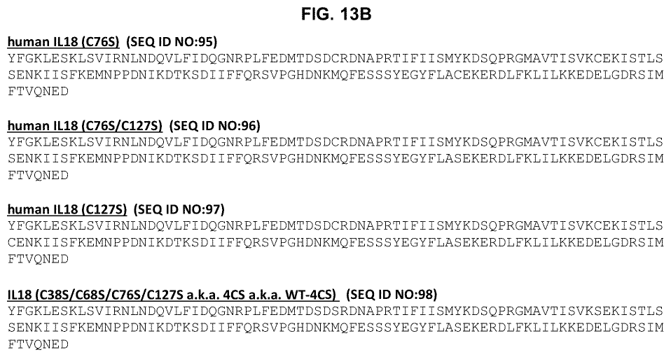

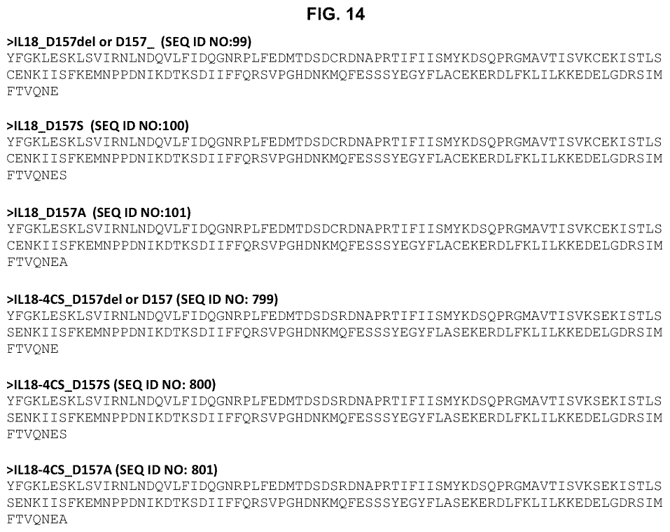

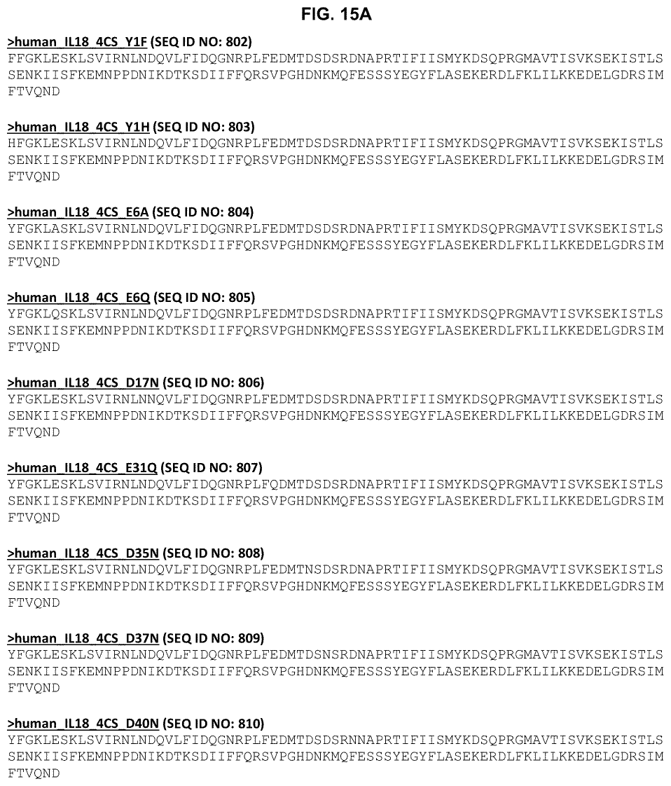

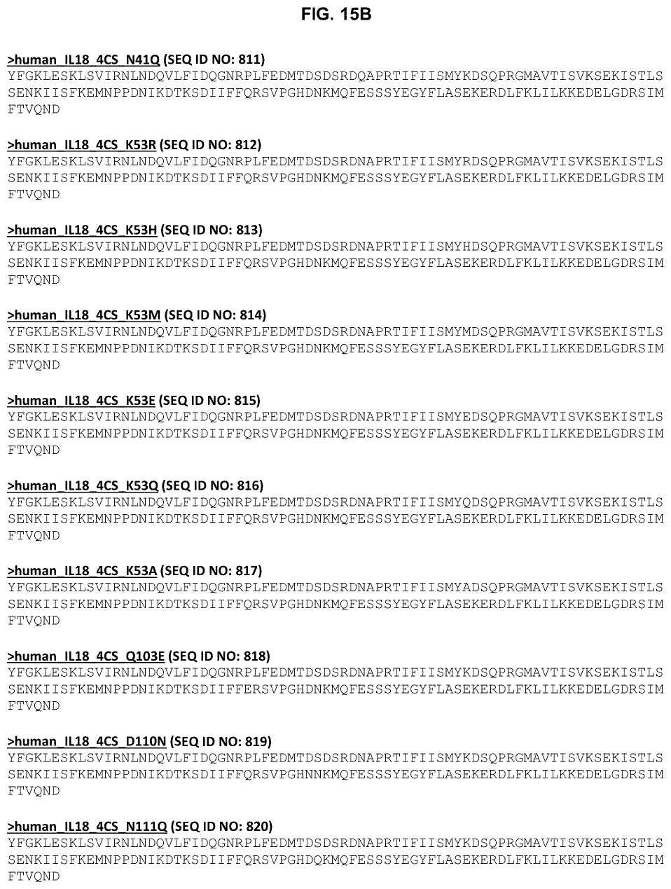

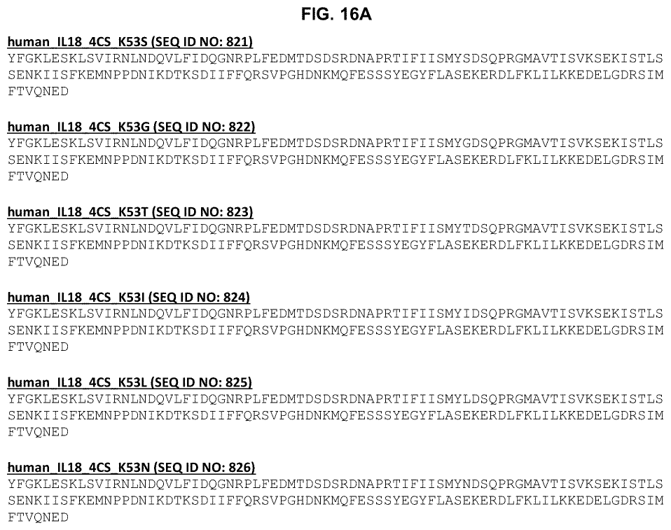

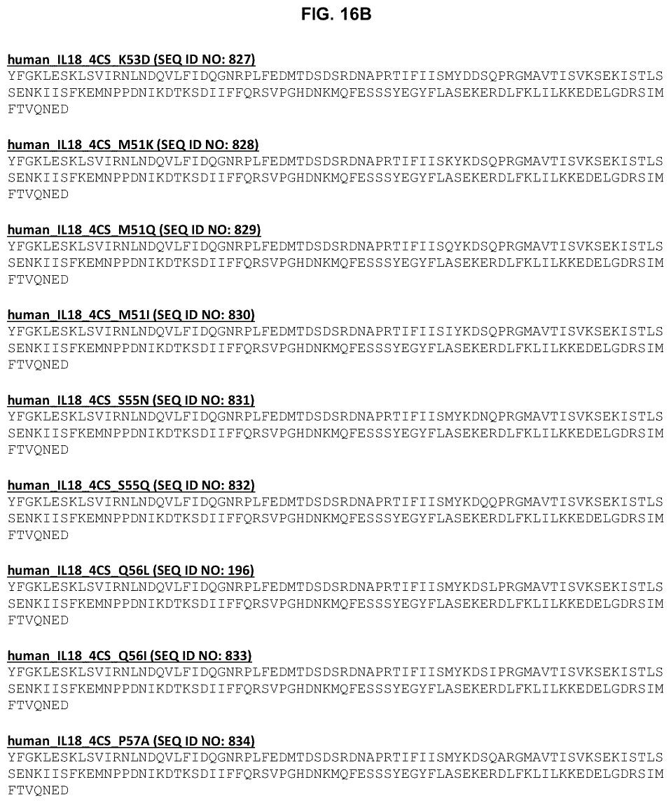

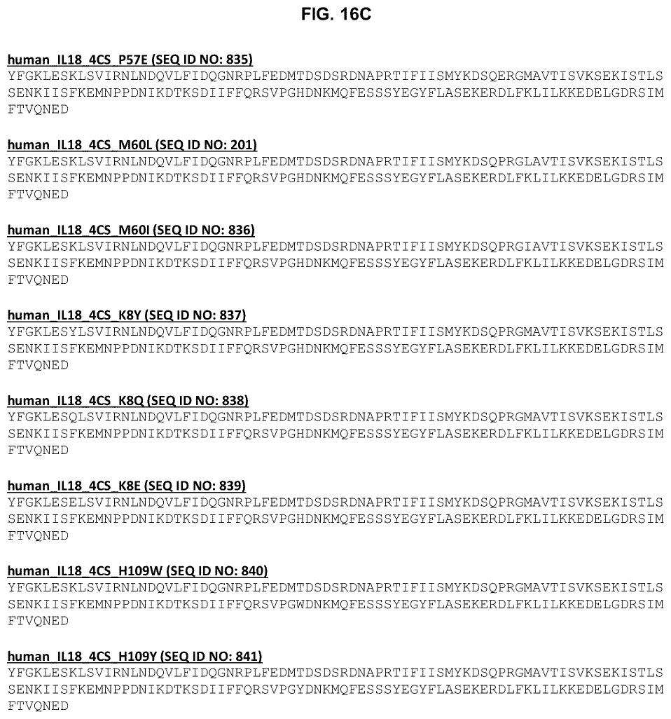

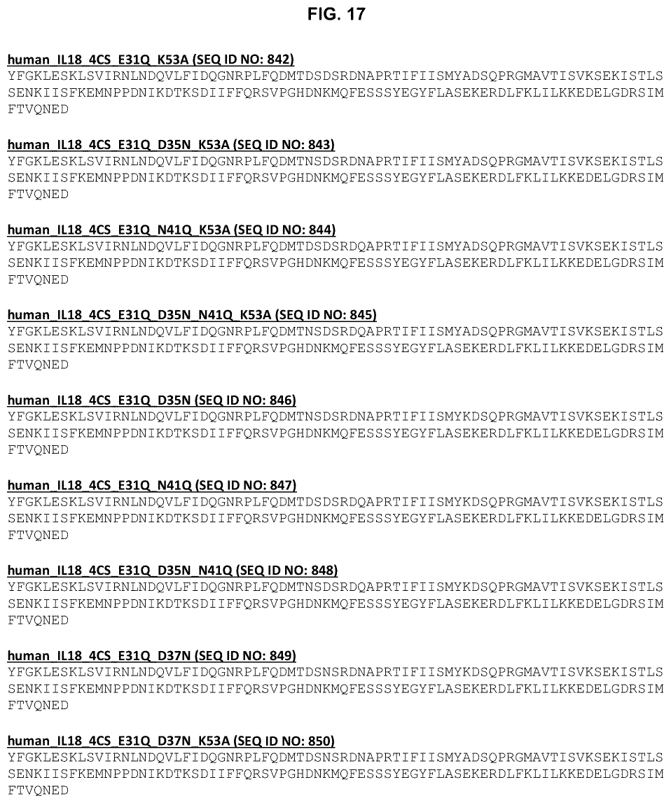

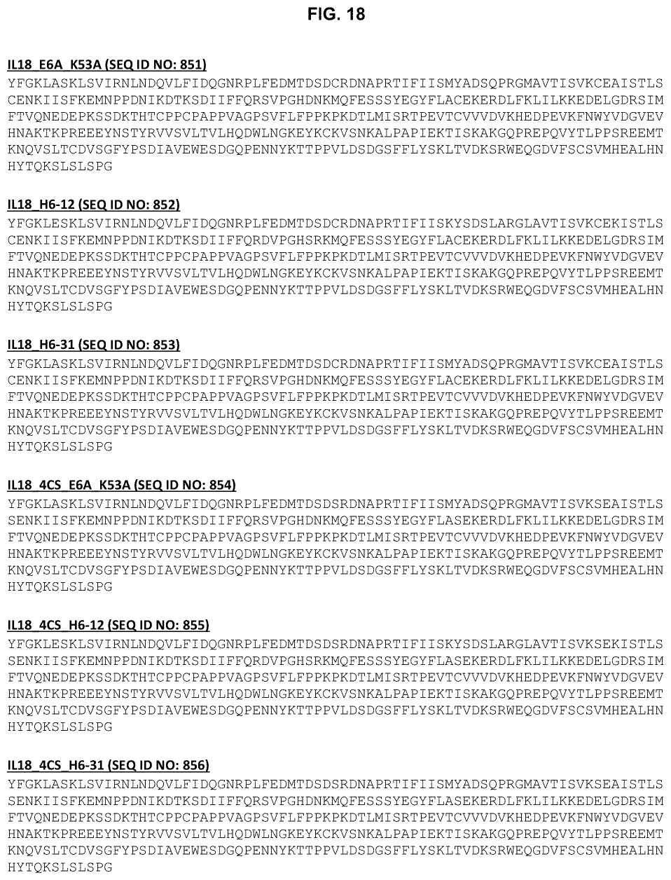

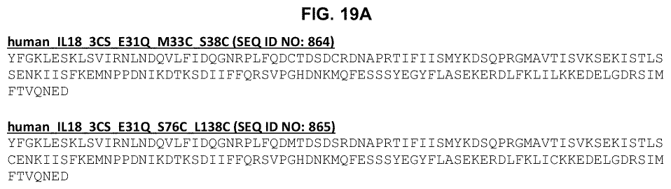

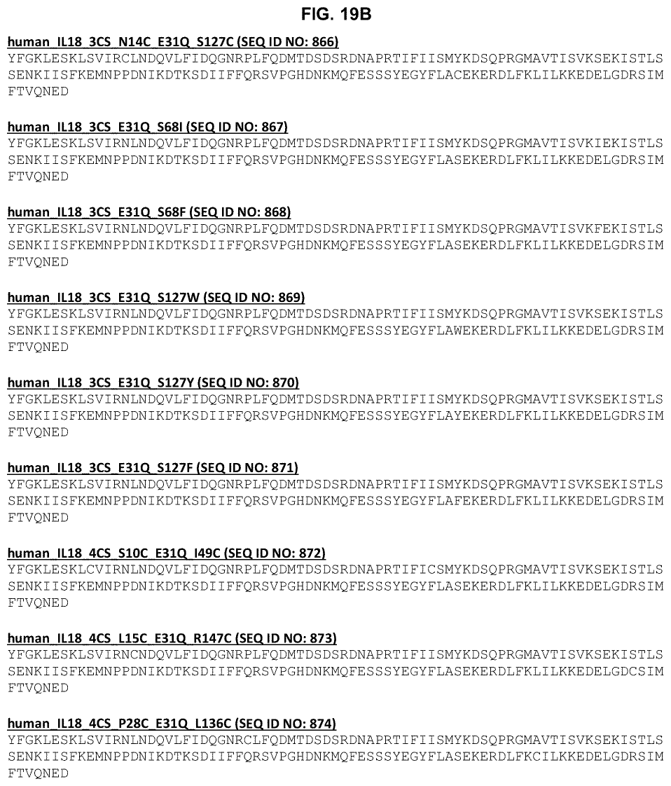

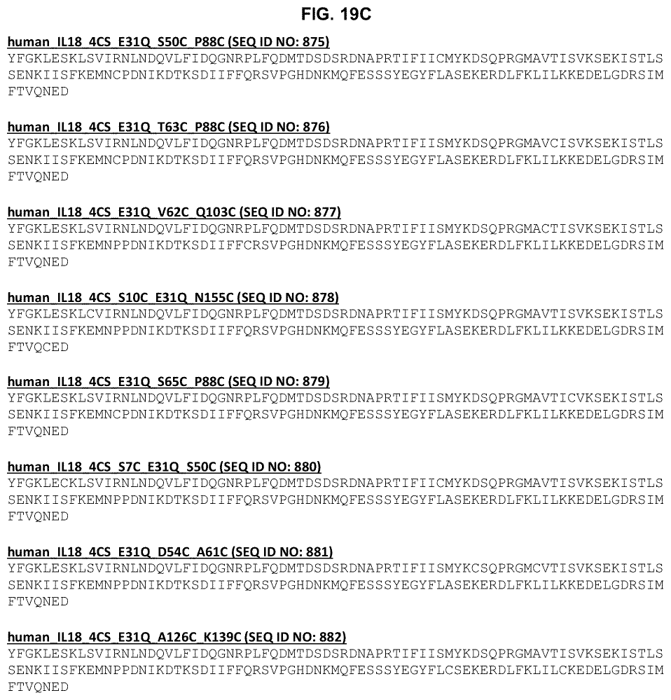

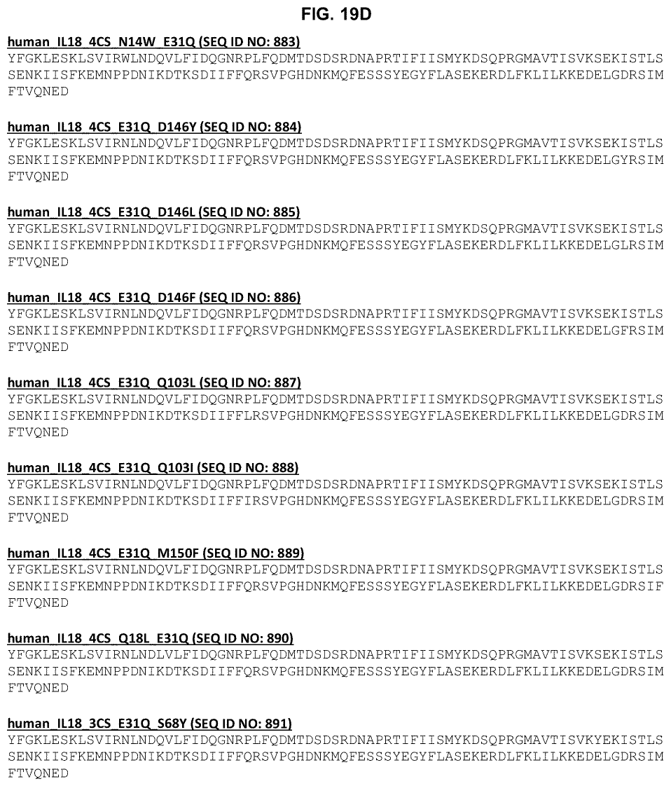

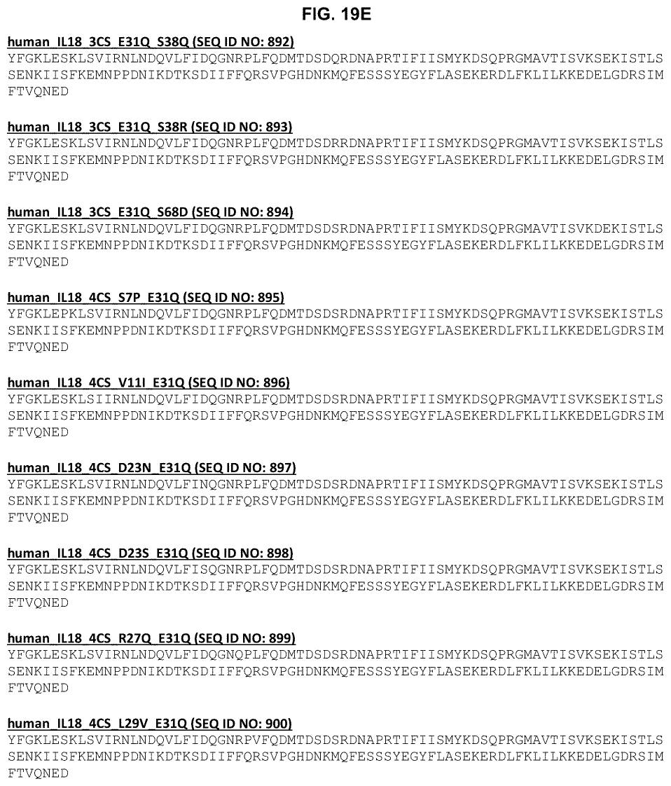

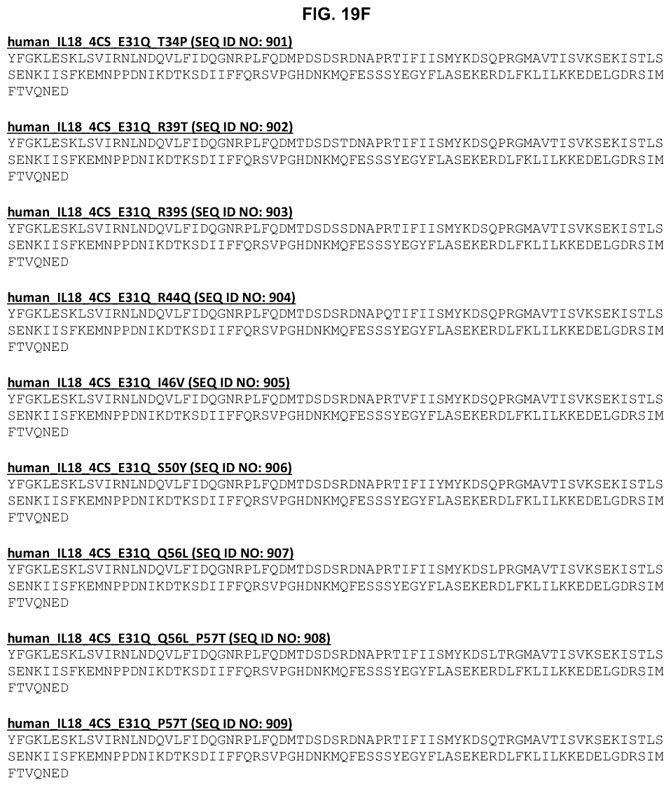

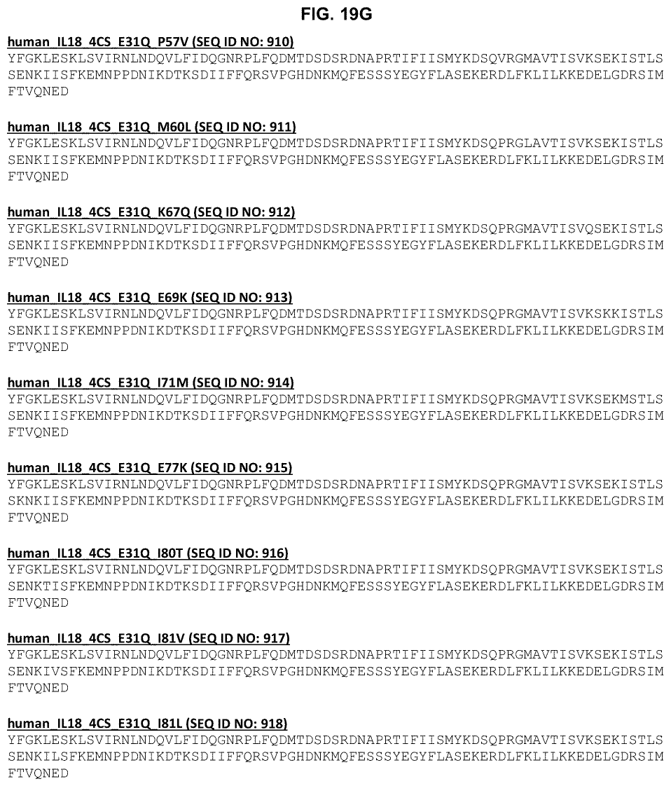

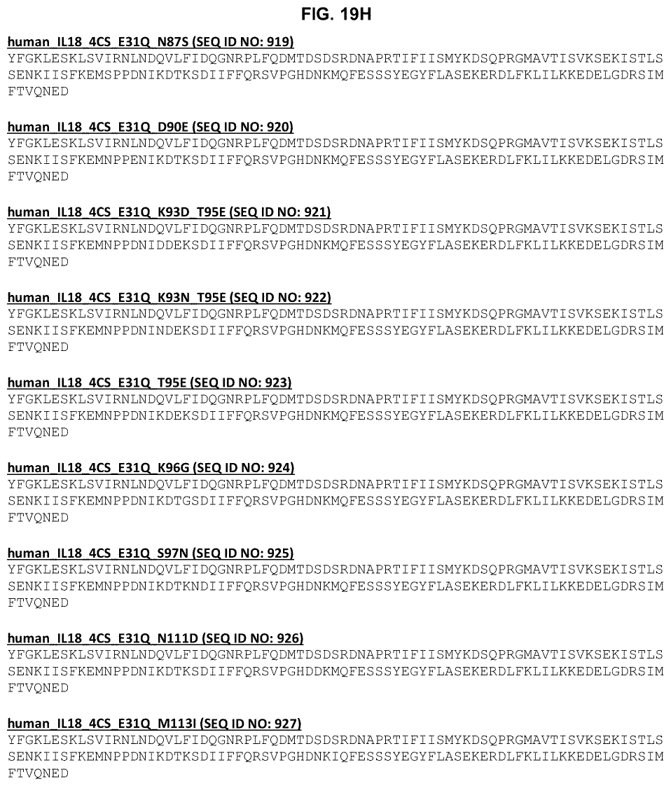

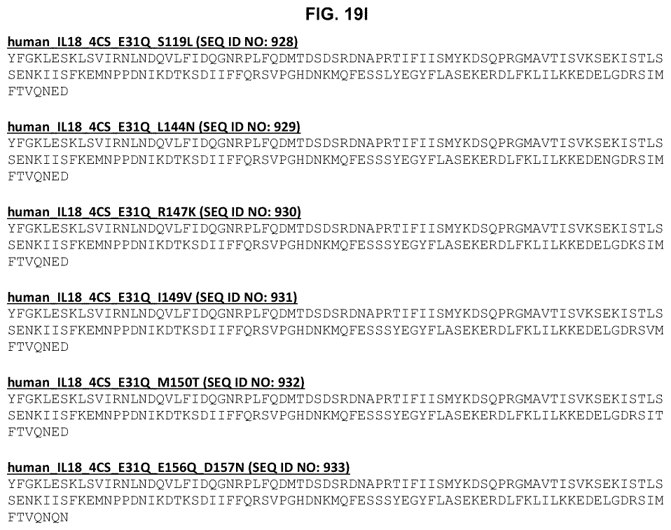

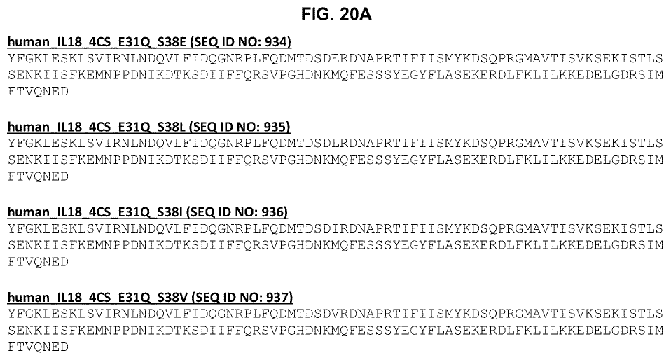

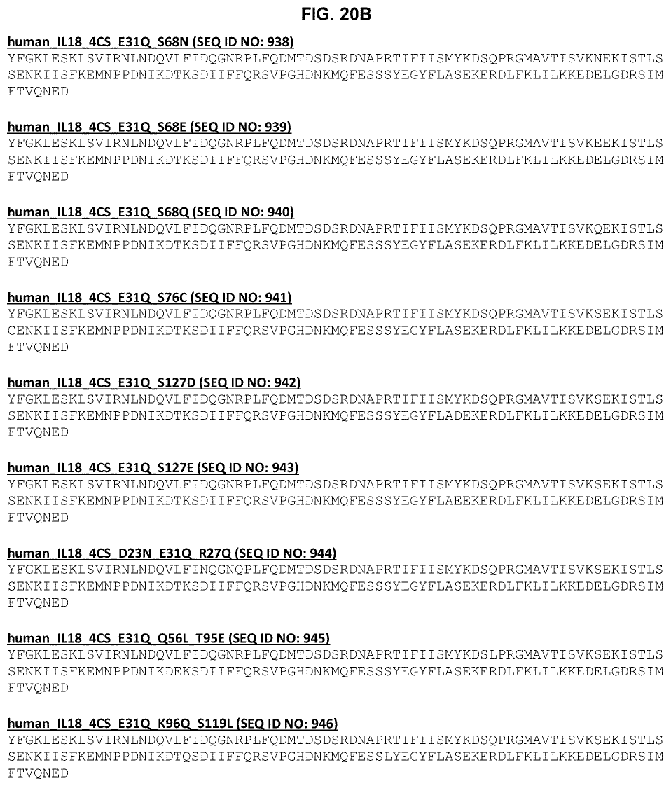

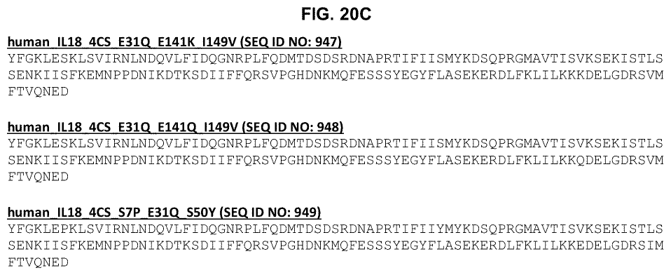

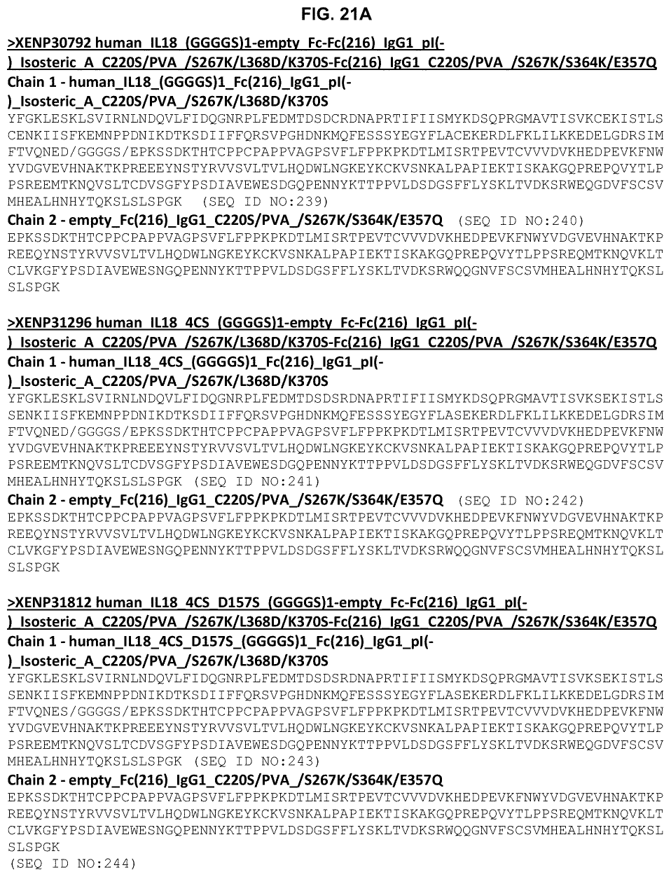

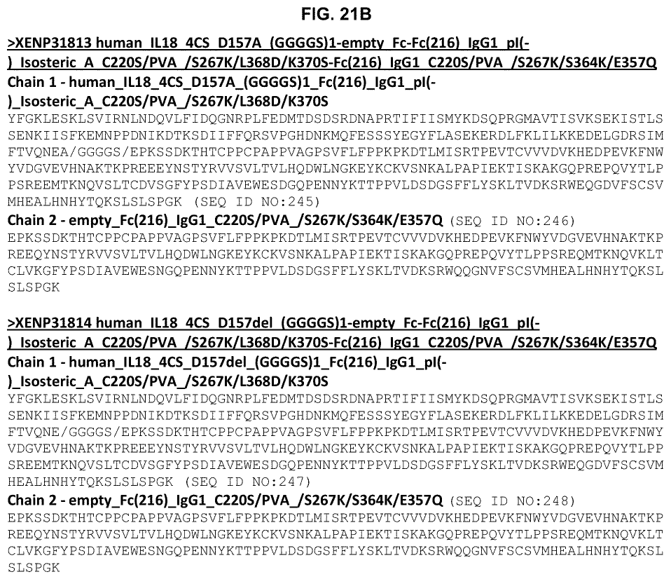

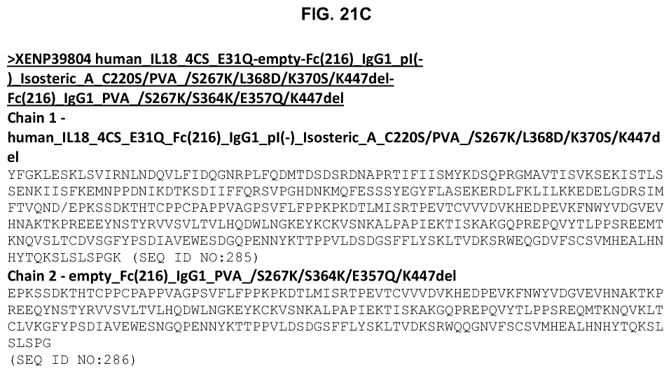

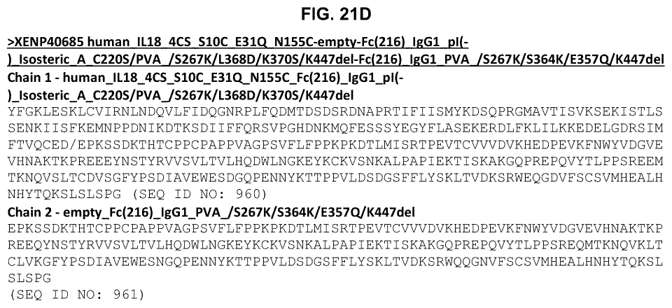

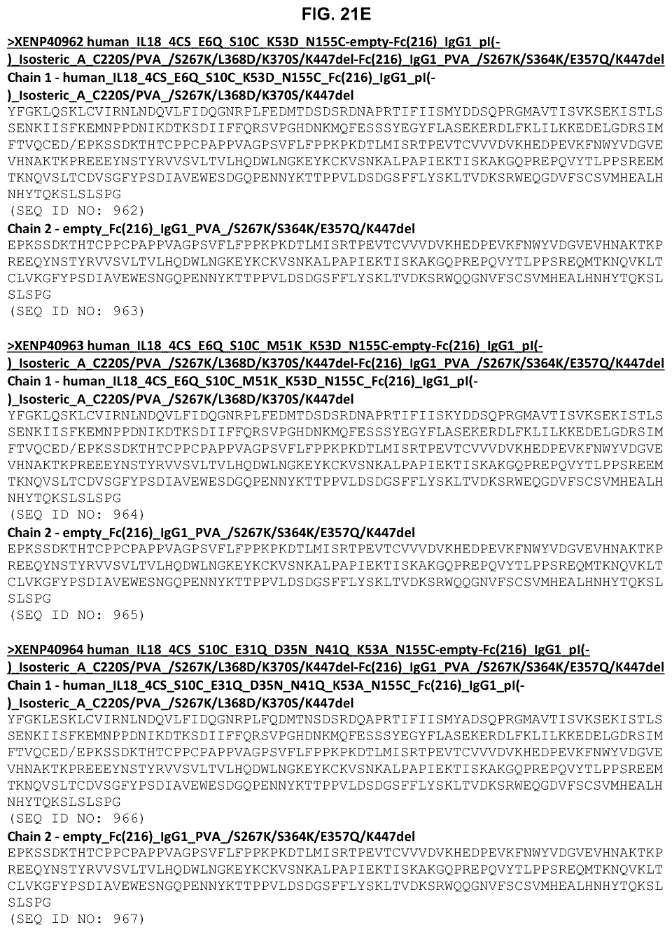

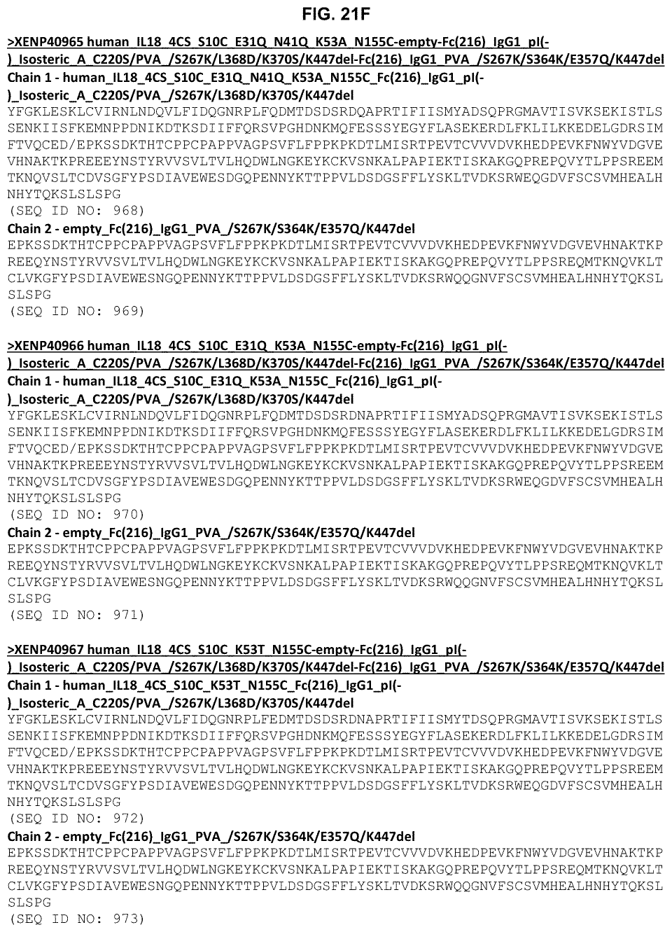

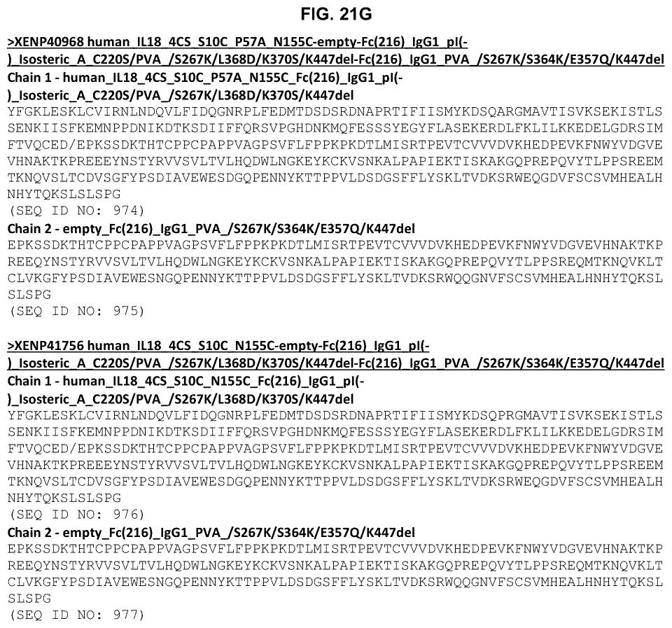

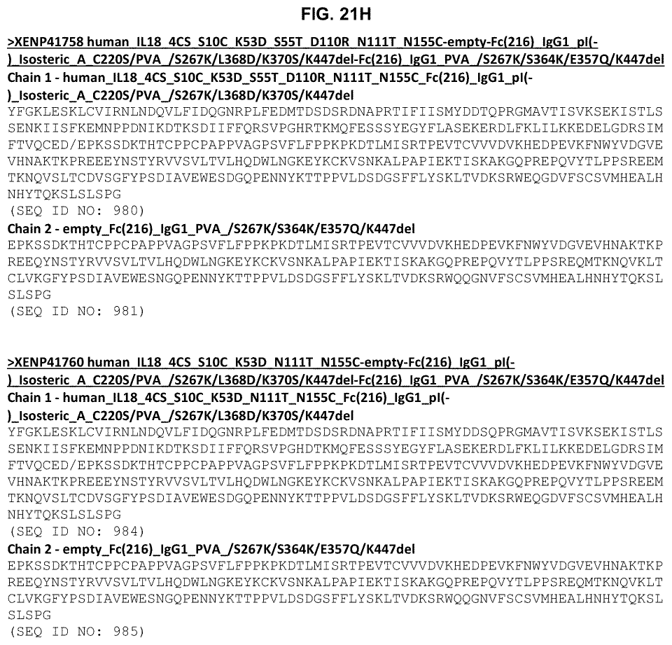

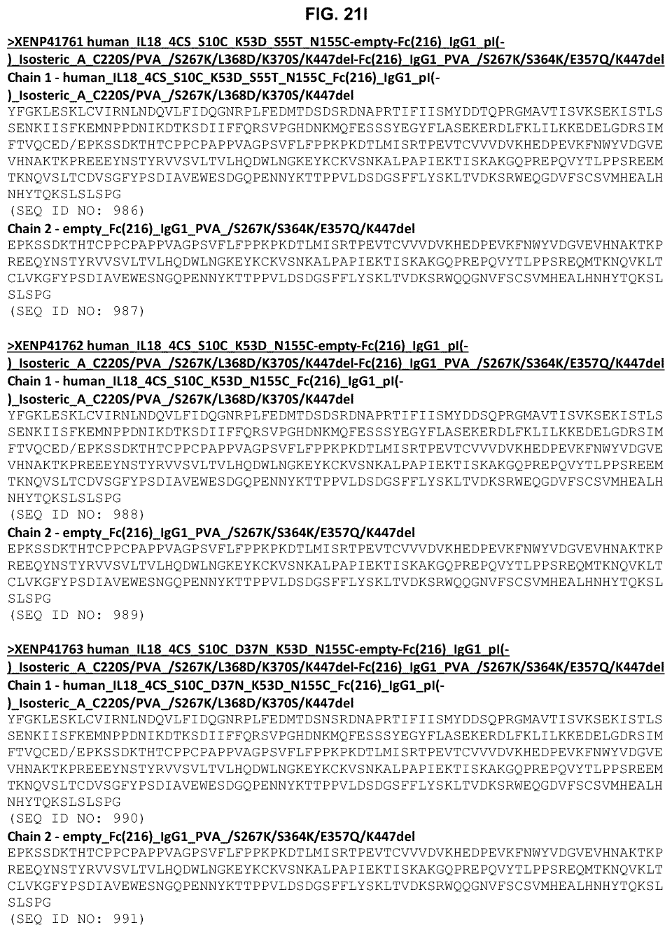

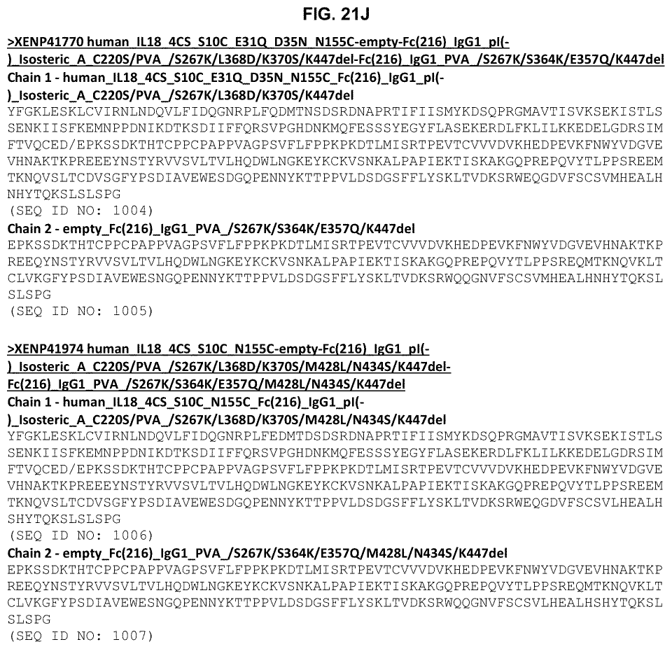

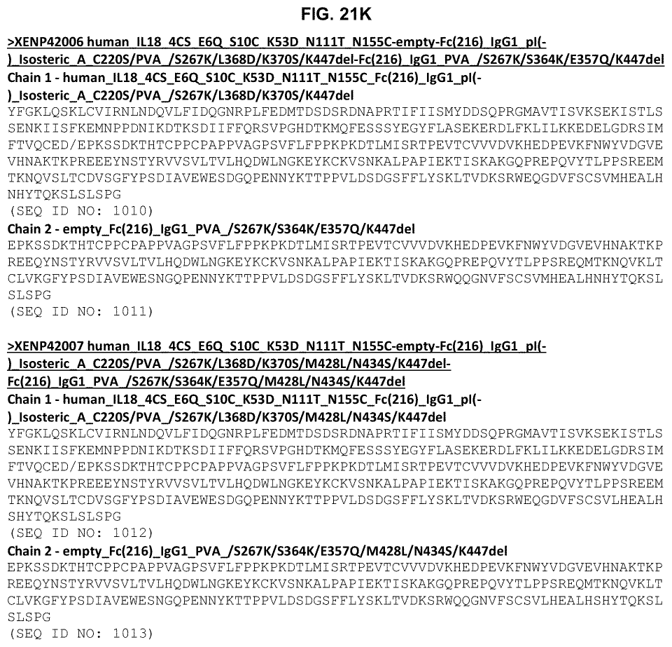

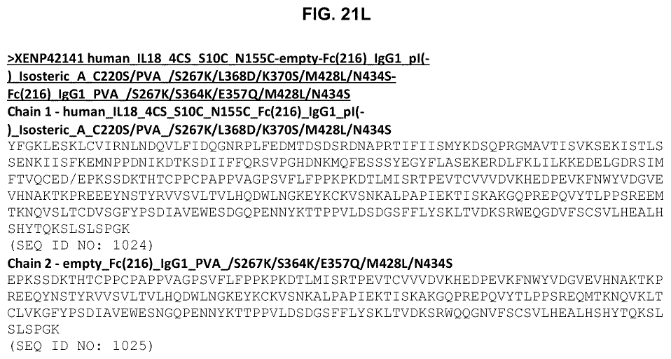

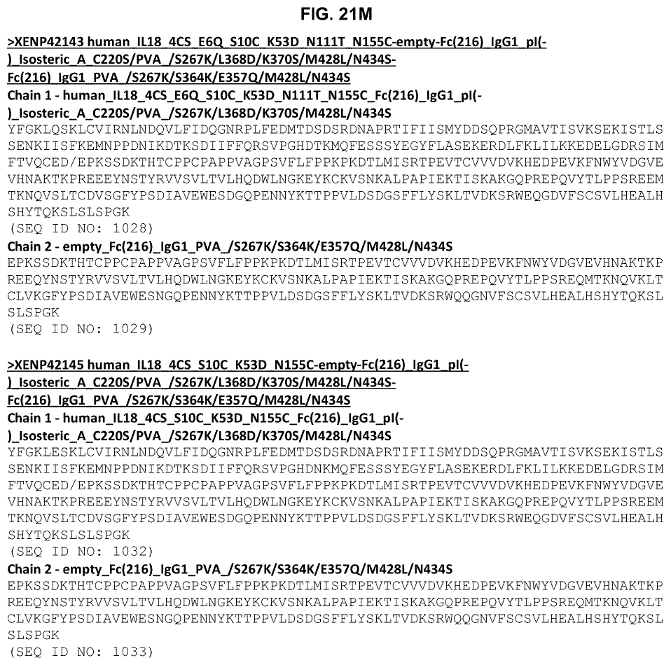

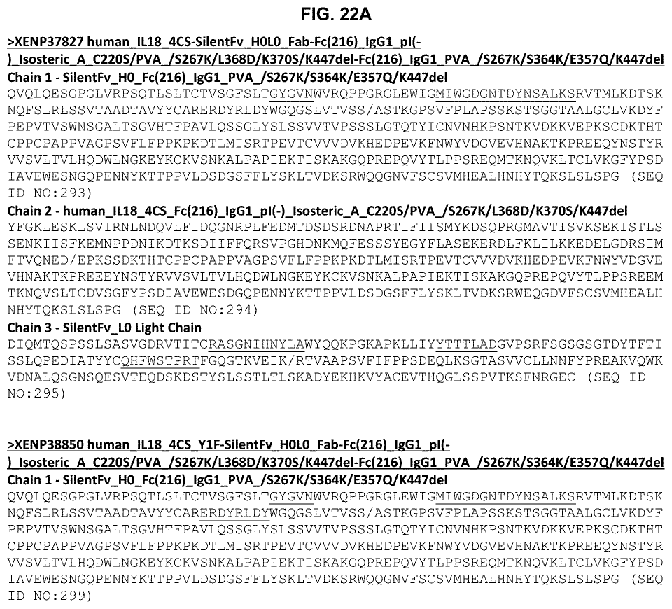

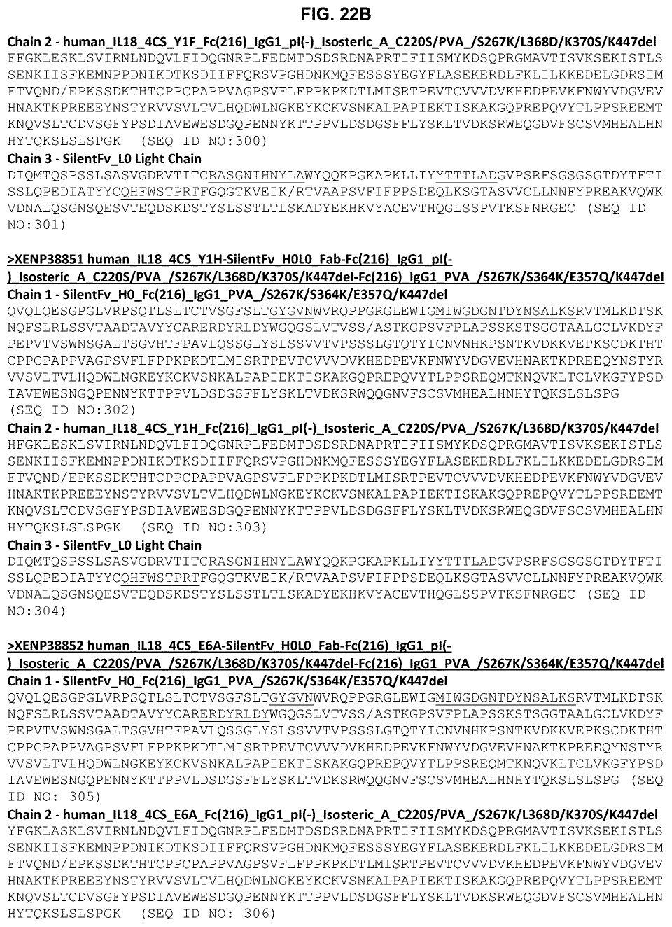

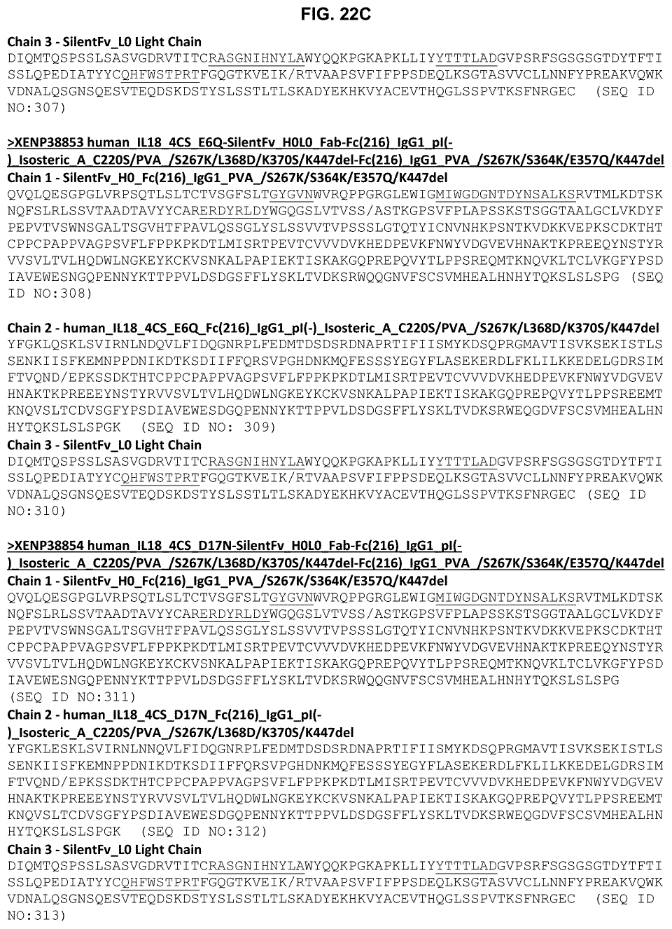

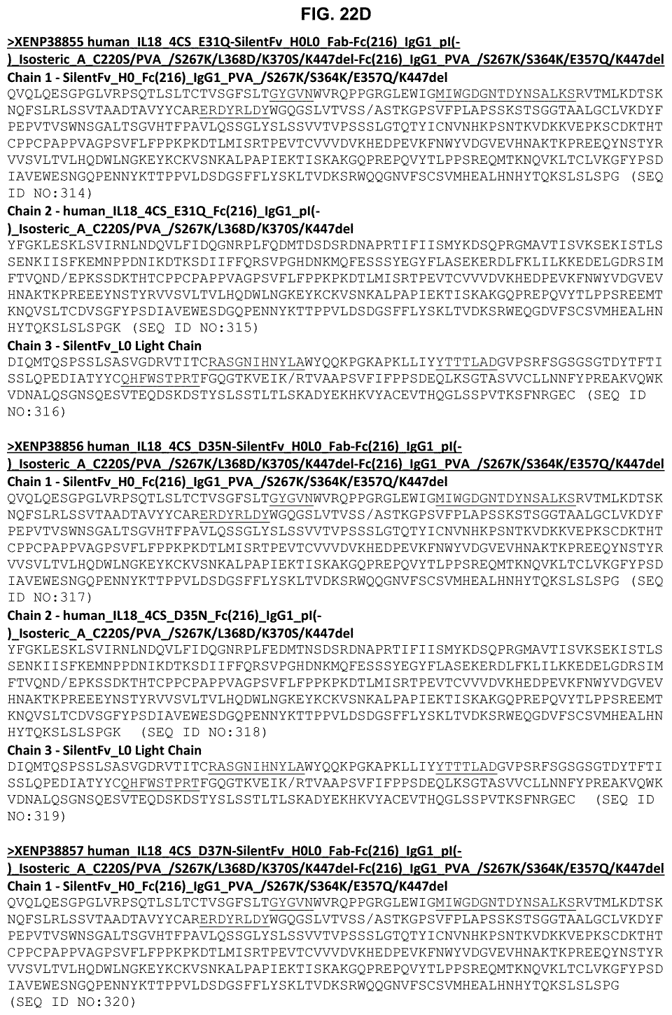

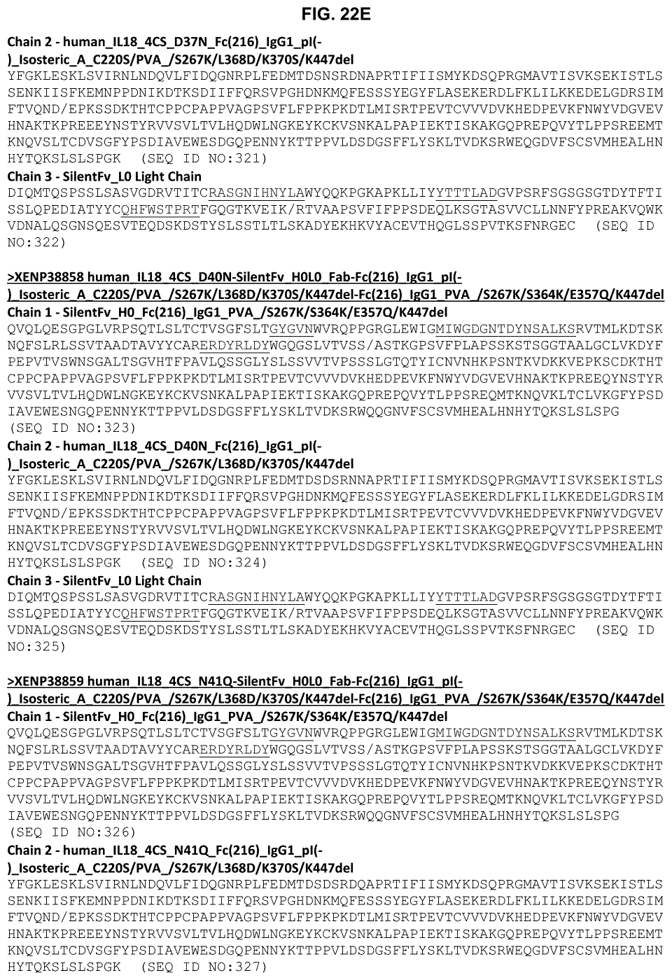

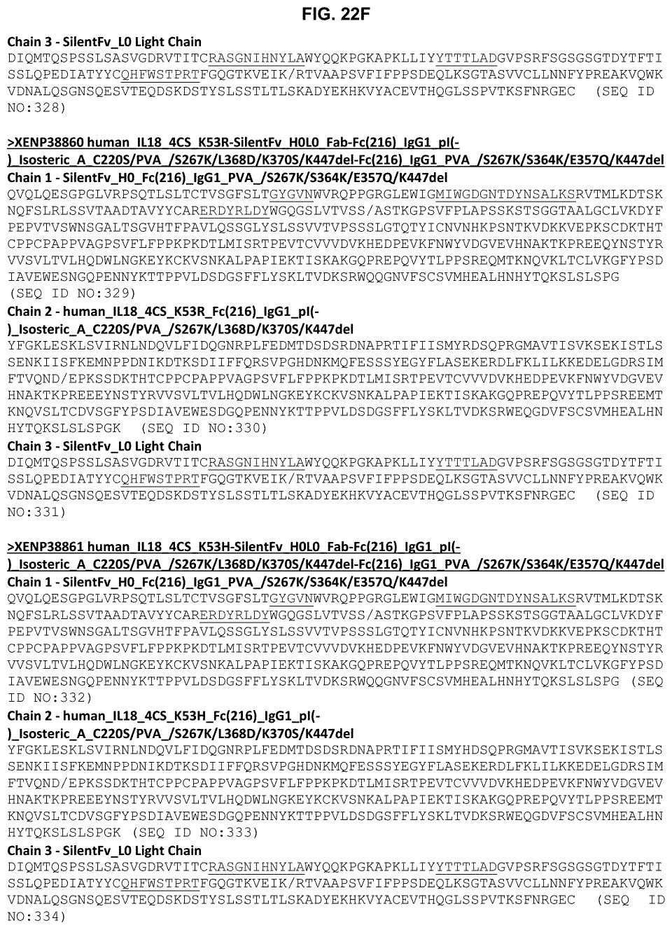

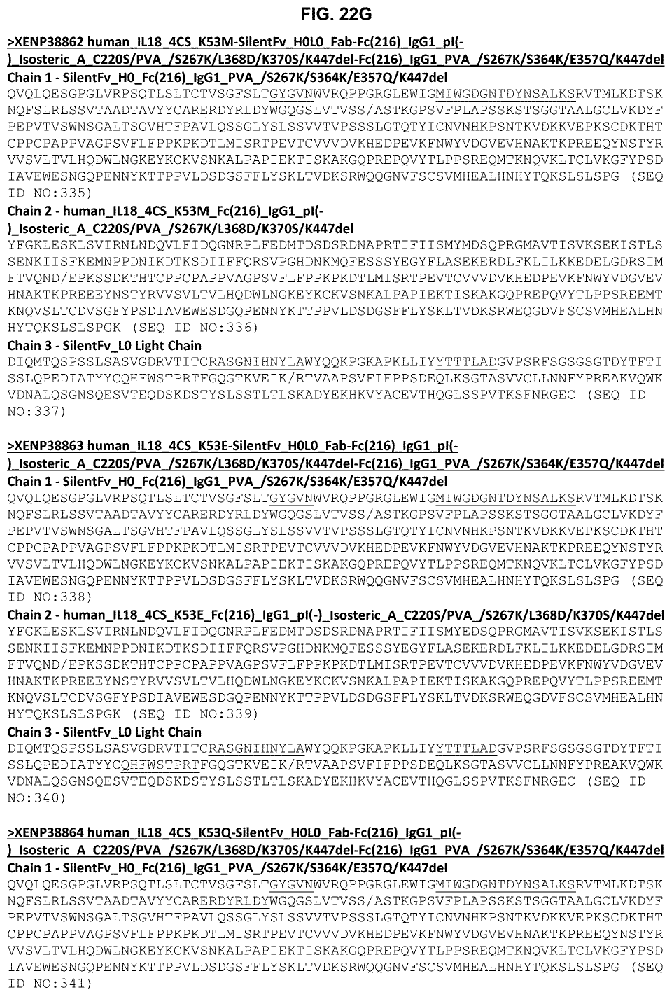

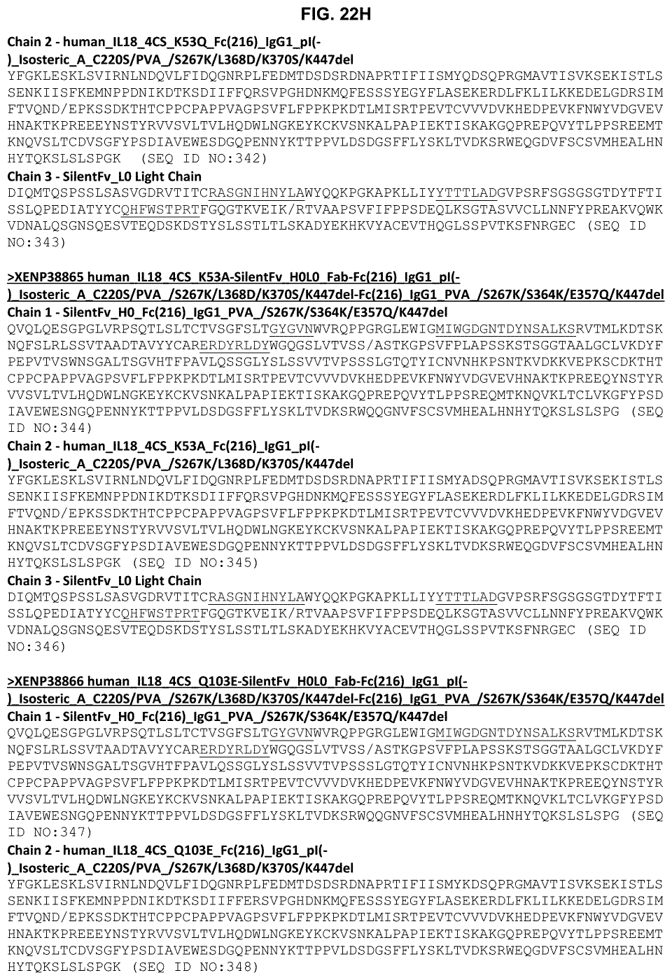

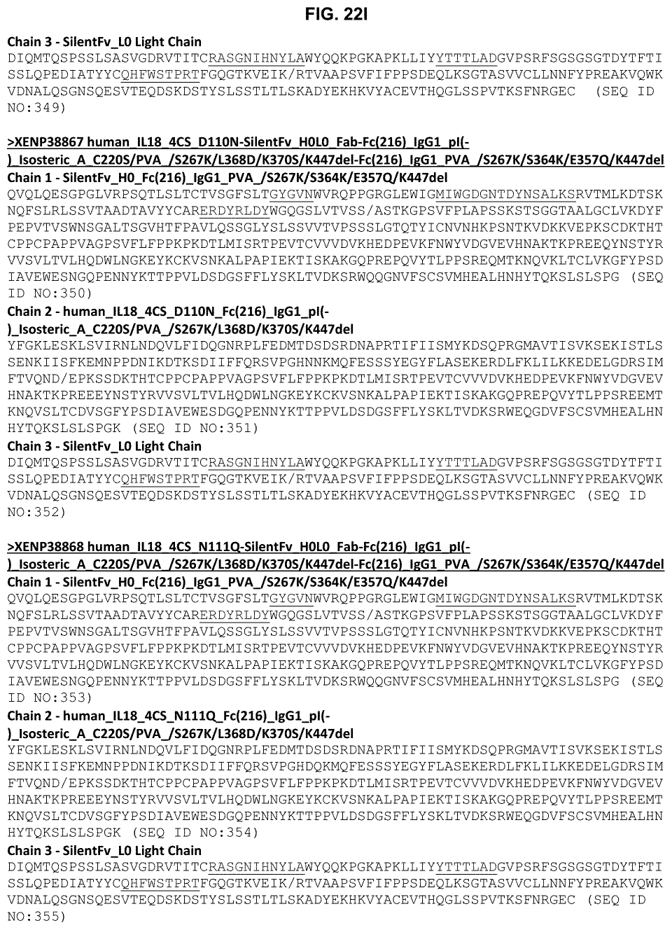

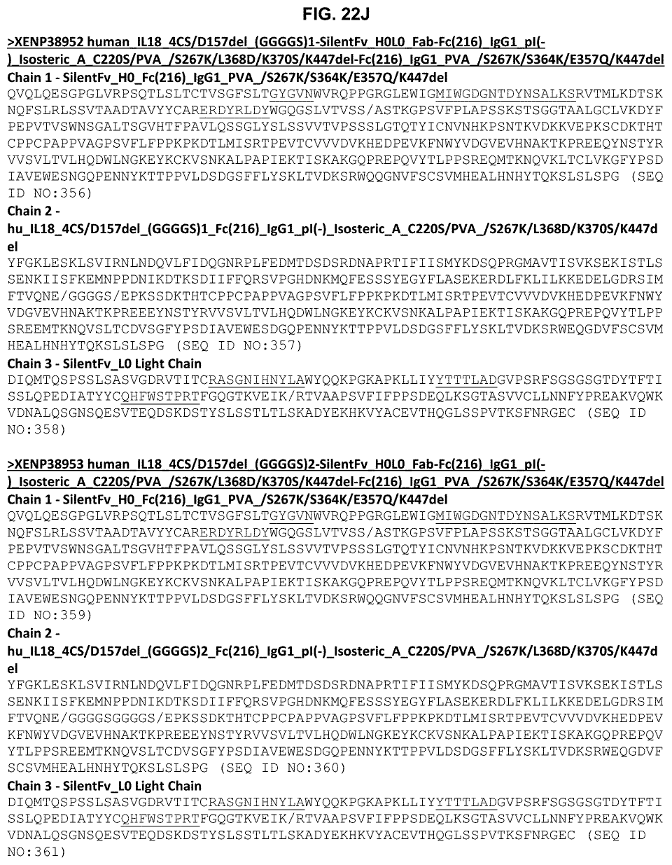

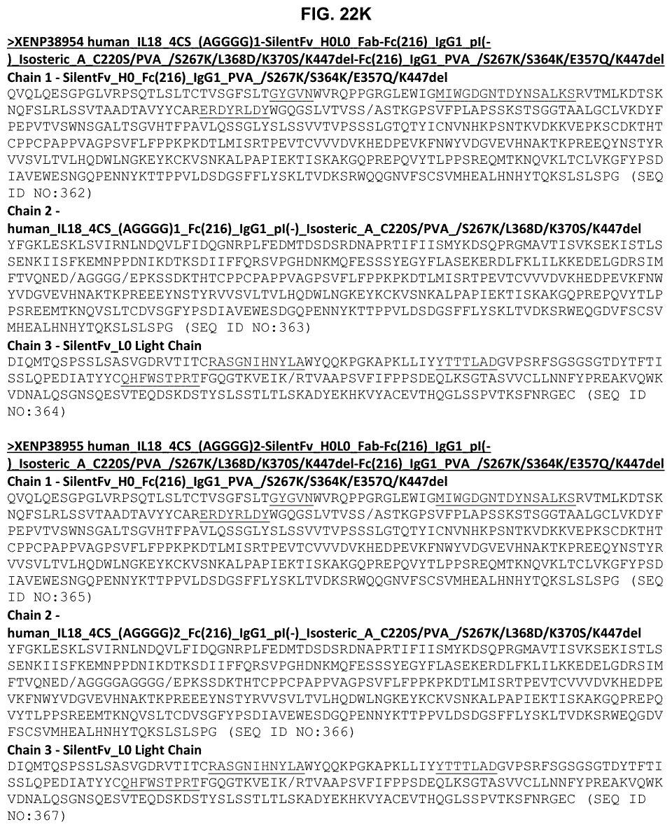

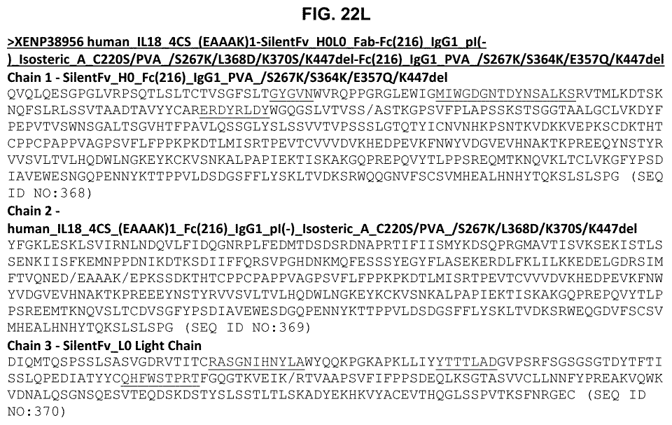

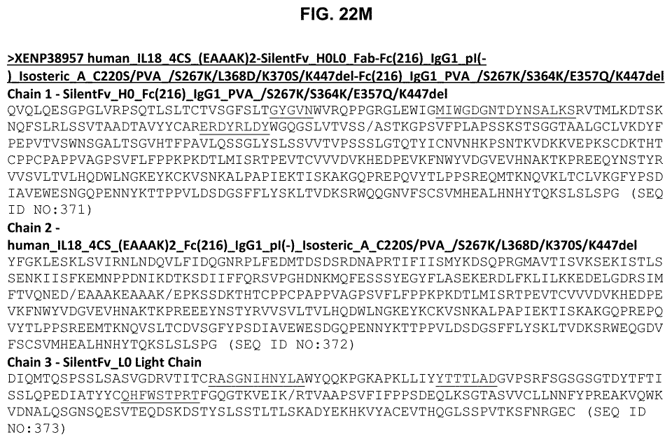

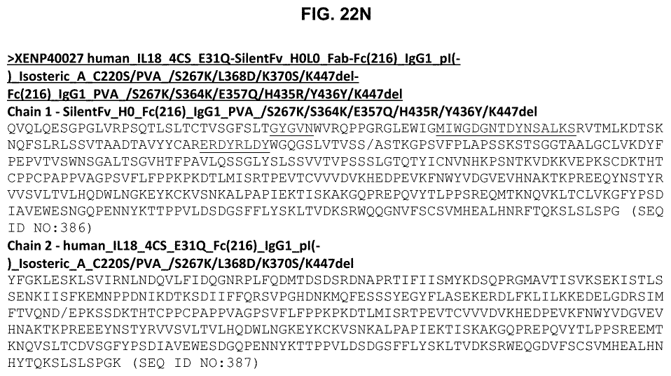

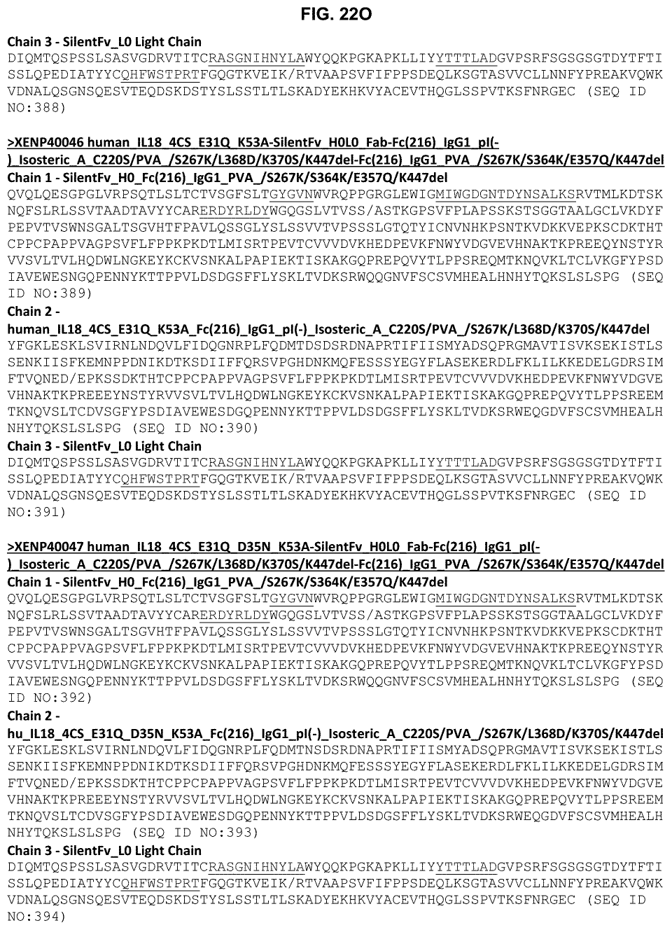

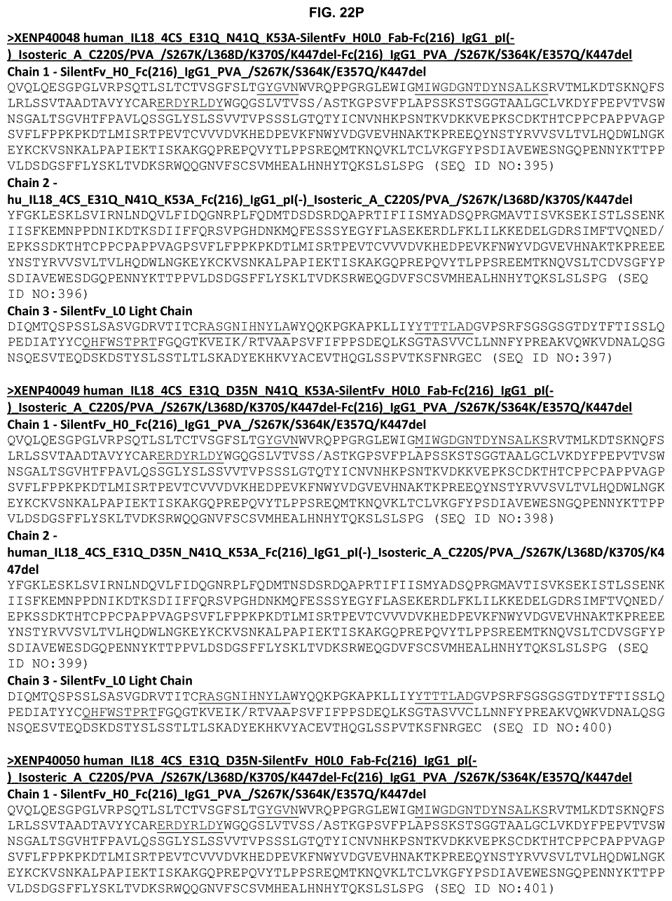

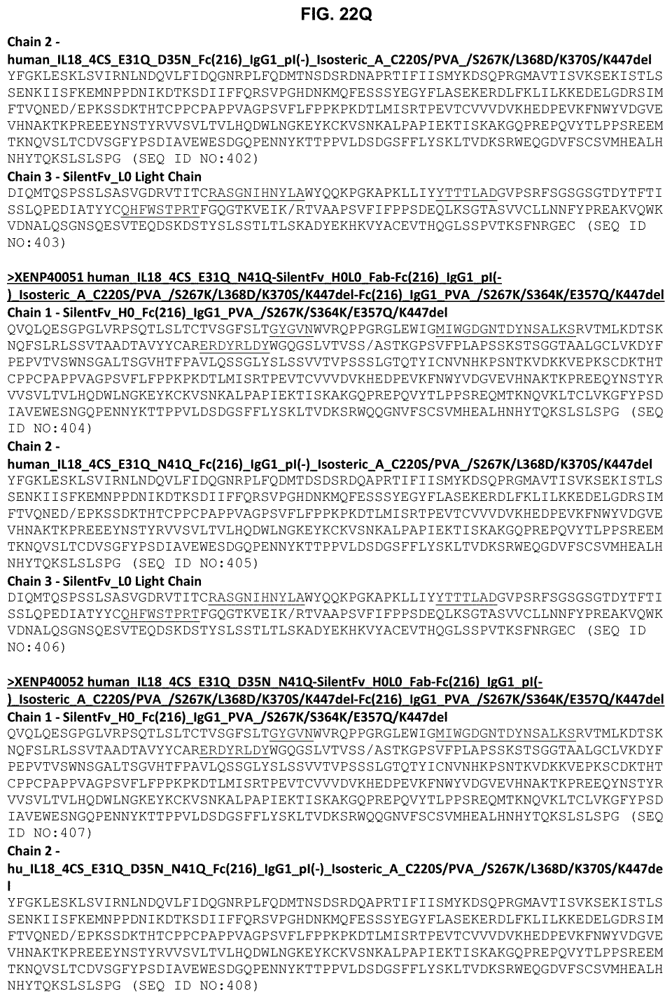

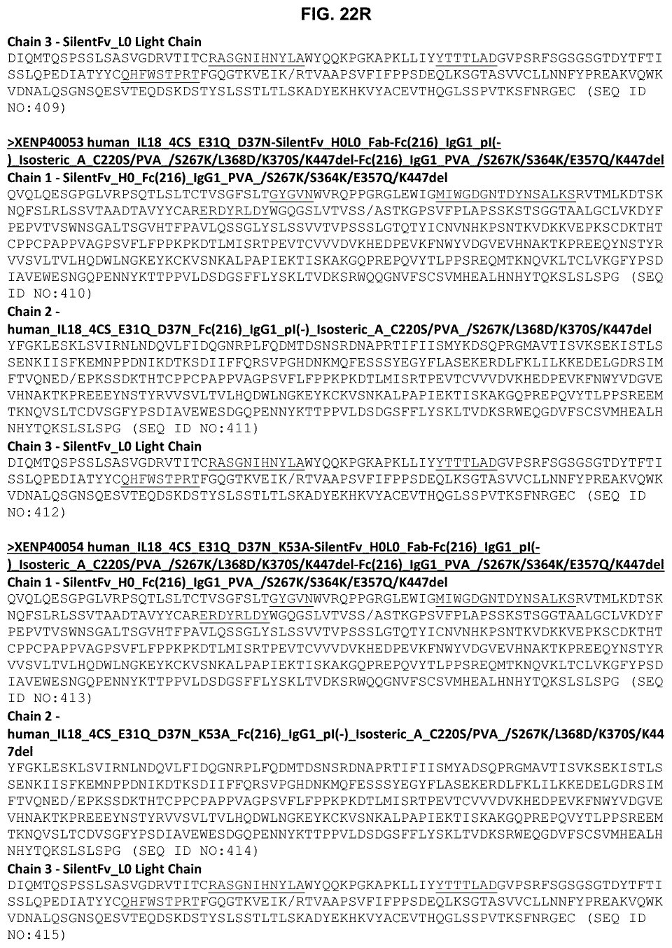

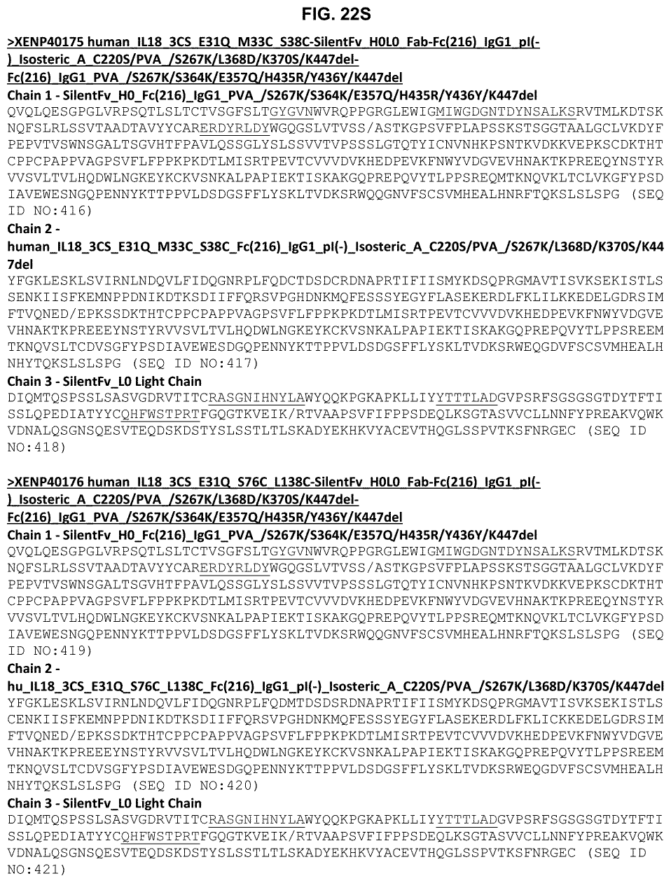

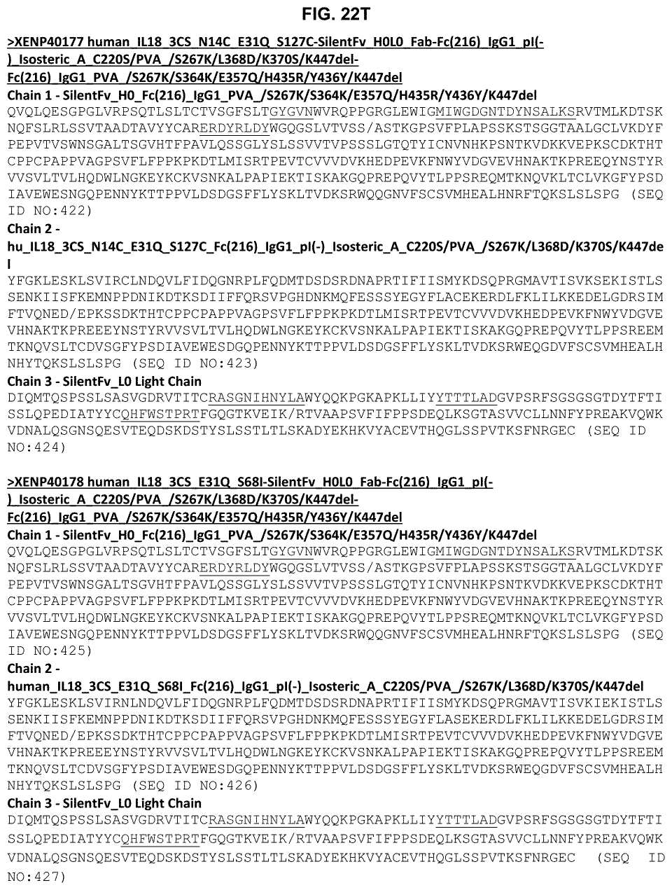

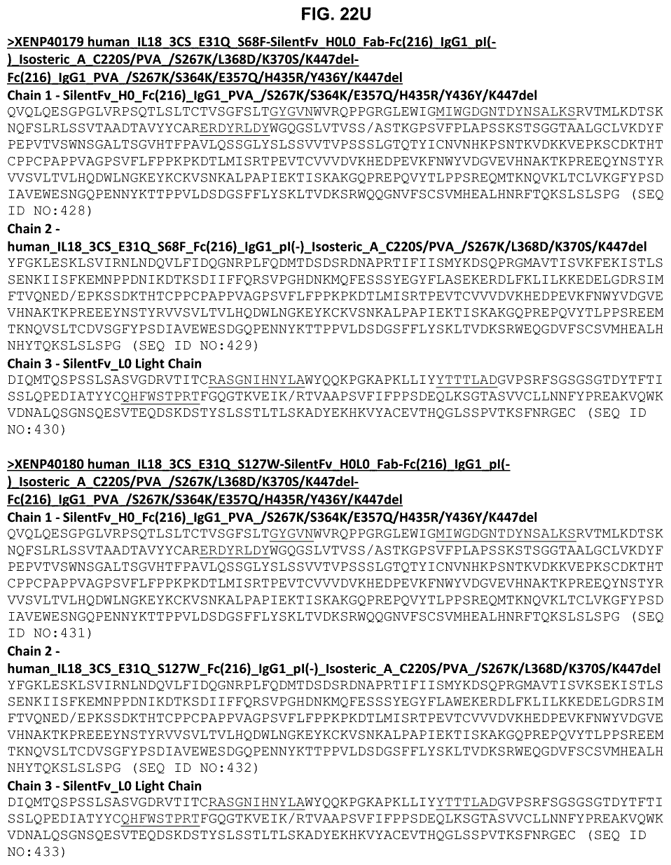

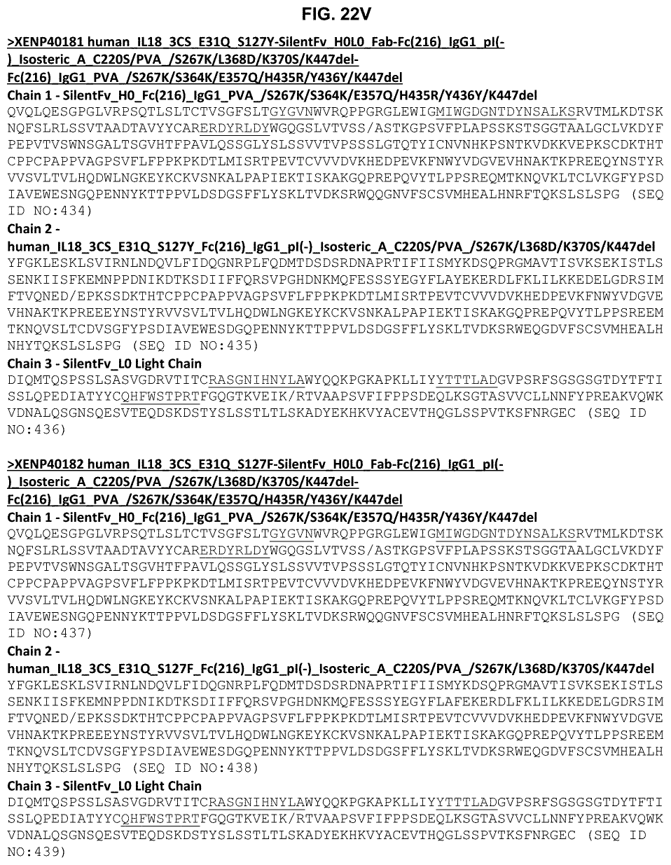

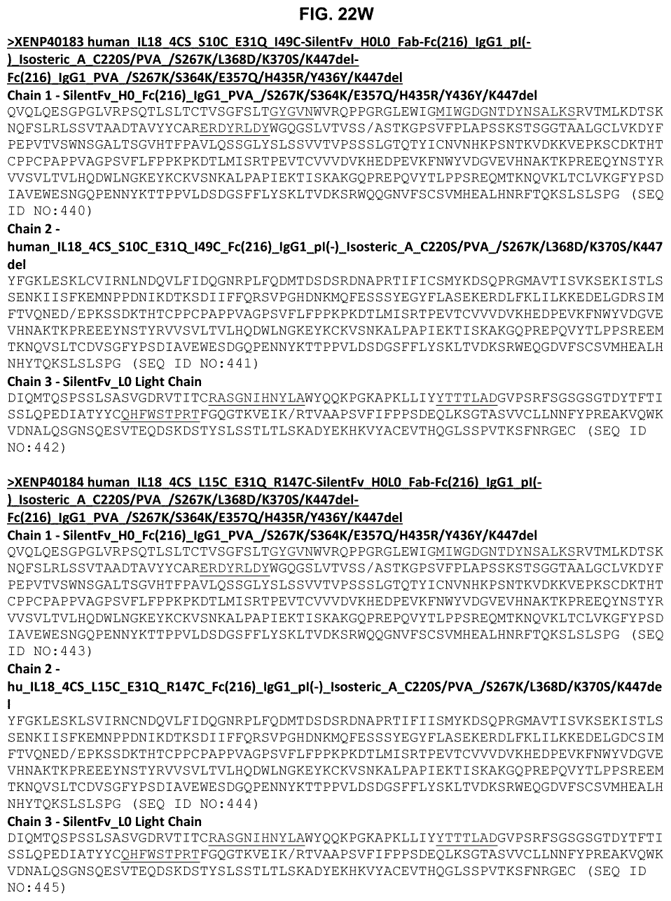

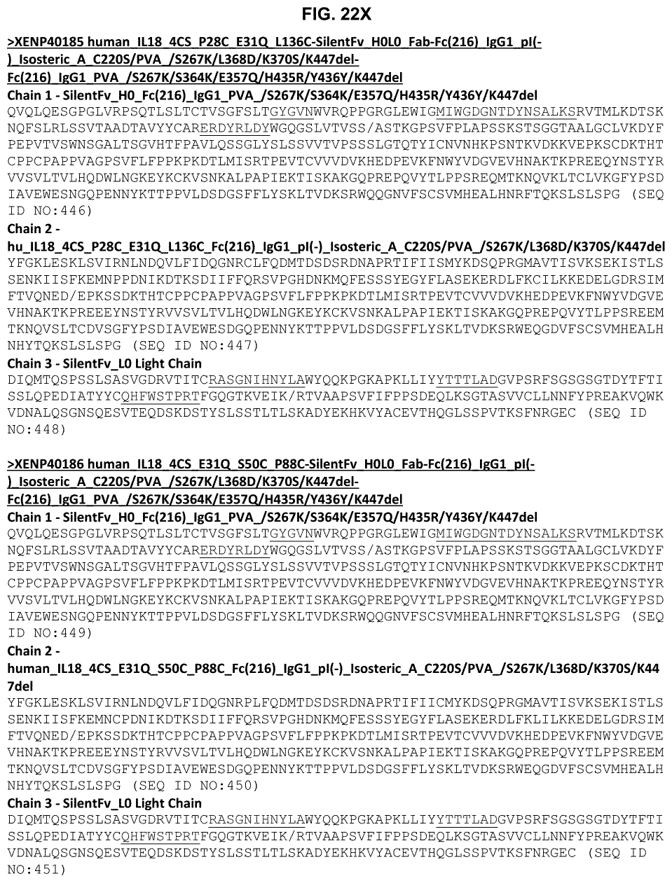

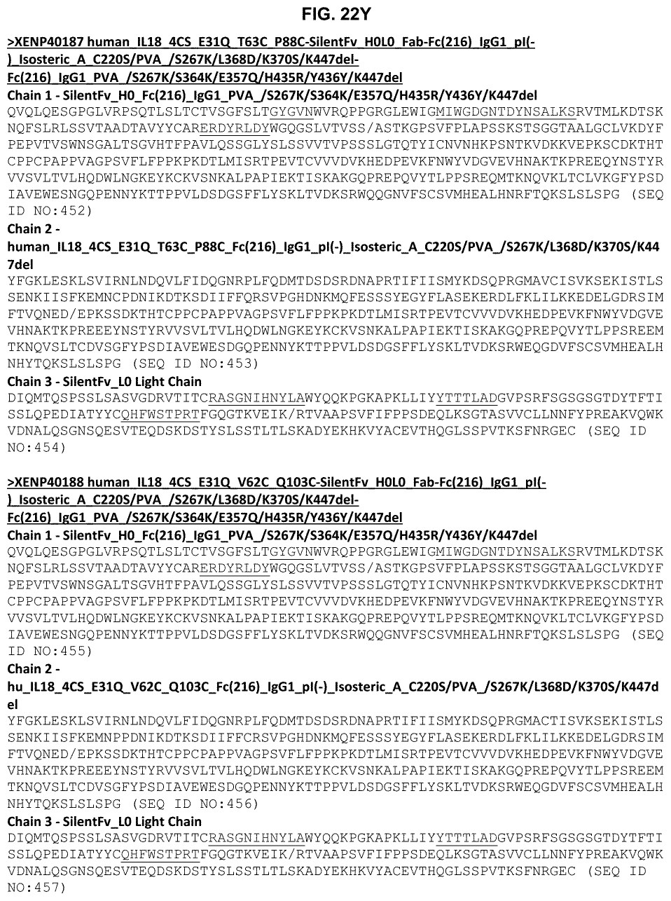

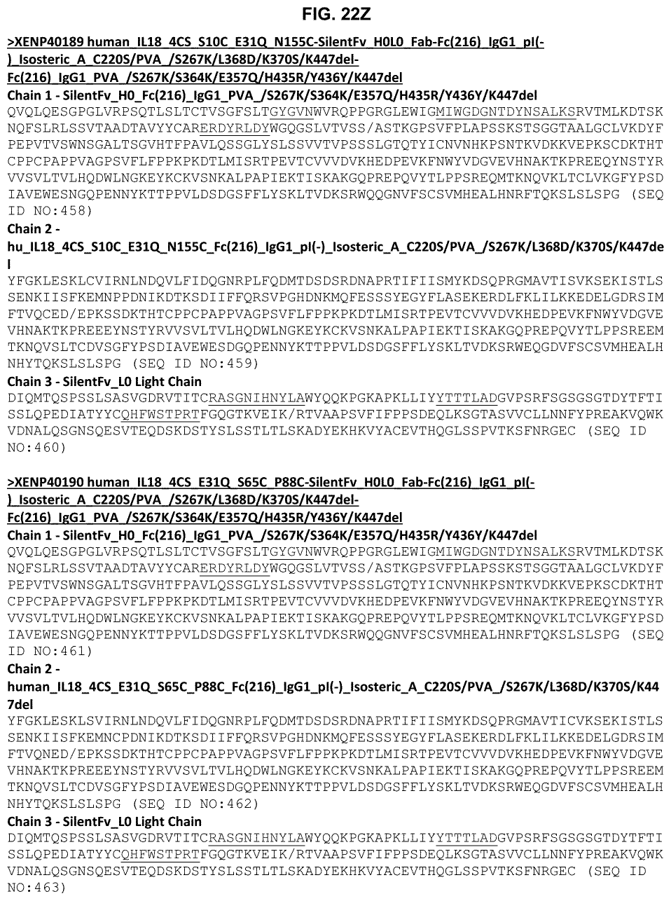

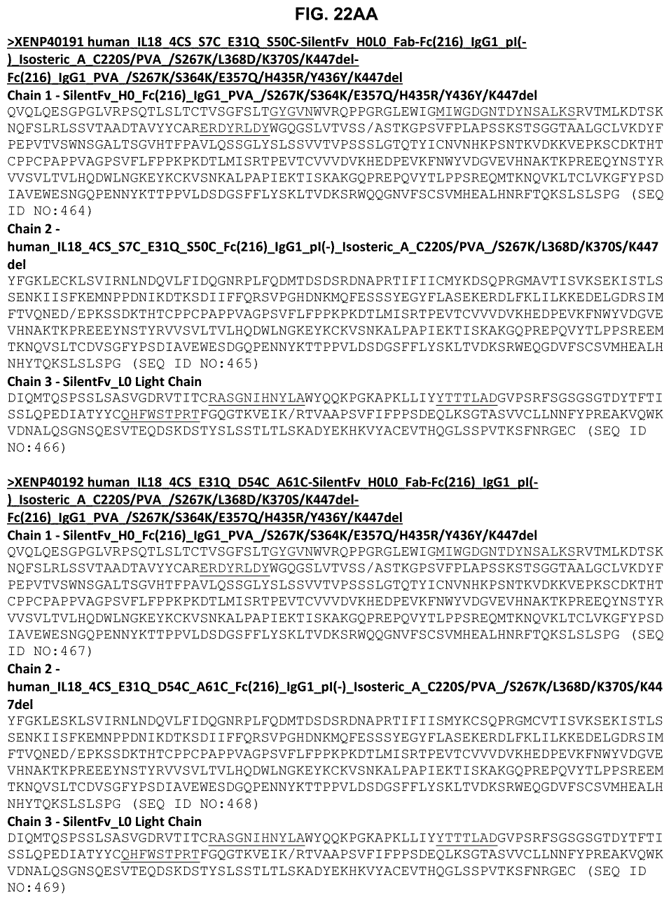

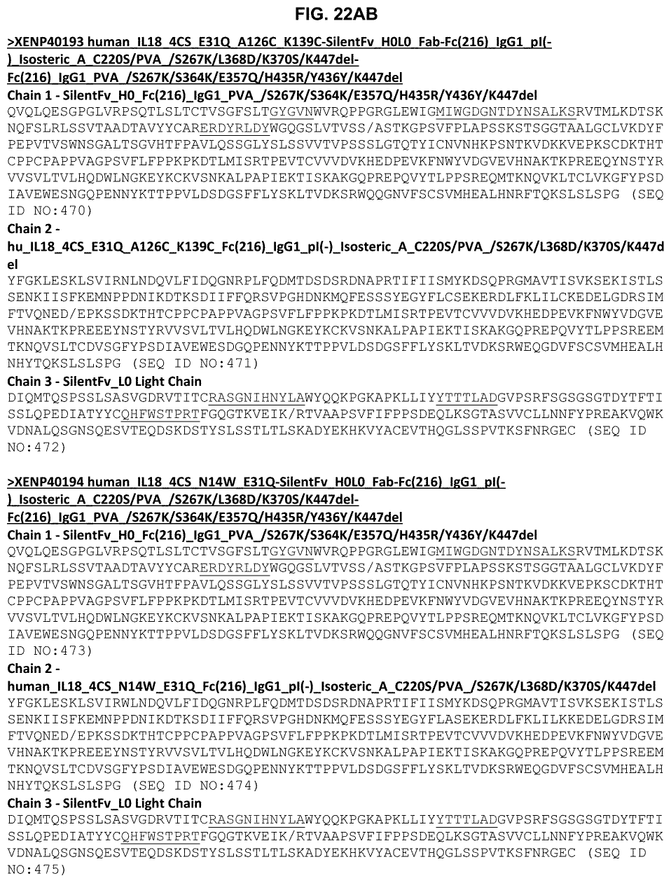

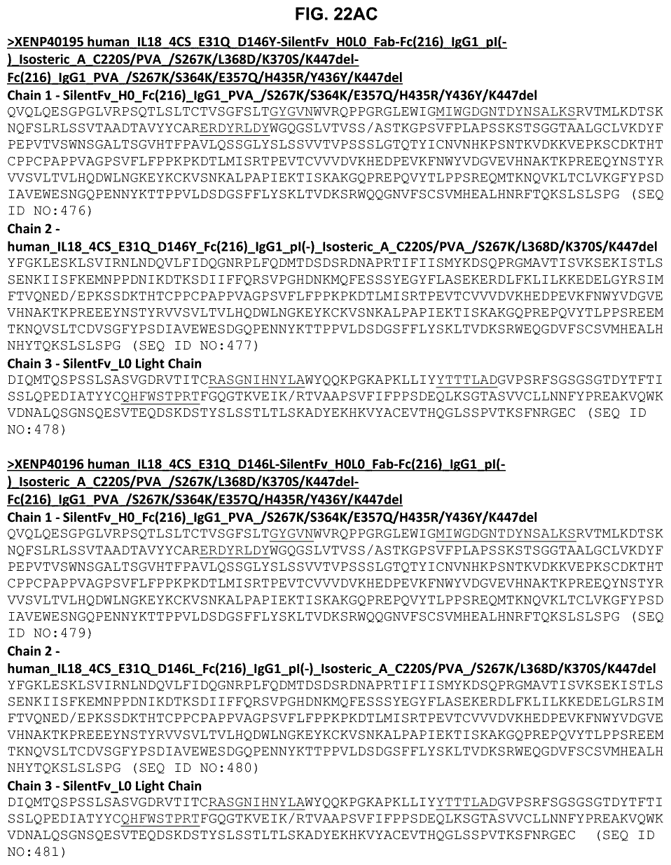

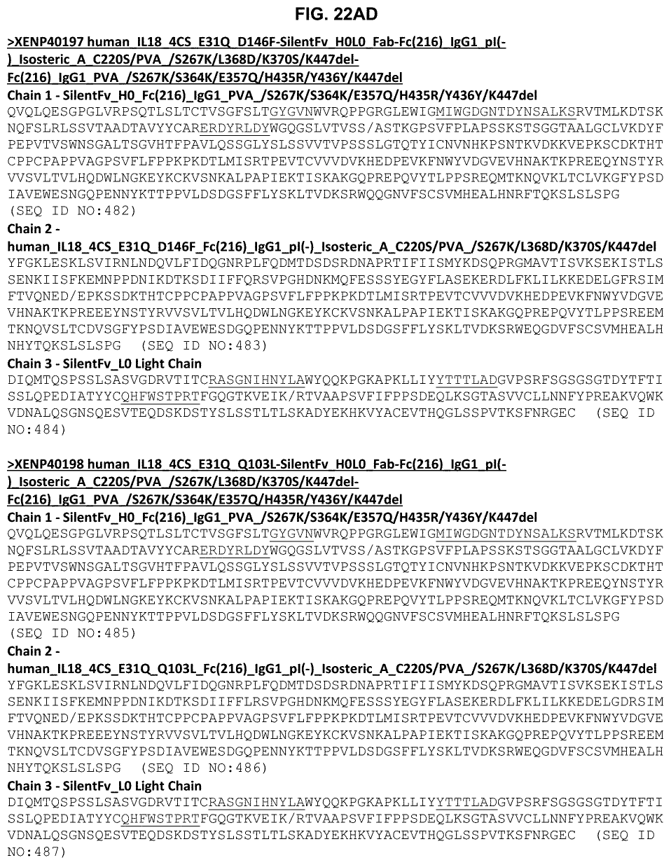

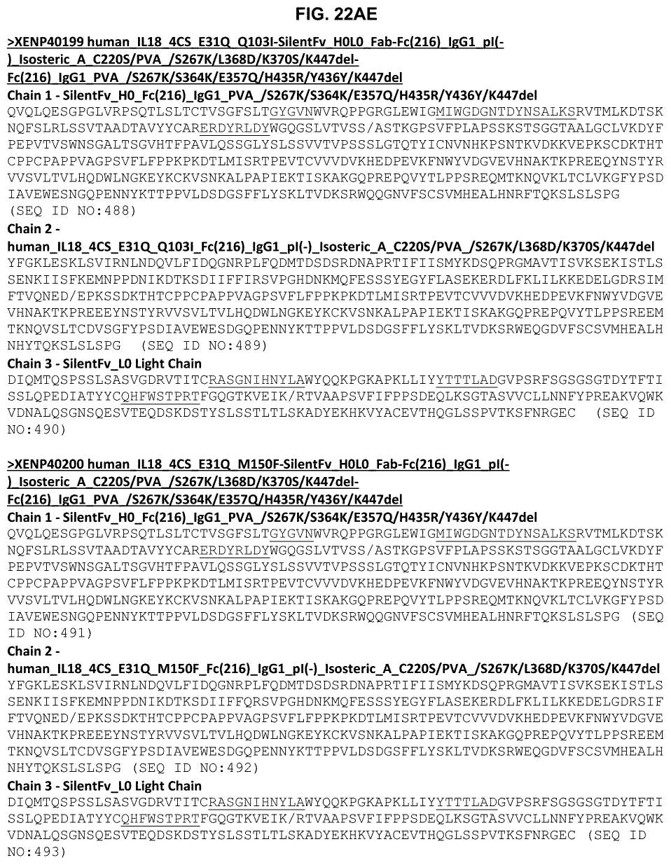

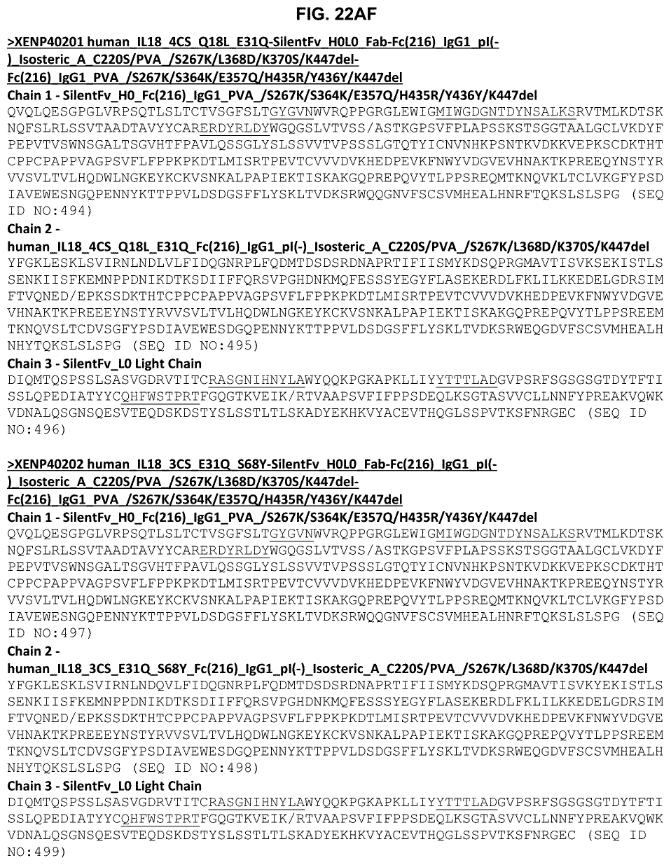

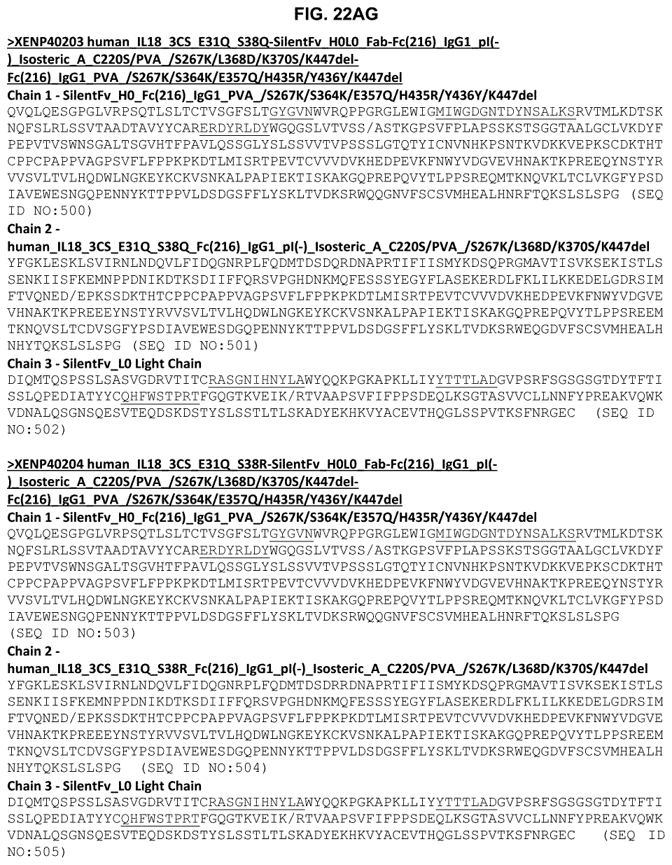

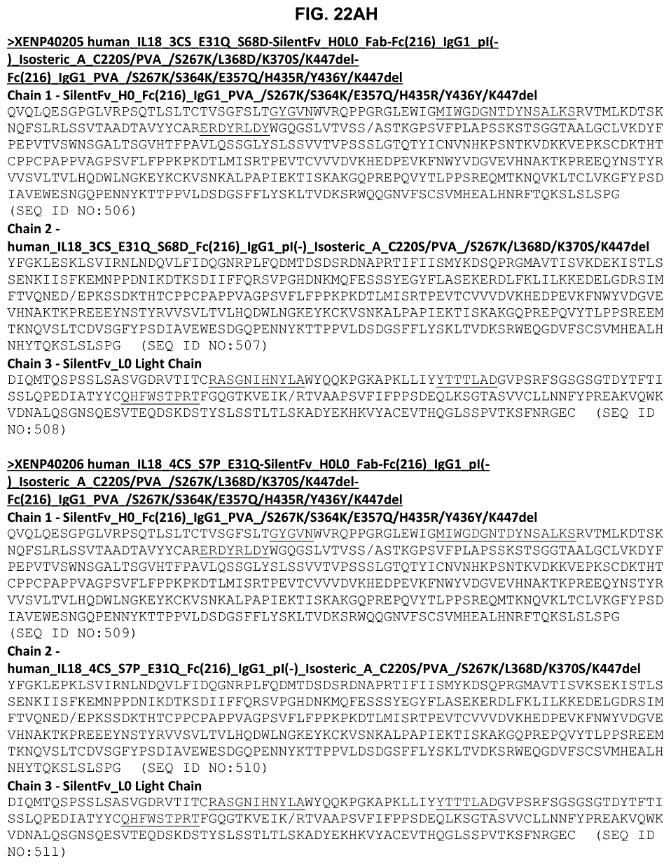

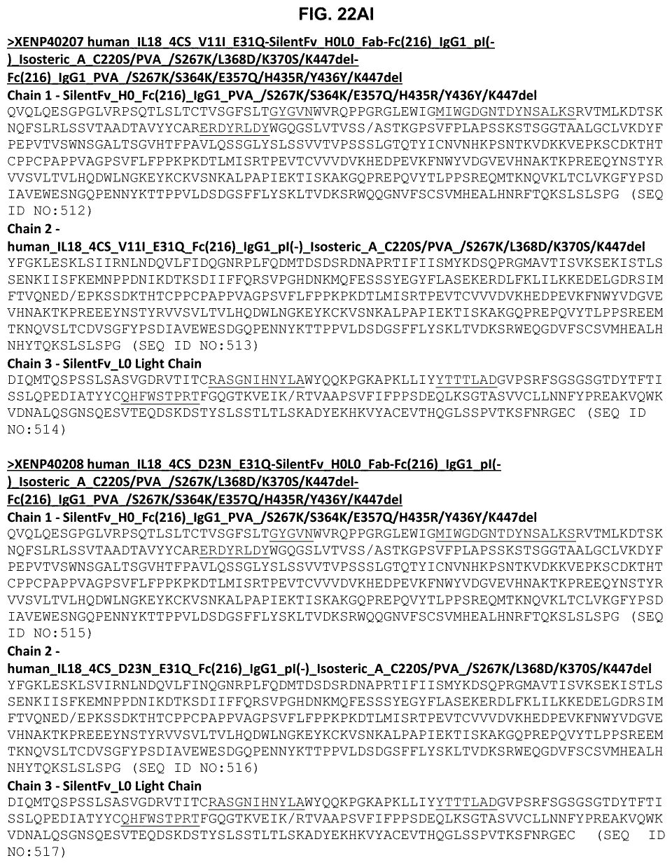

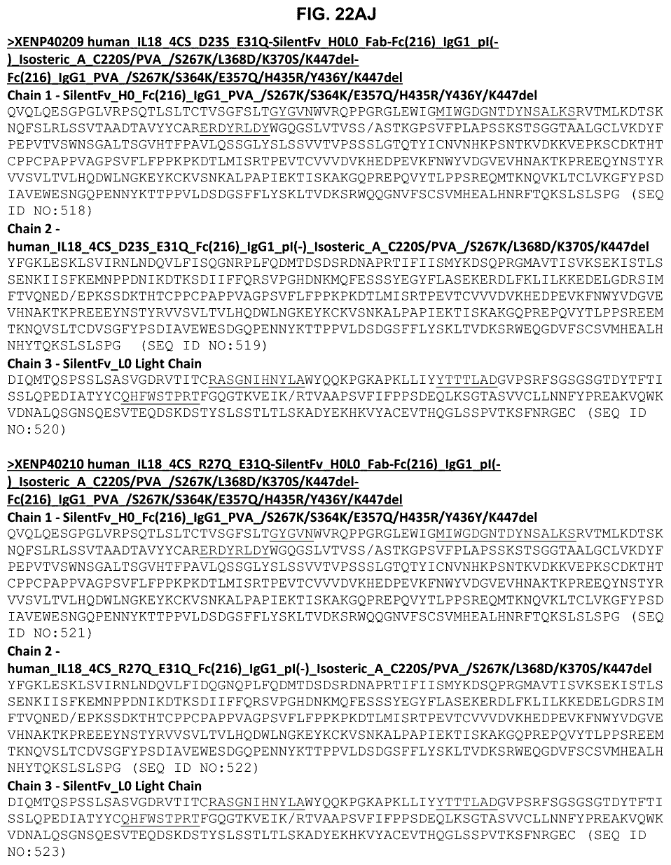

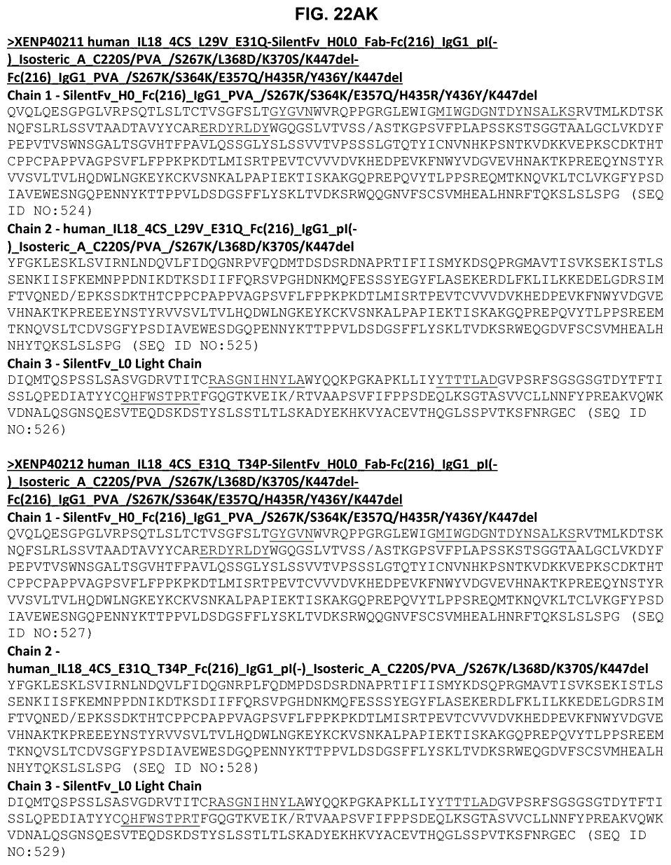

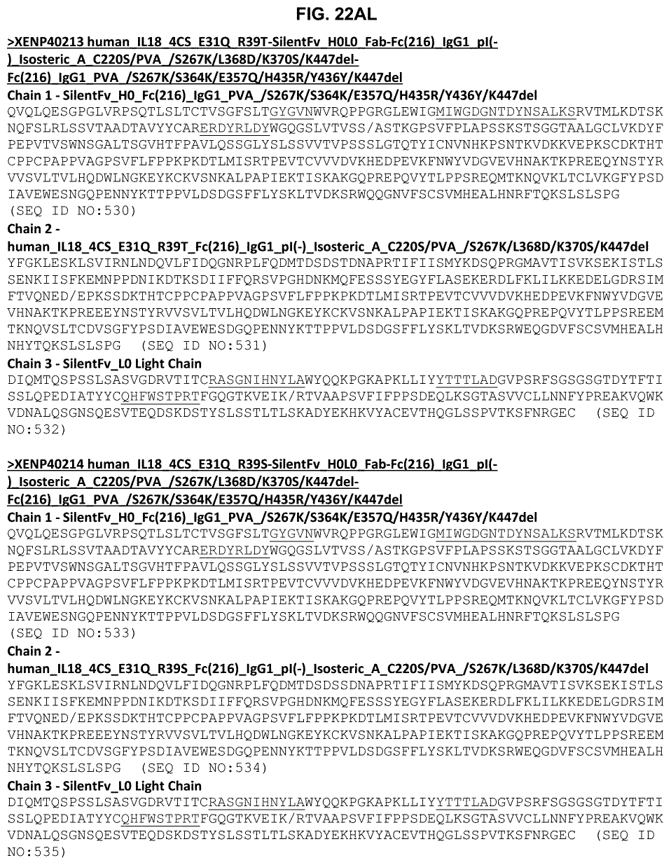

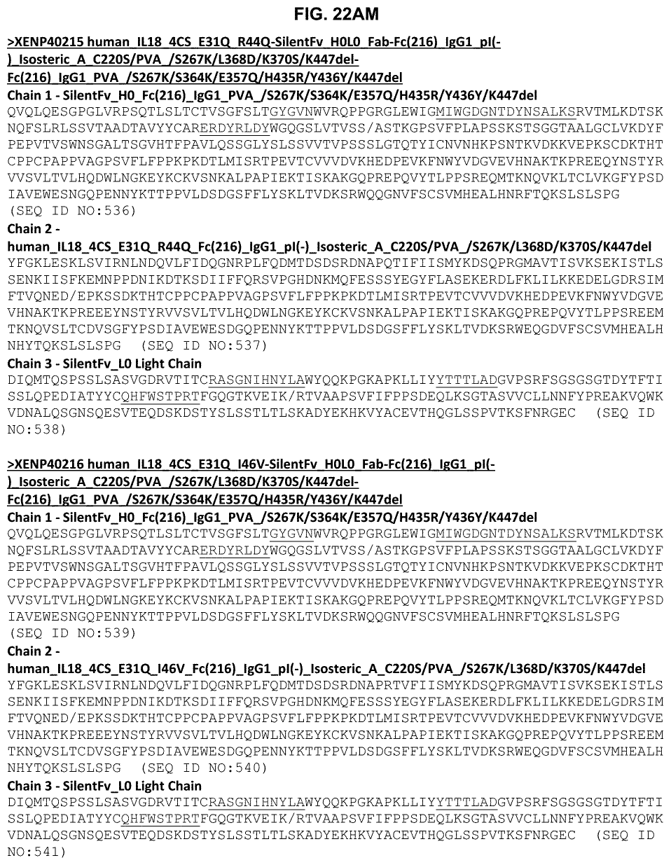

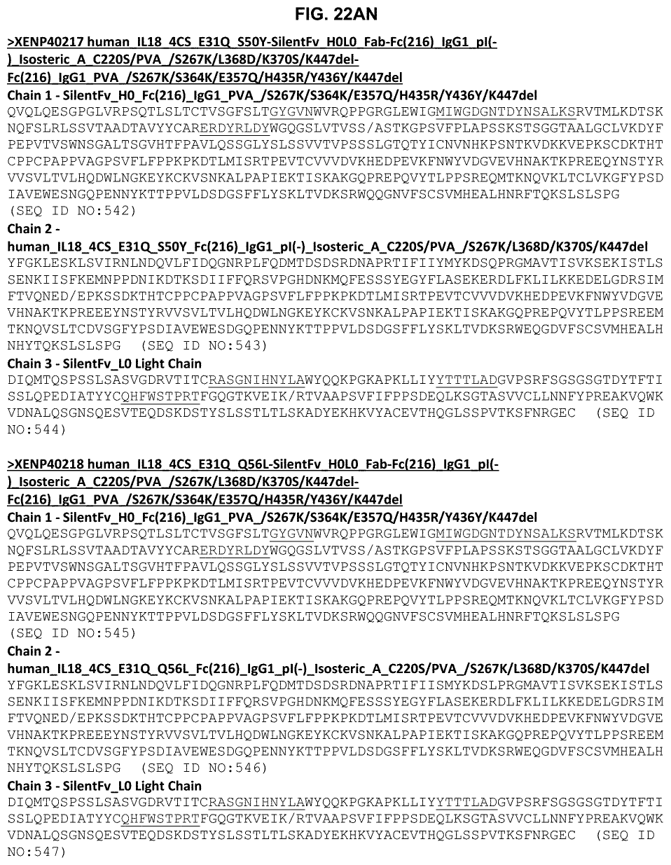

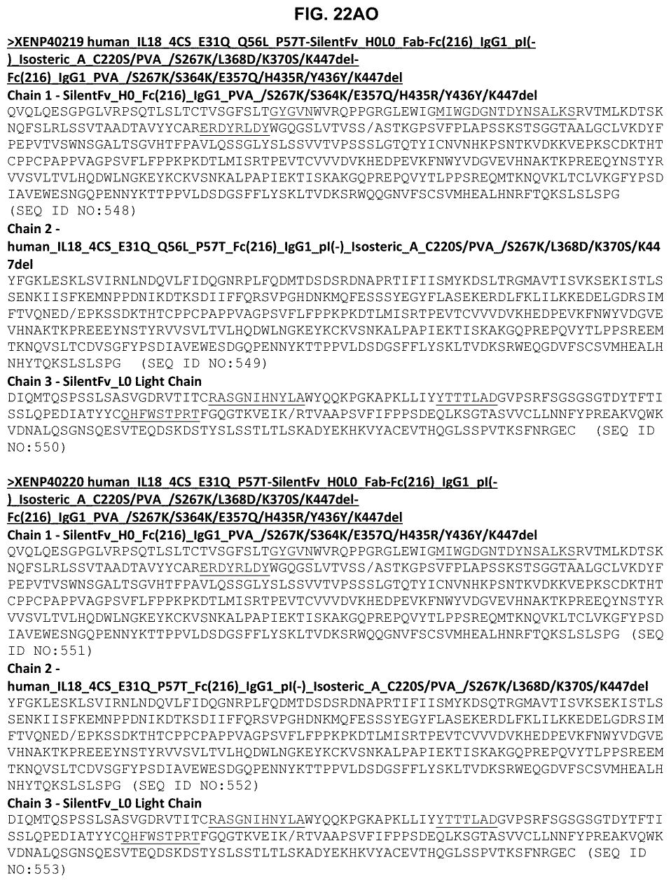

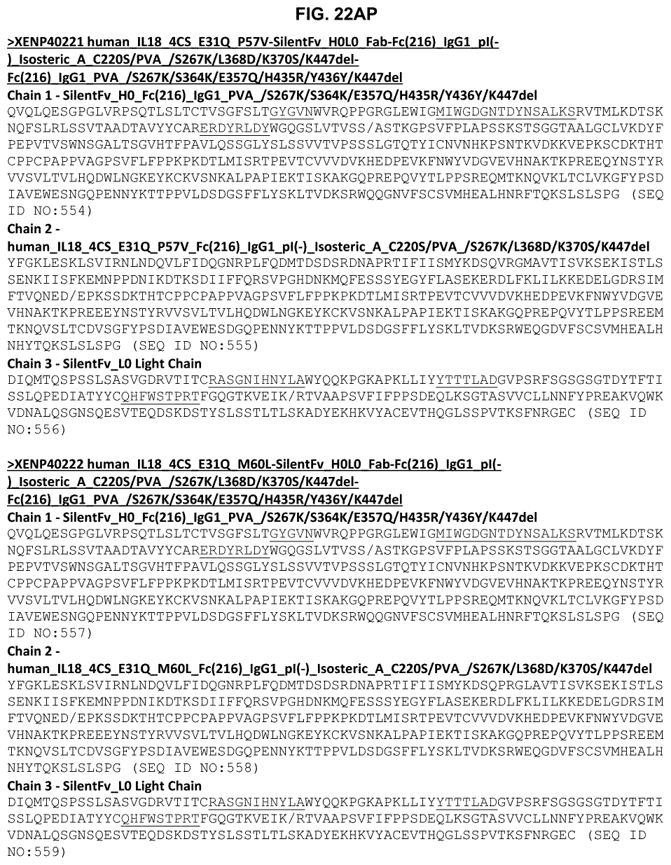

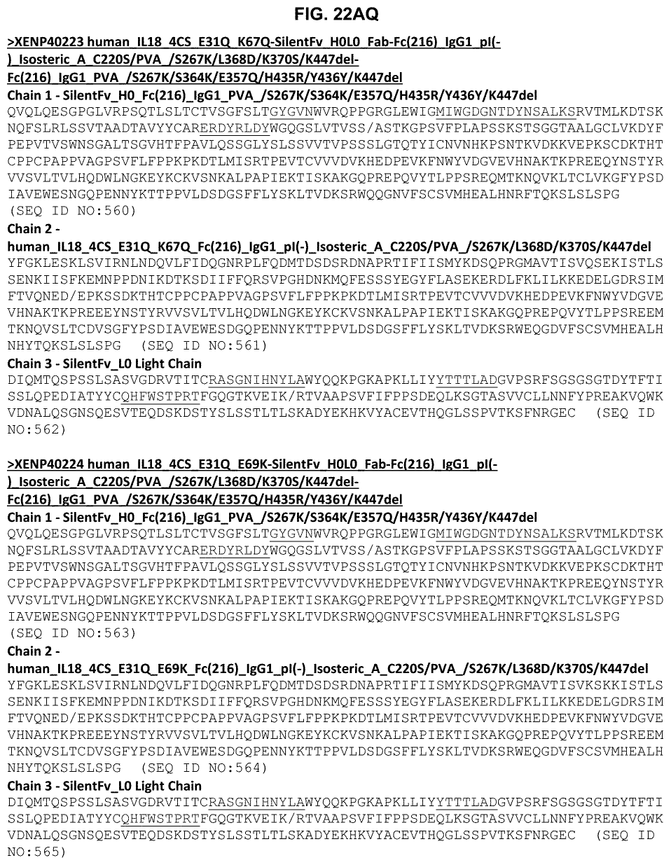

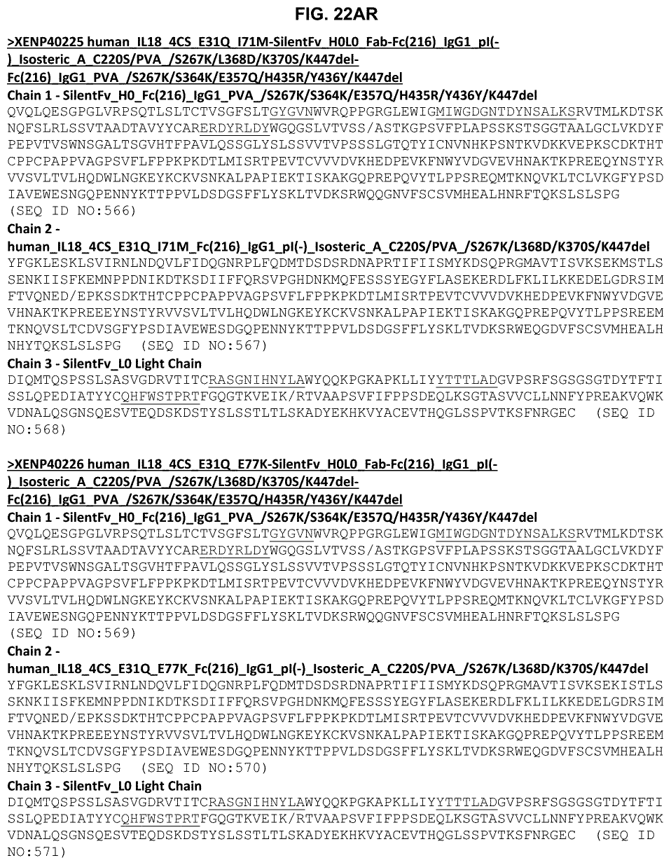

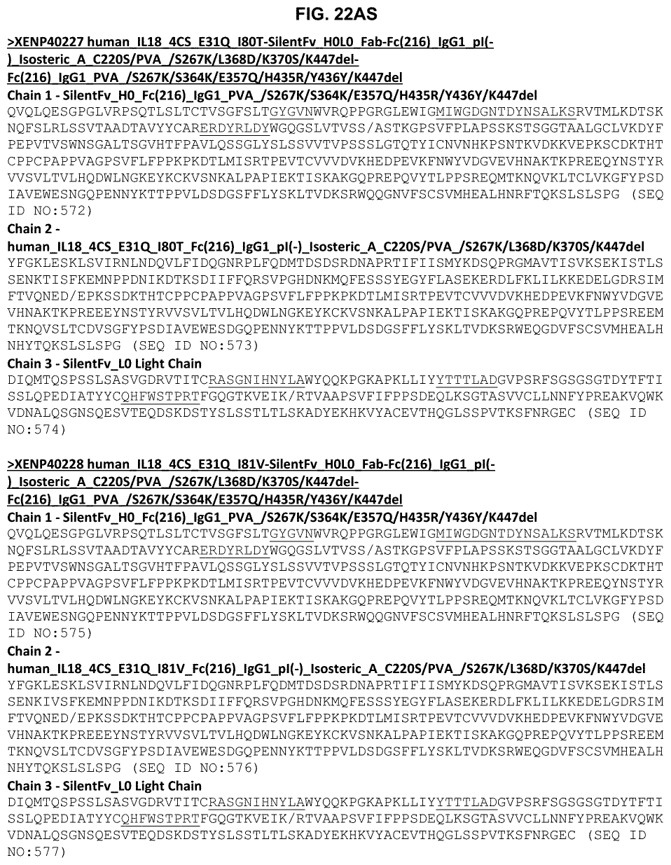

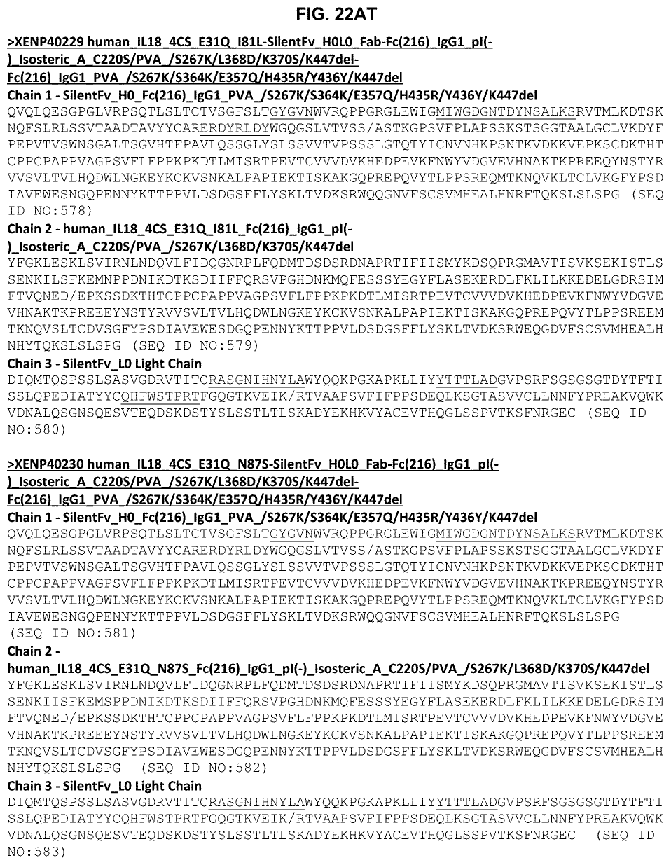

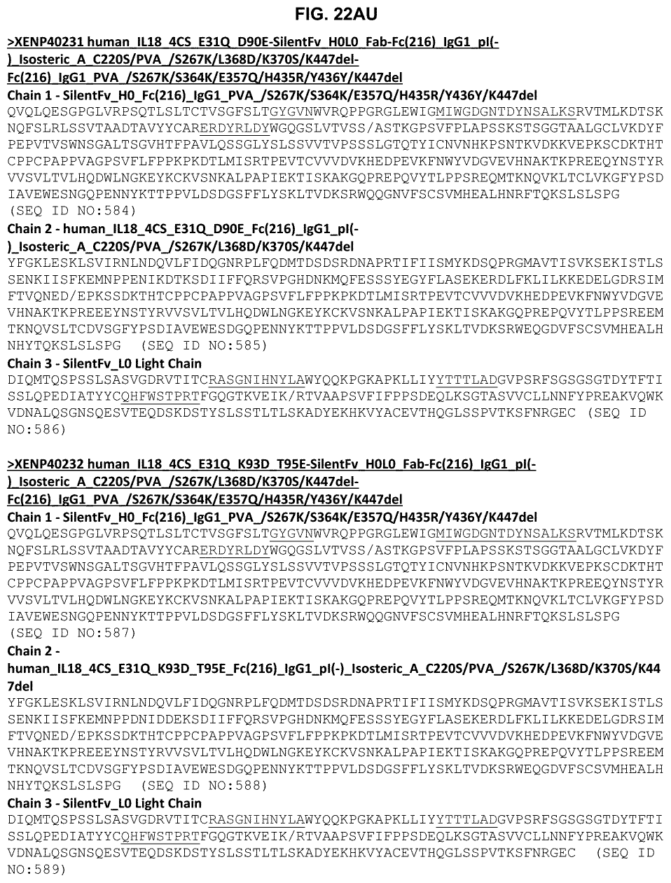

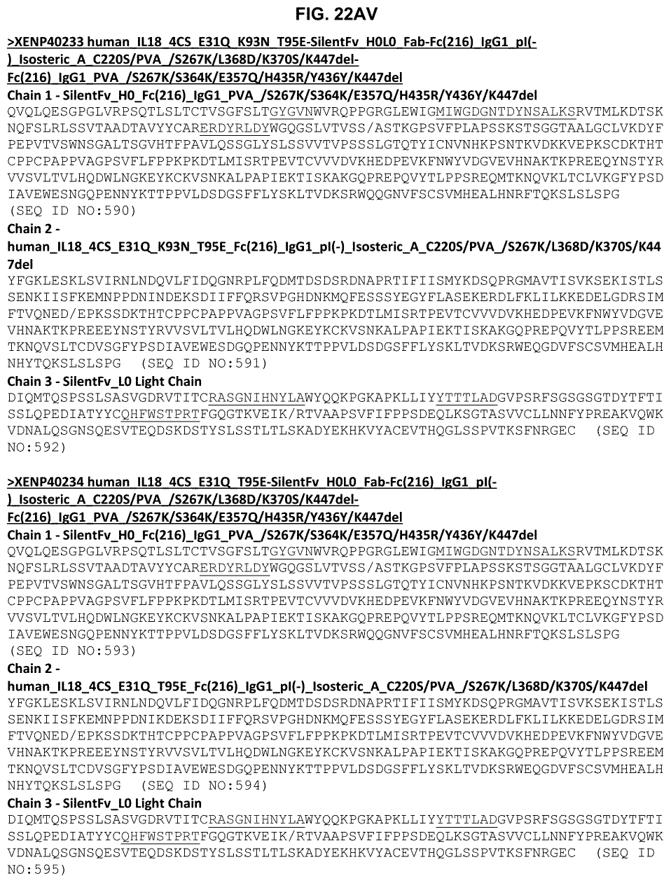

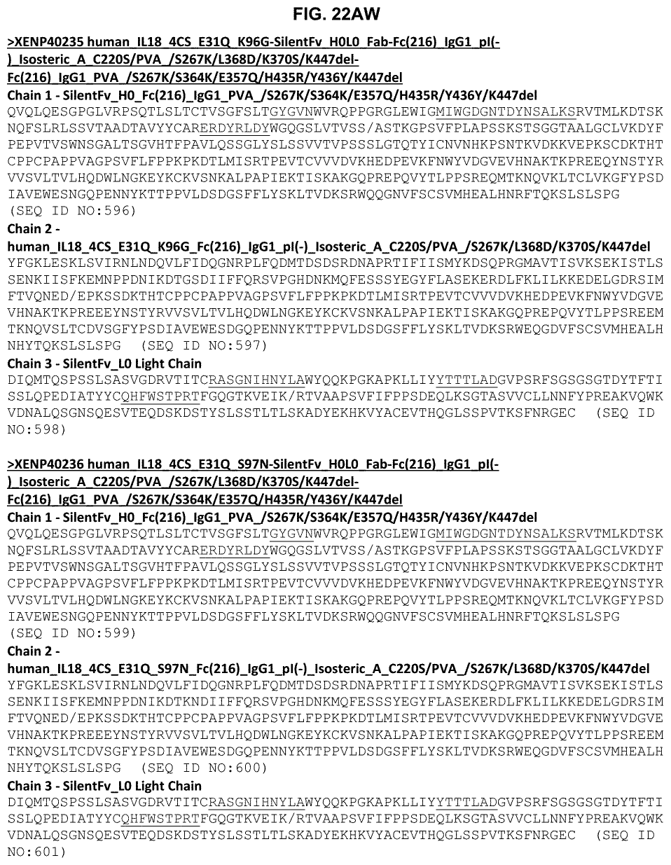

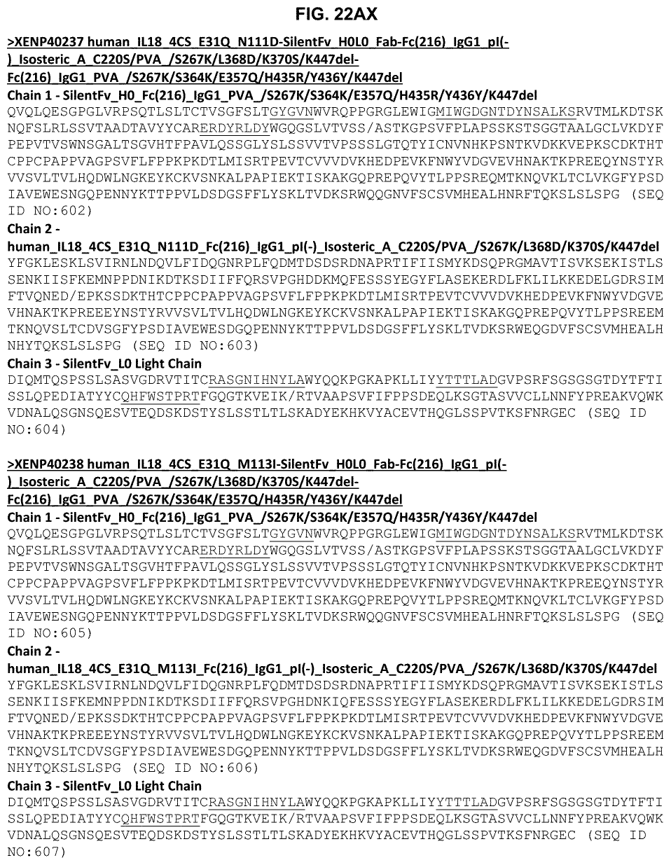

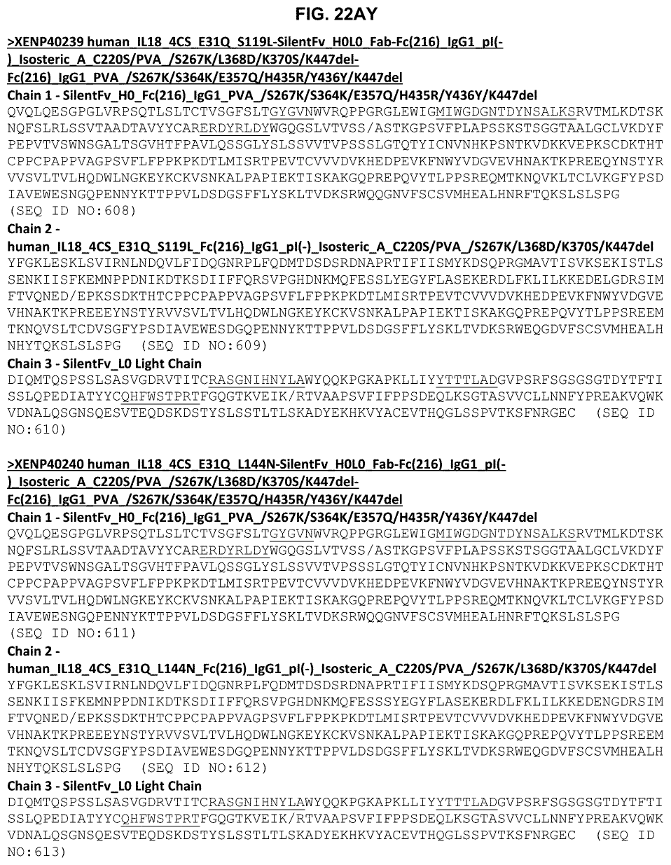

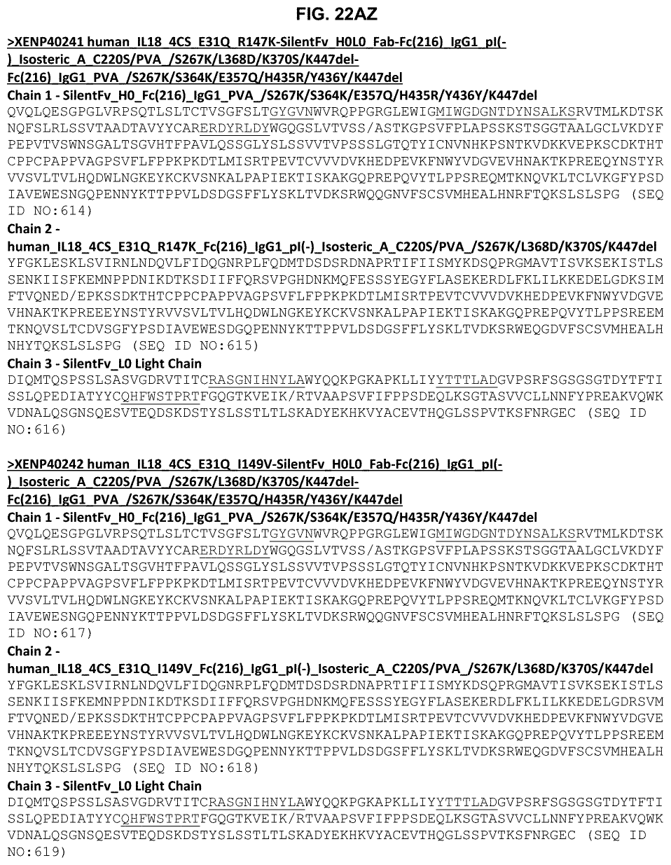

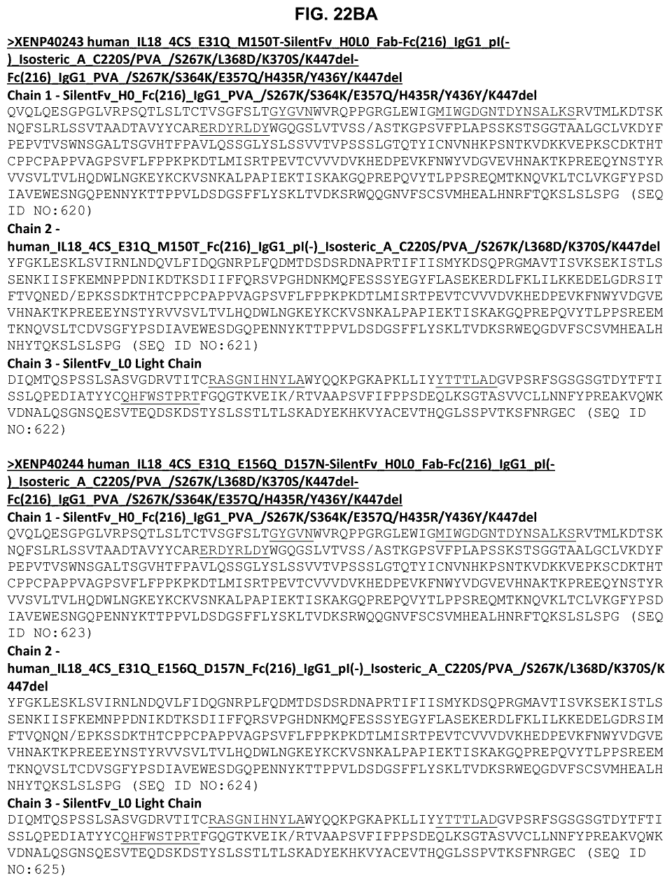

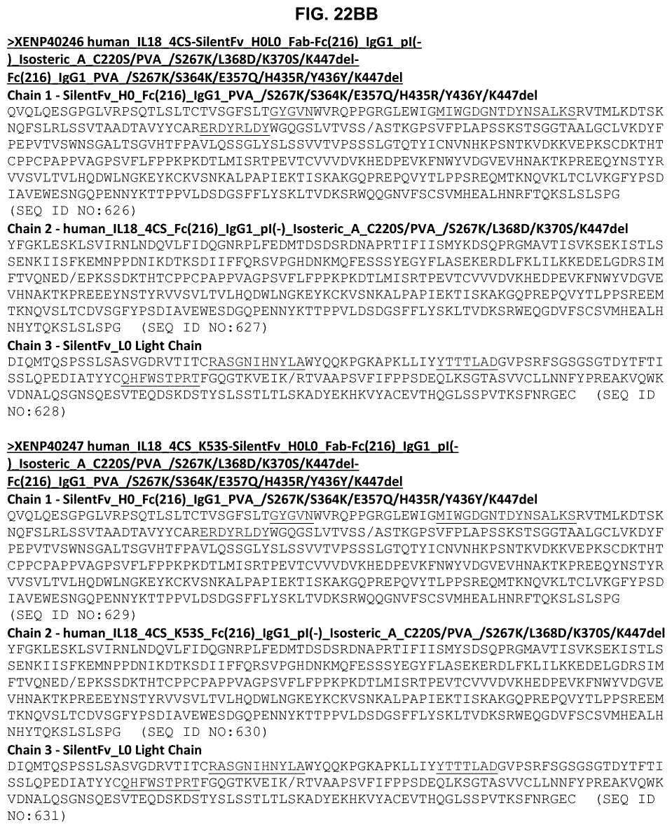

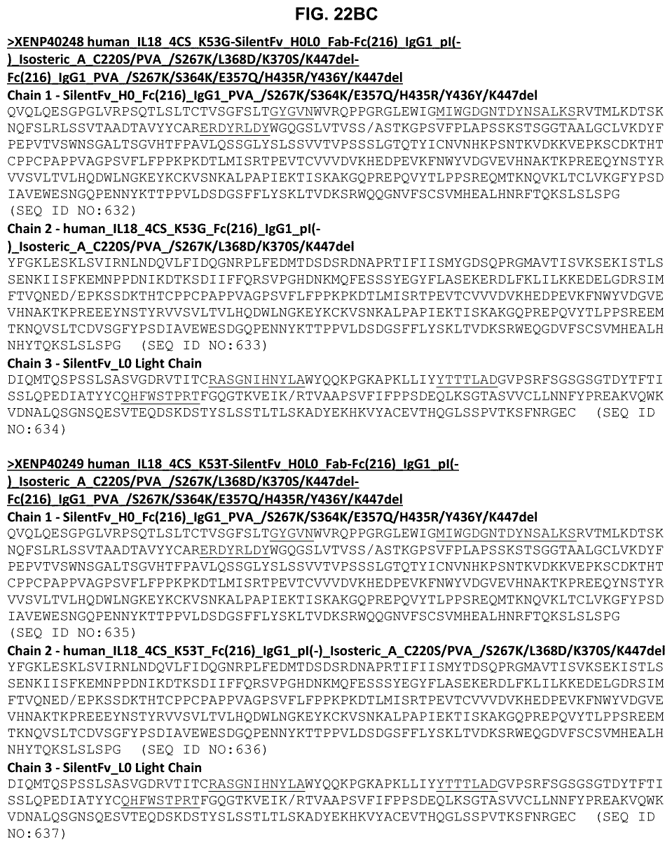

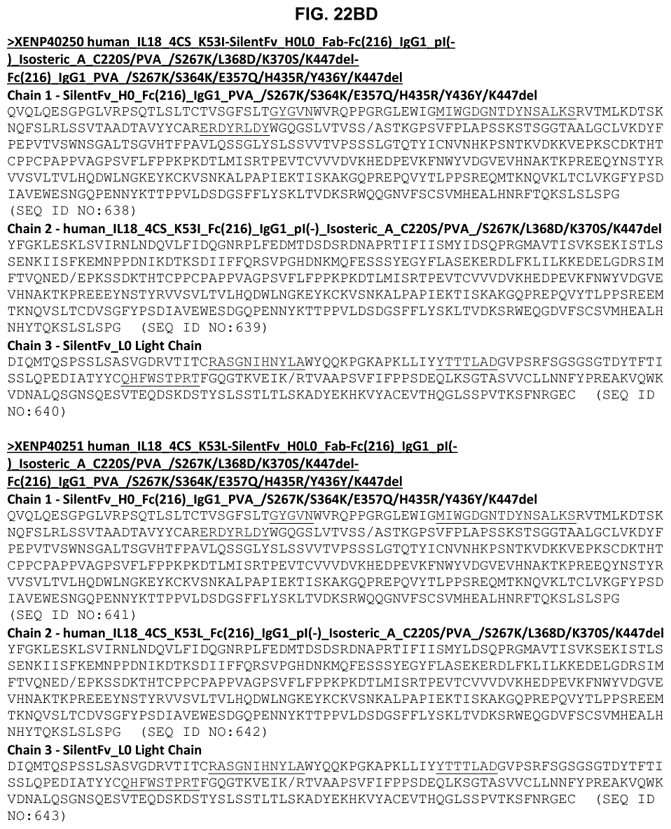

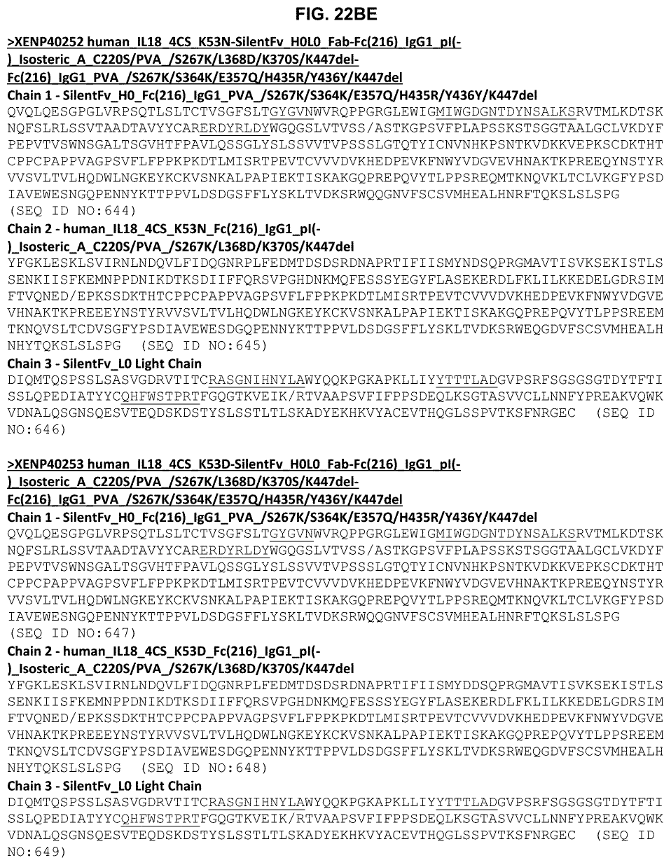

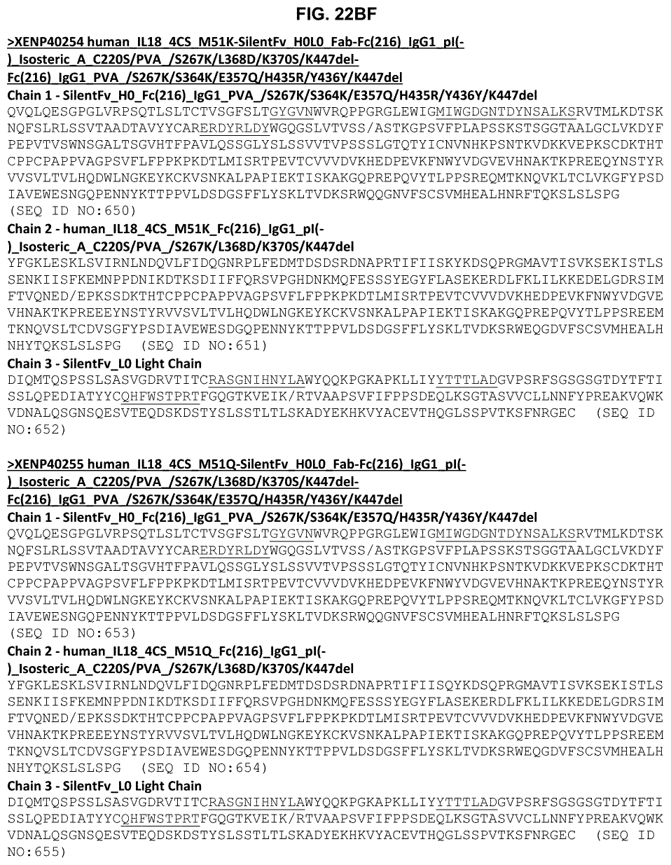

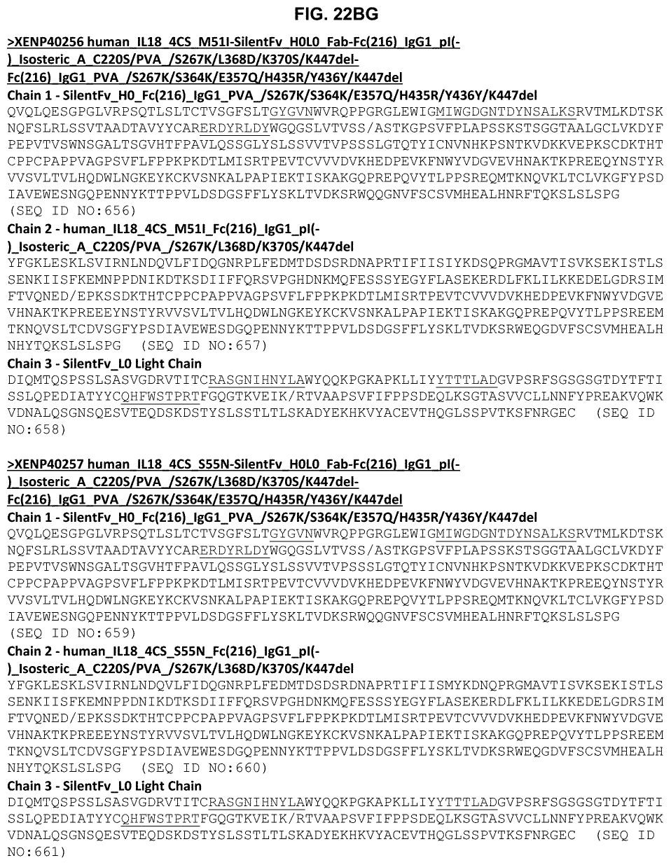

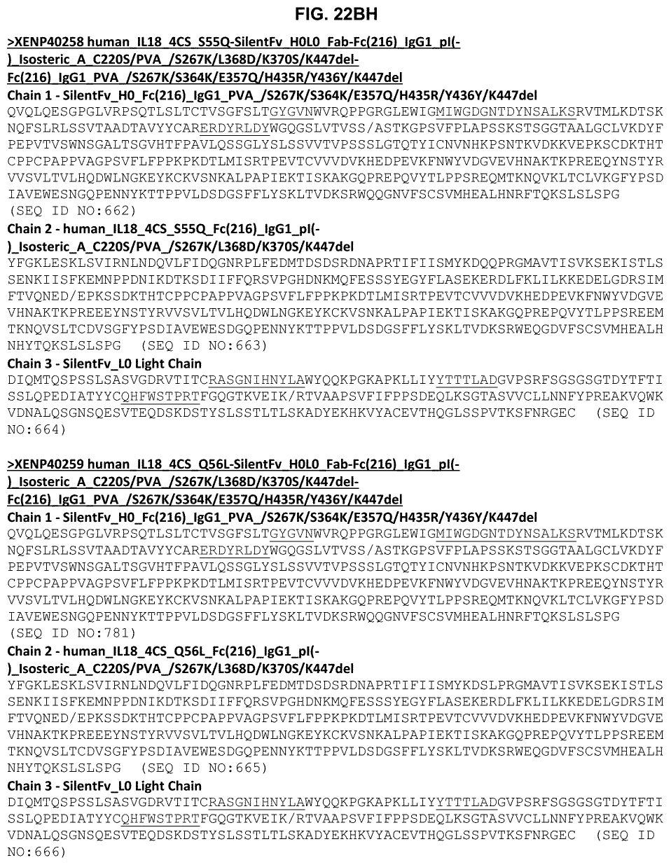

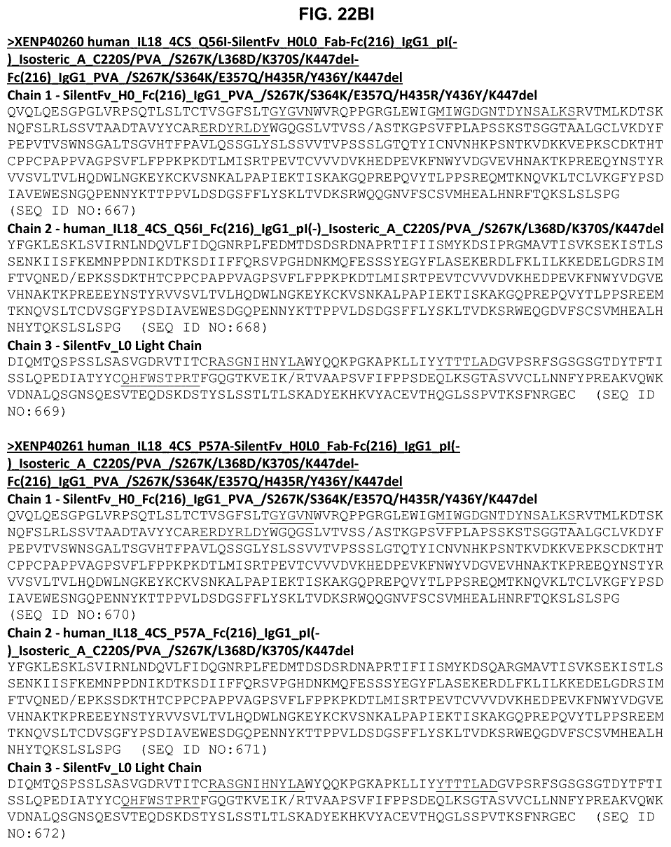

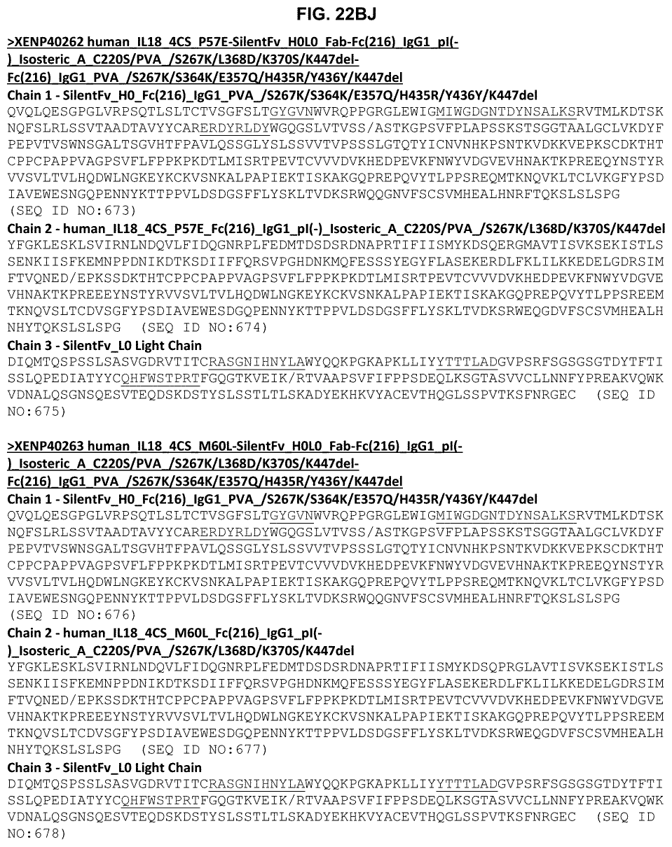

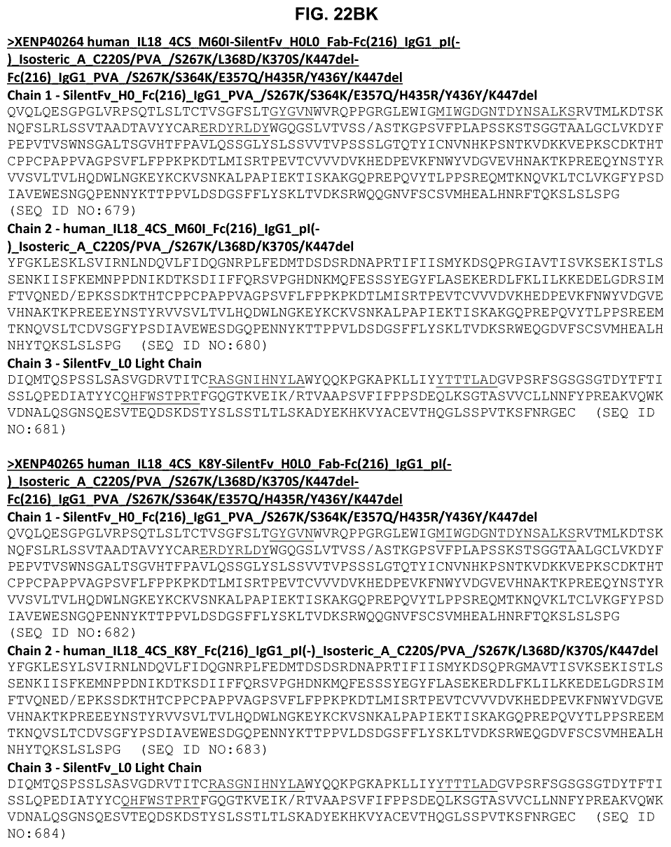

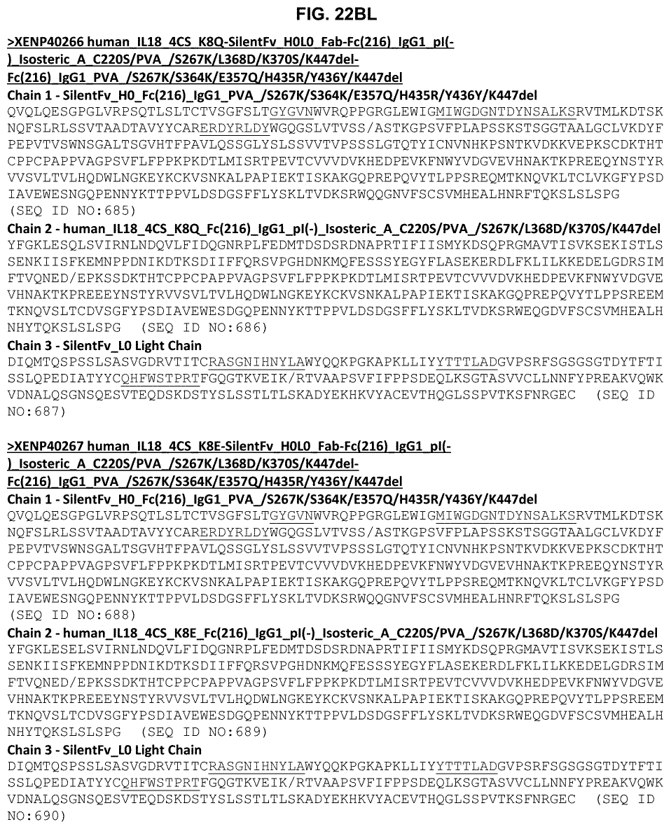

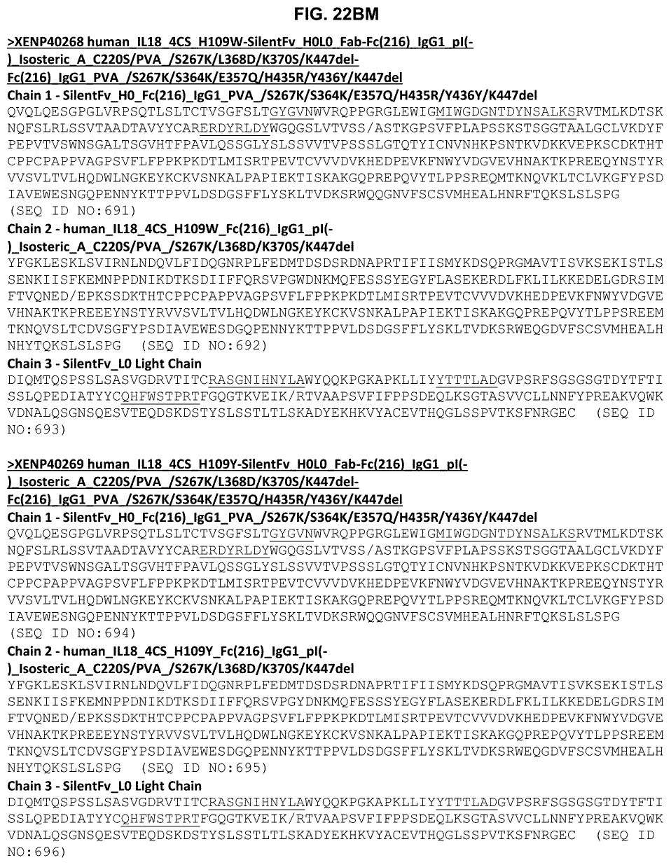

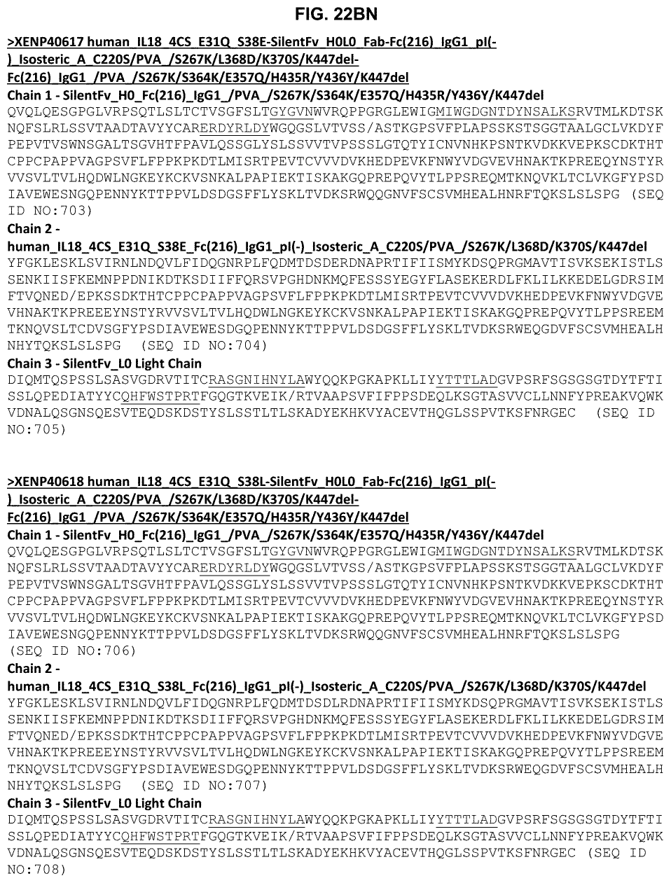

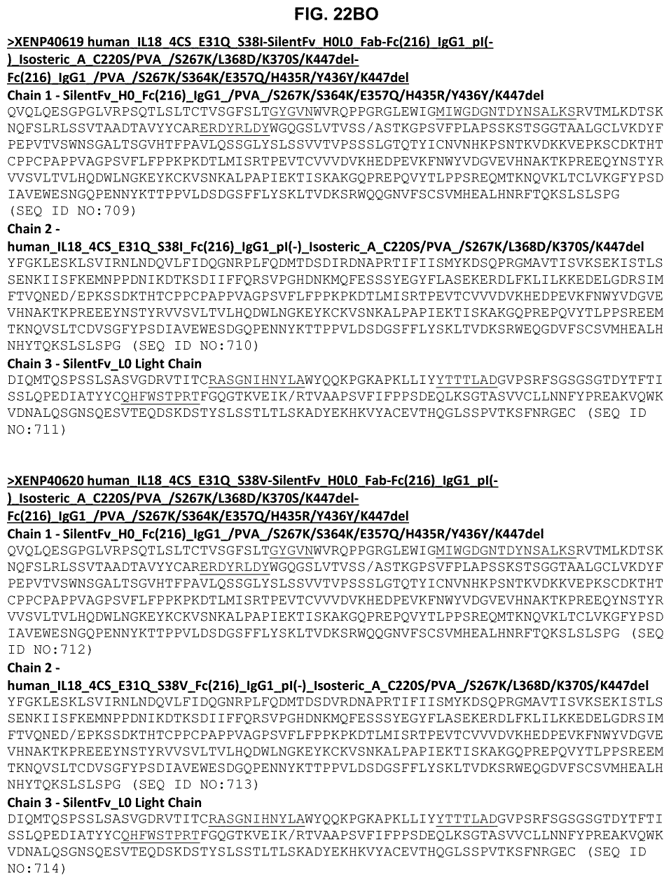

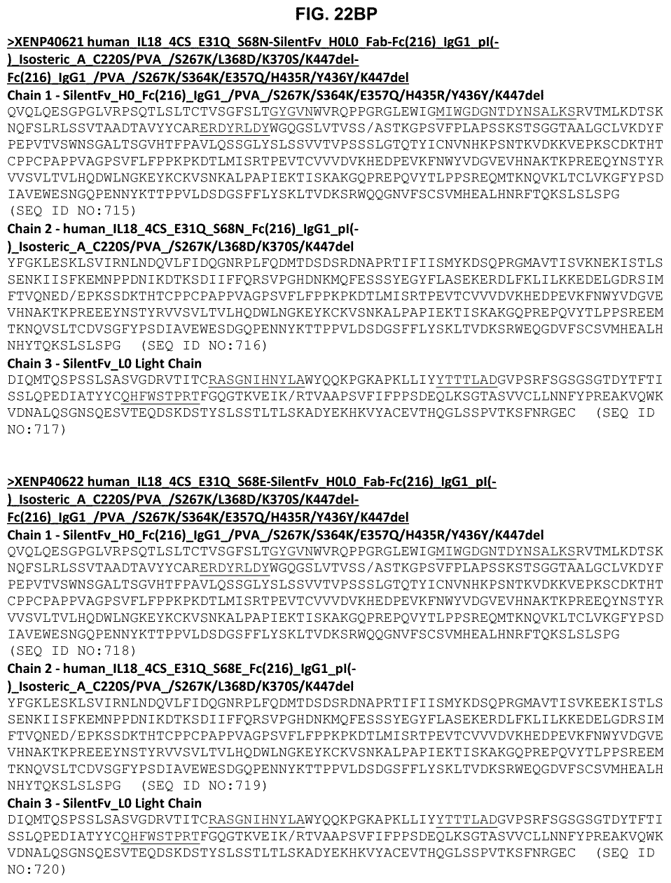

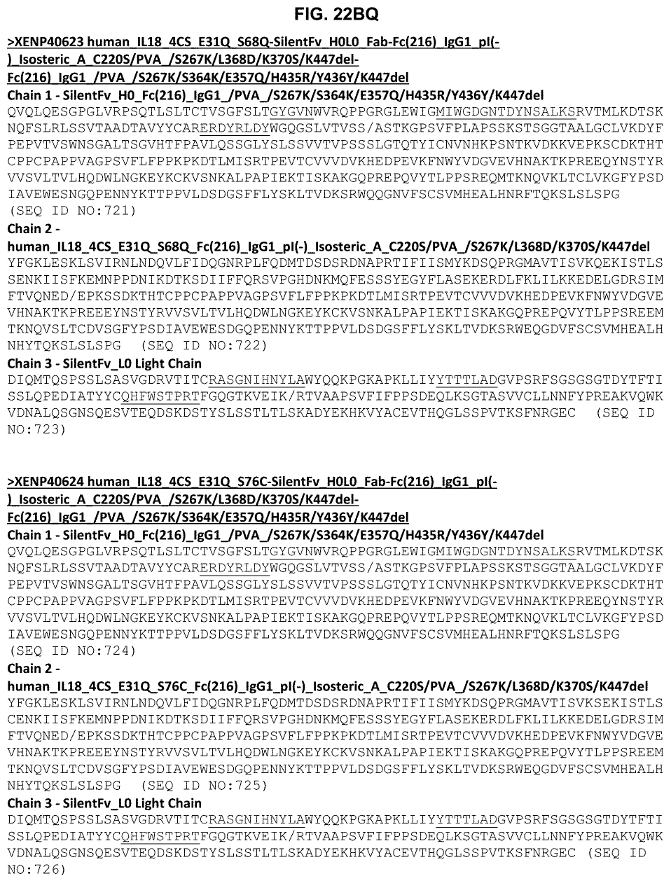

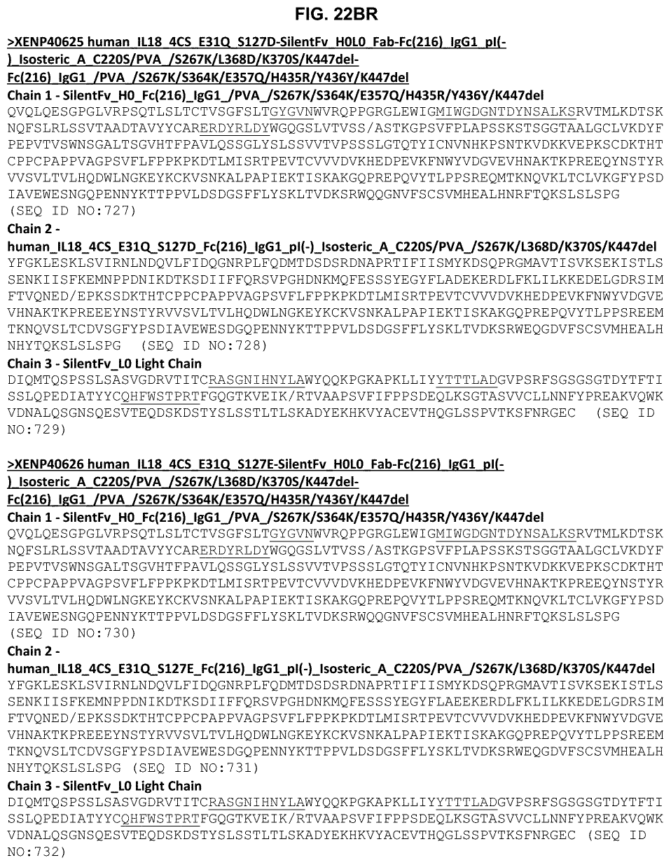

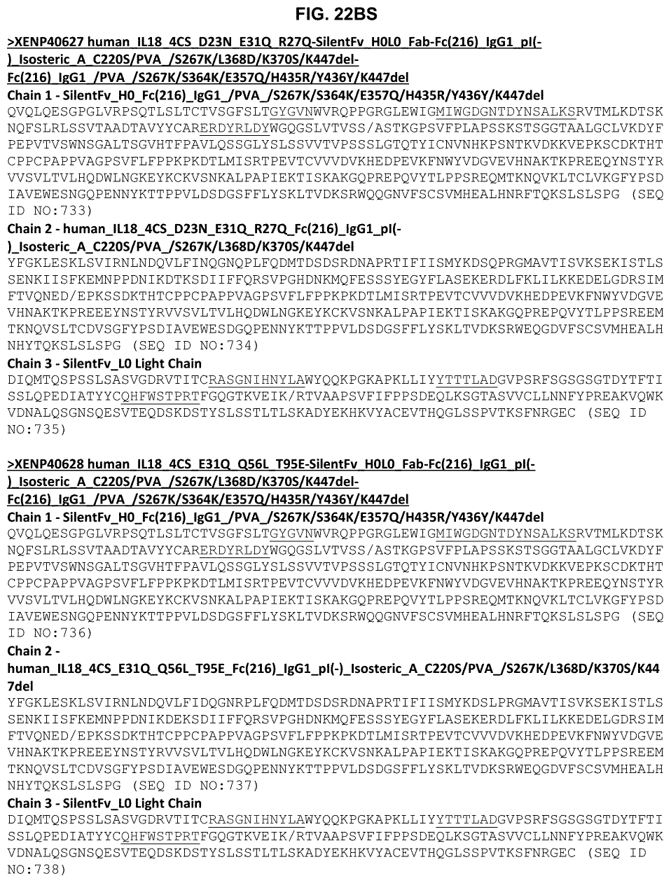

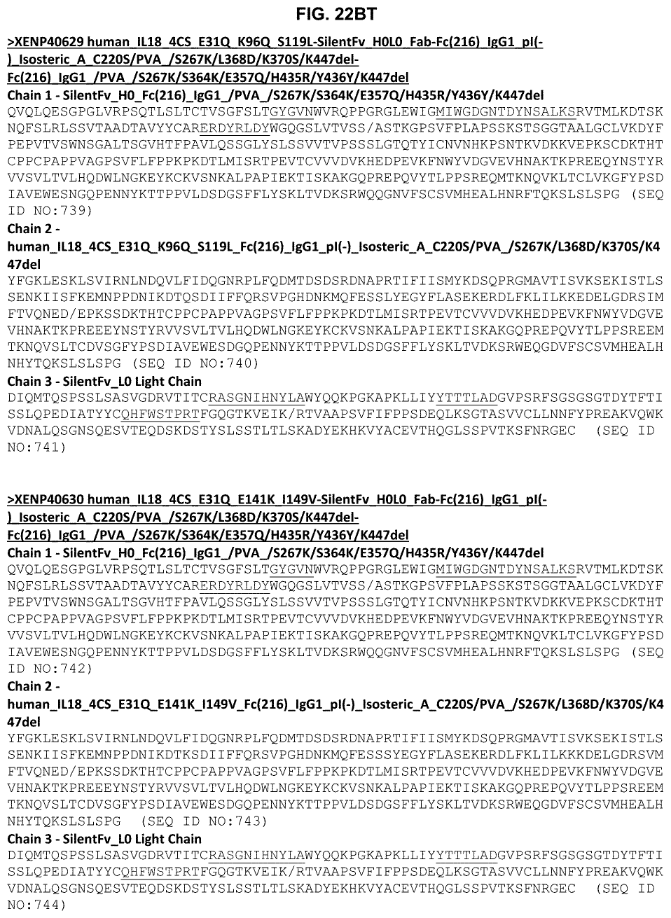

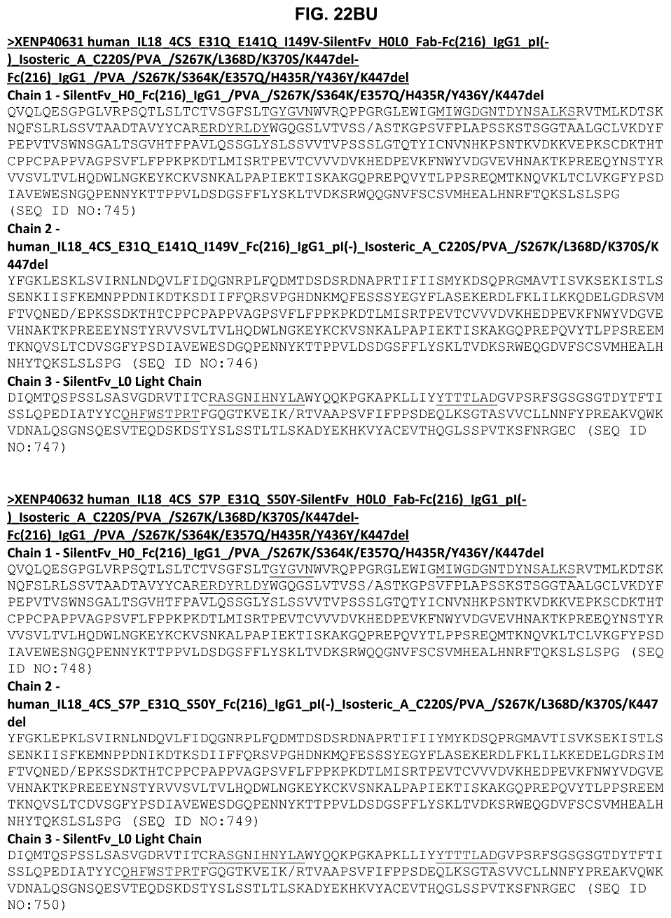

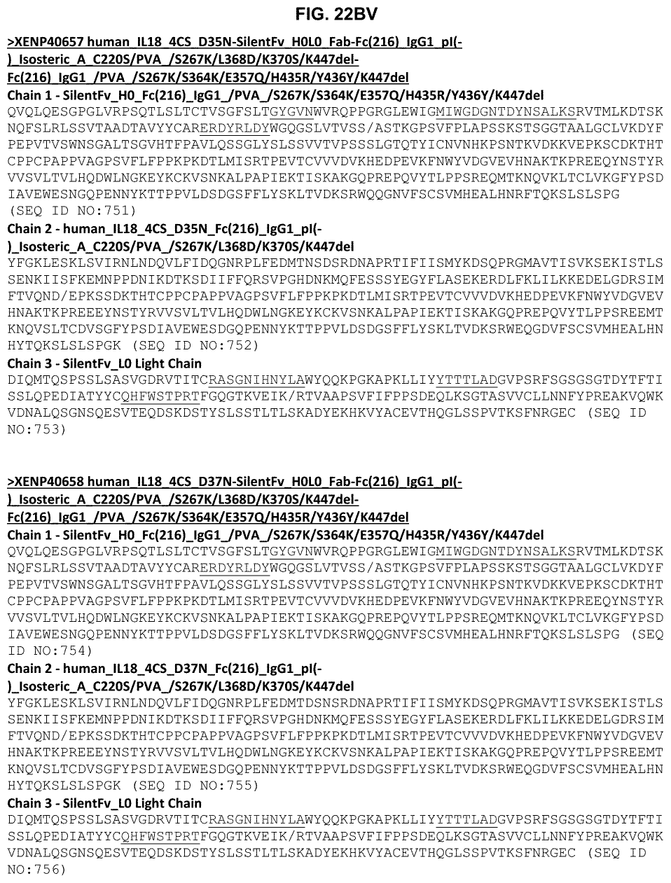

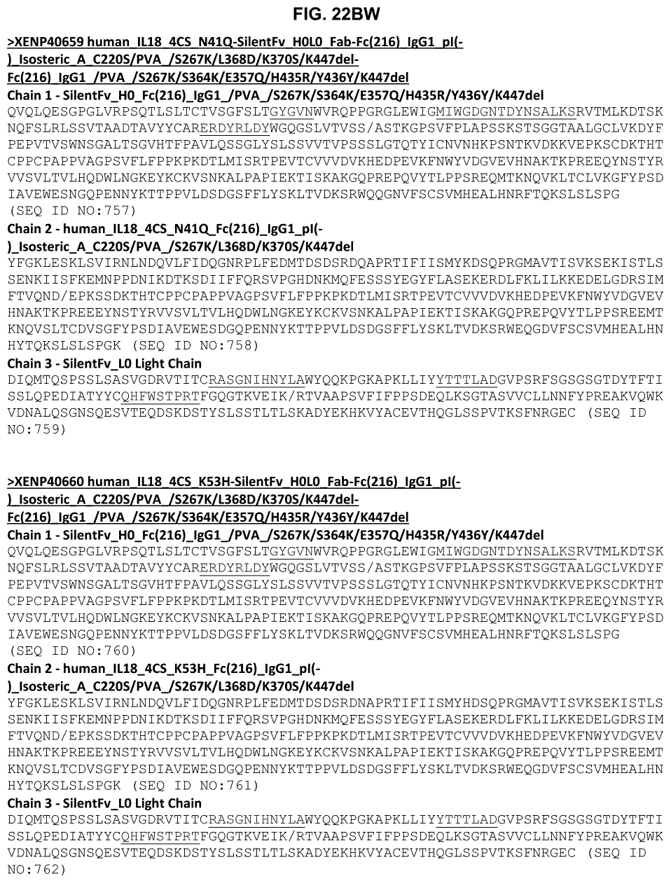

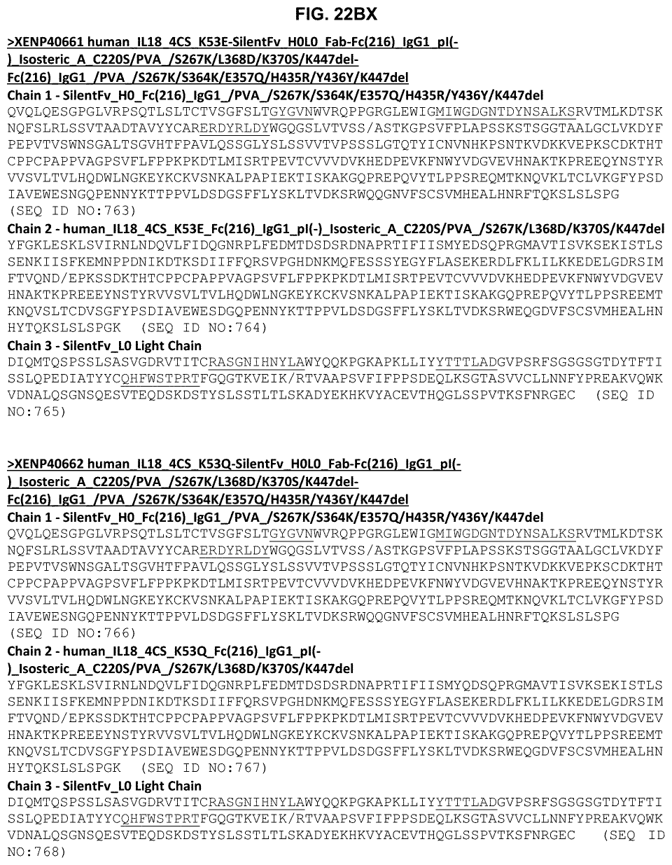

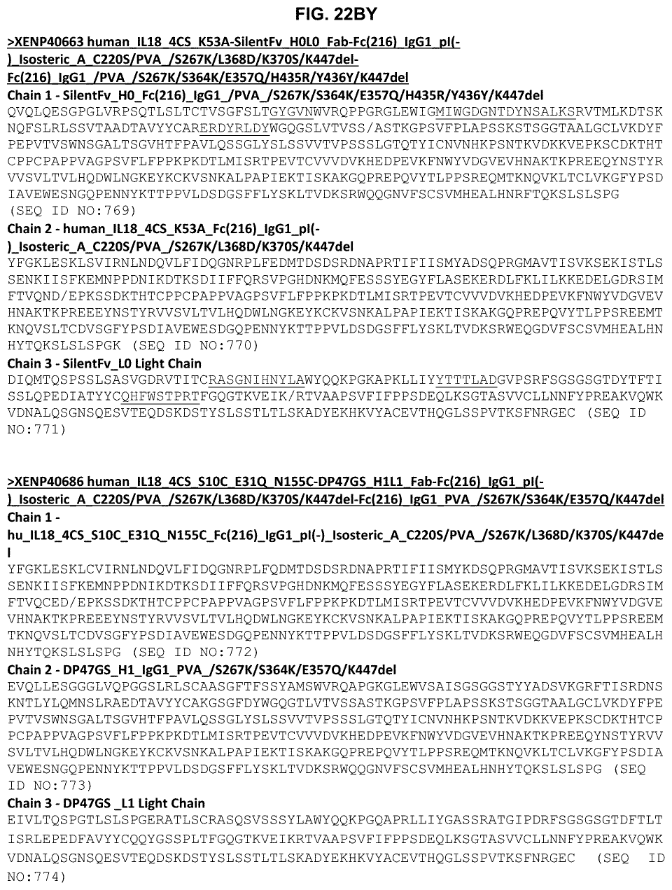

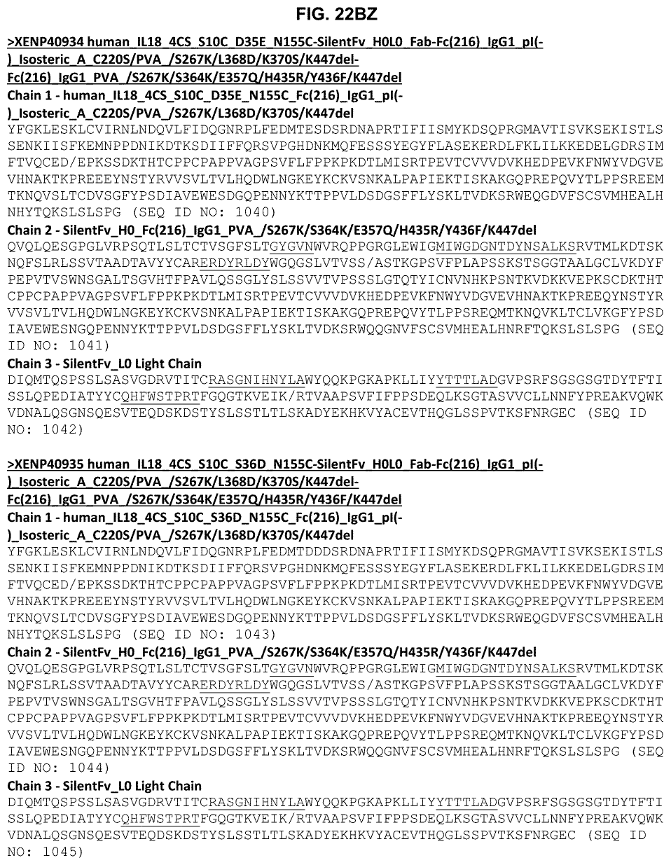

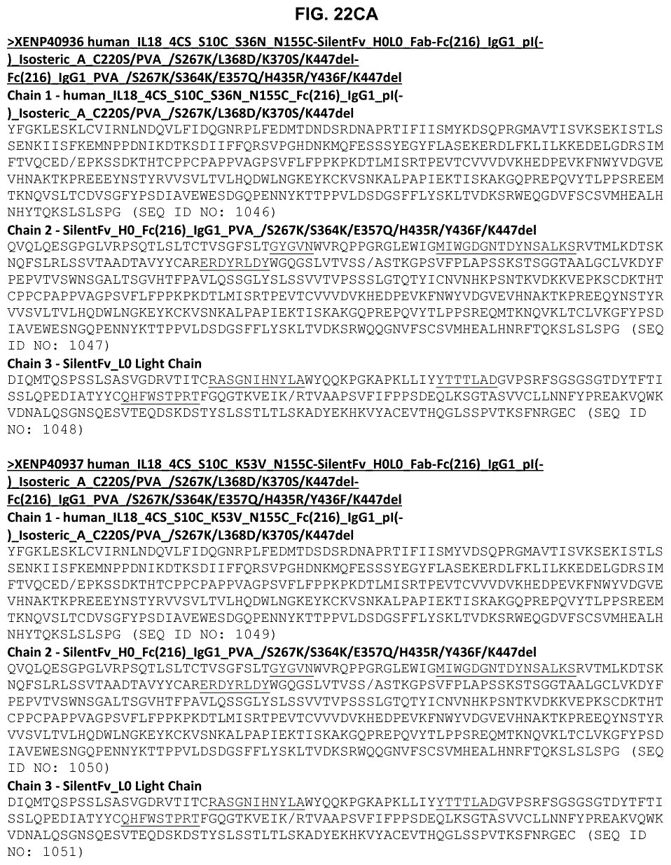

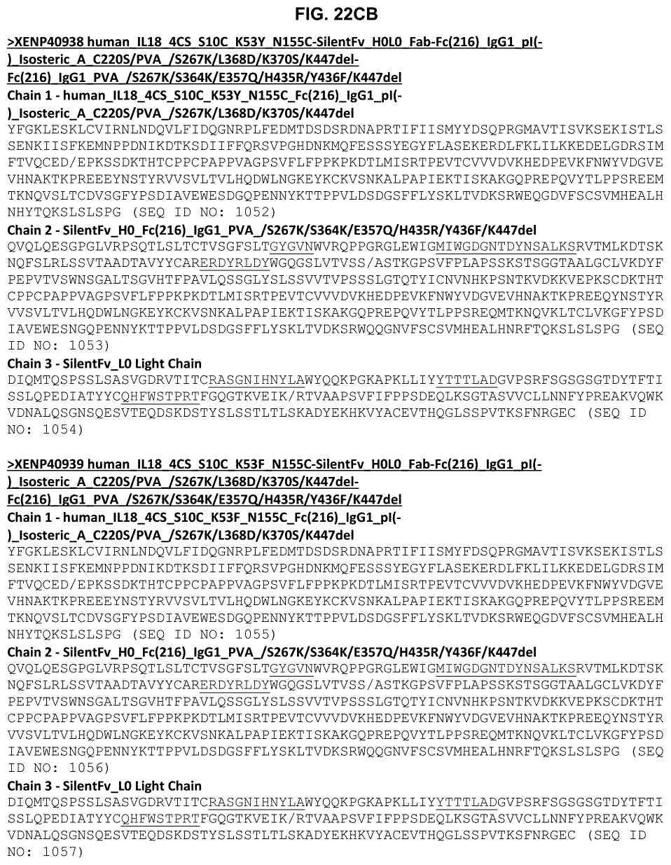

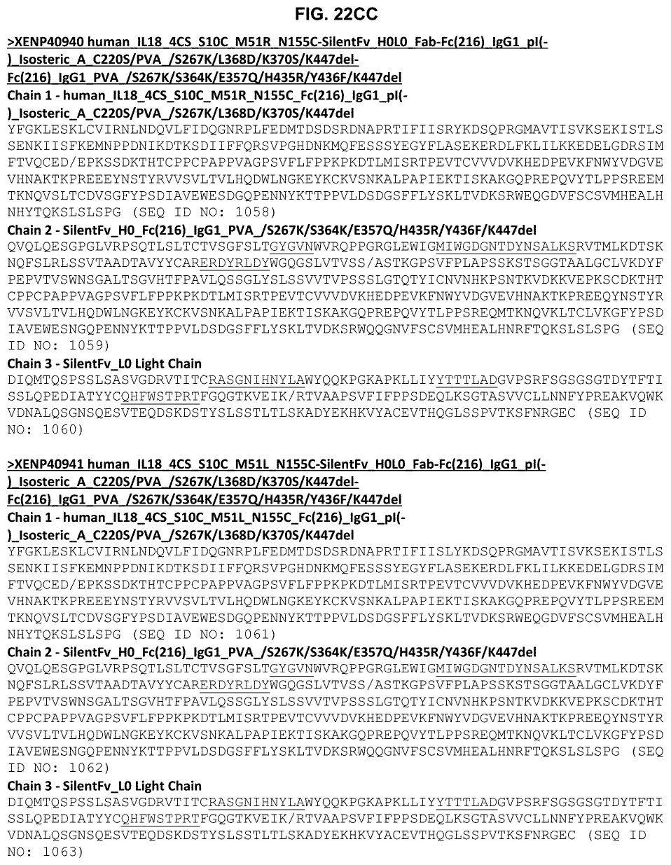

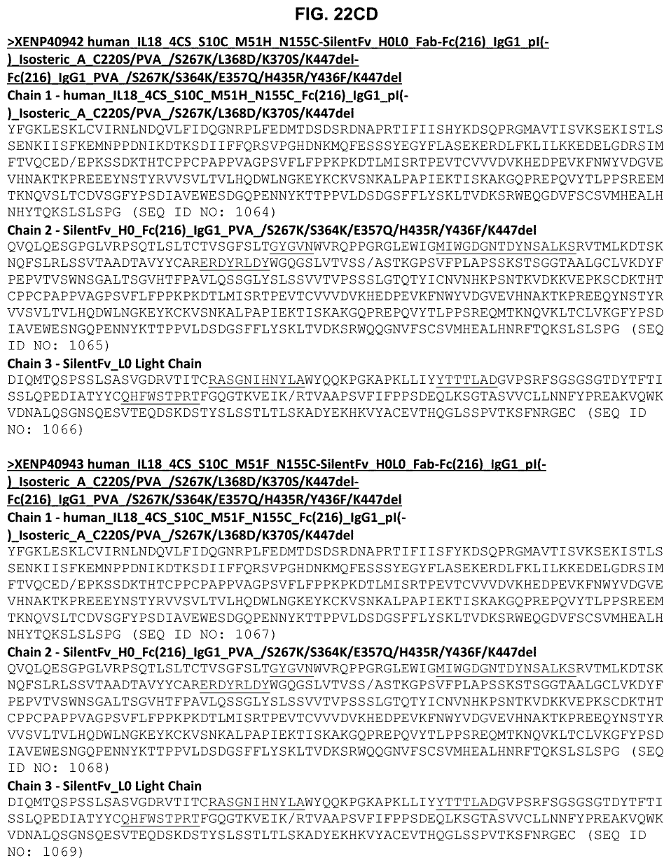

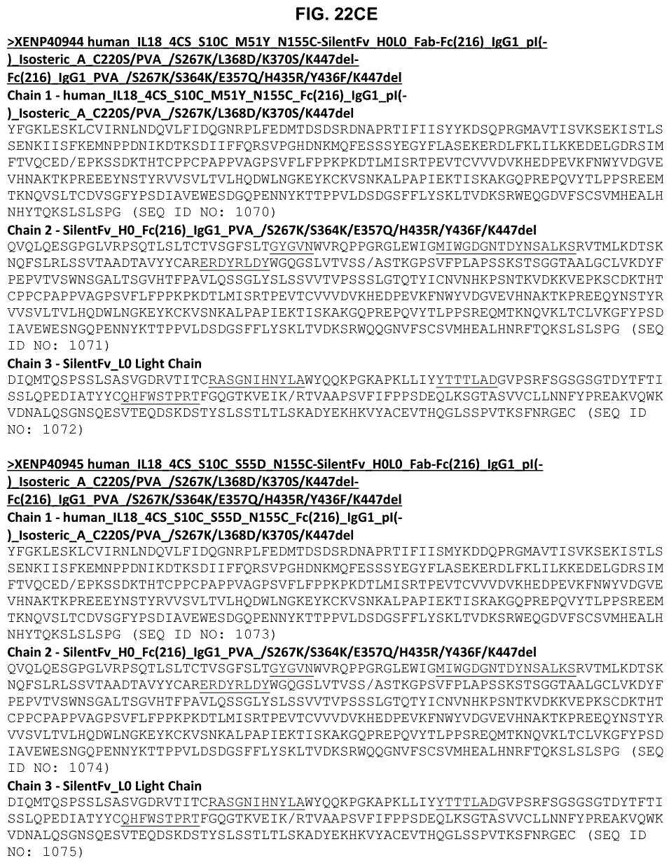

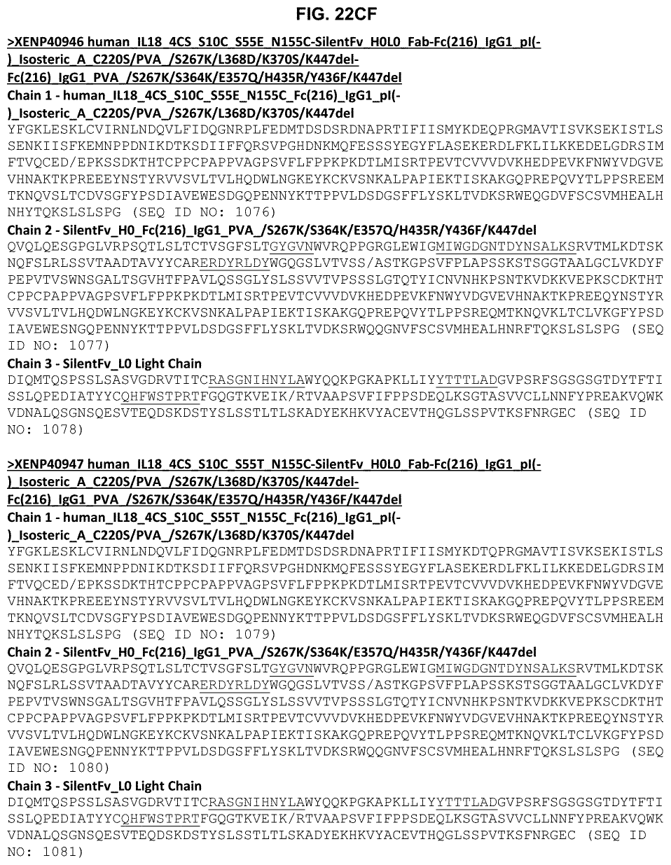

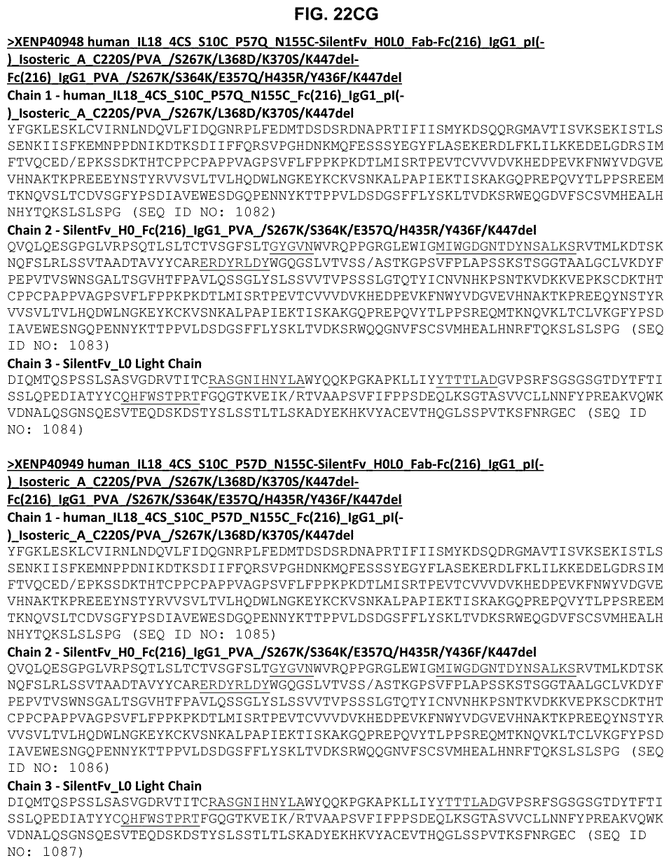

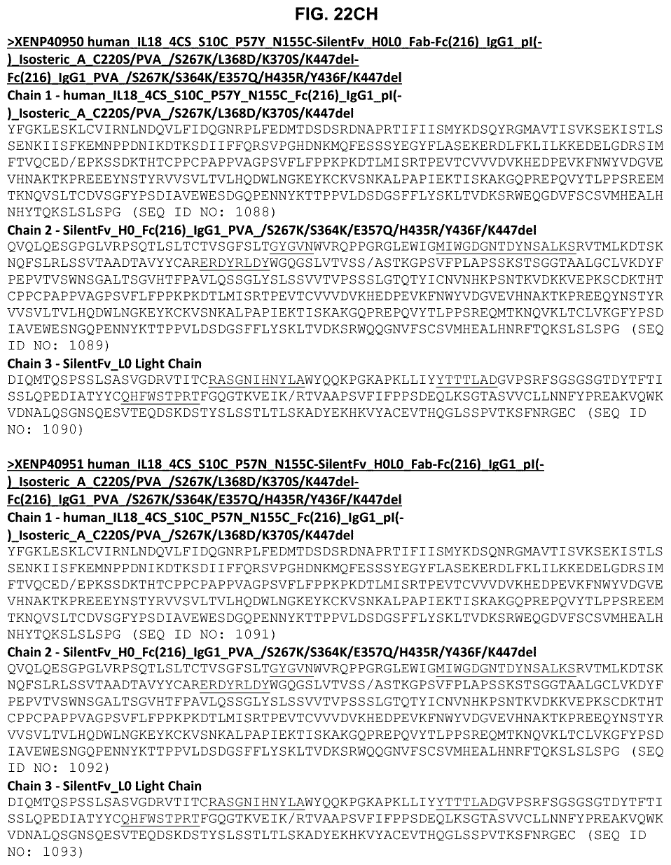

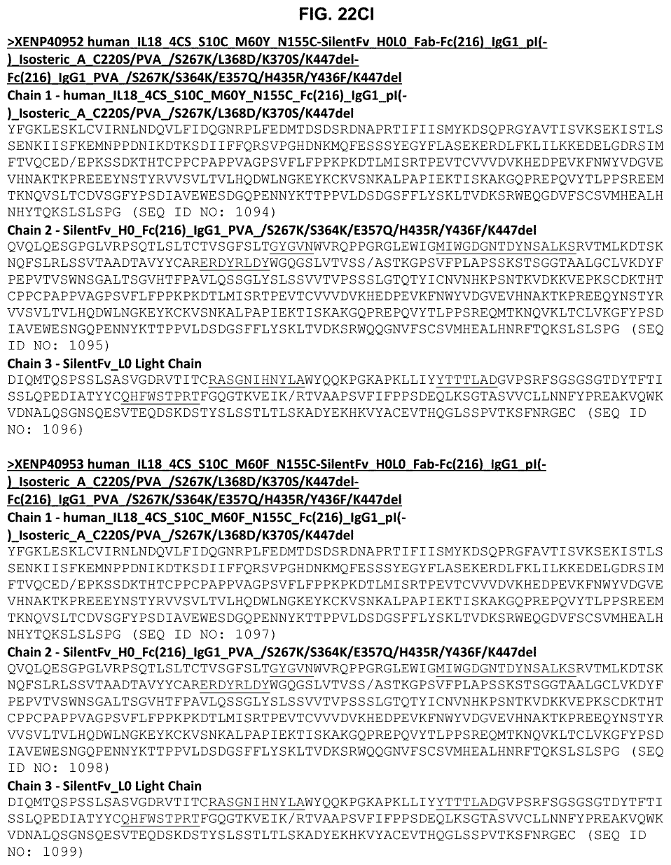

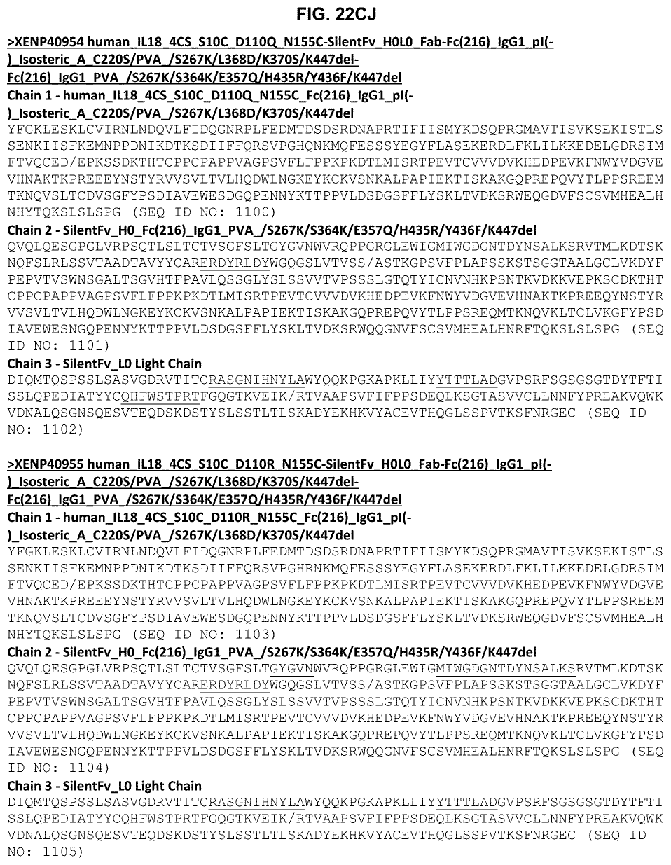

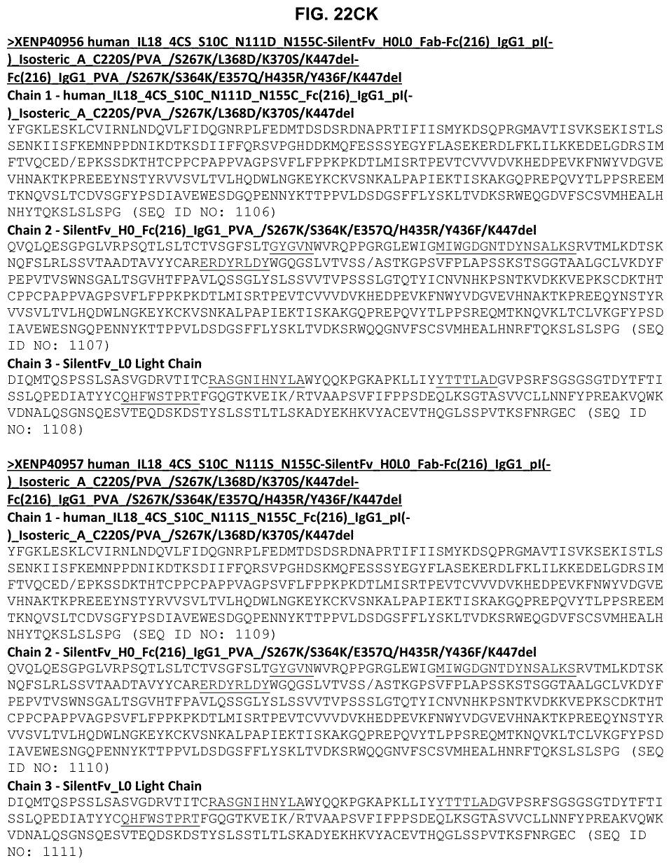

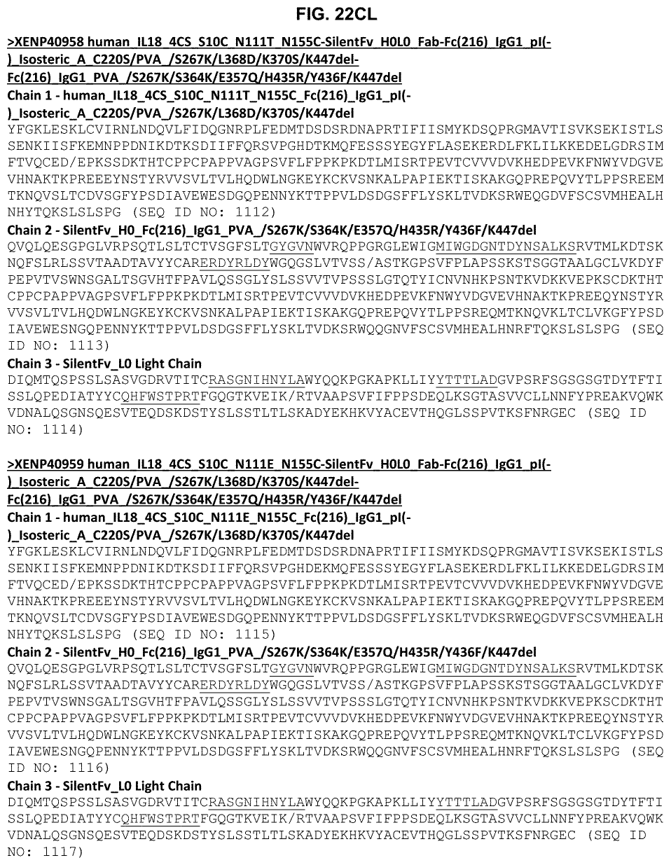

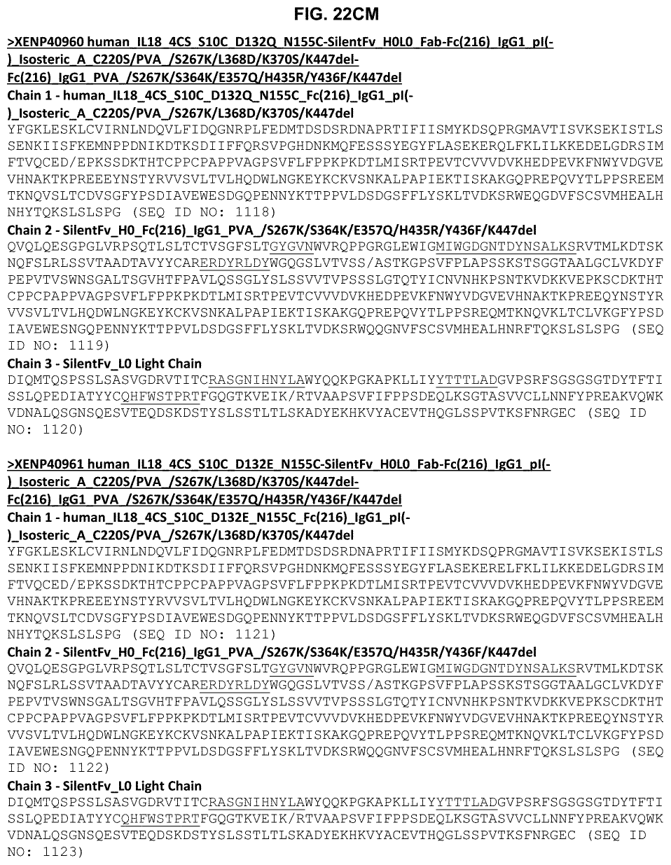

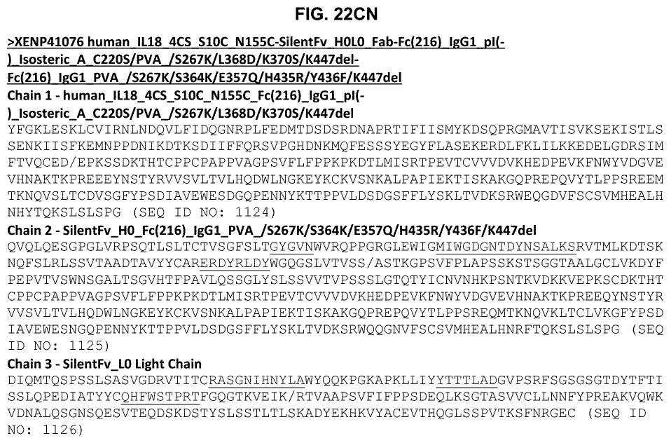

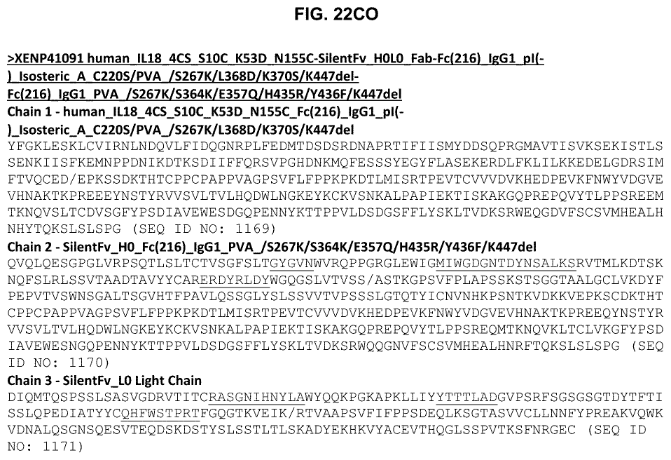

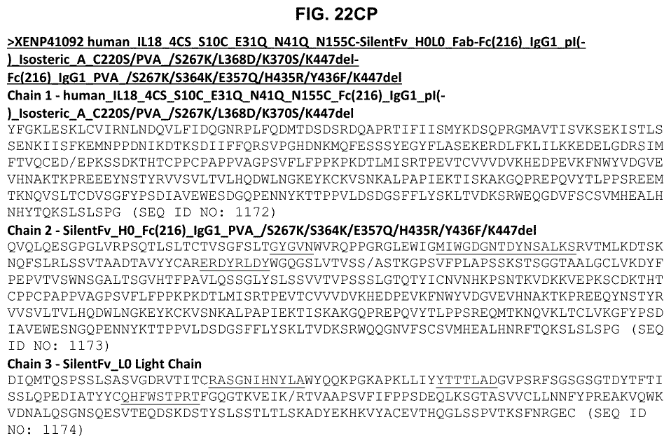

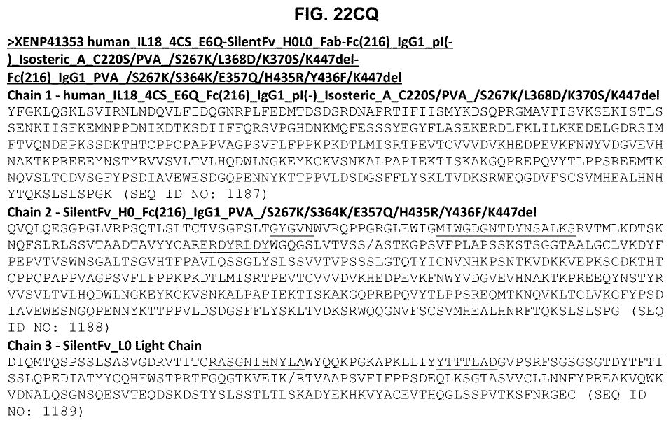

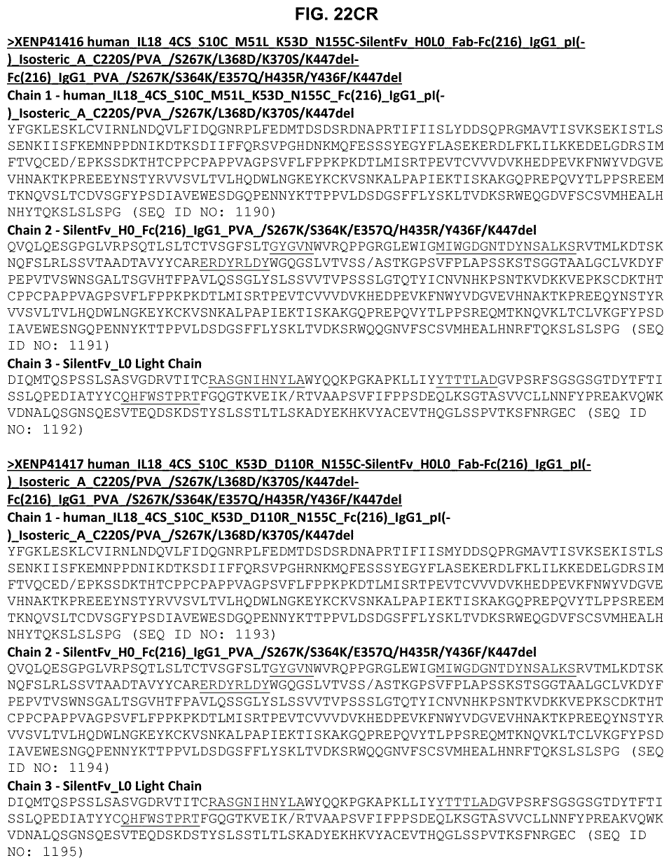

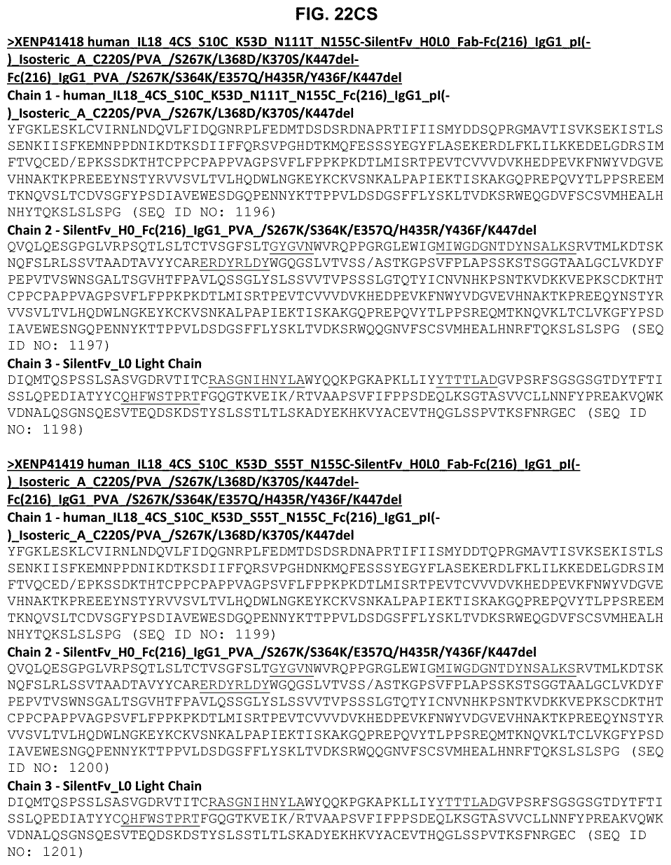

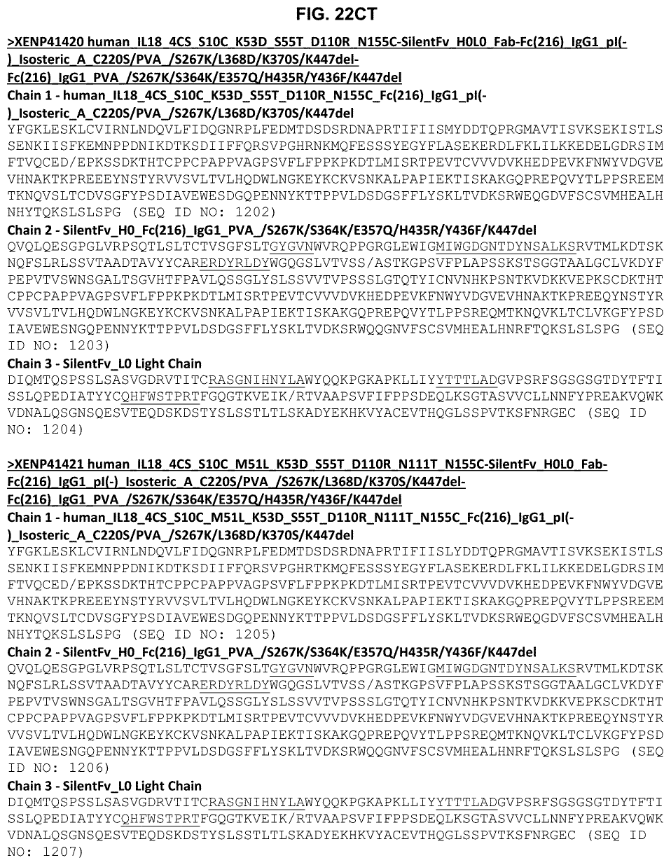

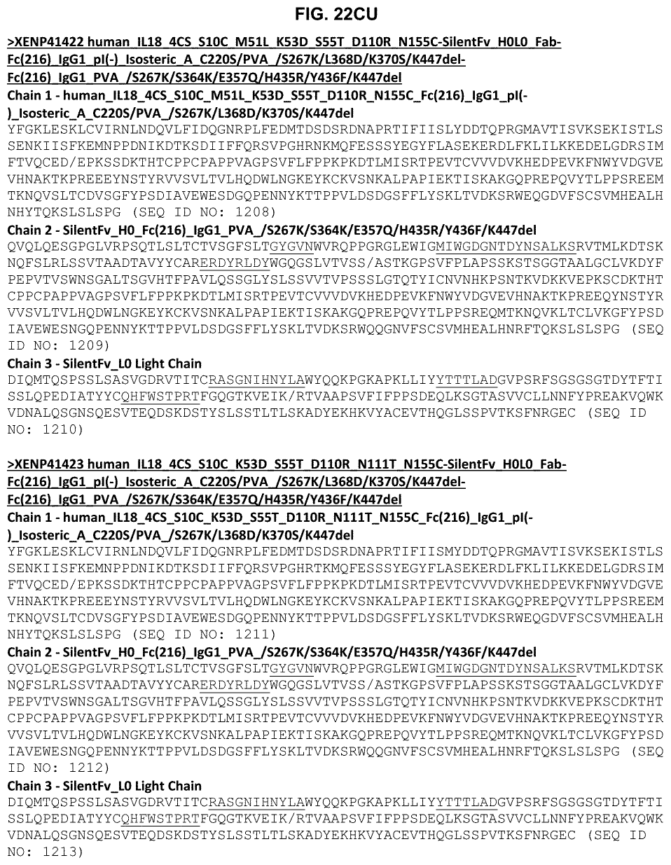

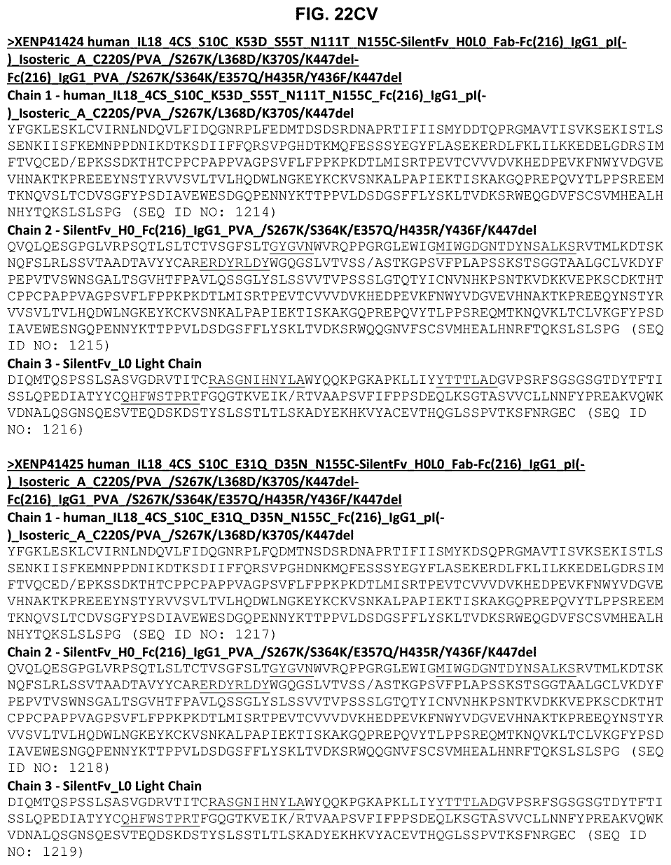

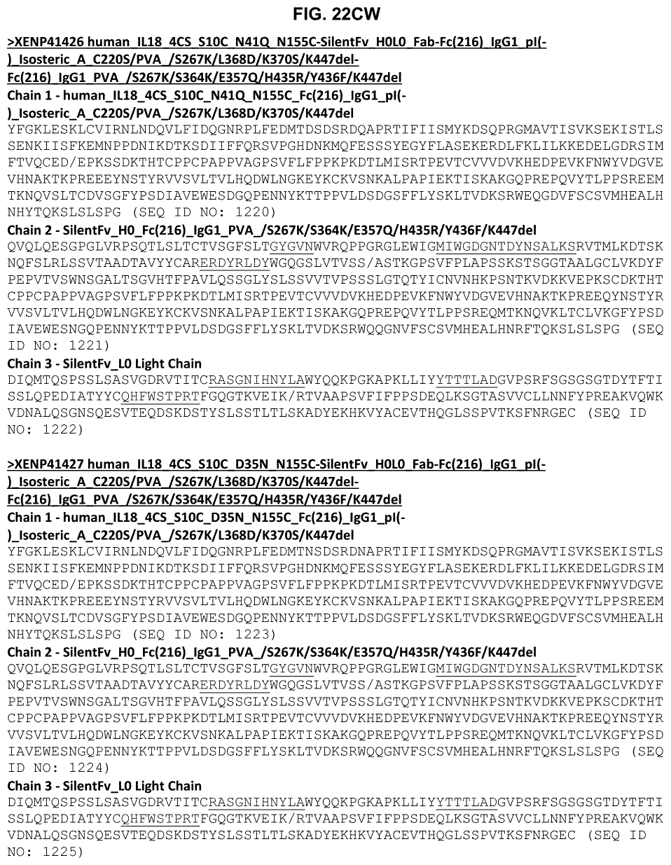

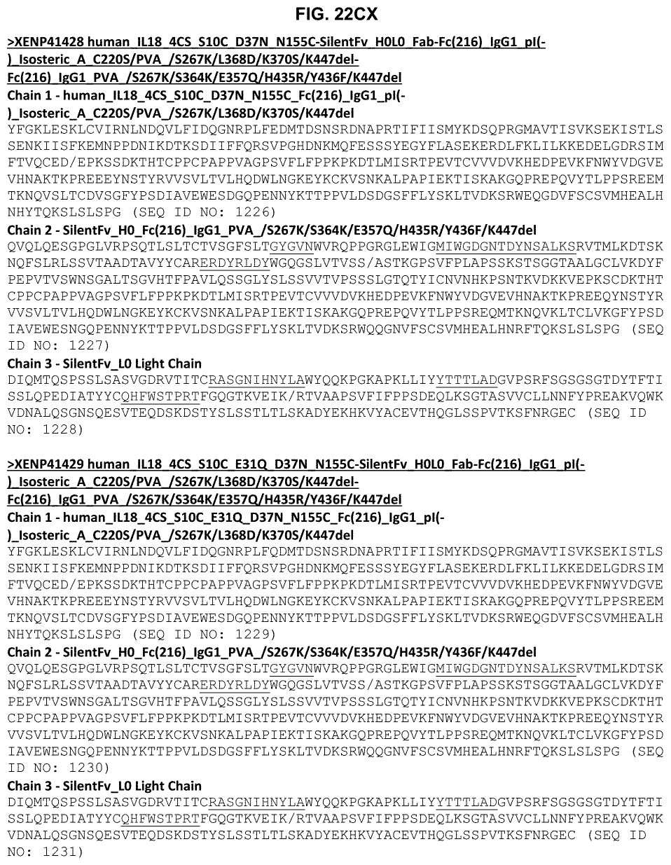

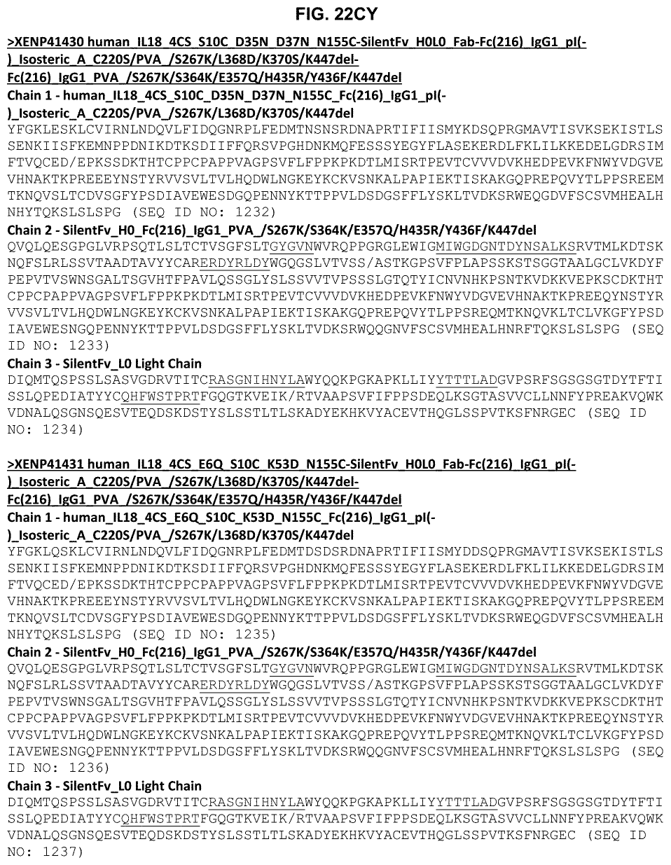

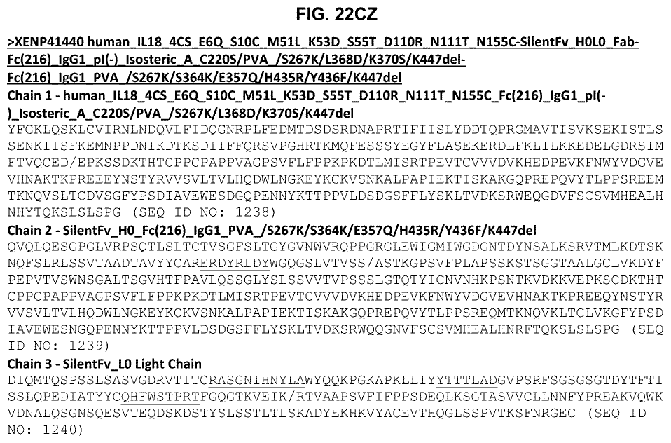

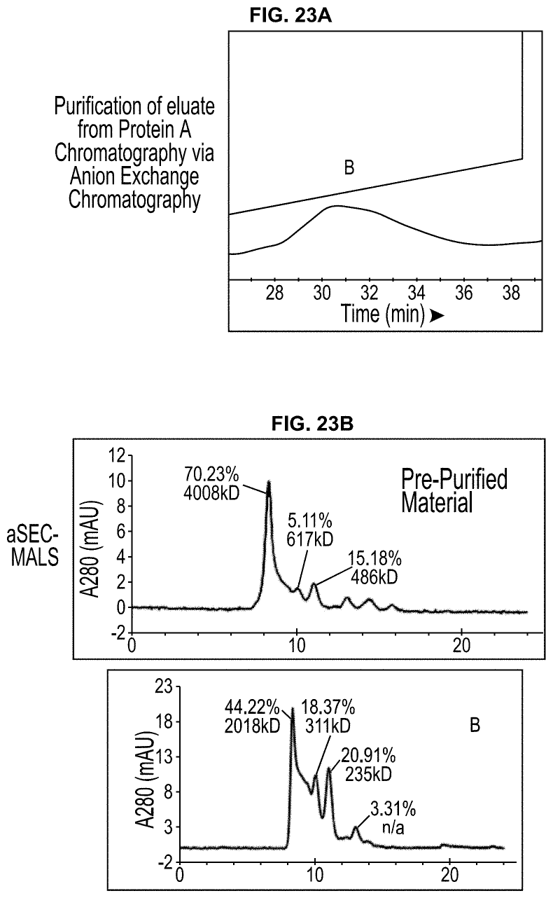

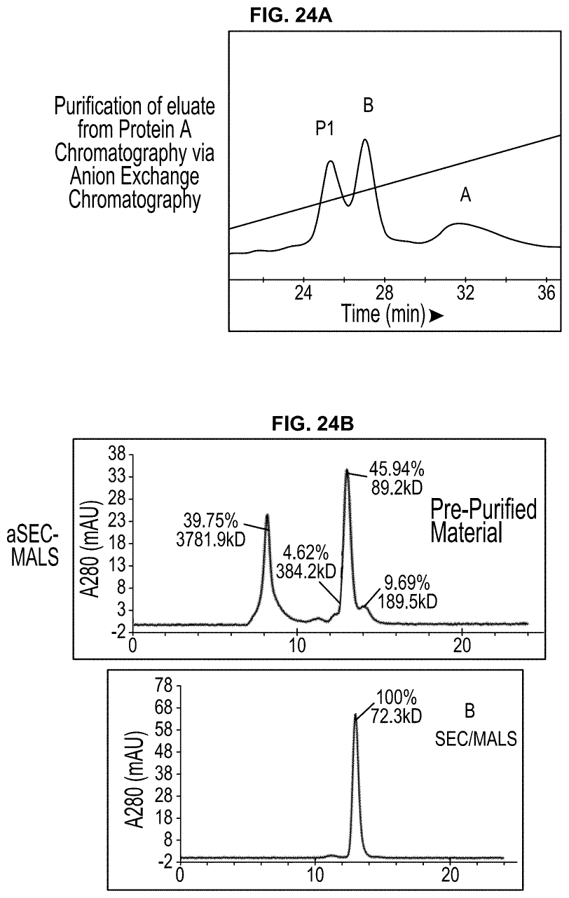

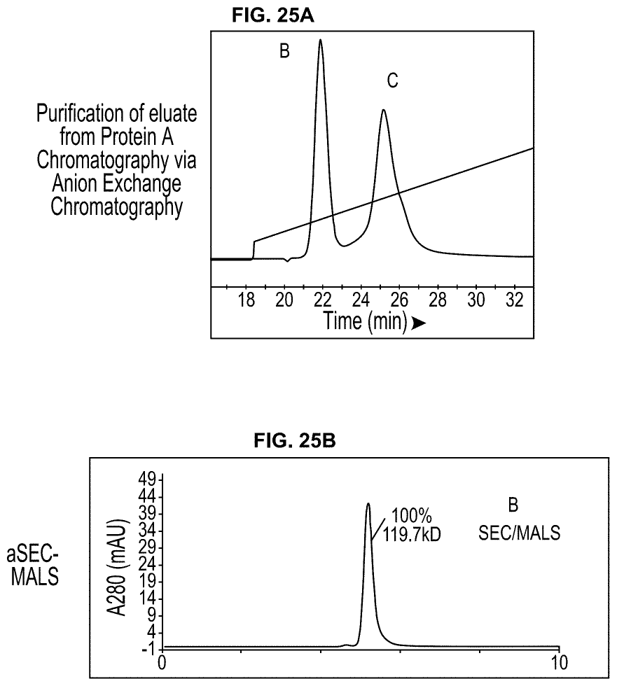

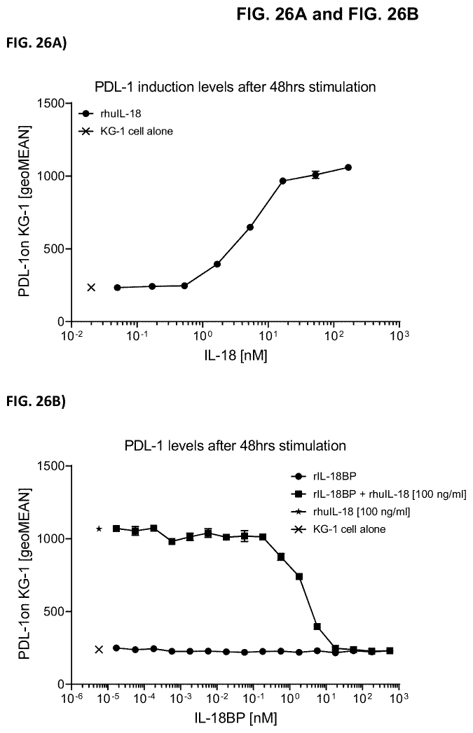

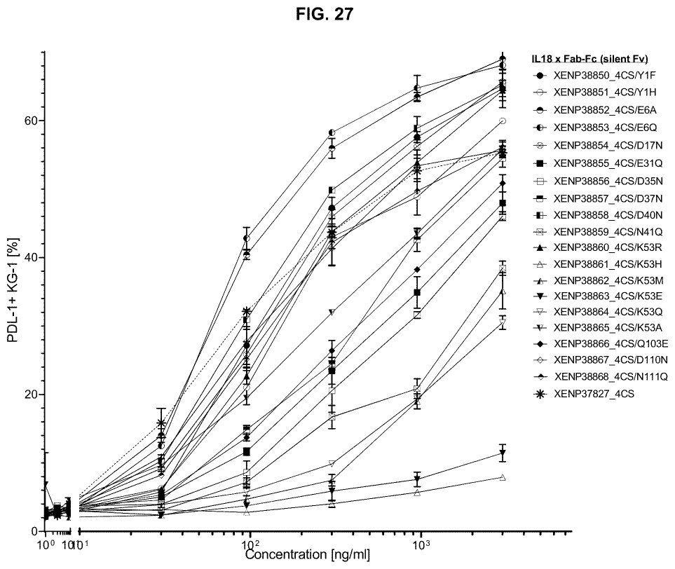

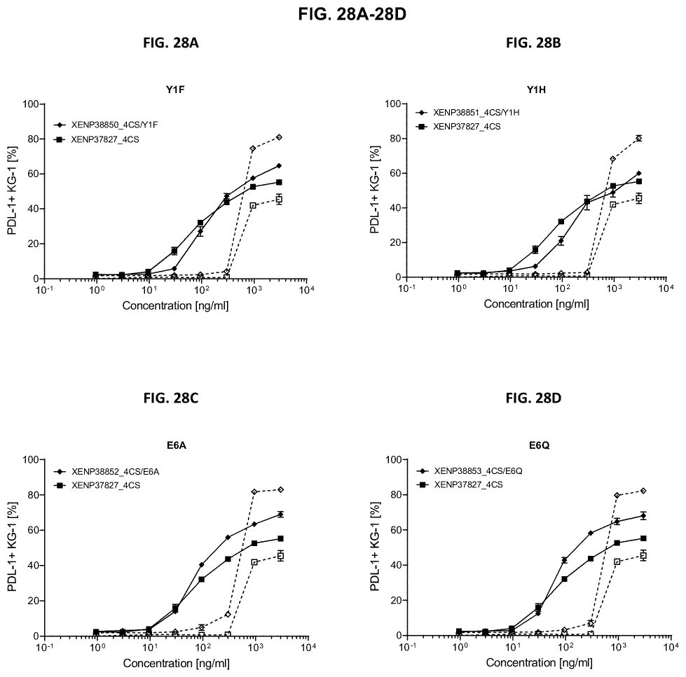

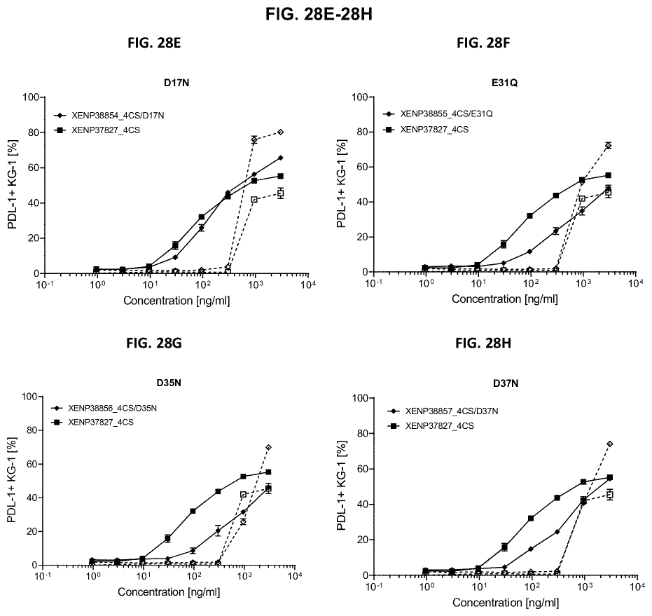

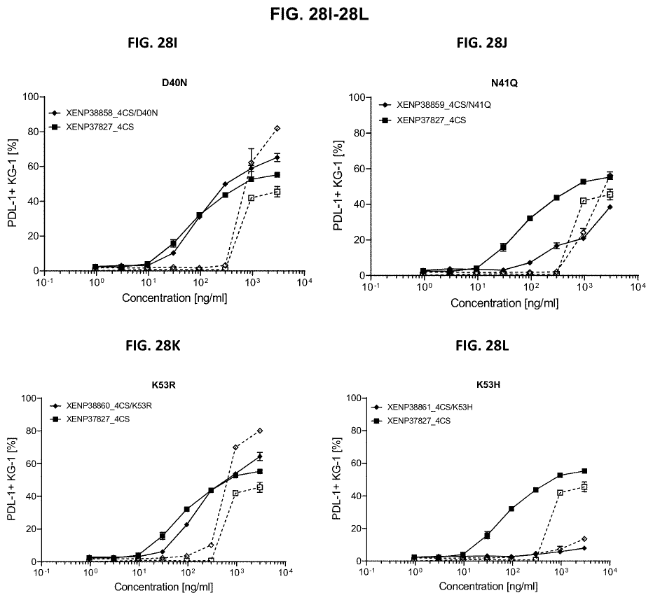

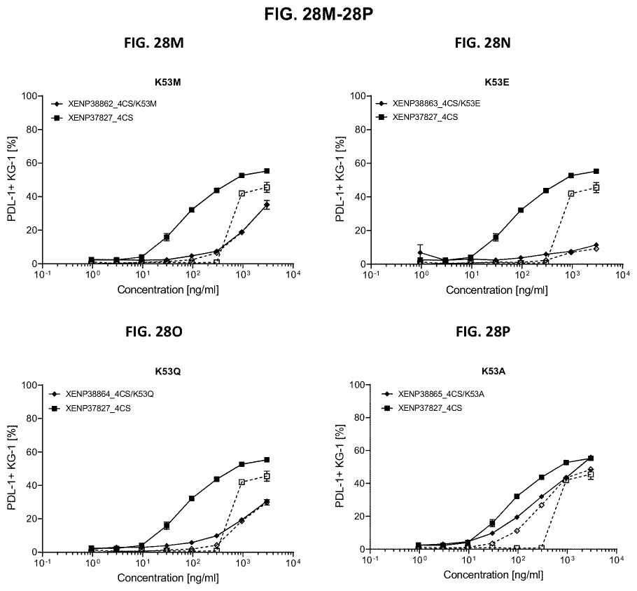

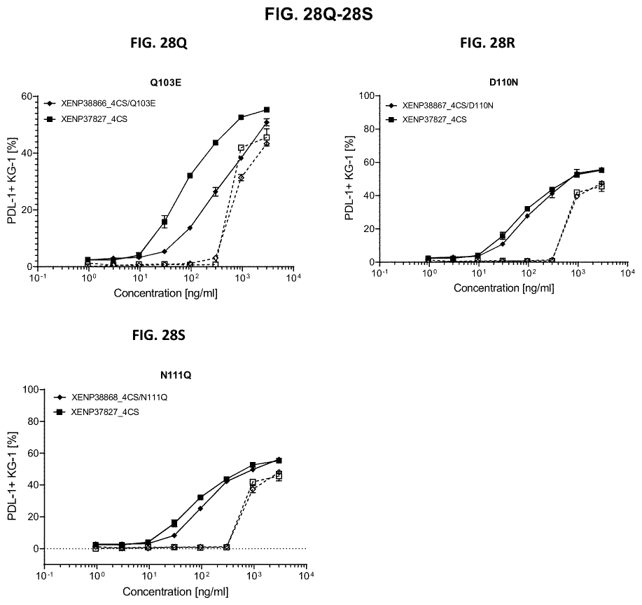

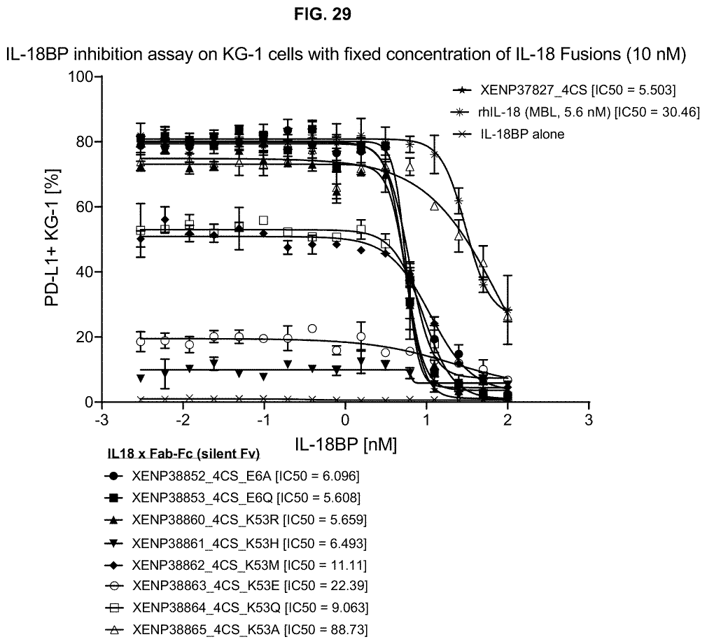

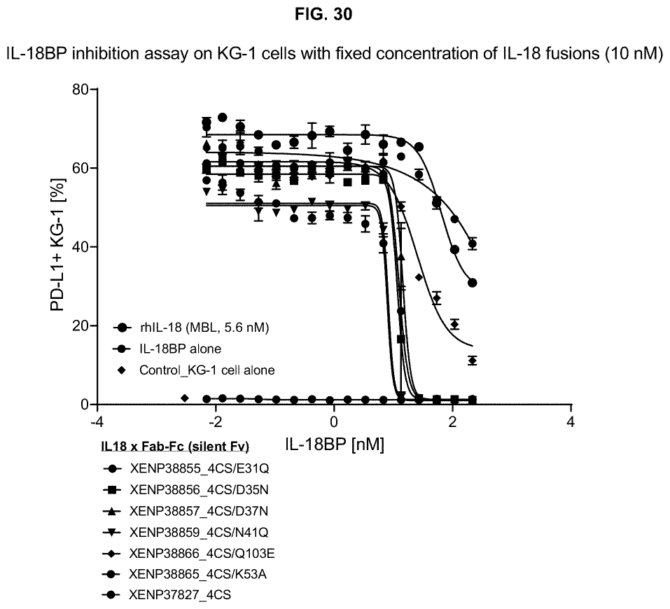

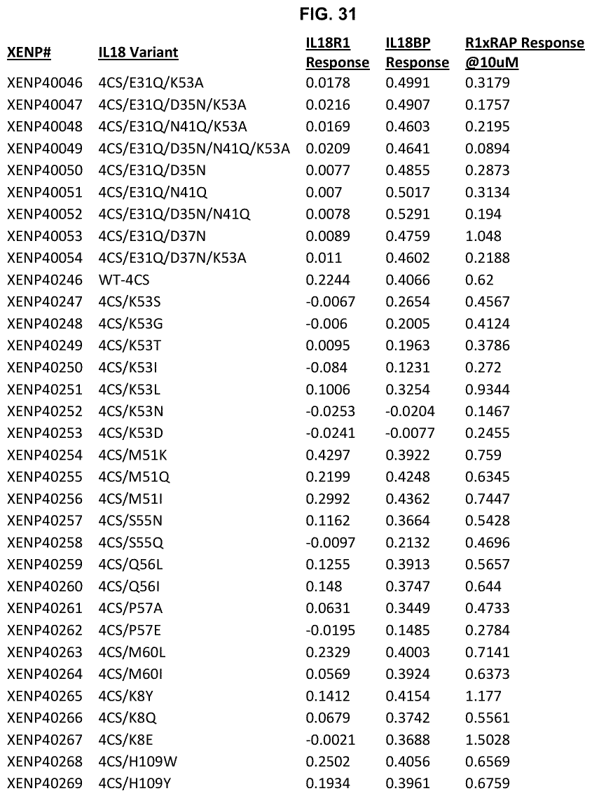

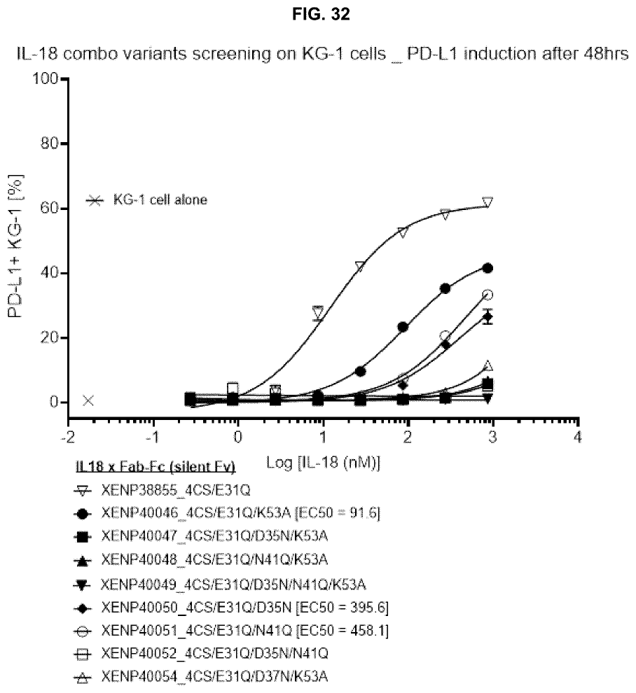

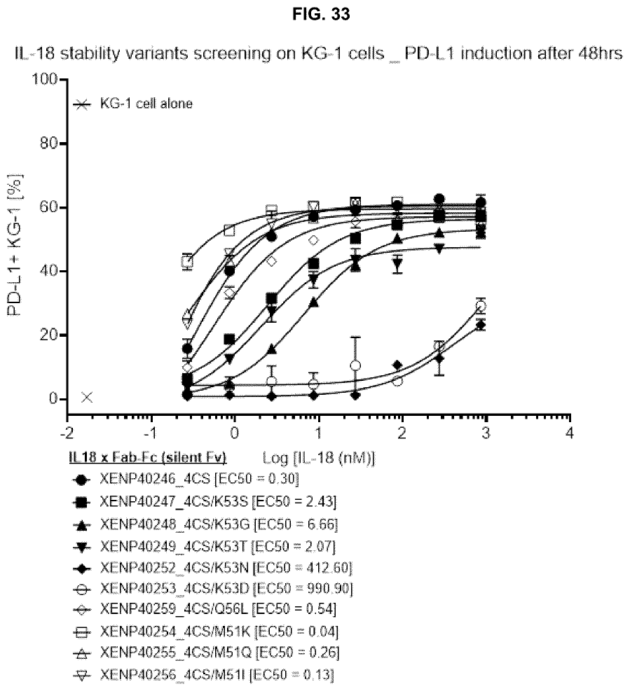

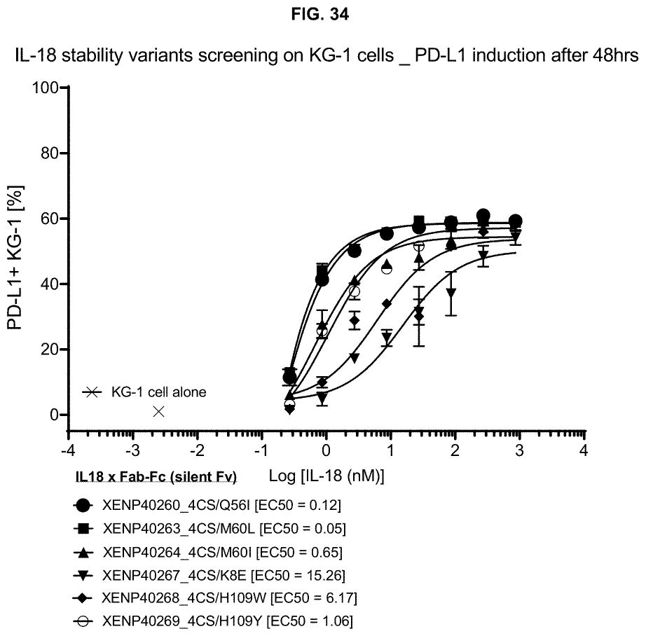

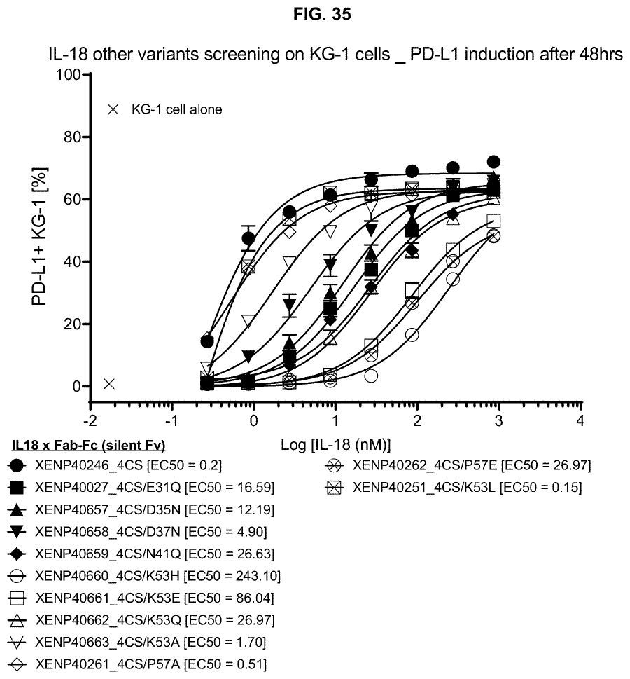

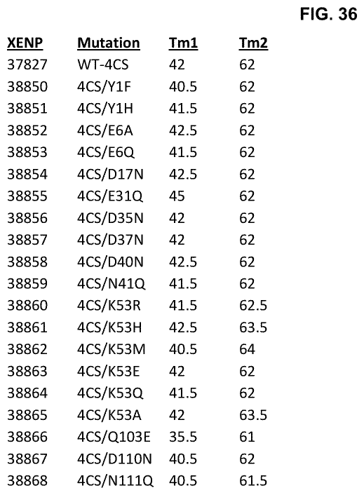

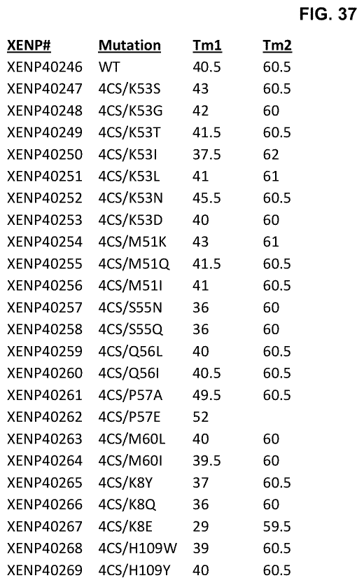

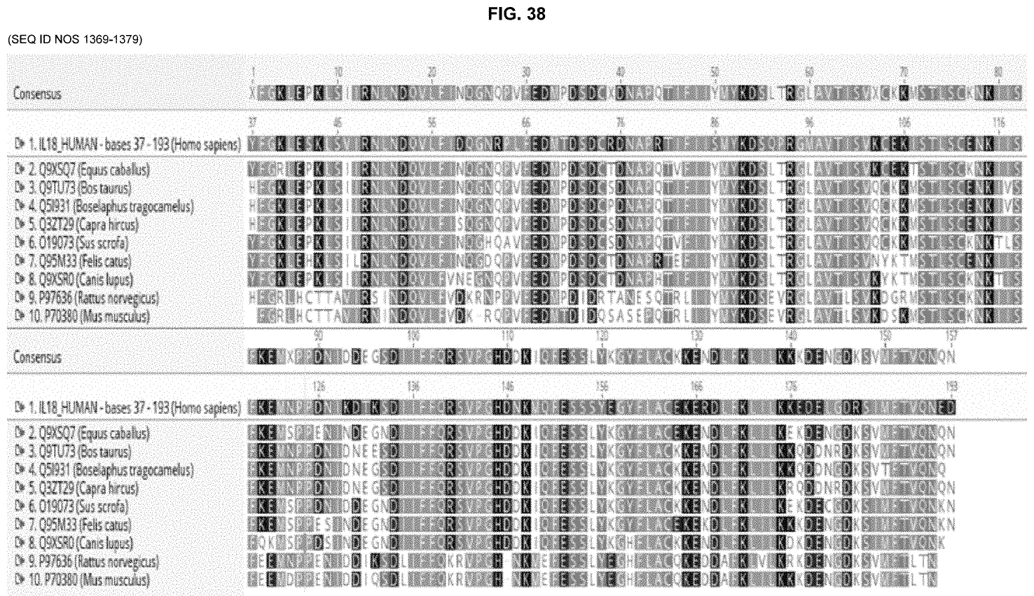

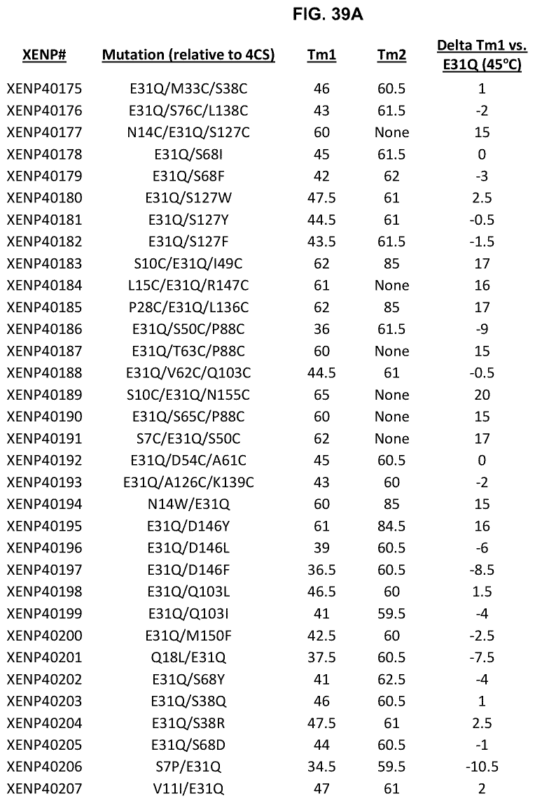

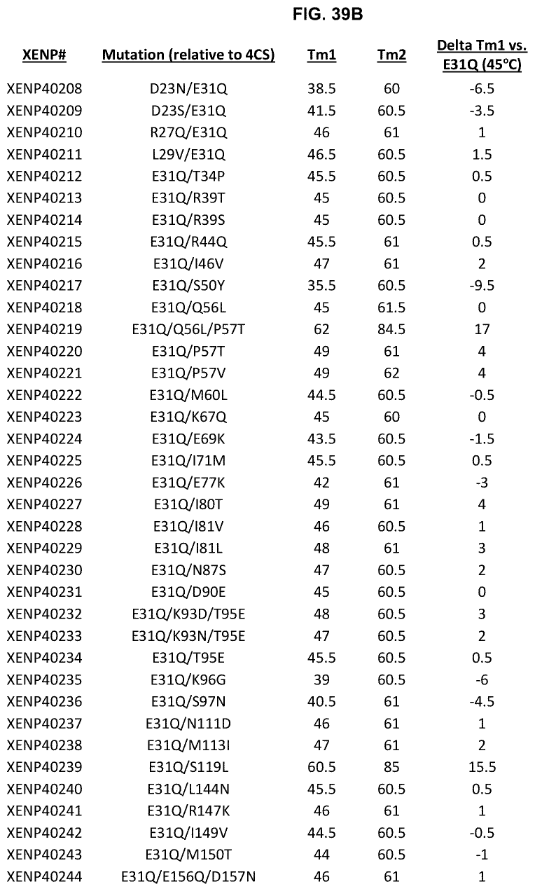

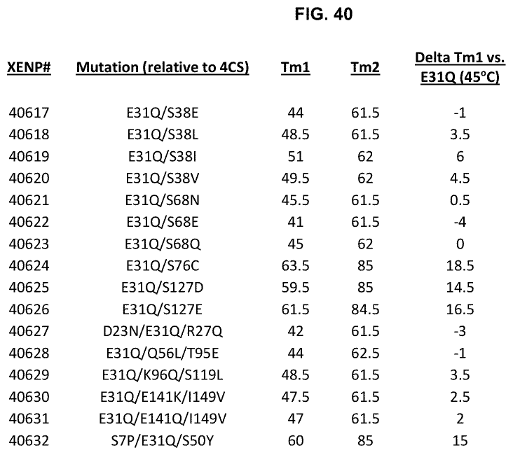

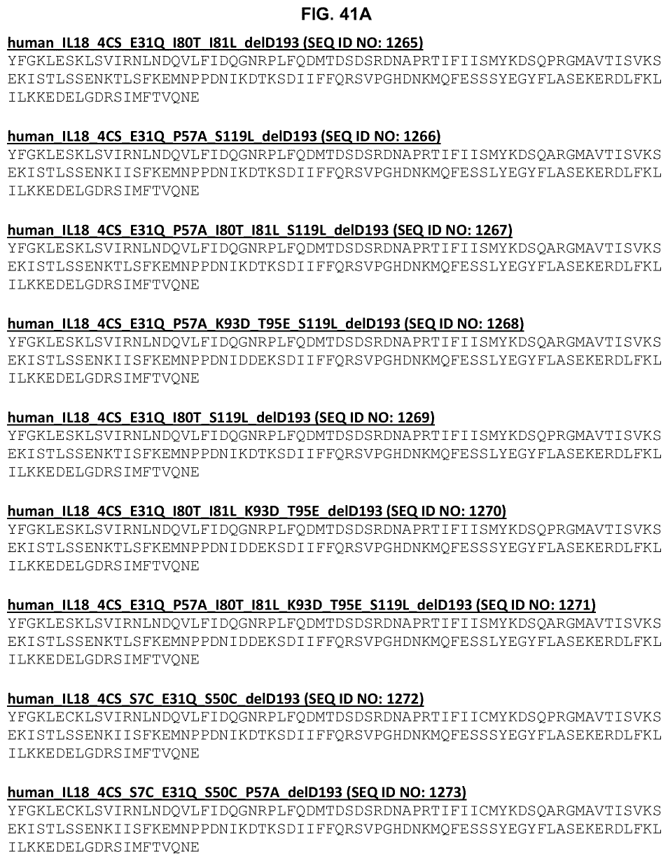

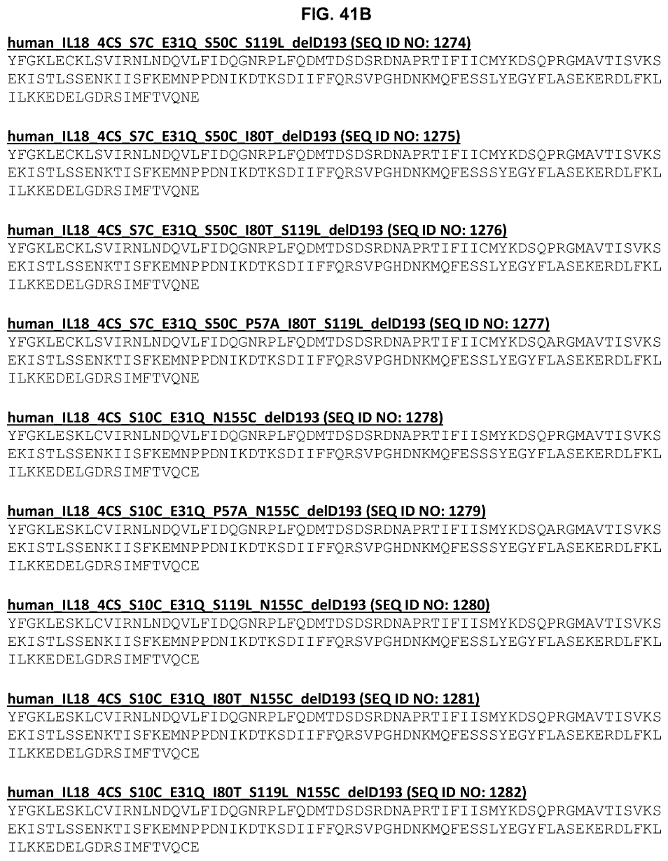

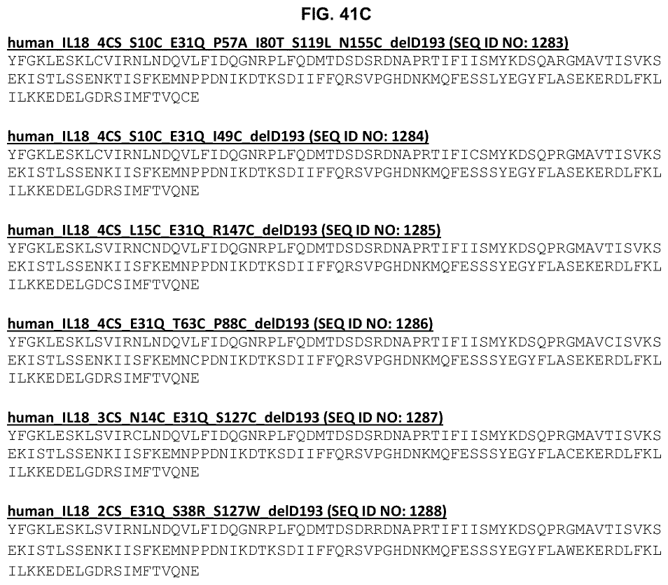

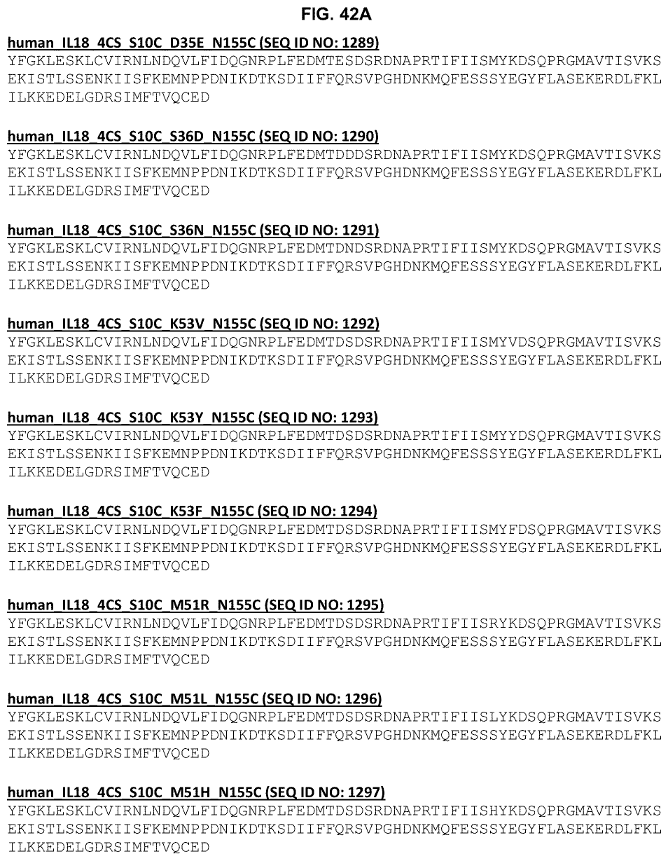

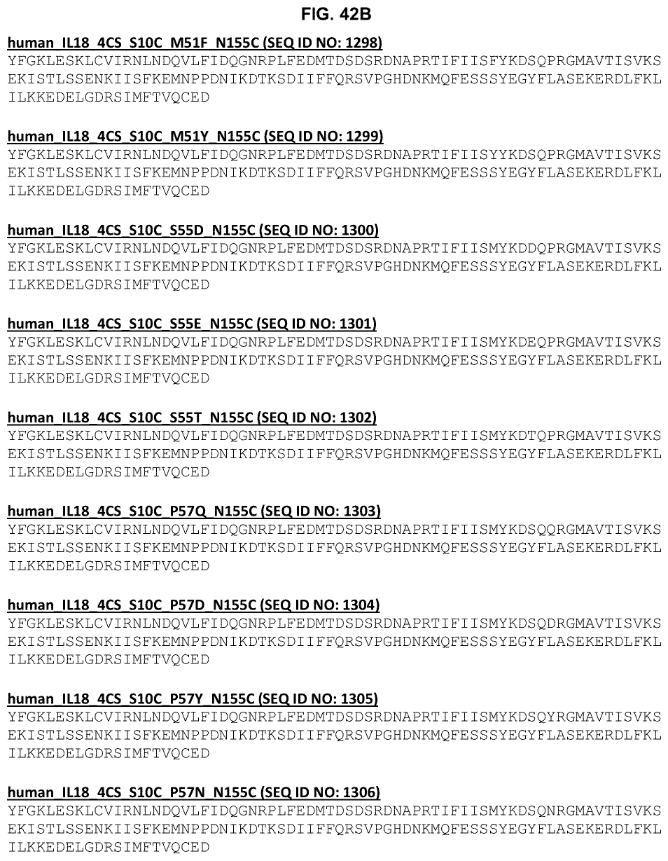

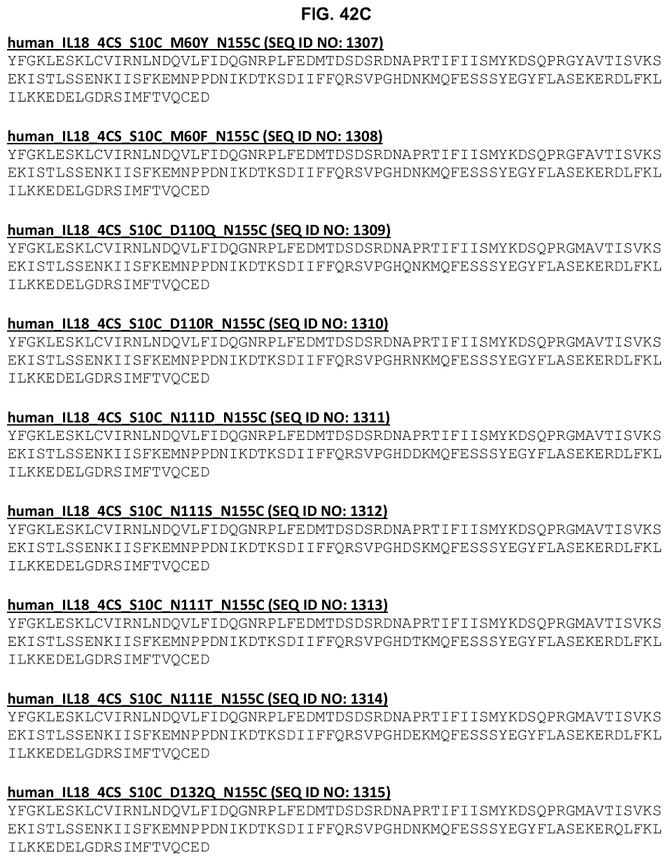

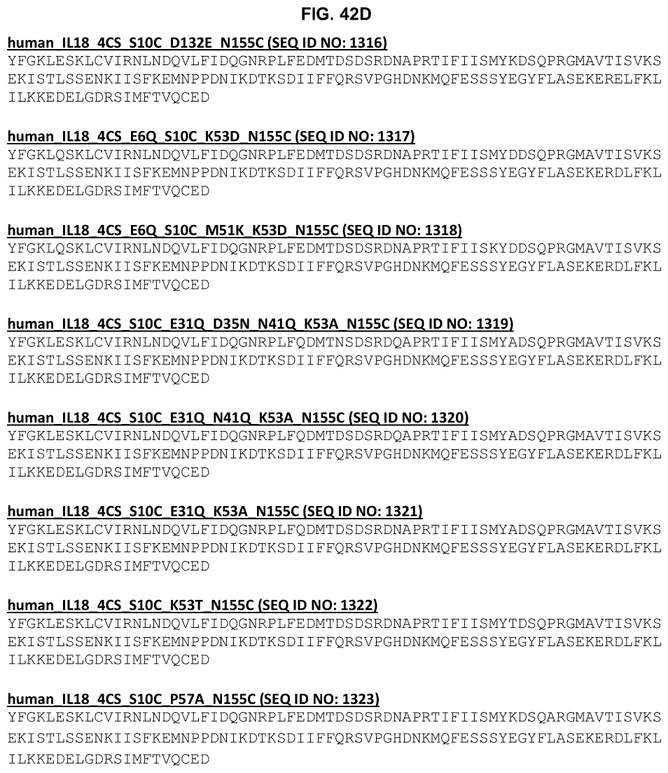

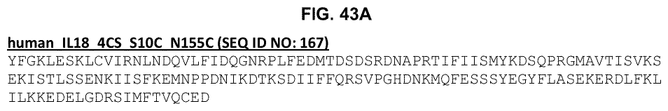

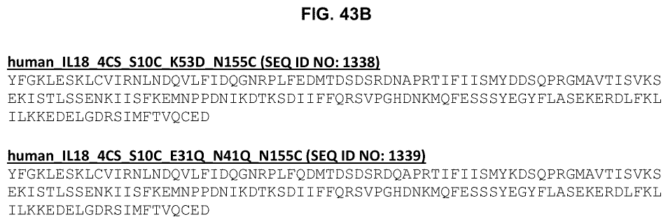

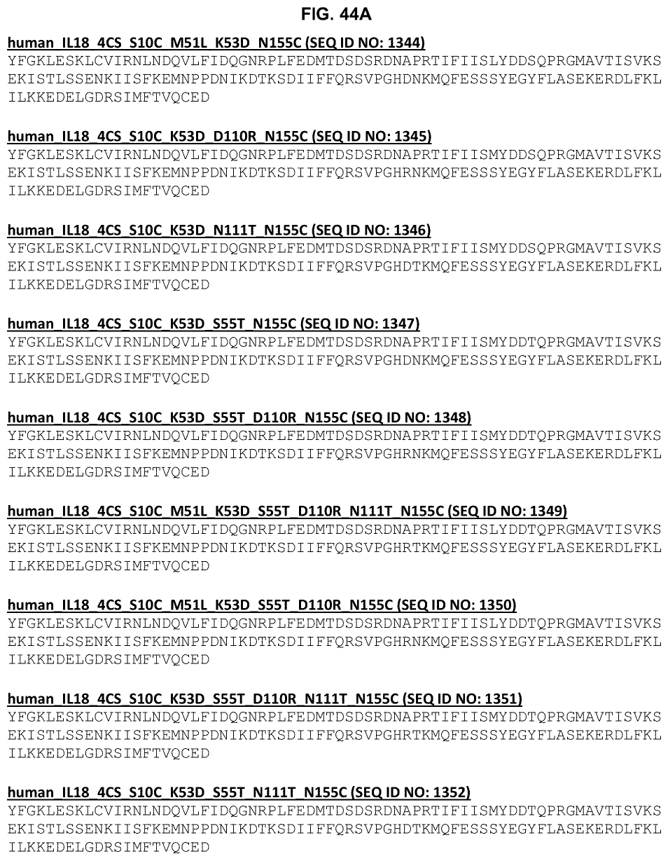

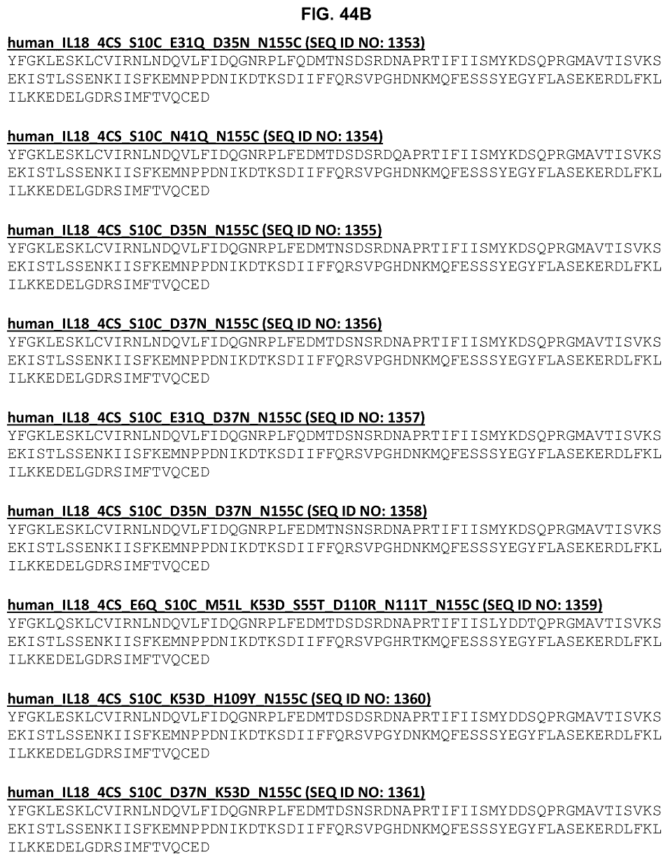

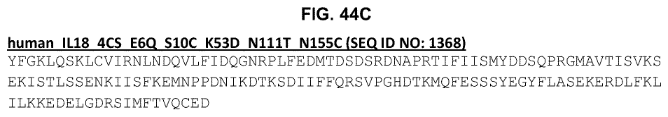

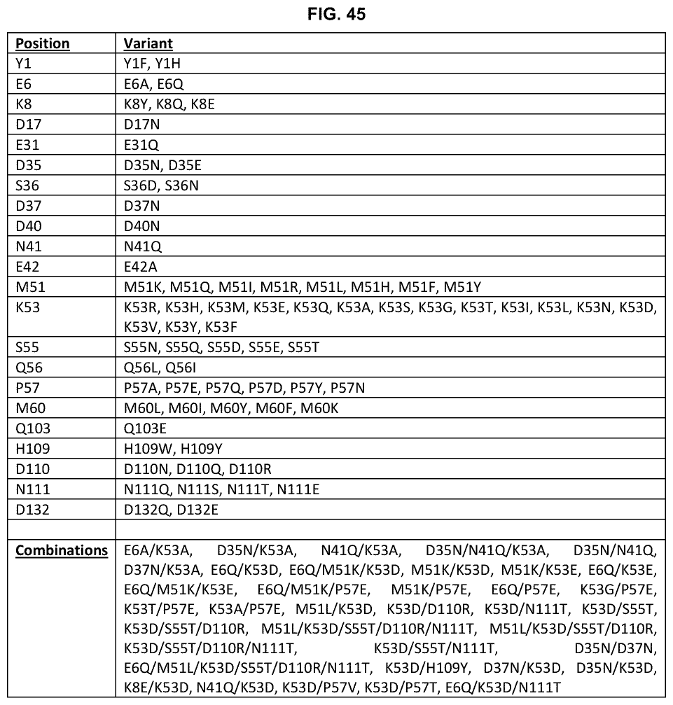

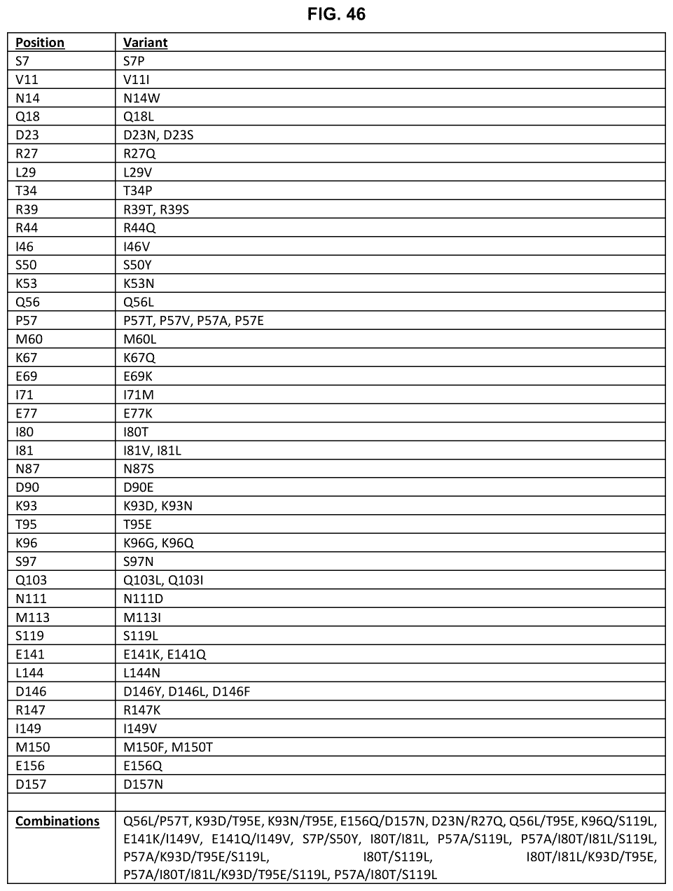

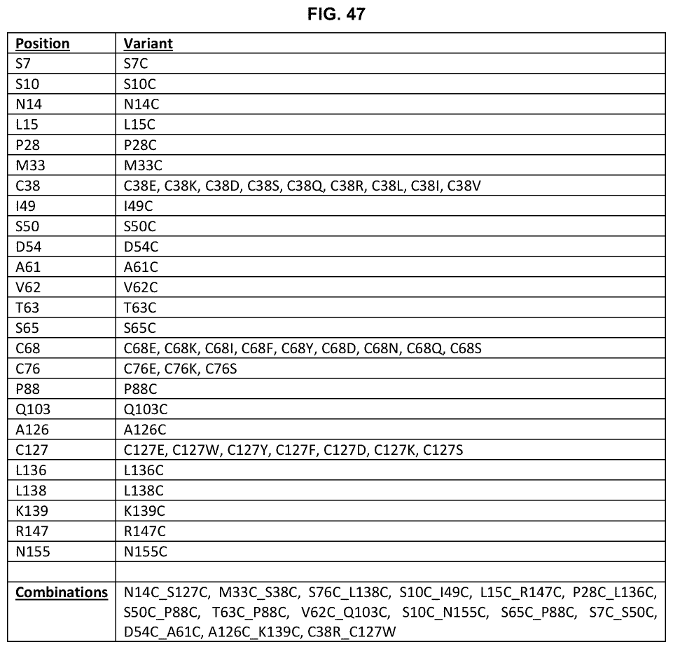

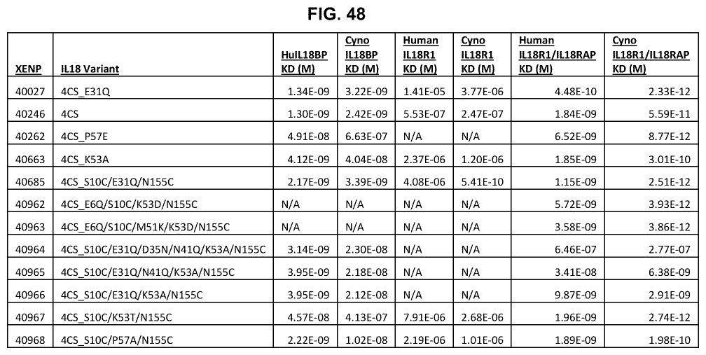

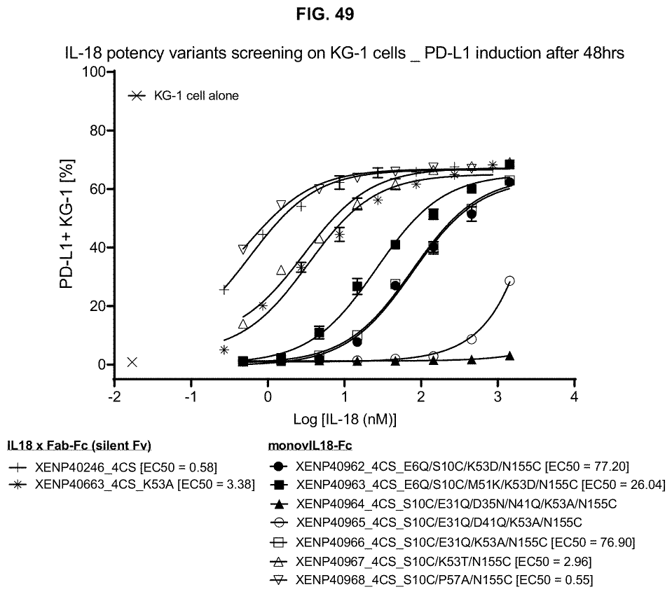

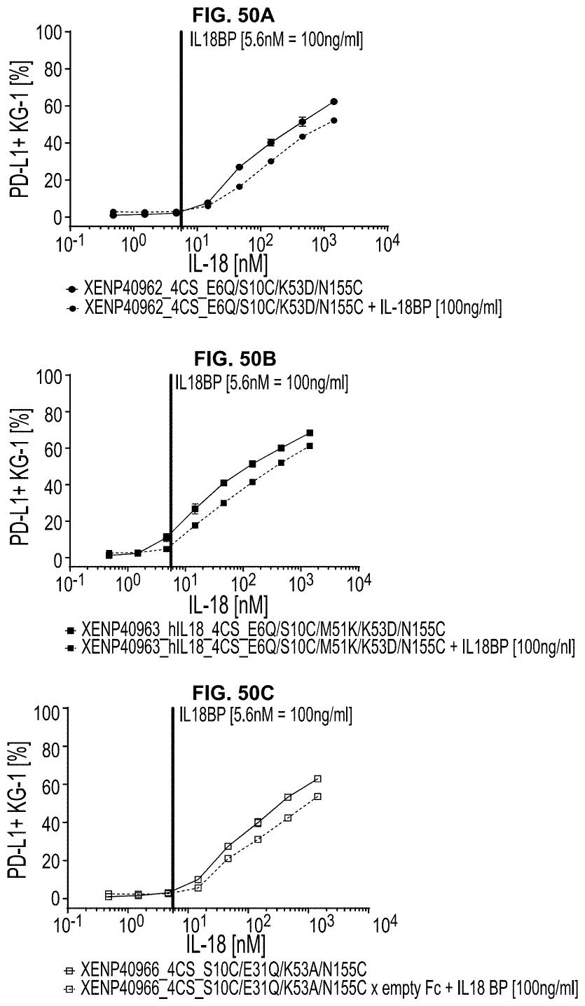

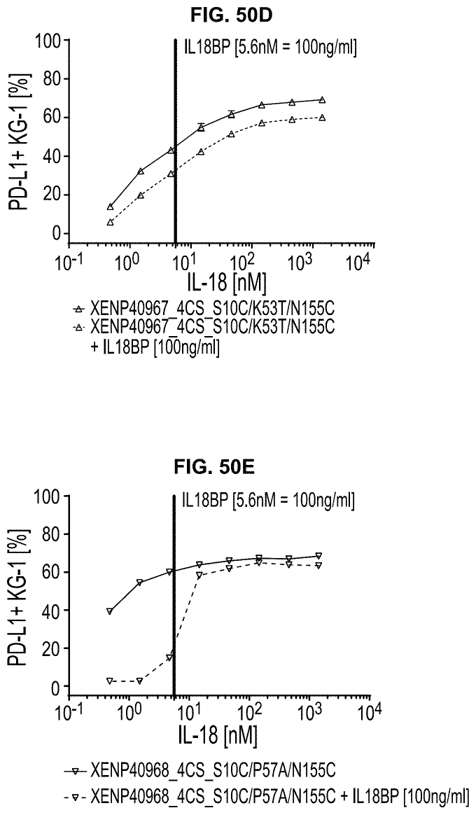

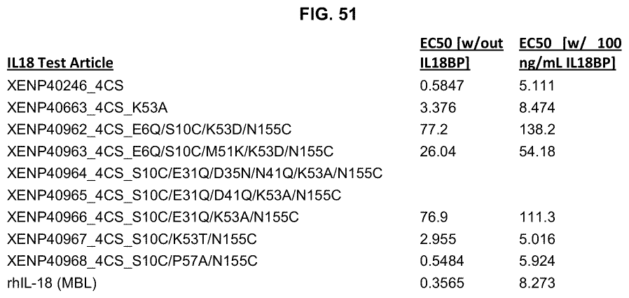

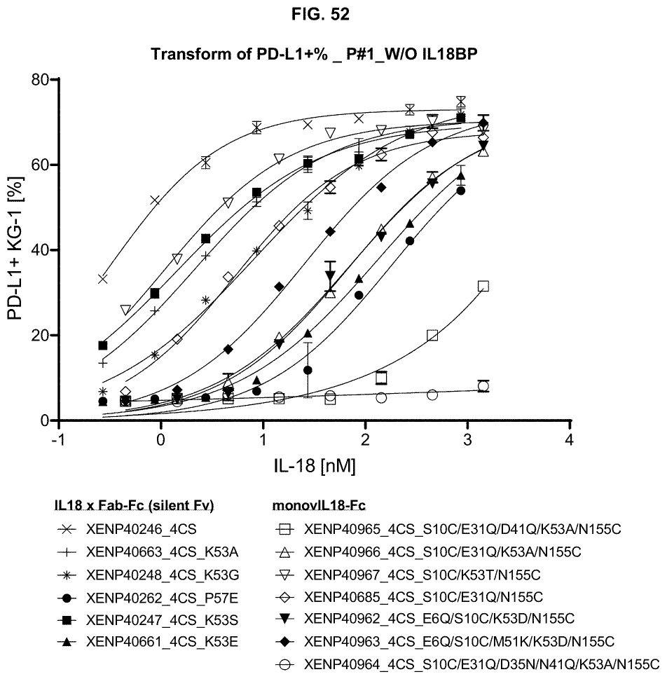

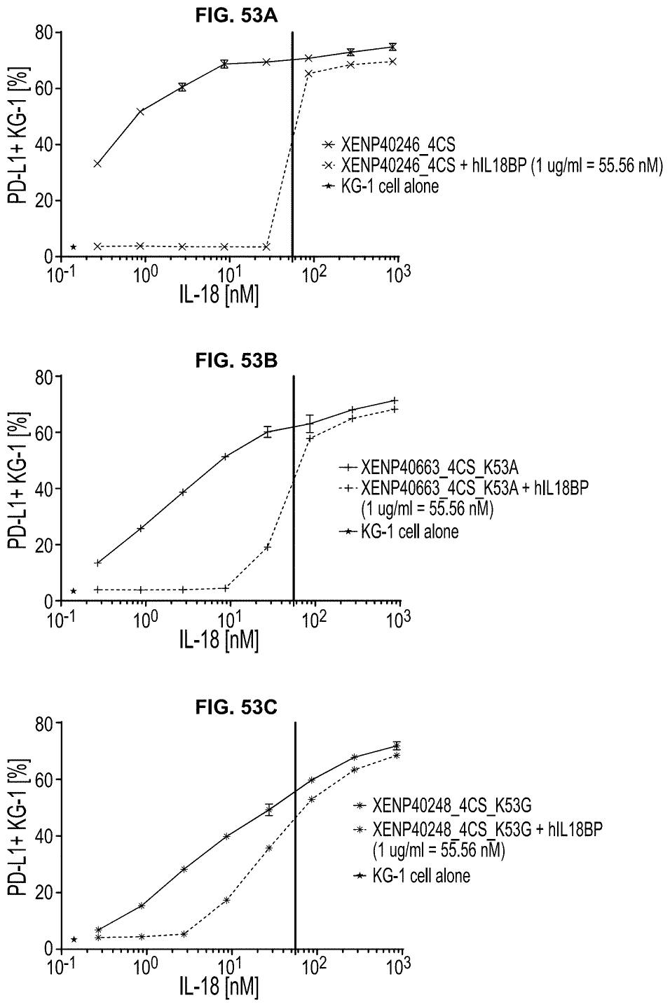

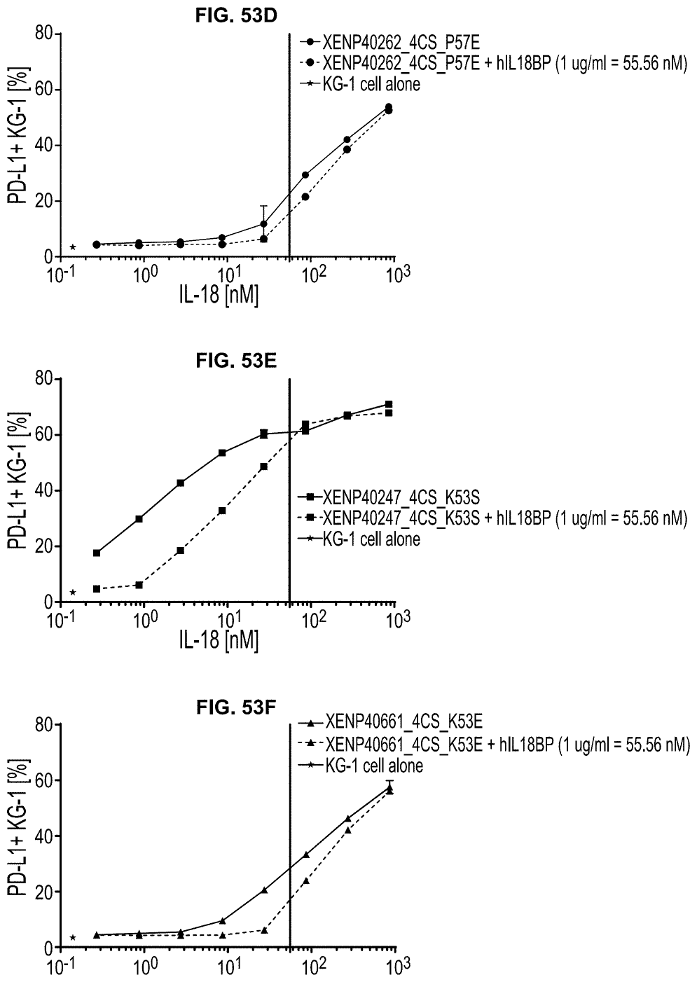

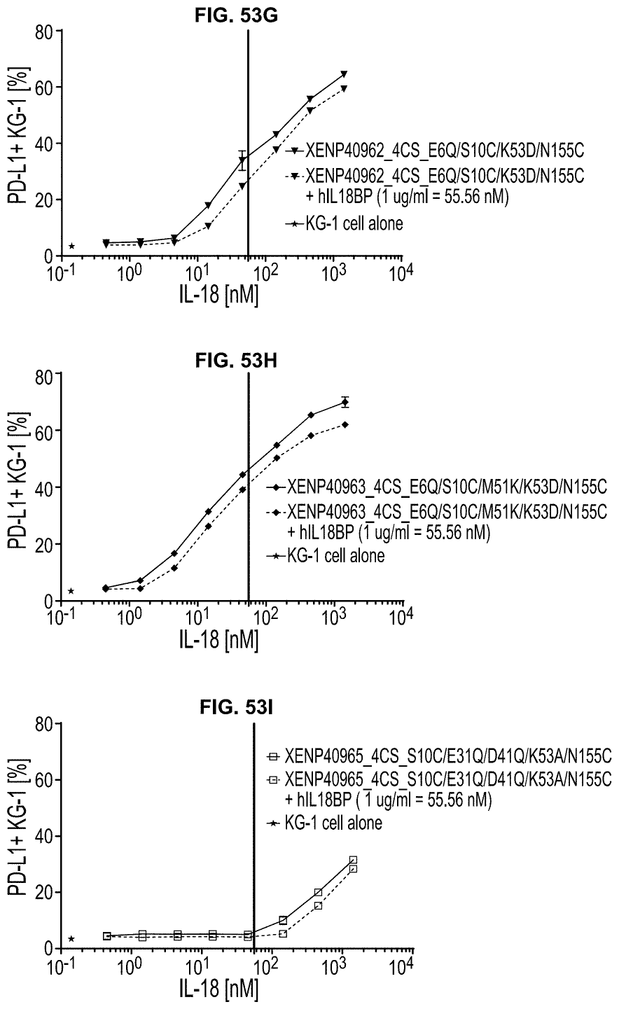

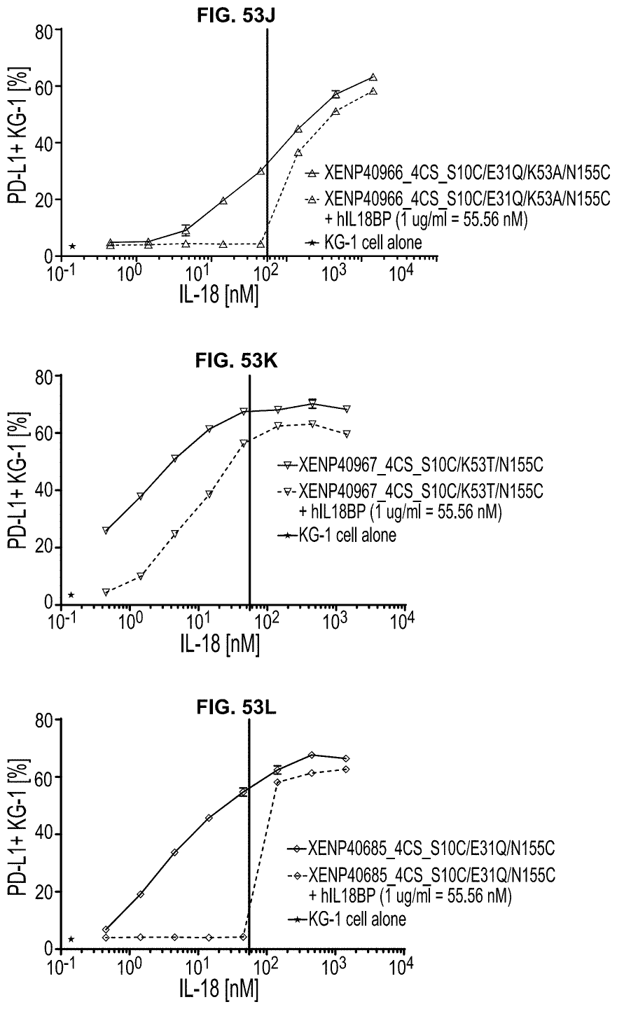

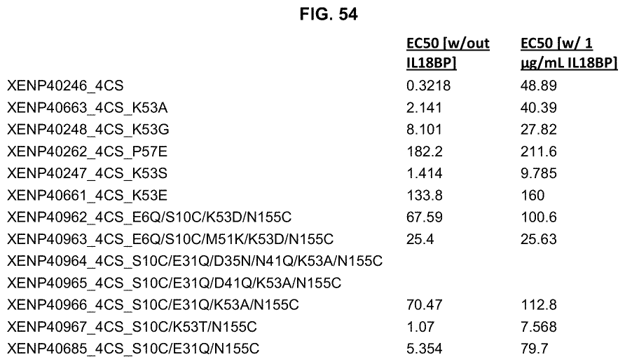

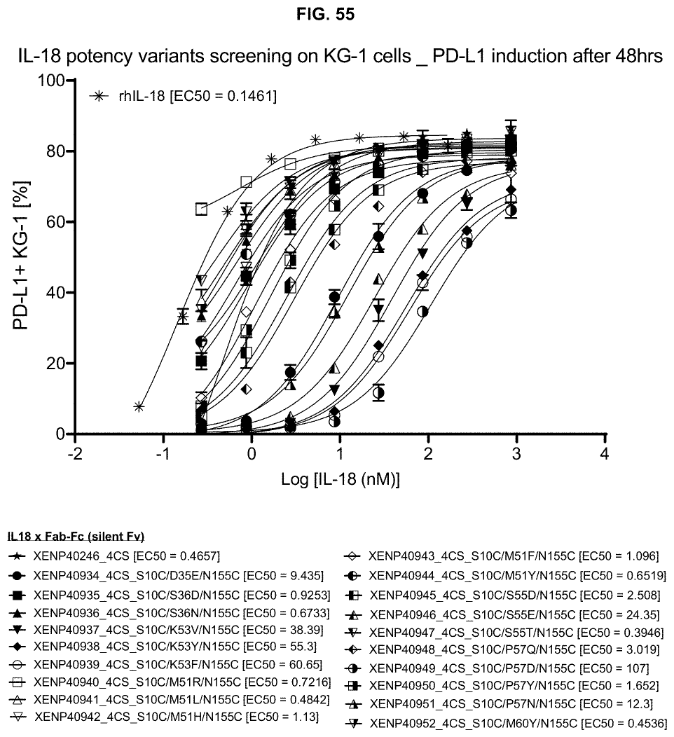

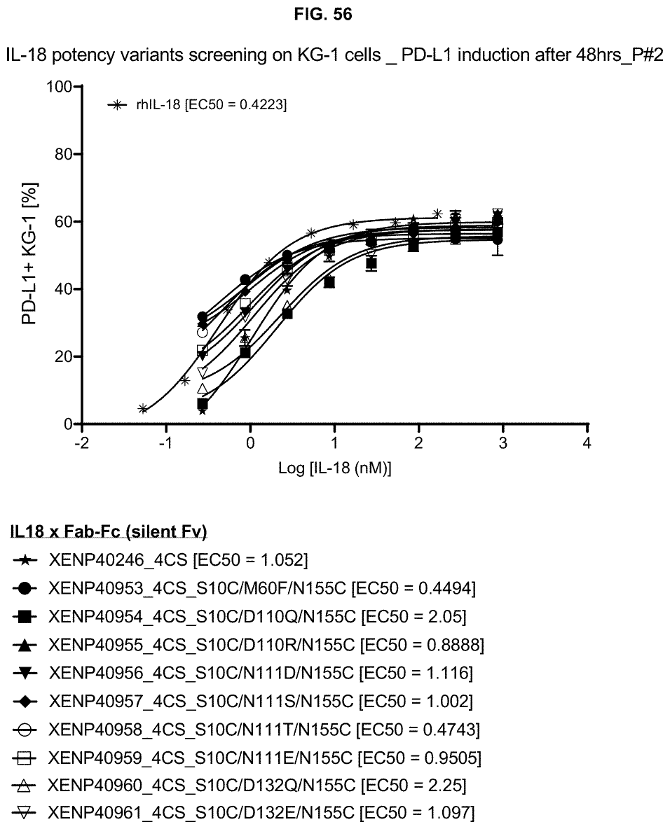

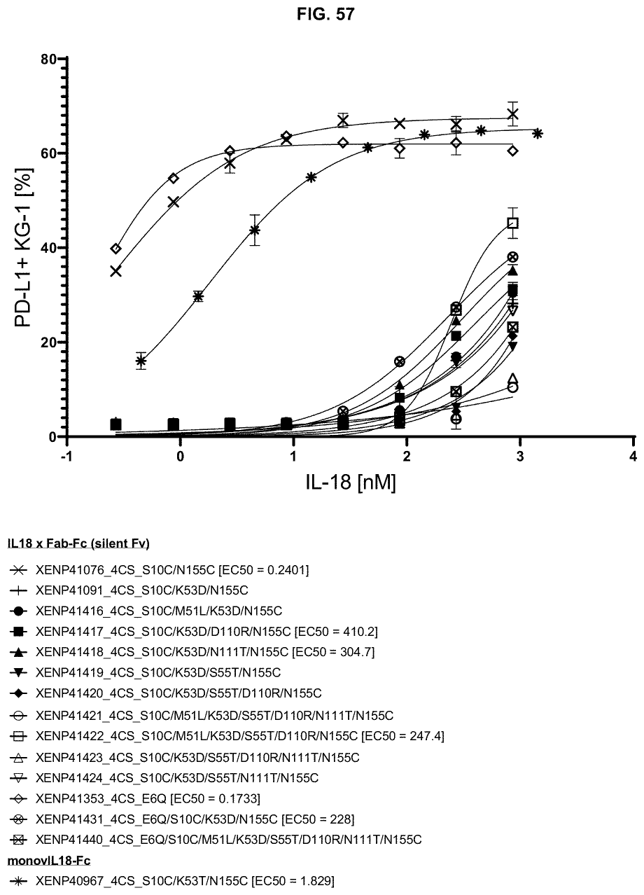

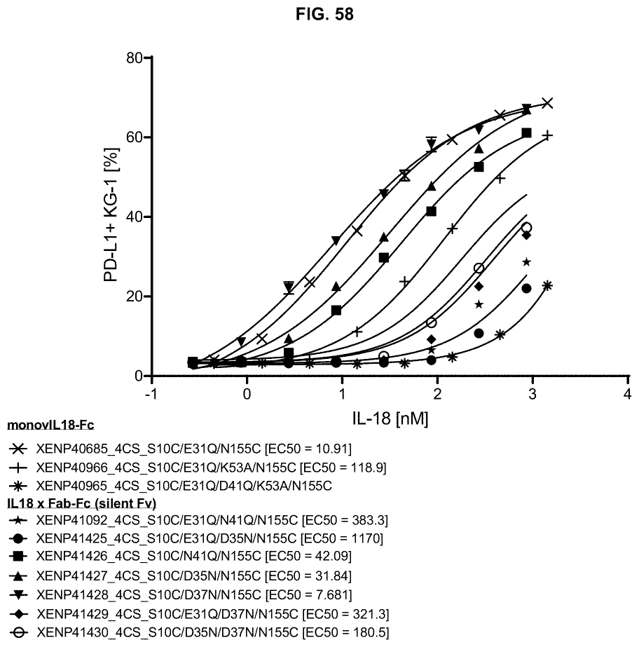

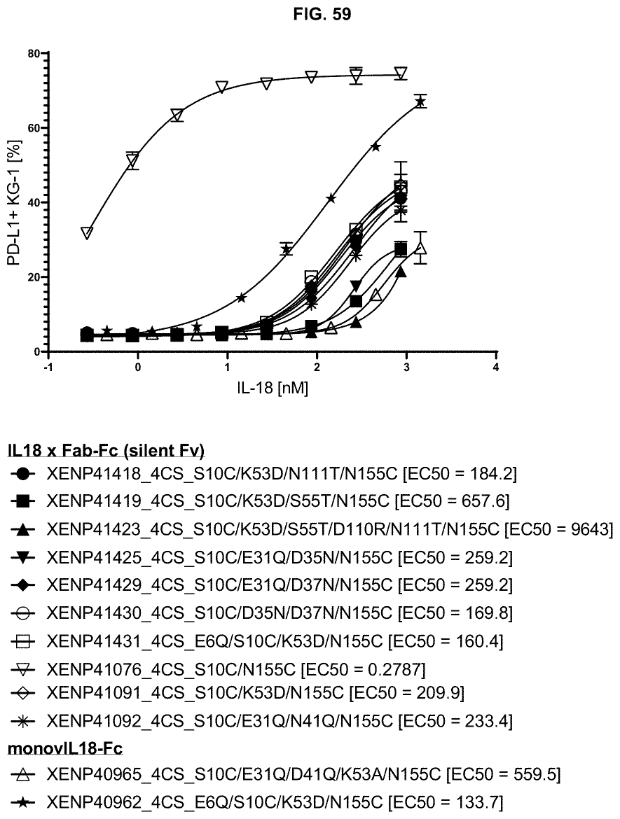

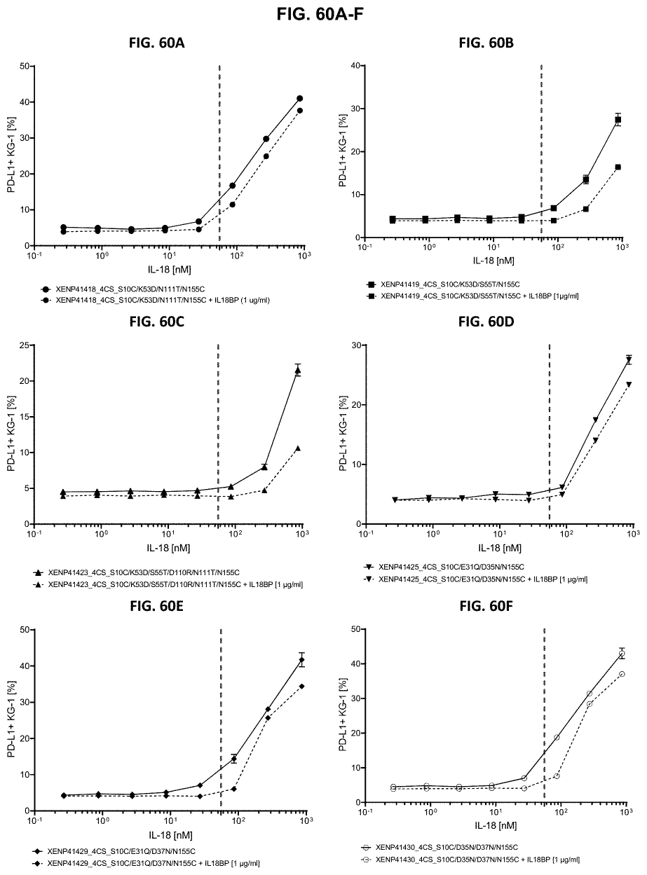

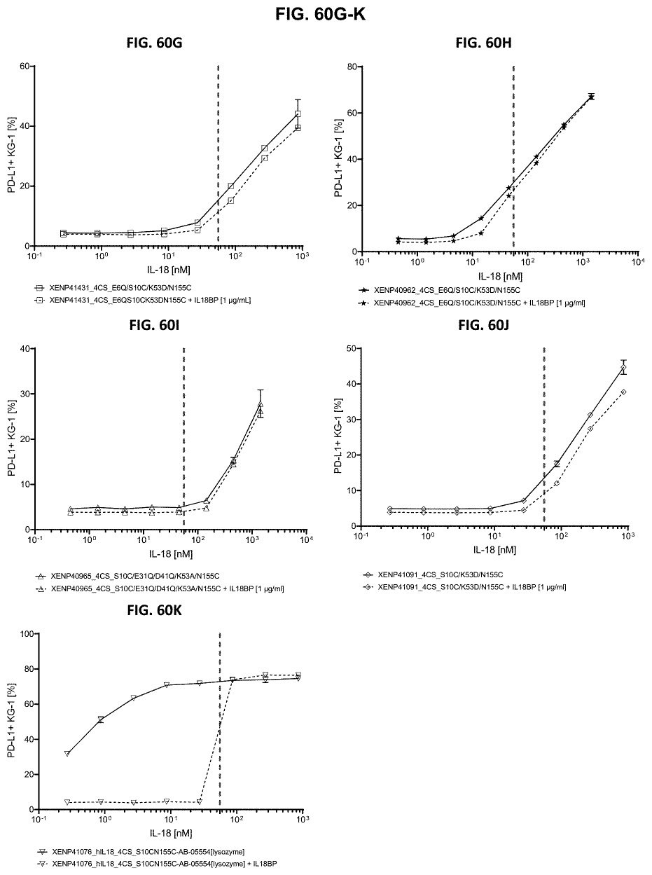

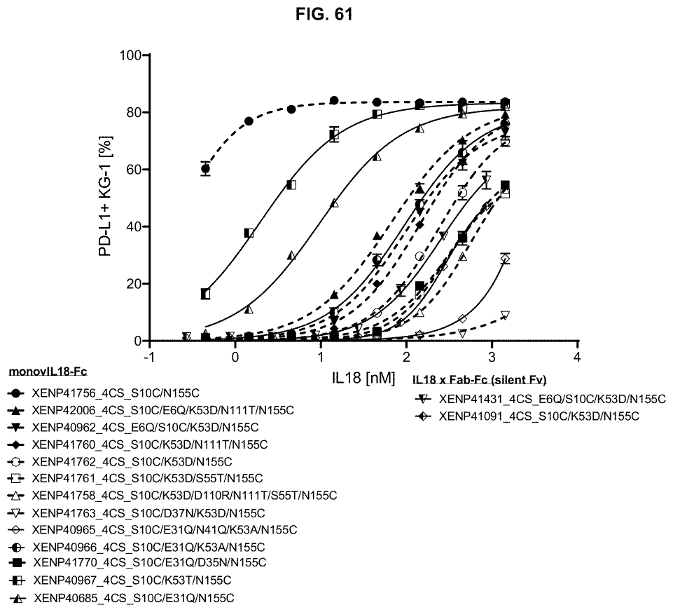

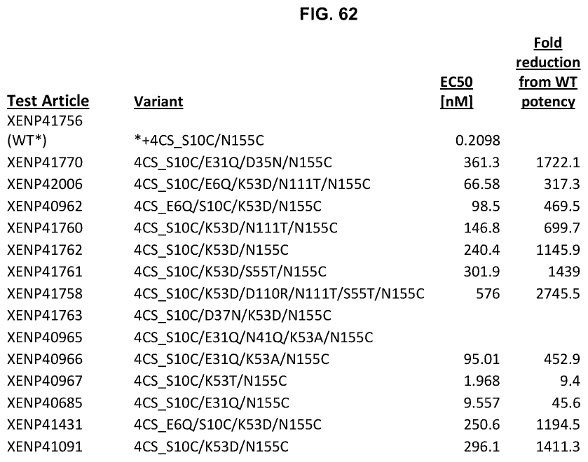

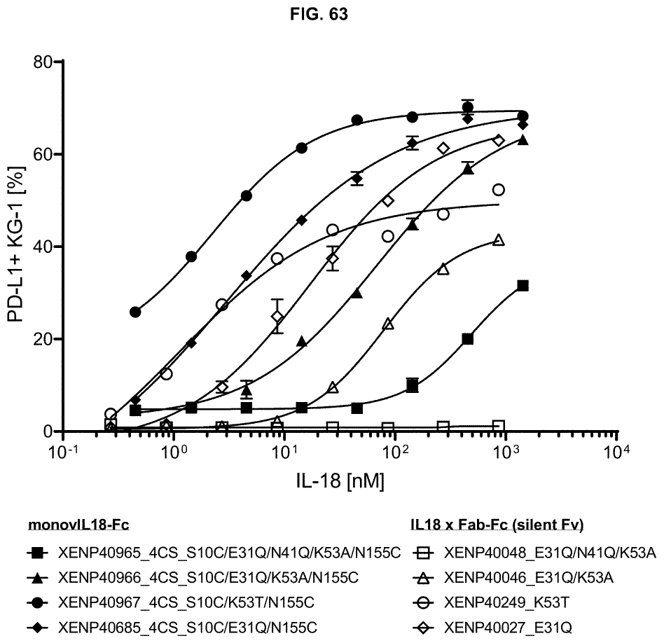

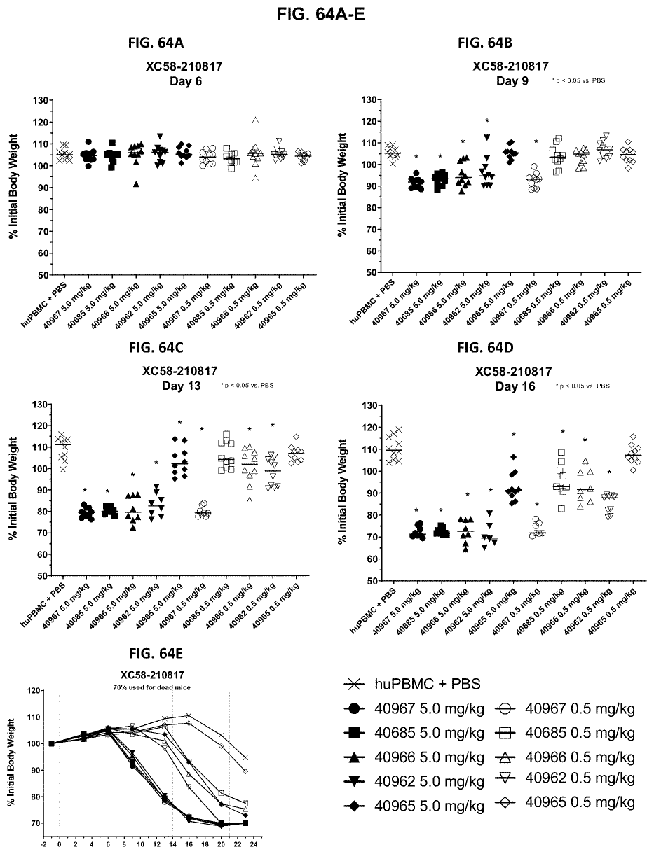

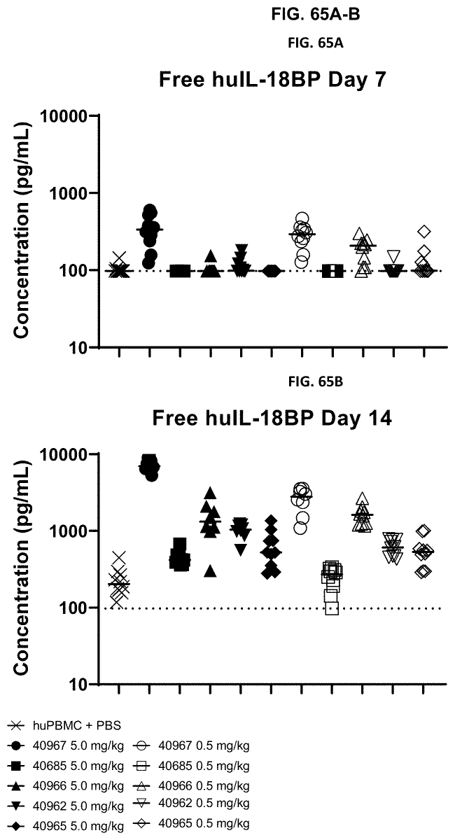

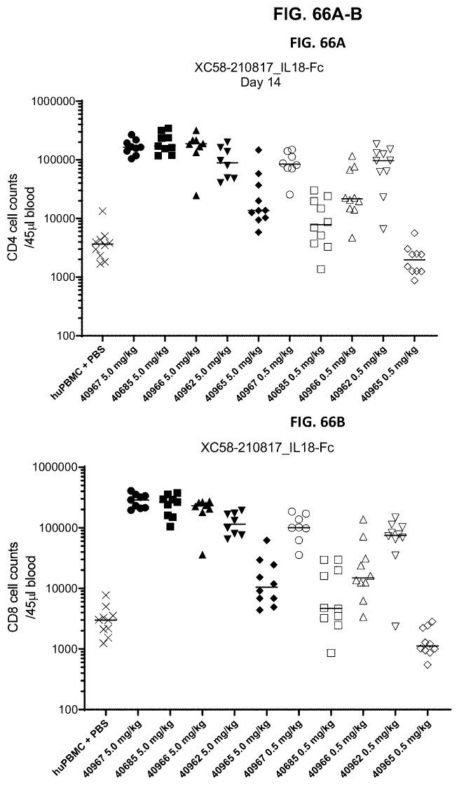

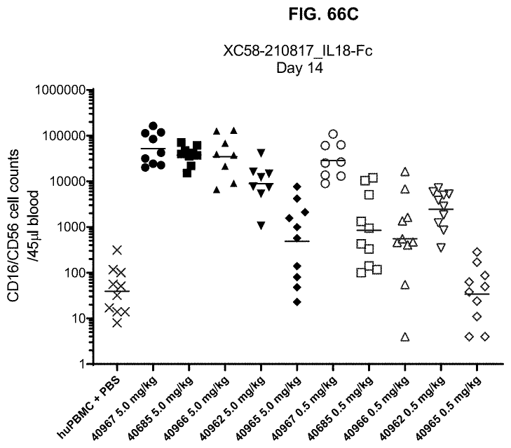

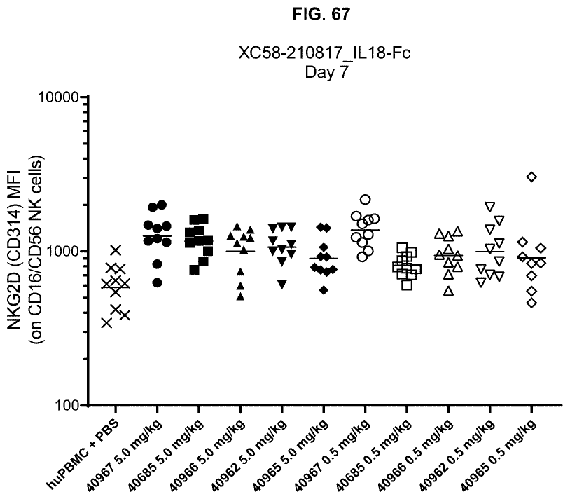

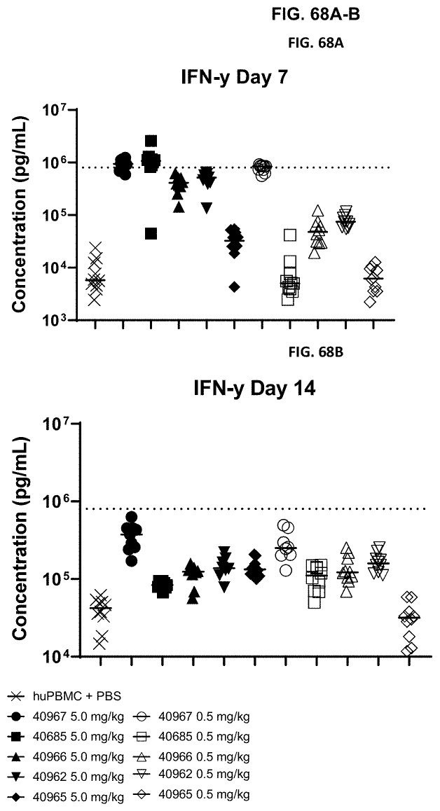

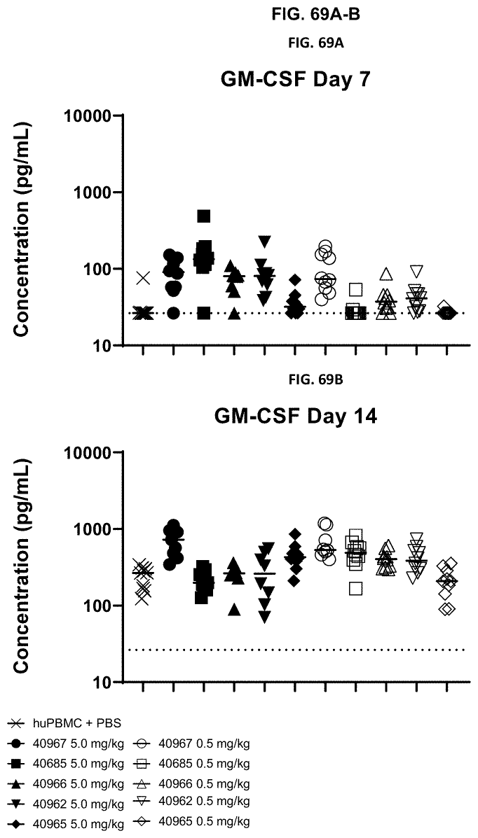

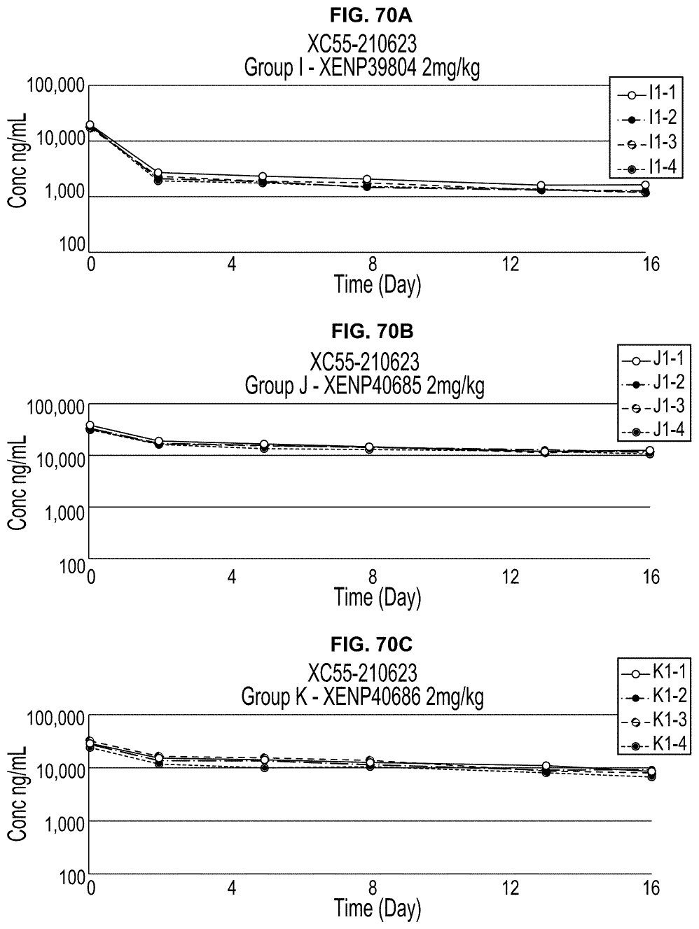

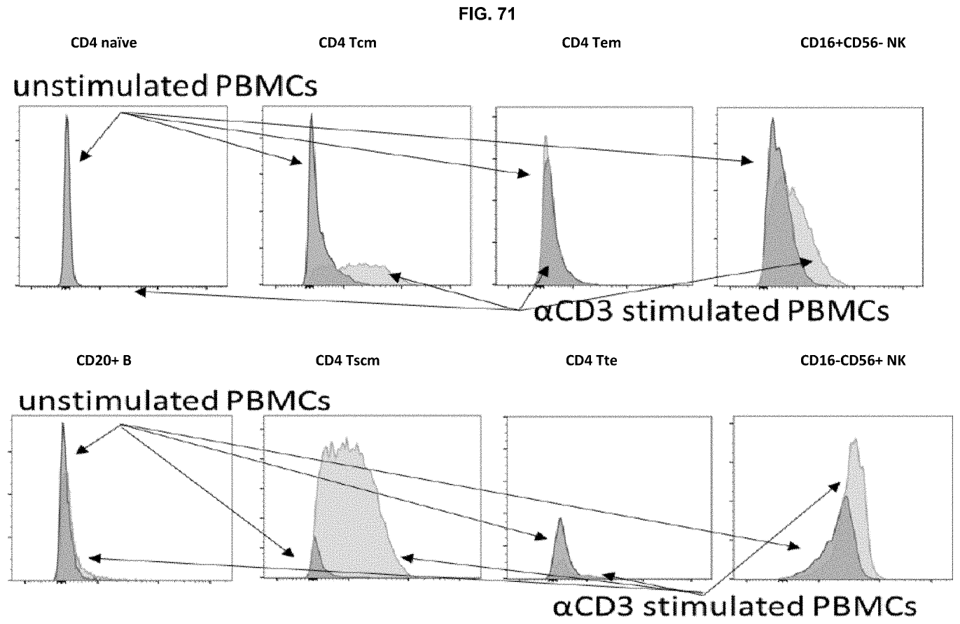

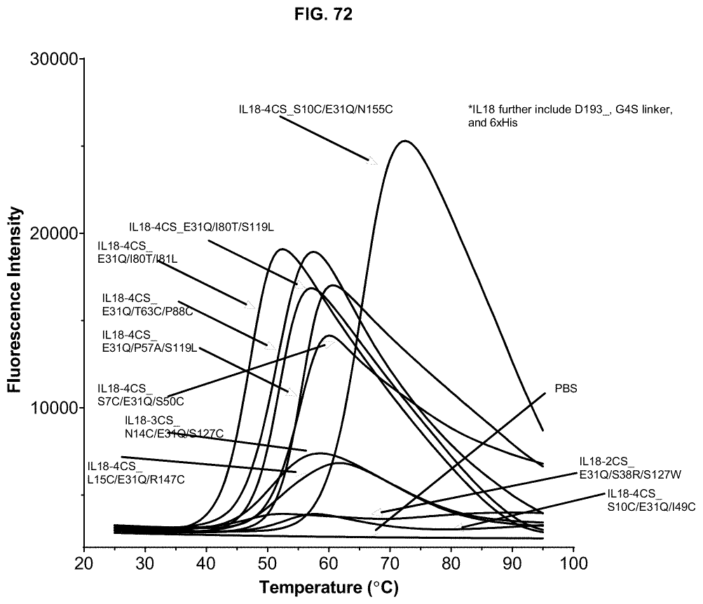

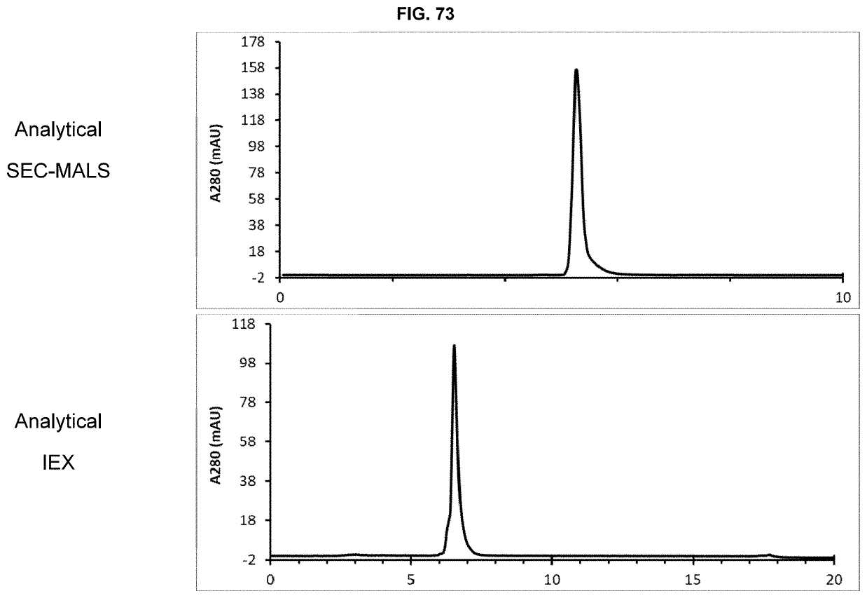

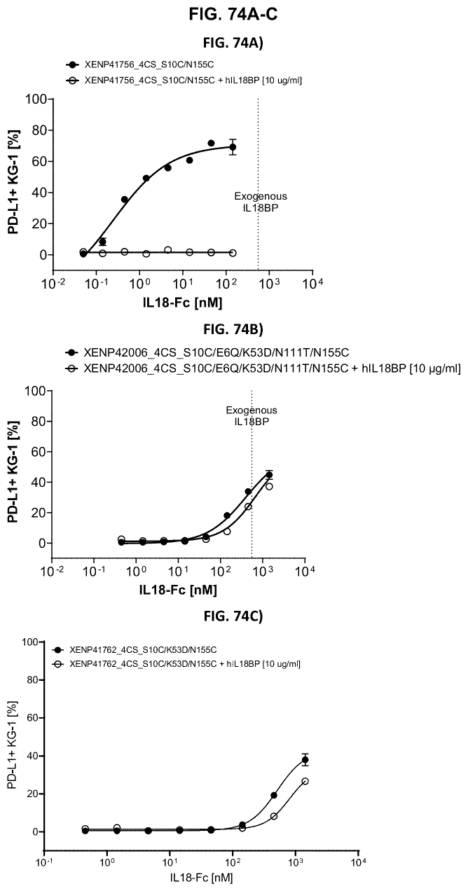

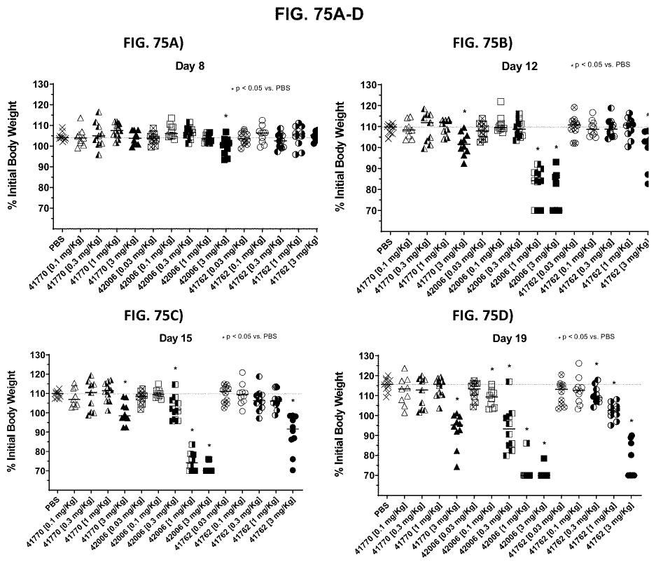

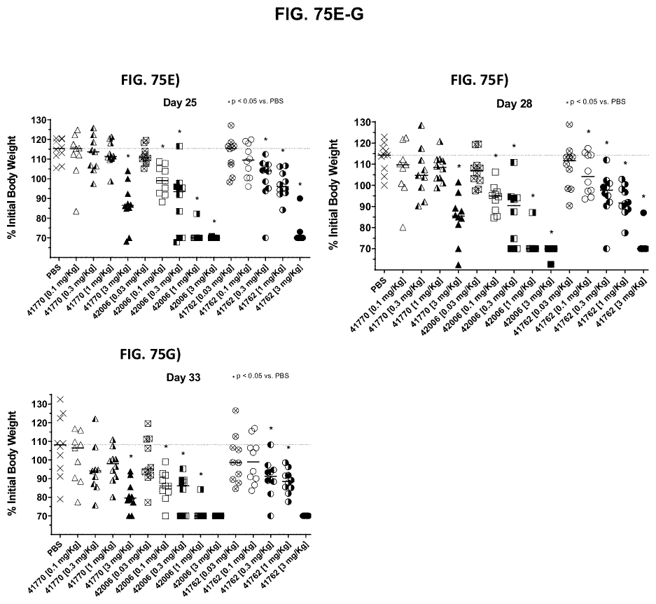

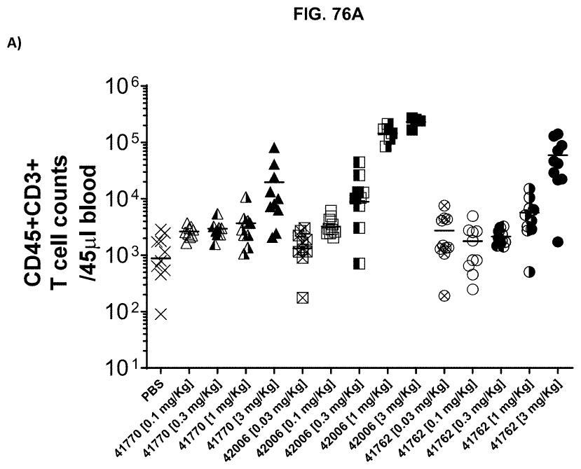

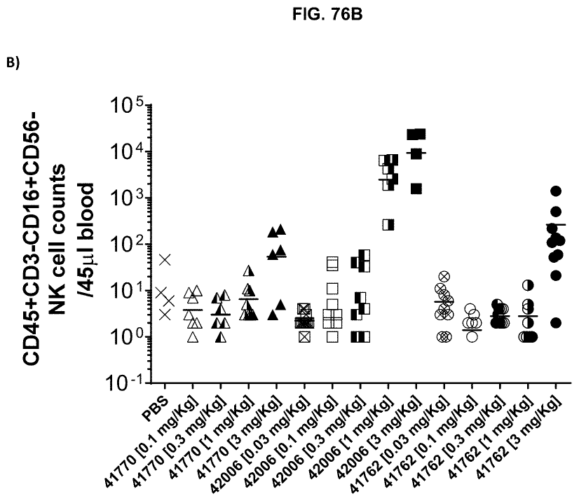

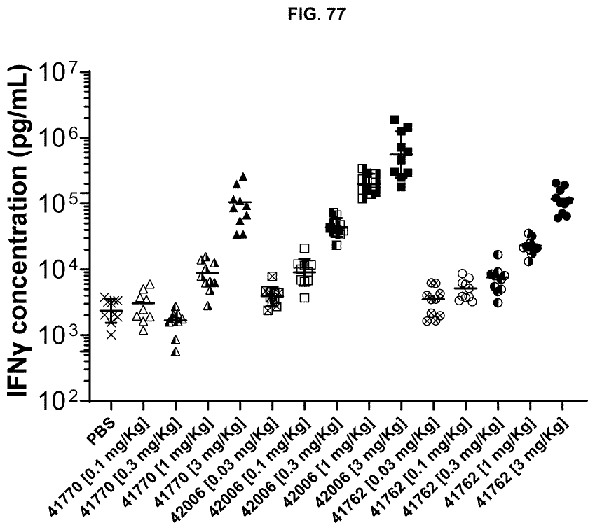

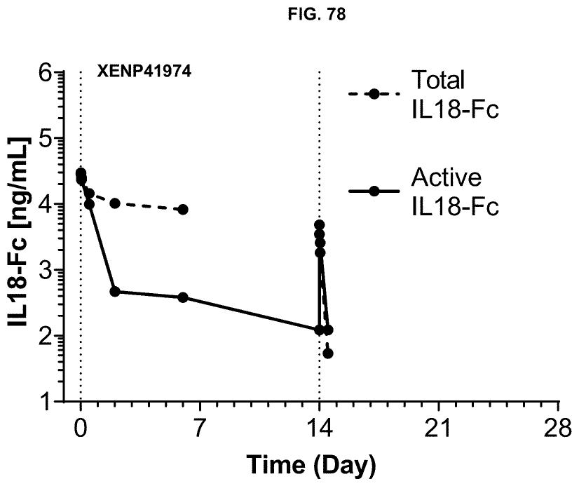

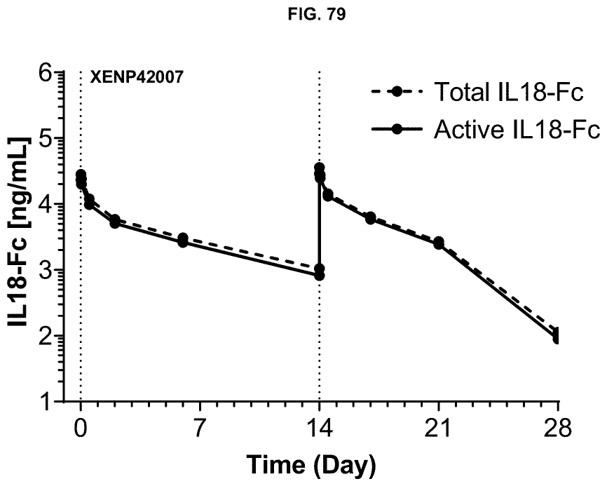

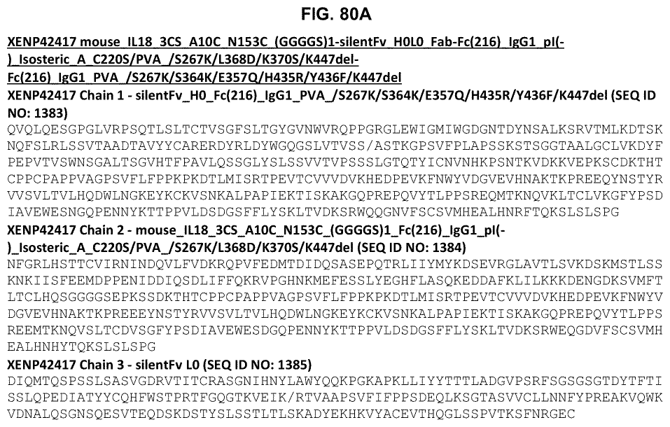

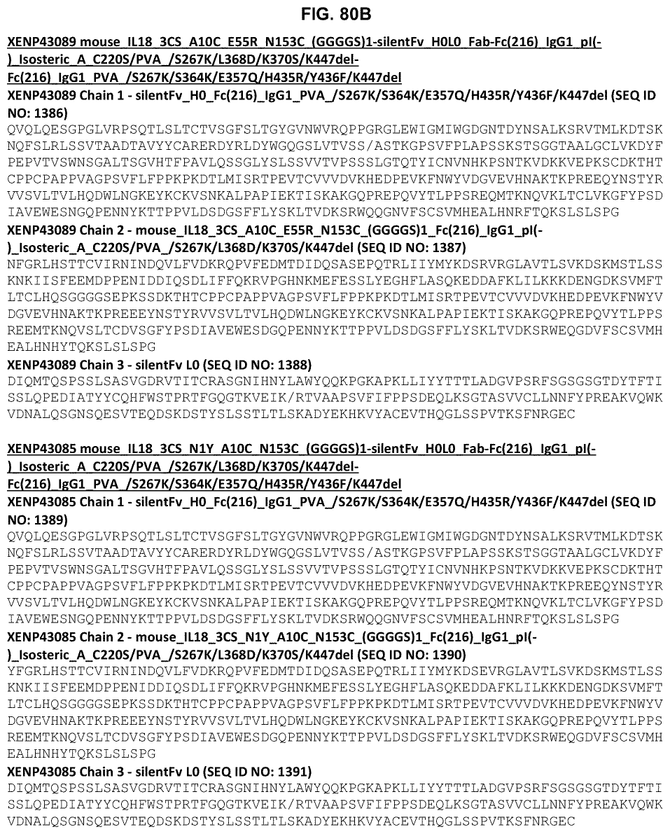

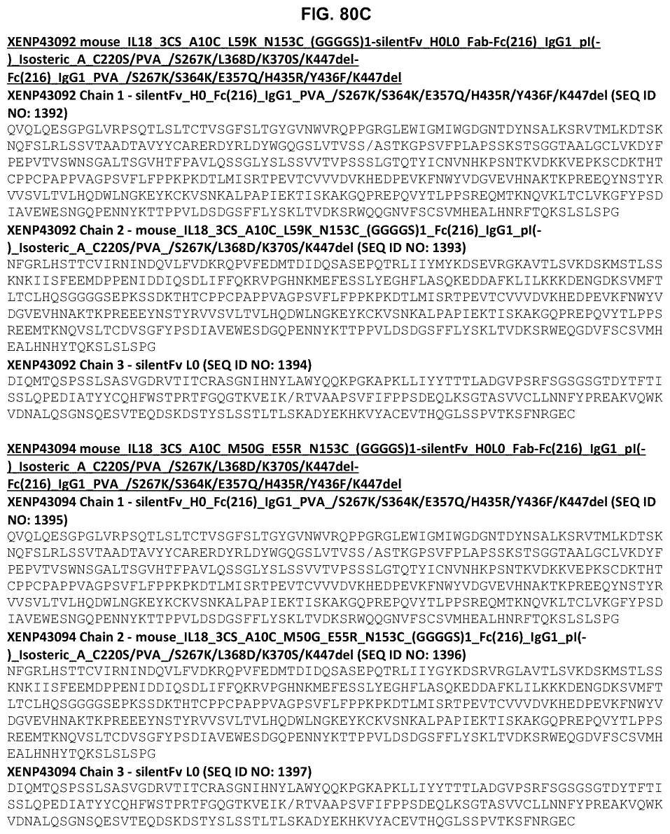

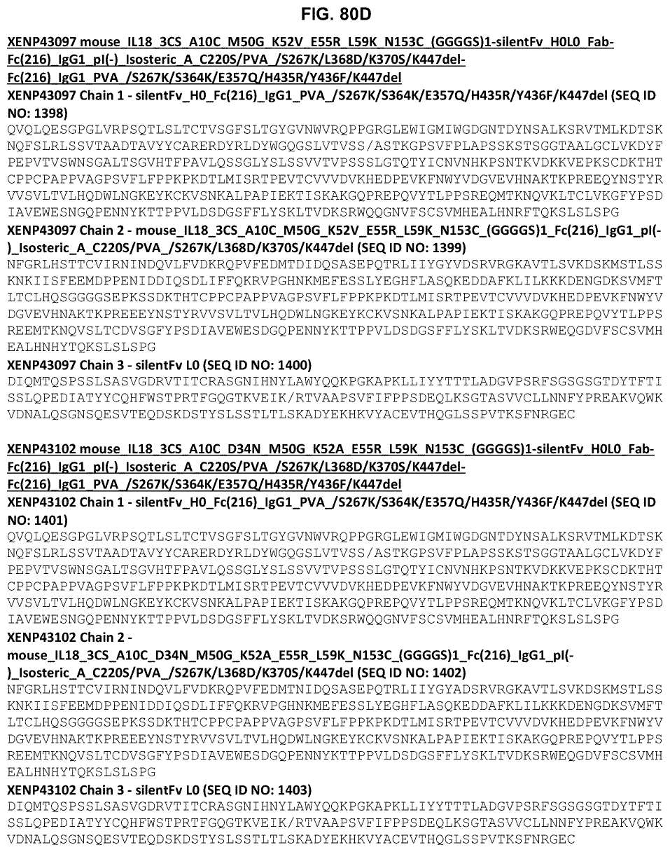

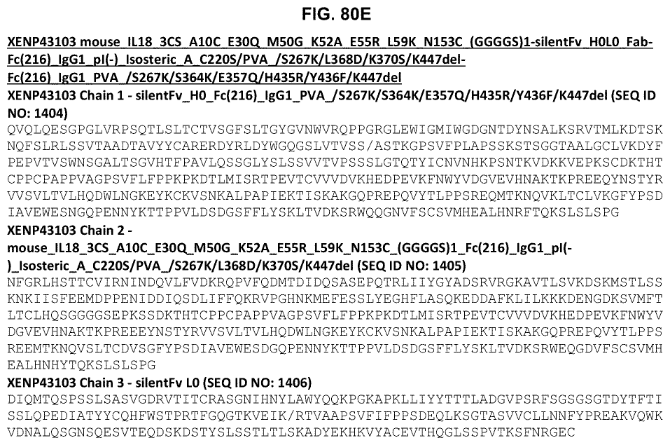

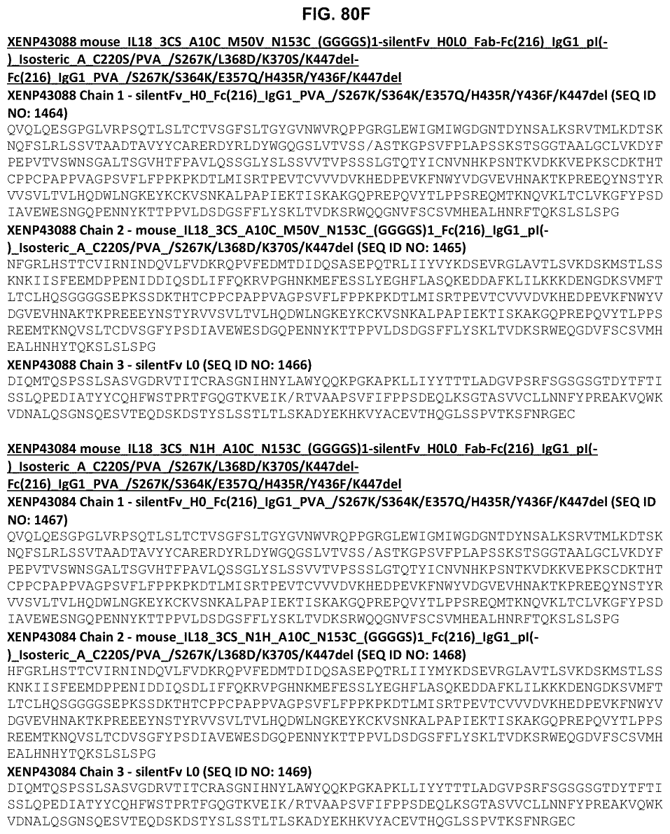

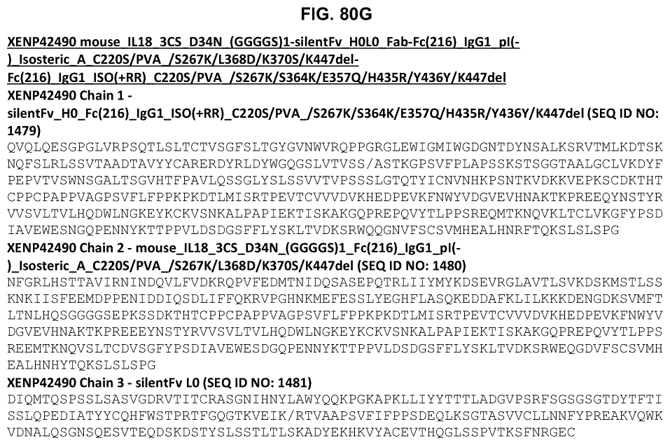

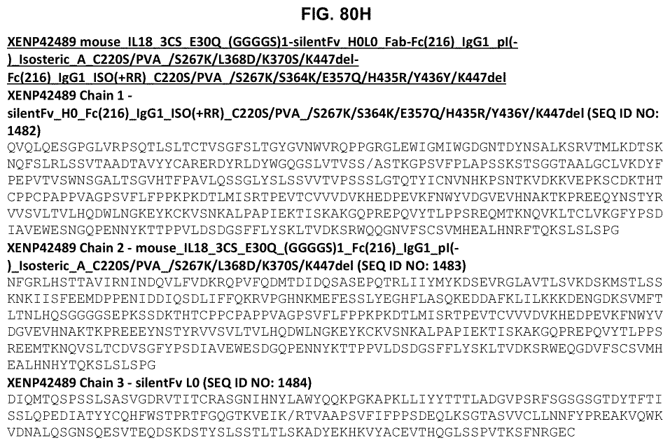

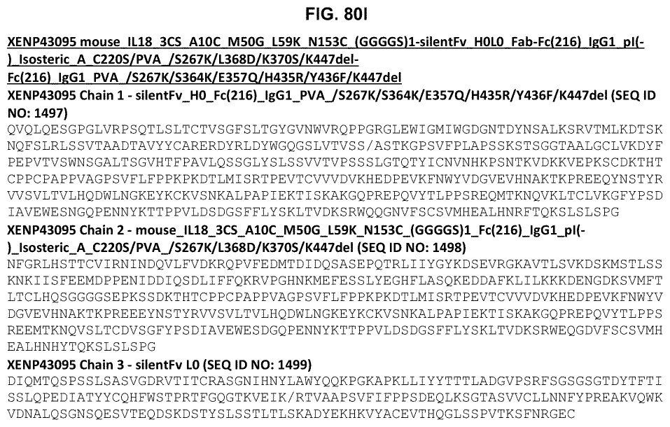

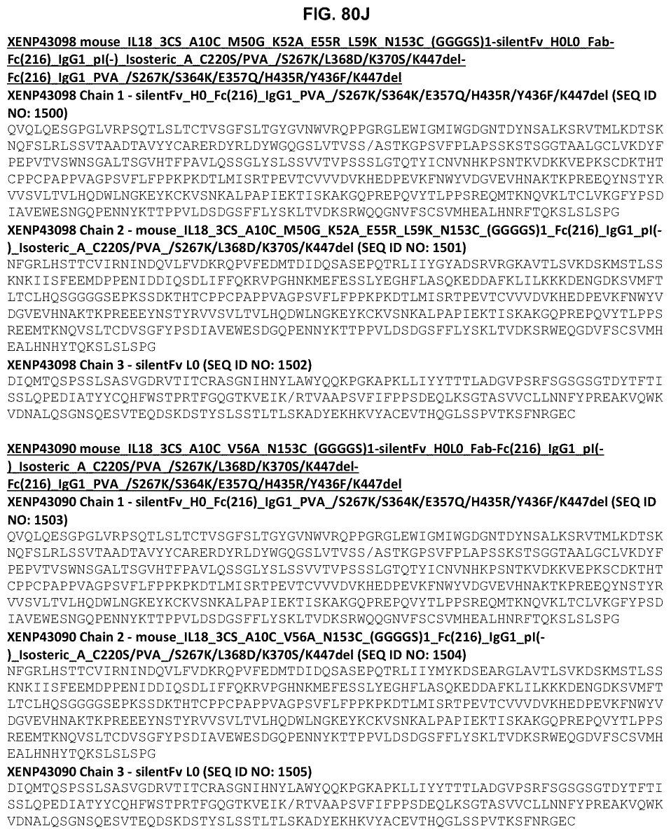

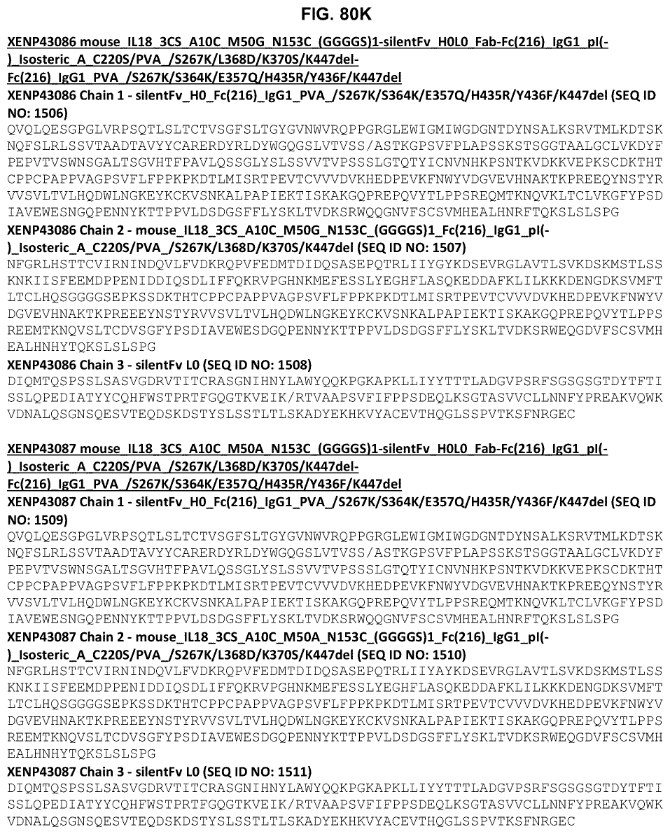

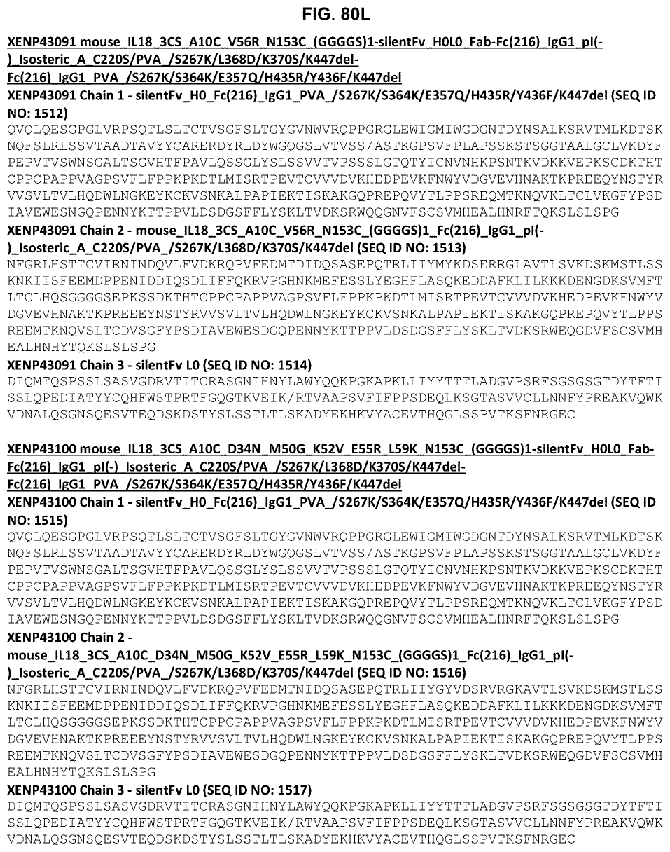

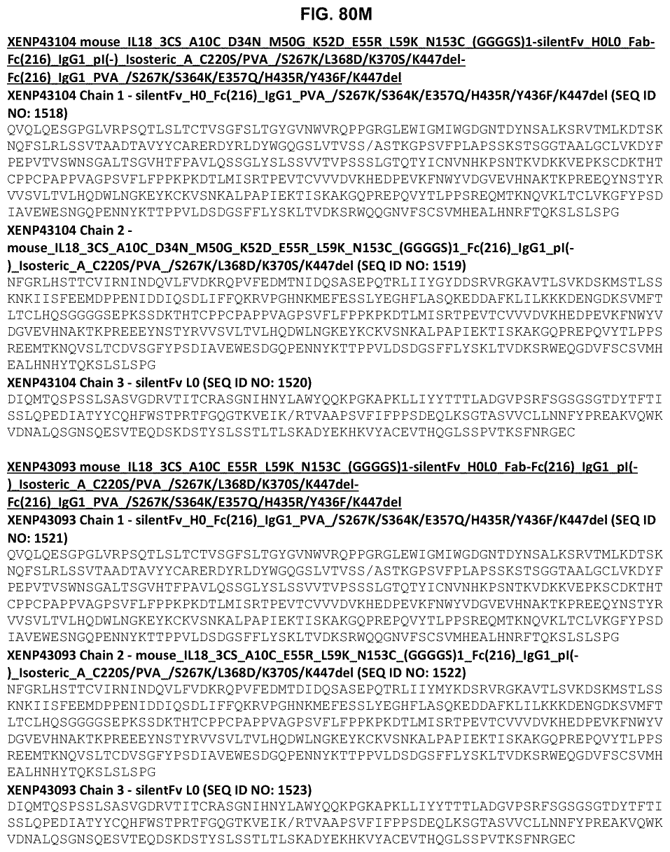

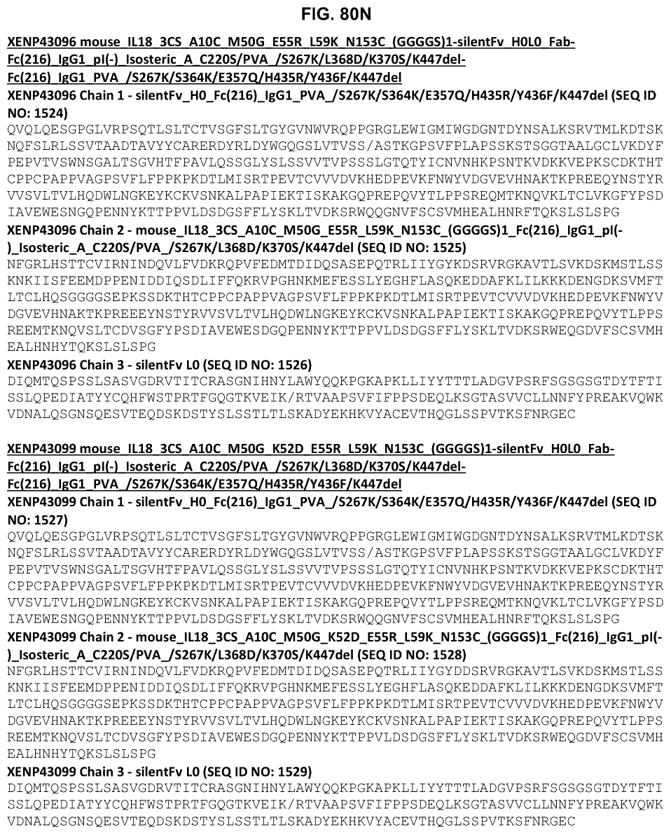

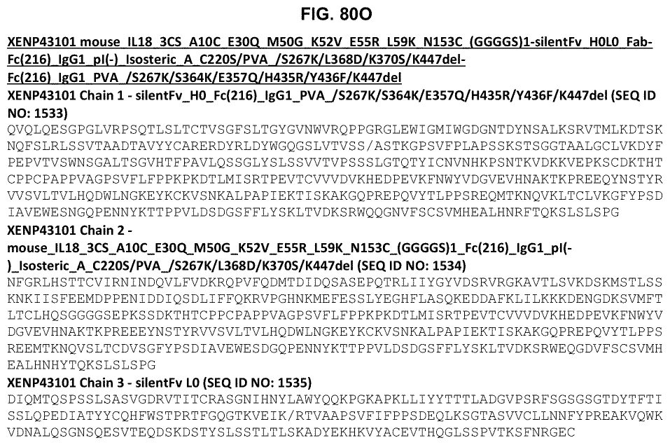

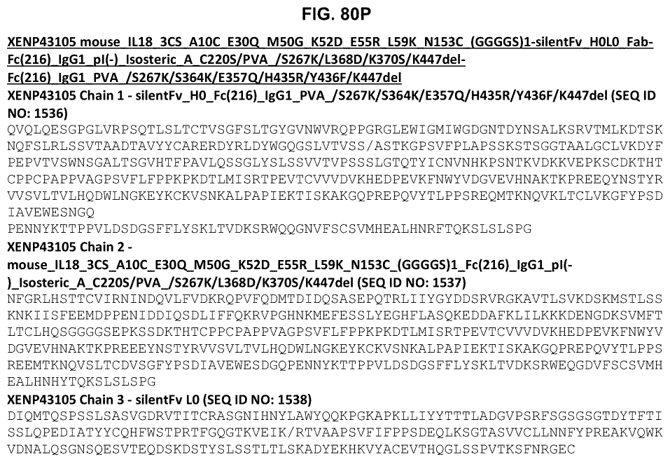

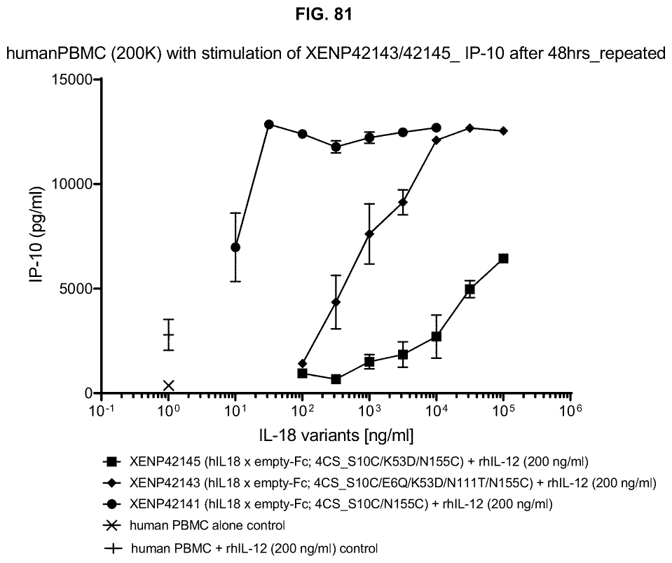

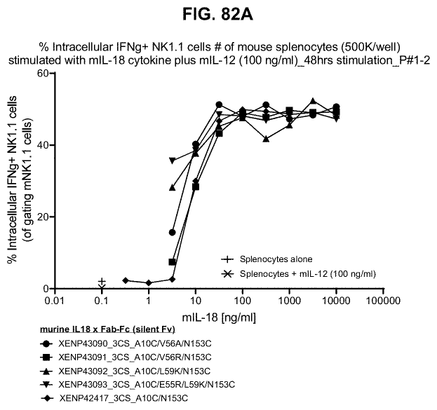

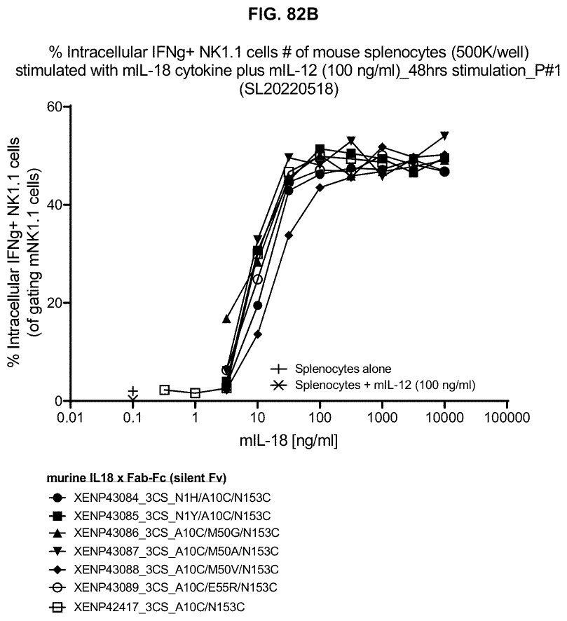

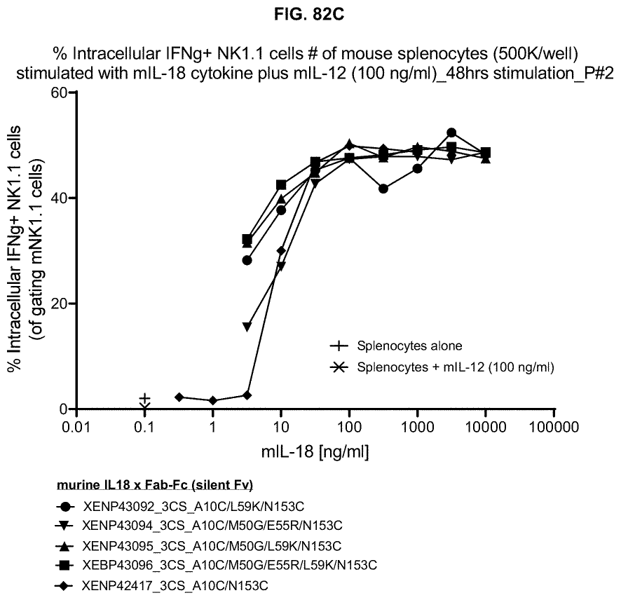

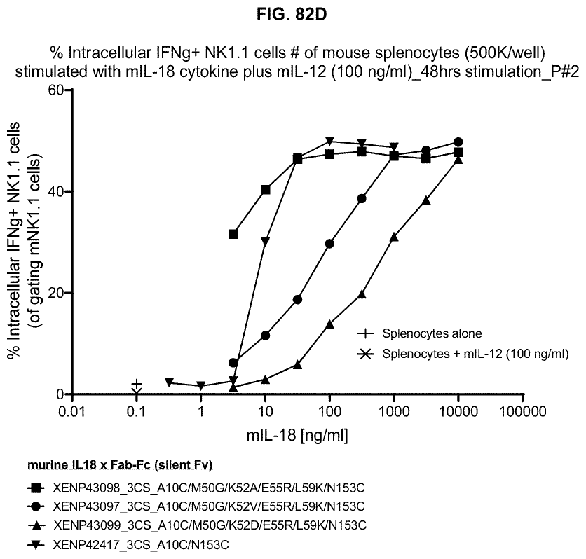

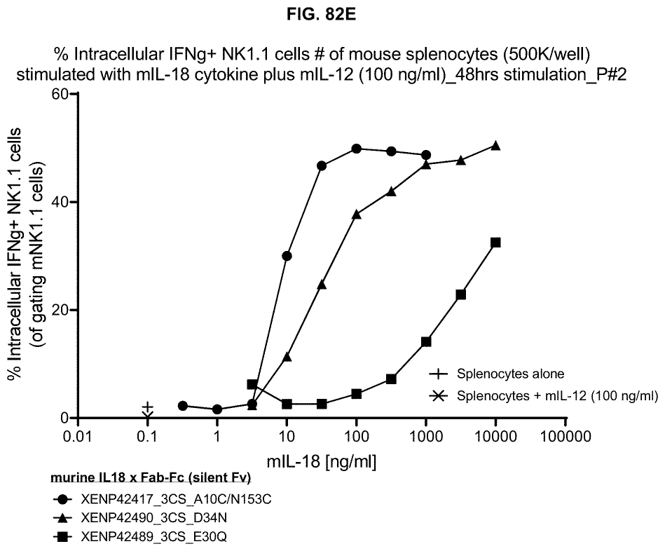

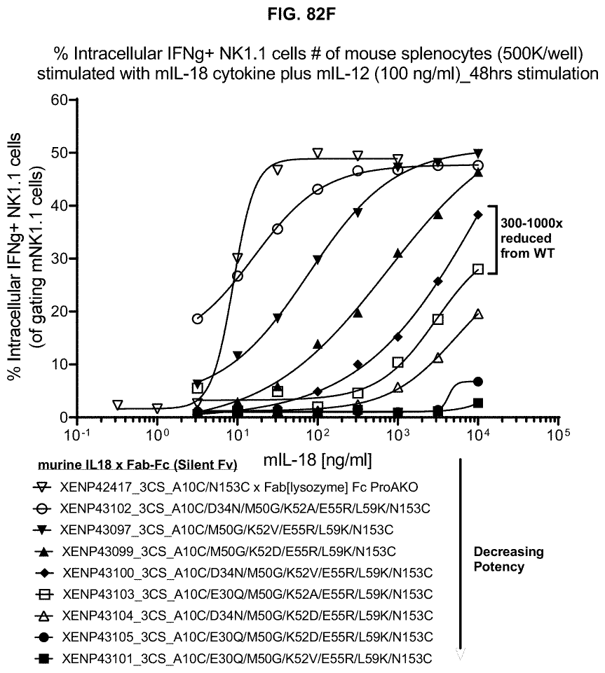

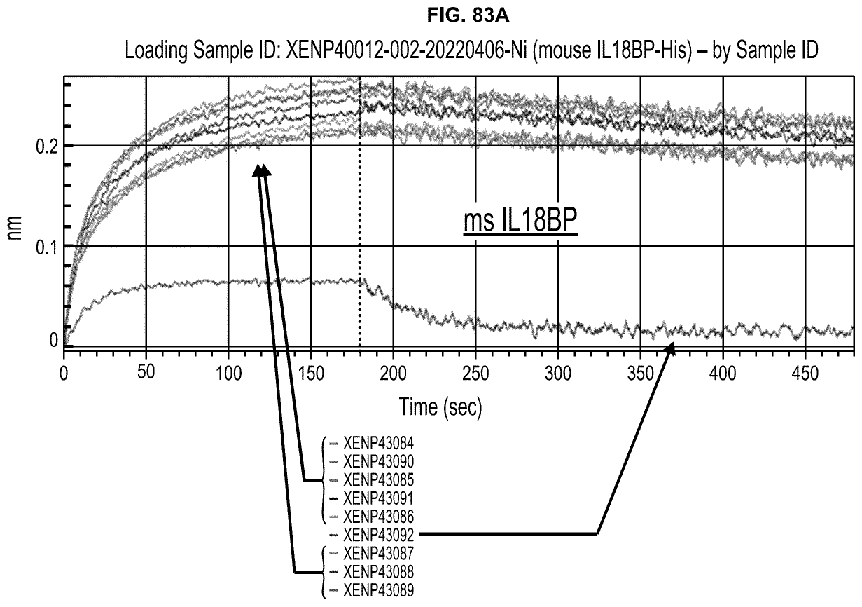

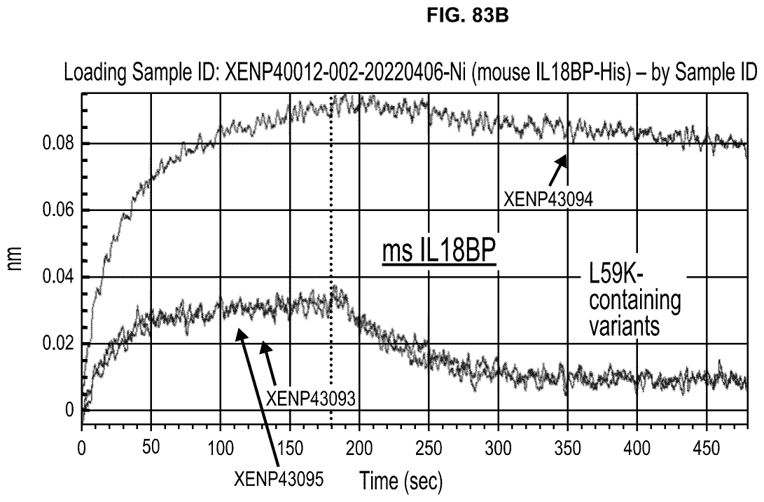

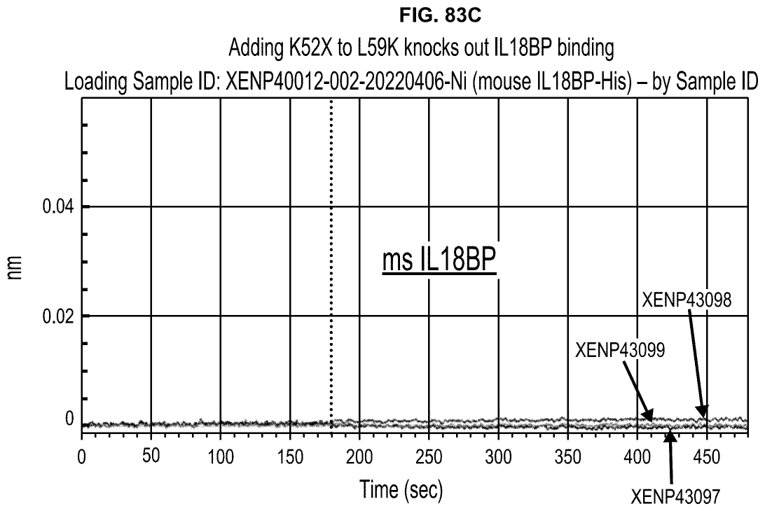

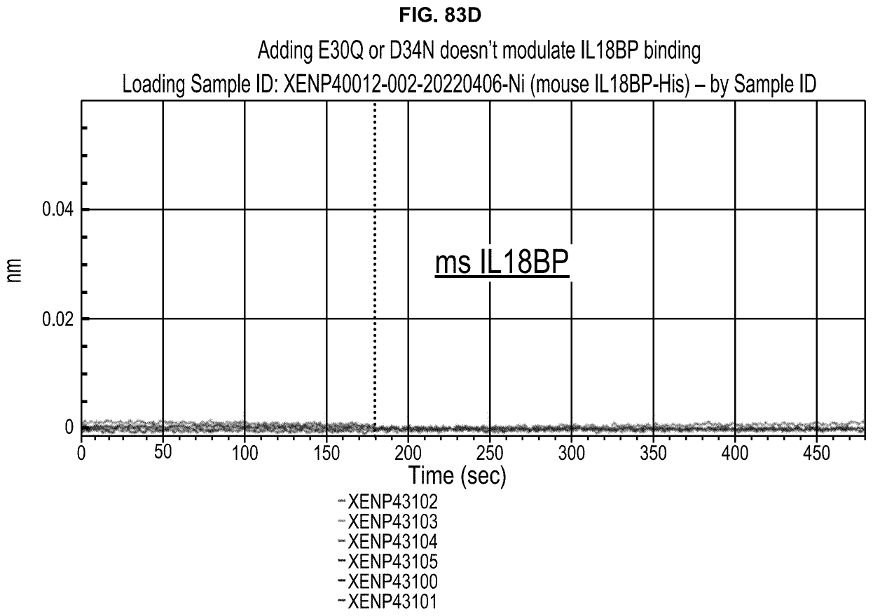

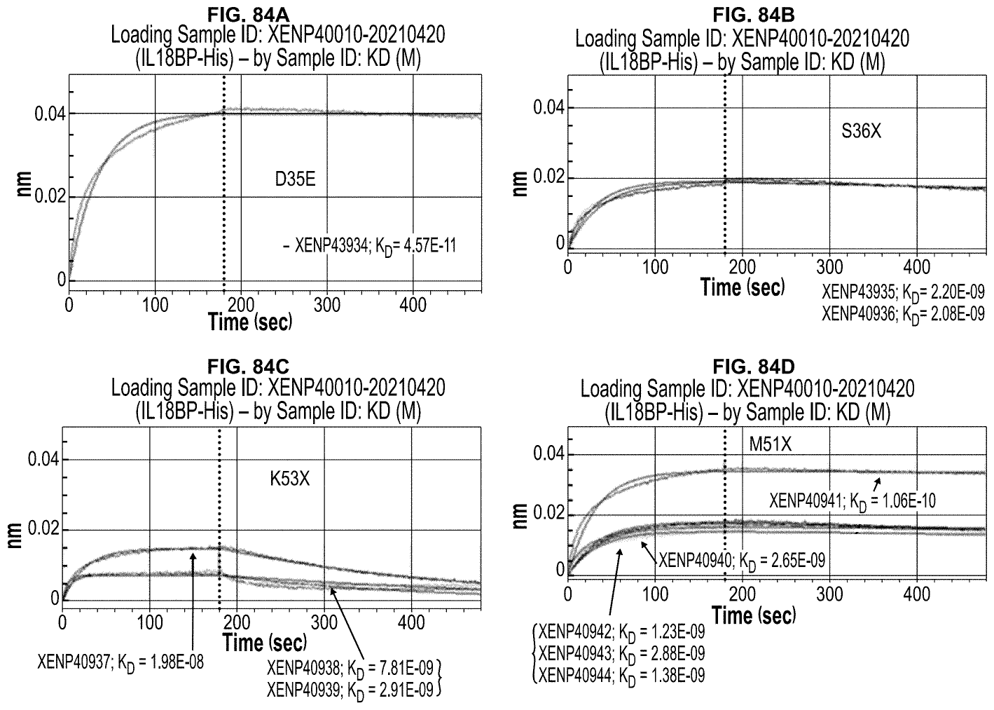

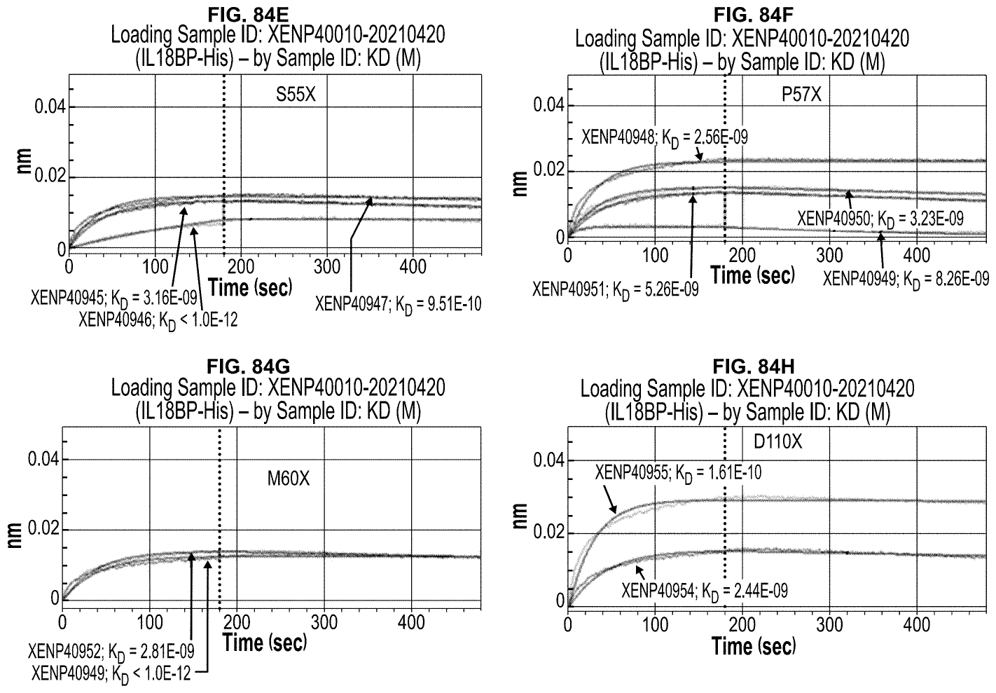

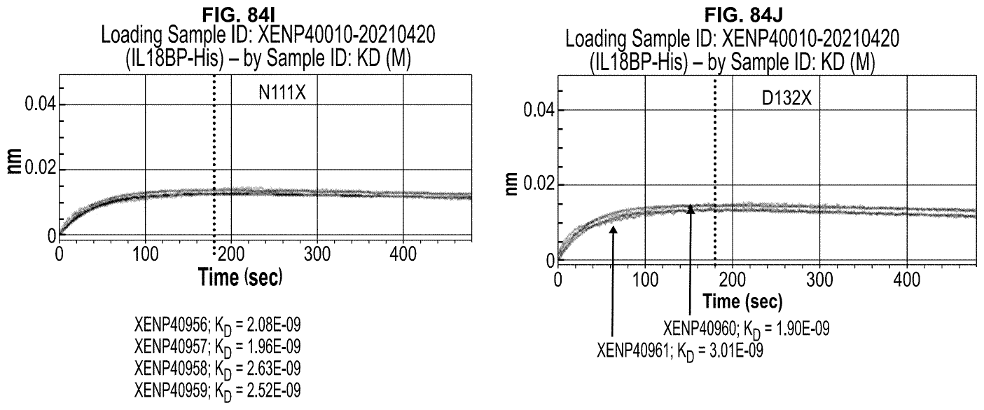

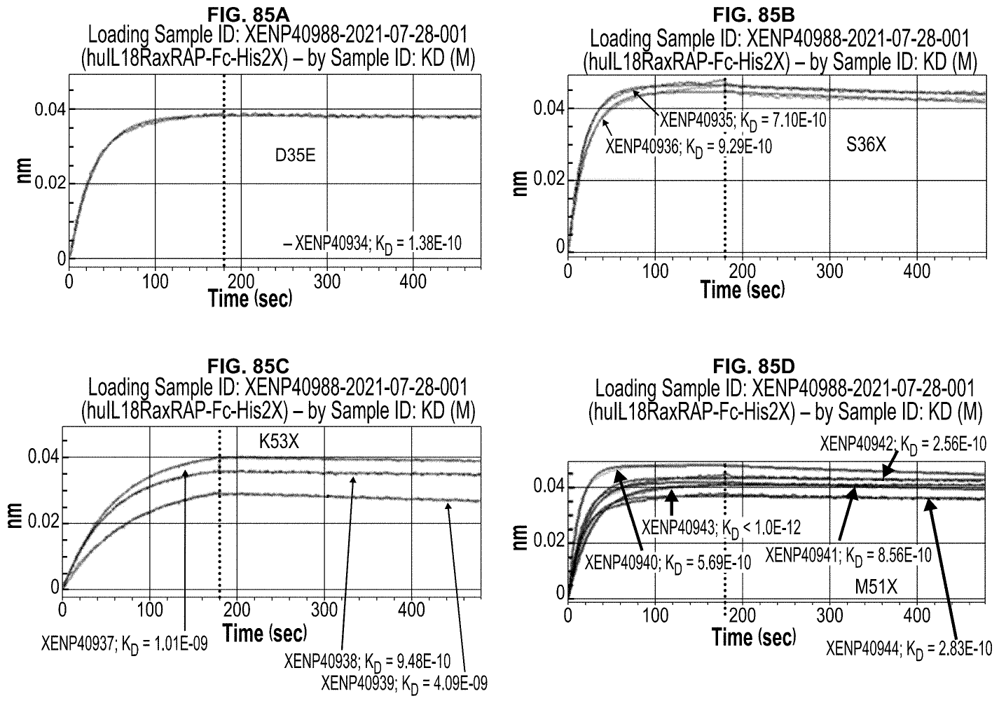

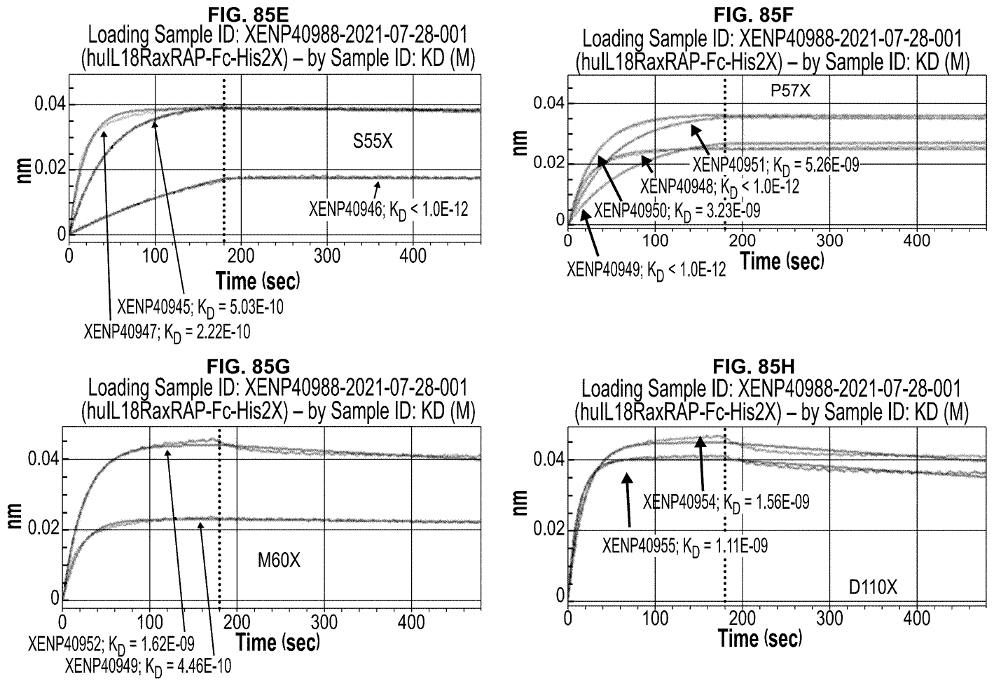

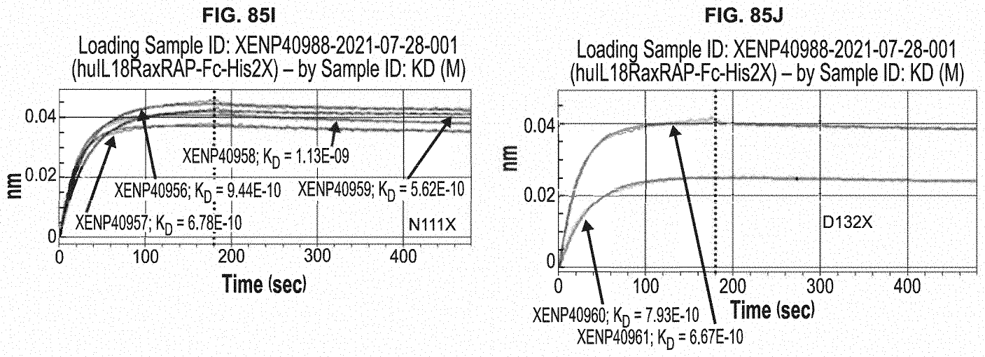

FIG. 10 depicts illustrative CH1 regions which may find use in covalently linking a variable domain to the backbones as depicted in FIG. 9A-E (optionally via a domain linker as depicted in FIG. 8) in the context of IL18 x Fab-Fc fusions.

FIG. 11 depicts the “non-Fv” backbone of cognate light chains (i.e. constant light chain) which find use in the IL18 x Fab-Fc fusion proteins of the invention.

FIGS. 12A-12B depicts illustrative formats for IL18 fusions of the invention. The monovIL18-Fc format (FIG. 12A) comprises a first monomer comprising an IL18 monomer covalently attached to the N-terminus of a first heterodimeric Fc chain (optionally via a domain linker) and a second monomer comprising a complementary second heterodimeric Fc chain that is “Fc-only” or “empty-Fc”. The IL18 x Fab-Fc format (FIG. 12B) comprises a first monomer comprising an IL18 monomer covalently attached to the N-terminus of a first heterodimeric Fc chain (optionally via a domain linker), a second monomer comprising a variable heavy (VH) region covalently attached to the N-terminus of a complementary second heterodimeric Fc chain, and a third monomer that is a corresponding light chain that forms a Fab with the second monomer.

FIGS. 13A-13B depict sequences for IL18 production variant comprising C38S, C68S, C76S and/or C127S.

FIG. 14 depicts sequences for IL18 production variants engineered to remove C-terminal aspartic acid.

FIGS. 15A-15B depict sequences for IL18 single substitution affinity variants (Library 1). It should be noted that each of these variants may include additional substitutions such as production variants, affinity variants, and/or stability variants.

FIGS. 16A-16C depict sequences for IL18 single substitution affinity variants (Library 2). It should be noted that each of these variants may include additional substitutions such as production variants, affinity variants, and/or stability variants.

FIGS. 17 depicts sequences for IL18 combo substitution affinity variants (Library 2). It should be noted that each of these variants may include additional substitutions such as production variants, affinity variants, and/or stability variants.

FIG. 18 depicts additional affinity variants which may find use in the IL18 fusion proteins of the invention.

FIGS. 19A-19I depicts sequences for IL18 stability variants (Library 1). It should be noted that each of these stability variants include the 4CS substitutions, unless reversion to cysteine is explicitly denoted i.e. S38C, S68C, S76C, and/or S127C. However, it should be noted that the variants at positions other than 38, 68, 76, or 127 may be used independently from the 4CS substitutions. Additionally, it should be noted that each of these variants may include additional substitutions such as production variants, affinity variants, and/or stability variants.

FIGS. 20A-20C depict sequences for IL18 stability variants (Library 2). It should be noted that each of these stability variants include the 4CS substitutions, unless alternative substitution at residues 38, 68, 76, and/or 127 is explicitly denoted e.g. S38E. However, it should be noted that the substitutions other than C38S, C68S, C76S, and C127S may be used independently from the 4CS substitutions. Additionally, it should be noted that each of these variants may include additional substitutions such as production variants, affinity variants, and/or stability variants.

FIG. 21A-21M depict the sequences for illustrative IL18 fusions of the monovIL18-Fc format comprising WT human IL18 or human IL18 variants. Slashes (/) indicate the border(s) between IL18 monomer, linkers, and Fc regions. It should be noted that IL18 sequences that are 90, 95, 98 and 99% identical (as defined herein), and/or contain from 1, 2, 3, 4, 5, 6, 7, 8, 9 or 10 additional amino acid substitutions and/or exclude amino acid substitutions, including production, affinity, and stability substitutions. Additionally, each of the monovIL18-Fc sequences may utilize alternative backbones (including, but not limited, to those depicted in FIG. 9A-E).

FIGS. 22A-22CZ depict the sequences for illustrative 11,18 fusions of the 11,18 x Fab-Fc format comprising WT human IL18 or human IL18 variants. Slashes (/) indicate the border(s) between IL18 monomer, linkers, and Fc regions. It should be noted that IL18 sequences that are 90, 95, 98 and 99% identical (as defined herein), and/or contain from 1, 2, 3, 4, 5, 6, 7, 8, 9 or 10 additional amino acid substitutions and/or exclude amino acid substitutions, including production, affinity, and stability substitutions. Additionally, each of the IL18 x Fab-Fc sequences may utilize alternative backbones (including, but not limited, to those depicted in FIG. 9A-E) or alternative variable heavy and variable light domains.

FIGS. 23A-23B depict A) chromatogram illustrating purification part 2 of XENP30792 (anion exchange chromatography following protein A chromatography), and the heterogeneity of pre-purified material and material from peak B isolated from anion exchange separation as depicted in FIG. 23A by B) analytical size-exclusion chromatography with multi-angle light scattering (aSEC-MALS). FIG. 23B also depicts the molecular weight of protein species as determined by multi-angle light scattering.

FIGS. 24A-24B depict A) chromatogram illustrating purification part 2 of XENP31296 (anion exchange chromatography following protein A chromatography), and the heterogeneity of pre-purified material and purity of material from peak B isolated from anion exchange separation as depicted in FIG. 24A by B) analytical size-exclusion chromatography with multi-angle light scattering (aSEC-MALS). FIG. 24B also depicts the molecular weight of protein species as determined by multi-angle light scattering.

FIGS. 25A-25B depict A) chromatogram illustrating purification part 2 of XENP37827 (anion exchange chromatography following protein A chromatography), and the purity material from peak B isolated from anion exchange separation as depicted in FIG. 25A by B) analytical size-exclusion chromatography with multi-angle light scattering (aSEC-MALS). FIG. 25B also depicts the molecular weight of protein species as determined by multi-angle light scattering.

FIGS. 26A-26B depict A) dose dependent activation of KG-1 cells (as indicated by PD-L1 expression) by recombinant human IL18 and B) dose dependent neutralization of IL18 activity on KG-1 cells (as indicated by PD-1 expression) by IL18BP in the presence of 0.86 nM (100 ng/ml) recombinant human IL18.

FIG. 27 depicts activation of KG-1 cells (as indicated by induction of PD-L1 expression) by IL18 affinity variants (in the context of IL18 x Fab-Fc fusions having a silent Fv). The data show that a number of substitutions enabled reduced activation potency in comparison to WT-4CS variant.

FIGS. 28A-28S depict activation of KG-1 cells (as indicated by induction of PD-L1 expression) by IL18 affinity variants (in the context of IL18 x Fab-Fc fusions having a silent Fv) in the absence of IL18BP (solid symbols/solid lines) or in the presence of 100 ng/ml IL18BP (open symbol/dotted lines). The data show that a number of substitutions enabled reduced activation potency in comparison to WT-4CS variant. For most of the variants, incubation with IL18BP shifts the activation potency; however, XENP38865 having the K53A appear minimally impacted by IL18BP indicating that the K53A substitution reduces binding affinity and sink by IL18BP.

FIG. 29 shows dose dependent inhibition of KG-1 cell activation (as indicated by induction of PD-L1 expression) by IL18BP and fixed concentration (10 nM) IL18 affinity variants (in the context of IL18 x Fab-Fc fusions having a silent Fv). The data show that some of the variants were very sensitive to IL18BP inhibition. However, XENP38865 having the K53A substitution was much less susceptible to IL18BP inhibition (as indicated by higher IC50 value of 88.73 nM in comparison to IC50 value of 5.503 for XENP37827 having WT-4CS IL18).

FIG. 30 shows dose dependent inhibition of KG-1 cell activation (as indicated by induction of PD-L1 expression) by IL18BP and fixed concentration (10 nM) IL18 affinity variants (in the context of IL18 x Fab-Fc fusions having a silent Fv).

FIG. 31 depicts BLI-response by IL18 x Fab-Fc fusions comprising IL18 affinity variants (Library 2) for IL18R1, IL18BP, and IL18R1xIL18RAP heterodimer as determined by Octet. The data show that the K53D and K53N single substitution variants exhibited weaker binding response to IL18R1 and IL18R1xIL18RAP but also no binding response to IL18BP. Several other substitutions at K53 also enabled weakened binding for both IL18R1 and IL18BP, albeit not to the same level of weaked IL18BP binding as K53D and K53N; S55N and S55Q enabled weakened binding to IL18R1 and IL18BP, although at the expense of stability (as depicted in FIGS. 35); and M41K enabled enhanced binding for IL18R1.

FIG. 32 depicts activation of KG-1 cells (as indicated by induction of PD-L1 expression) by IL18 affinity variants (in the context of IL18 x Fab-Fc fusions having a silent Fv) in the absence of IL18BP.

FIG. 33 depicts activation of KG-1 cells (as indicated by induction of PD-L1 expression) by IL18 affinity variants (in the context of IL18 x Fab-Fc fusions having a silent Fv) in the absence of IL18BP. The M51K variant appears to improve IL18 response.

FIG. 34 depicts activation of KG-1 cells (as indicated by induction of PD-L1 expression) by IL18 affinity variants (in the context of IL18 x Fab-Fc fusions having a silent Fv) in the absence of IL18BP.

FIG. 35 depicts activation of KG-1 cells (as indicated by induction of PD-L1 expression) by IL18 affinity variants (in the context of IL18 x Fab-Fc fusions having a silent Fv) in the absence of IL18BP.

FIG. 36 depicts melting temperature of IL18 affinity variants (Library 1) in the context of IL18 x Fab-Fc fusions having a silent Fv.

FIG. 37 depicts melting temperature of IL18 affinity variants (Library 2) in the context of IL18 x Fab-Fc fusions having a silent Fv and RP variants. K53N, P57A, and P57E increased Tm1 respectively by 5, 9.5, and 12° C. Several substitutions, including alternative substitutions as the aforementioned positions (e.g. K53I), decreased Tm1 by as much as 11.5° C.

FIG. 38 depicts sequence alignment of IL18 from various species.

FIGS. 39A-39B depict melting temperature of IL18 stability variants (Library 1) in the context of IL18 x Fab-Fc fusions having a silent Fv and RP variants.

FIG. 40 depicts melting temperature of IL18 stability variants (Library 2) in the context of IL18 x Fab-Fc fusions having a silent Fv and RP variants.

FIGS. 41A-41C depicts sequences for Further Variants (Library 1) which explored combinations of favorite variants from Stability Variants (Libraries 1 and 2). It should be noted that each of these variants may include additional substitutions such as production variants, affinity variants, and/or additional stability variants. Additionally, while these variants were produced with delD193 as they were assessed in the context of His-tagged molecules, these IL18 variants may be produced without delD193.

FIGS. 42A-42D depicts sequences for Further Variants (Library 2) which explored further substitutions at positions identified from Affinity Variants (Libraries 1 and 2) as well as at newly identified positions S36 and D132. It should be noted that each of these variants may include additional substitutions such as production variants, additional affinity variants, and/or stability variants.

FIGS. 43A-43b depicts sequences for Further Variants (Library 3) which explored combinations of favorite affinity variants with S10C/N155C. It should be noted that each of these variants may include additional substitutions such as production variants, affinity variants, and/or stability variants.

FIGS. 44A-44C depicts sequences for Further Variants (Library 4) which explored combinations of favorite affinity variants. In particular in certain combinations, variants enhanced in IL18 receptor binding were combined with K53D to restore its reduced IL18 receptor binding. It should be noted that each of these variants may include additional substitutions such as production variants, affinity variants, and/or stability variants.

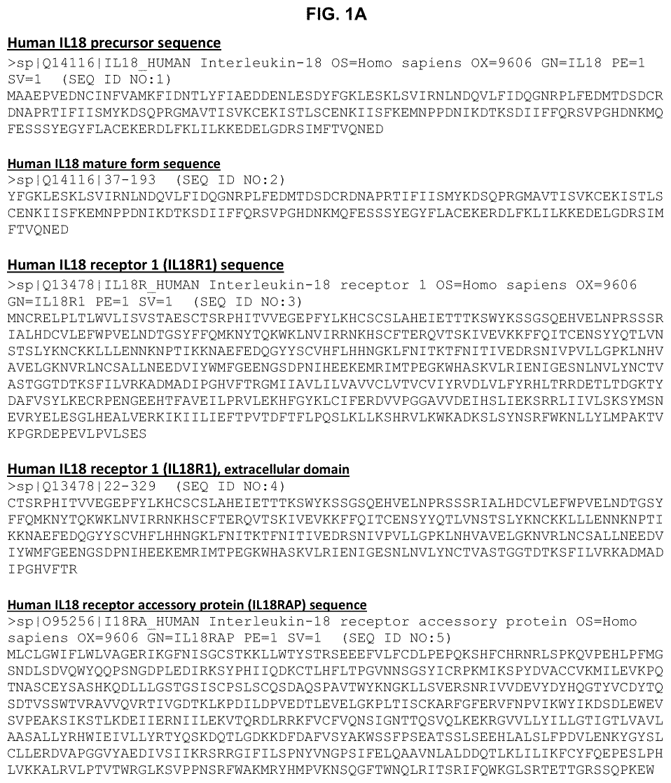

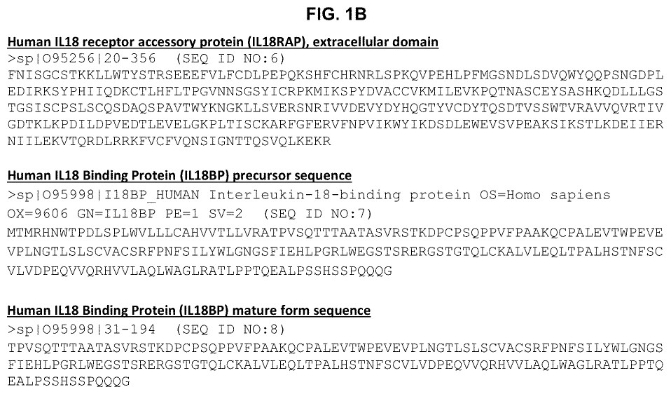

FIG. 45 depicts IL18 positions and substitutions for modulating IL18 binding affinity for IL18 receptors and/or IL18BP. It should be noted that these substitutions can be combined with any other substitutions such as production variants, additional affinity variants, and/or stability variants. The numbering is in the context of Human IL18 mature form sequence as depicted in FIG. 1.

FIG. 46 depicts IL18 positions and substitutions for improving stability. It should be noted that these substitutions can be combined with any other substitutions such as production variants, affinity variants, and/or additional stability variants. The numbering is in the context of Human IL18 mature form sequence as depicted in FIG. 1.

FIG. 47 depicts additional IL18 positions and substitutions for improving stability, specifically in the context of cysteine engineering. These substitutions remove existing cysteine residues or introduce new cysteine residues. It should be noted that these substitutions can be combined with any other substitutions such as production variants, affinity variants, and/or additional stability variants. The numbering is in the context of Human IL18 mature form sequence as depicted in FIG. 1.

FIG. 48 depicts binding to human IL18BP, cynomolgus IL18BP, human IL18R1, cynomolgus IL18R1, human IL18R1/IL18RAP complex, and cynomolgus IL18R1/IL18RAP complex by additional IL18 affinity variants (in the context of 4CS and S10C/N155C variants). The data show that K53D is the most important residue related to detuning IL18BP binding, and K53A and P57E are the next two most important.

FIG. 49 depicts activation of KG-1 cells (as indicated by induction of PD-L1 expression) by additional IL18 combination affinity variants (in the context of monovIL18-Fc fusions). Affinity variants were engineered in the context of IL18-4CS further including S10C/N155C stability variants.

FIG. 50A-50E depicts activation of KG-1 cells (as indicated by induction of PD-L1 expression) by additional 11,18 combination affinity variants in the absence of IL18BP (solid lines) or in the presence of 100 ng/ml IL18BP (dotted lines). Affinity variants were engineered in the context of IL18-4CS further including S10C/N155C stability variants. Vertical line indicates 100 ng/mL IL18BP =5.56 nM.

FIG. 51 depicts EC50 of activation of KG-1 cells (as indicated by induction of PD-L1 expression) by additional 11,18 combination affinity variants in the absence of IL18BP (solid lines) or in the presence of 100 ng/ml IL18BP (dotted lines). Affinity variants were engineered in the context of IL18-4CS further including S10C/N155C stability variants.

FIG. 52 depicts activation of KG-1 cells (as indicated by induction of PD-L1 expression) by additional 11,18 combination affinity variants. New affinity variants were engineered in the context of IL18-4CS further including S10C/N155C stability variants.

FIG. 53A-53L depicts activation of KG-1 cells (as indicated by induction of PD-L1 expression) by additional 11,18 combination affinity variants in the absence of IL18BP (solid lines) or in the presence of 1 pg/ml IL18BP (dotted lines). New affinity variants were engineered in the context of IL18-4CS further including S10C/N155C stability variants. Vertical line indicates 1 pg/mL IL18BP =55.56 nM.

FIG. 54 depicts EC50 of activation of KG-1 cells (as indicated by induction of PD-L1 expression) by additional 11,18 combination affinity variants in the absence of IL18BP or in the presence of 1 μg/ml IL18BP. Affinity variants were engineered in the context of IL18-4CS further including S10C/N155C stability variants.

FIG. 55 depicts activation of KG-1 cells (as indicated by induction of PD-L1 expression) by additional single substitution 11,18 affinity variants. New affinity variants were engineered in the context of IL18-4CS further including S10C/N155C stability variants.

FIG. 56 depicts activation of KG-1 cells (as indicated by induction of PD-L1 expression) by additional single substitution 11,18 affinity variants. New affinity variants were engineered in the context of IL18-4CS further including S10C/N155C stability variants.

FIG. 57 depicts activation of KG-1 cells (as indicated by induction of PD-L1 expression) by additional 11,18 combination affinity variants. New affinity variants were engineered in the context of IL18-4CS further including S10C/N155C stability variants.

FIG. 58 depicts activation of KG-1 cells (as indicated by induction of PD-L1 expression) by additional 11,18 combination affinity variants. New affinity variants were engineered in the context of IL18-4CS further including S10C/N155C stability variants.

FIG. 59 depicts activation of KG-1 cells (as indicated by induction of PD-L1 expression) by additional 11,18 combination affinity variants. New affinity variants were engineered in the context of IL18-4CS further including S10C/N155C stability variants.

FIGS. 60A-60K depicts activation of KG-1 cells (as indicated by induction of PD-L1 expression) by additional 11,18 combination affinity variants in the absence of IL18BP (solid lines) or in the presence of 1 μg/ml IL18BP (dotted lines). New affinity variants were engineered in the context of IL18-4CS further including S10C/N155C stability variants. Vertical line indicates 1 μg/mL IL18BP =55.56 nM.

FIG. 61 depicts activation of KG-1 cells (as indicated by induction of PD-L1 expression) by additional 11,18 combination affinity variants. New affinity variants were engineered in the context of IL18-4CS further including S10C/N155C stability variants.

FIG. 62 depicts EC50 of activation of KG-1 cells (as indicated by induction of PD-L1 expression) by additional 11,18 combination affinity variants and their fold reduction from WT* (*note: 11,18 comprising 4CS and S10C/N155C).

FIG. 63 depicts activation of KG-1 cells (as indicated by induction of PD-L1 expression) by selected IL18 potency variants in the context of monovIL18-Fc (solid symbol) or IL18 x Fab-Fc (open symbol). The data suggest that IL18 x Fab-Fc fusions have lower Ymax (efficacy).

FIGS. 64A-64E depicts change in body weight (as an indicator of GVHD) by A) Day 6, B) Day 9, C) Day 13, D) Day 16, and E) over time in huPBMC-engrafted NSG mice dosed with PBS or potency reduced IL18 fusion proteins at 5.0 mg/kg or 0.5 mg/kg. Test articles included XENP40967 (3-fold reduction from WT), XENP40685 (17-fold reduction from WT), XENP40966 (219-fold reduction from WT), XENP40962 (210-fold reduction from WT), and XENP40965 (9220-fold reduction from WT).

FIGS. 65A-65B depicts serum huIL-18BP on A) Day 7 and B) Day 14 in huPBMC-engrafted NSG mice dosed with PBS or potency reduced IL18 fusion proteins at 5.0 mg/kg or 0.5 mg/kg. Test articles included XENP40967 (3-fold reduction from WT), XENP40685 (17-fold reduction from WT), XENP40966 (219-fold reduction from WT), XENP40962 (210-fold reduction from WT), and XENP40965 (9220-fold reduction from WT). The data show that the IL18 fusion proteins induced huIL18BP over time.

FIGS. 66A-66C depicts expansion of A) human CD4 T cells, B) human CD8 T cell, and C) human CD16/CD56 NK cells by Day 14 in huPBMC-engrafted NSG mice dosed with PBS or potency reduced IL18 fusion proteins at 5.0 mg/kg or 0.5 mg/kg. Test articles included XENP40967 (3-fold reduction from WT), XENP40685 (17-fold reduction from WT), XENP40966 (219-fold reduction from WT), XENP40962 (210-fold reduction from WT), and XENP40965 (9220-fold reduction from WT). The data show T cell expansion generally correlated with IL18 potency (and was dose dependent).

FIG. 67 depicts activation of human CD16/CD56 NK cells (as indicated by NKG2D expression) by Day 7 in huPBMC-engrafted NSG mice dosed with PBS or potency reduced IL18 fusion proteins at 5.0 mg/kg or 0.5 mg/kg. Test articles included XENP40967 (3-fold reduction from WT), XENP40685 (17-fold reduction from WT), XENP40966 (219-fold reduction from WT), XENP40962 (210-fold reduction from WT), and XENP40965 (9220-fold reduction from WT).

FIGS. 68A-68B depicts serum IFNy on A) Day 7 and B) Day 14 in huPBMC-engrafted NSG mice dosed with PBS or potency reduced IL18 fusion proteins at 5.0 mg/kg or 0.5 mg/kg. Test articles included XENP40967 (3-fold reduction from WT), XENP40685 (17-fold reduction from WT), XENP40966 (219-fold reduction from WT), XENP40962 (210-fold reduction from WT), and XENP40965 (9220-fold reduction from WT). The data show that the IL18 fusion proteins induced IFNy over time.

FIGS. 69A-69B depicts serum GM-CSF on A) Day 7 and B) Day 14 in huPBMC-engrafted NSG mice dosed with PBS or potency reduced IL18 fusion proteins at 5.0 mg/kg or 0.5 mg/kg. Test articles included XENP40967 (3-fold reduction from WT), XENP40685 (17-fold reduction from WT), XENP40966 (219-fold reduction from WT), XENP40962 (210-fold reduction from WT), and XENP40965 (9220-fold reduction from WT). The data show that the IL18 fusion proteins induced serum GM-CSF over time.

FIGS. 70A-70C depicts serum concentration of IL18 fusion proteins over time in C57/B16 mice after dosing with 2 mg/kg XENP39804 (monovIL18-Fc with 4CS/E31Q variant), XENP40685 (monovIL18-Fc with 4CS/E31Q variant and further including S10C/N155C), or XENP40686 (IL18 x Fab-Fc with 4CS/E31Q variant and further including S10C/N155C). The data show improvement in serum levels upon introduction of the S10C/N155C disulfide variant into the E31Q base (i.e. XENP40685 vs. XENP39804). The IL18 x Fab-Fc format did not appear to confer any further improvements (i.e. XENP40686 vs. XENP40685).

FIG. 71 depicts human PBMCs stimulated with 500 ng/mL plate-bound anti-CD3 (OKT3) for 48 h and then analyzed by flow cytometry. Gates were selected for naïve (CD28+CD95−), memory (CD28+CD95+), and effector (CD28-CD95mid) T cells. Central Memory (Tcm) and Stem-cell like memory (Tscm) T cells were further gated by CD45RA- or CD45RA+, respectively. Counts and IL18R1 expression were consistent between CD4 and CD8 populations. Data show that IL18R1 expression is biased towards NKs and memory T cell sub sets.

FIG. 72 depicts the unfolding transition of various IL18 variants as determined by differential scanning fluorimetry.

FIG. 73 depicts the stabilization of IL18 that enabled improved solution behavior and facile purification. Analytical SEC and analytical IEX chromatograms of purified, stabilized IL18-Fc material are depicted in FIG. 73A and 73B.

FIG. 74A-74C depicts activation of KG-1 cells (as indicated by induction of PD-L1 expression) by A) XENP41756 (4CS S10C/N155C), B) XENP42006 (4CS E6Q/S10C/K53D/N111T/N155C), and C) XENP41762 (4CS S10C/K53D/N155C) in the absence of IL18BP (solid circle) or in the presence of 10 pg/ml IL18BP (open). Vertical line indicates 10 pg/mL IL18BP=555 nM.

FIG. 75A-75G depicts change in body weight (as an indicator of GVHD) by A) Day 8, B) Day 12, C) Day 15, D) Day 19, E) Day 25, F) Day 28, and G) Day 33 in huPBMC-engrafted NSG mice dosed with PBS or indicate concentrations of XENP41770 (4CS S10C/E31Q/D35N/N155C), XENP42006 (4CS E6Q/S10C/K53D/N111T/N155C), and XENP41762 (4CS S10C/K53D/N155C).

FIG. 76A-B depicts expansion of A) human CD3 T cells and B) human NK cells on Day 14 in huPBMC-engrafted NSG mice dosed with PBS or indicate concentrations of XENP41770, XENP42006, and XENP41762.

FIG. 77 depicts serum IFNy on Day 7 in huPBMC-engrafted NSG mice dosed with PBS or indicate concentrations of XENP41770, XENP42006, and XENP41762.

FIG. 78 depicts serum concentration of XENP41974 (4CS S10C/N155C) (total IL18-Fc vs. active IL18-Fc) over time in cynomolgus monkeys. Total IL18-Fc includes IL18-Fc with and without bound IL18BP, and active IL18-Fc is unbound.

FIG. 79 depicts serum concentration of XENP42007 (4CS E6Q/S10D/K53D/N111T) (total IL18-Fc vs. active IL18-Fc) over time in cynomolgus monkeys. Total IL18-Fc includes IL18-Fc with and without bound IL18BP, and active IL18-Fc is unbound.

FIGS. 80A-80P depicts the amino acid sequences of IL18-Fc fusion of the disclosure.

FIG. 81 depicts secretion of IP10 by human PBMCs upon stimulation with IL18-Fc fusions XENP42141, XENP42143, and XENP42145.

FIG. 82A-82F depict activation of mouse NK1.1 cells (as indicated by induction of intracellular IFNy) by murine IL18-Fc fusion having murine IL18 variants.

FIG. 83A-83D depict binding to murine IL18BP by murine IL18-Fc fusion having murine IL18 variants. The data show that incorporating L59K dramatically lowers affinity to IL18BP (XENP43092 in FIG. 83A and FIG. 83B), adding K52X to L59K abrogates IL18BP binding (FIG. 83C), and adding E30Q or D34N does not modulate IL18BP binding (FIG. 83D).

FIG. 84A-84J Sensorgrams depicting binding by IL18-Fc fusions for IL18BP as determined by Octet. It should be noted that this experiment was intended to ascertain binding and not intended for accurate KD measurements, but it does show relative ranking of binding enabled by various IL18 variants.

FIG. 85A-85J Sensorgrams depicting binding by IL18-Fc fusions for IL18R1xIL18RAP heterodimer as determined by Octet. It should be noted that this experiment was intended to ascertain binding and not intended for accurate KD measurements, but it does show relative ranking of binding enabled by various IL18 variants.

### DETAILED DESCRIPTION

I. Overview

Provided herein are IL18-Fc fusion proteins that include an empty-Fc domain and an IL18 protein connected to another Fc domain. Also provided herein are additional IL18-Fc fusion proteins such as IL18 x Fab-Fc fusion proteins that include a first monomer containing an IL18 protein connected to a first Fc domain, a second monomer containing a variable heavy chain connected to a second Fc domain, and a variable light chain, such that the variable heavy and light chains form an empty Fab. Such IL18-Fc fusion proteins exhibit IL18 biological activity and long serum half-lives. Due to the long serum half-lives, the fusion proteins advantageously do not require high doses for use in treatments, thereby minimizing any potential systemic toxicity associated with increased IL18 levels. The IL18-Fc fusion proteins can be used for applications where increased IL18 activity is useful, for example, for increasing an immune response which can be useful for mounting an anti-cancer response in a subject in need thereof. Also described herein are various variant IL18 proteins with modifications to improve production (e.g., by improving yield and/or reduce heterogeneity), improve stability, reduce sink, and/or reduced affinity/potency.

II. Definitions

In order that the application may be more completely understood, several definitions are set forth below. Such definitions are meant to encompass grammatical equivalents.

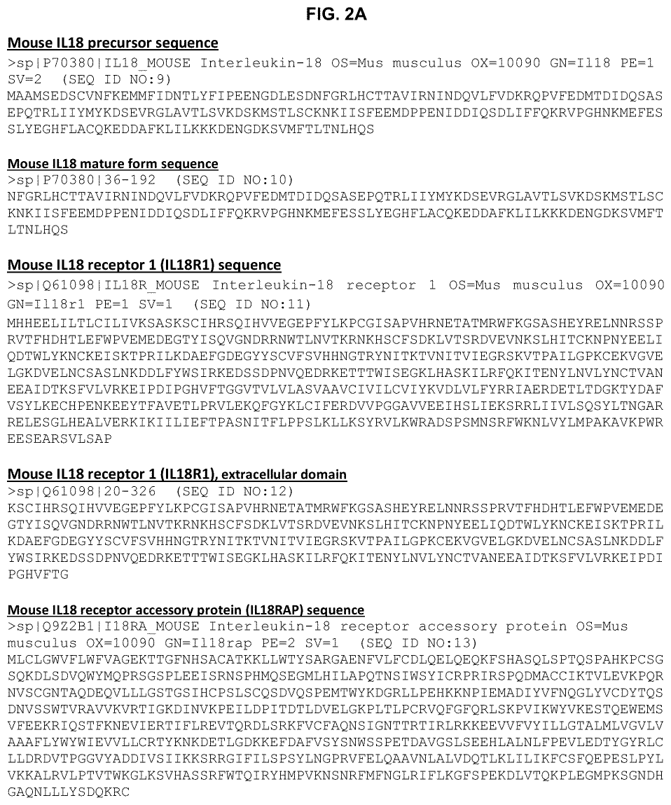

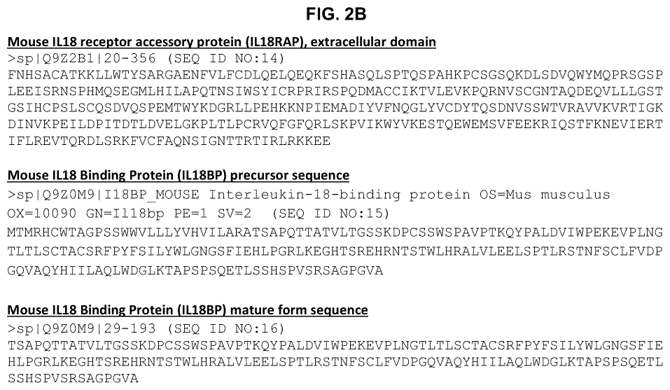

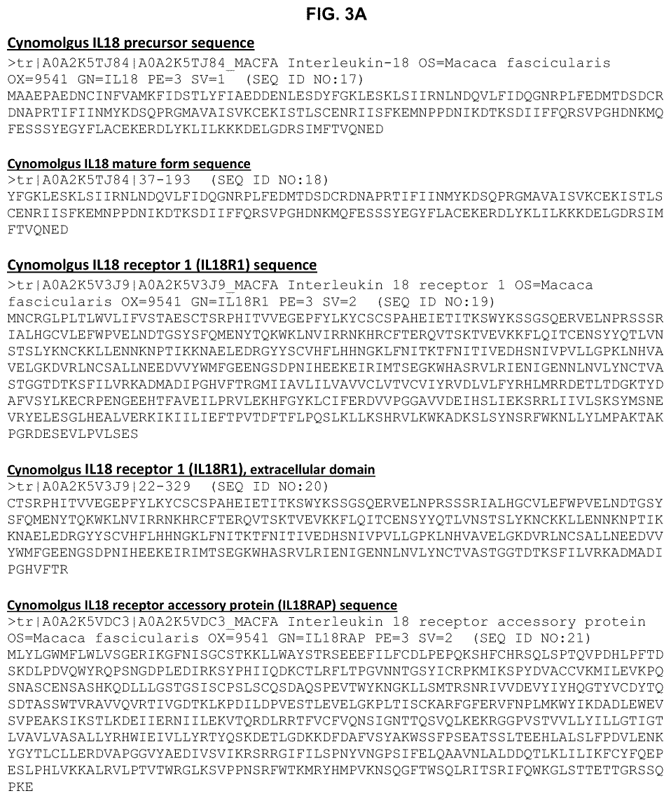

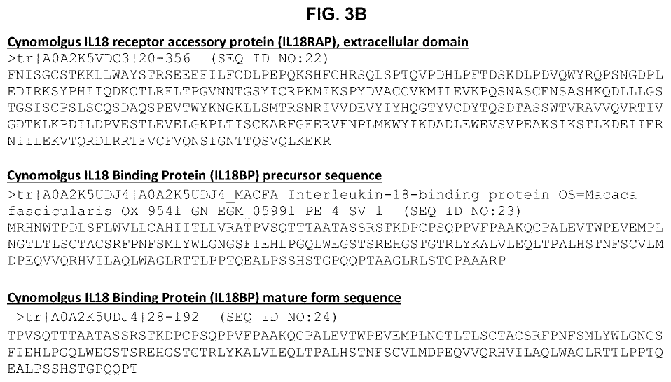

By “IL-18,” “Interleukin-18,” and “IL18” herein is meant a proinflammatory cytokine that binds to IL18 receptor (IL18R1). IL18 is capable of stimulating IFNy production and regulating Th1 and Th2 responses. Sequences of various IL18 proteins, corresponding IL18 receptors, IL18 receptor accessory proteins (IL18RAPs), and IL18 binding proteins (IL18BPs) are shown in FIGS. 1-3. Sequences of exemplary wildtype human precursor and mature IL-18, as well as the IL-18 receptor subunits are included in FIGS. 1A-1B.

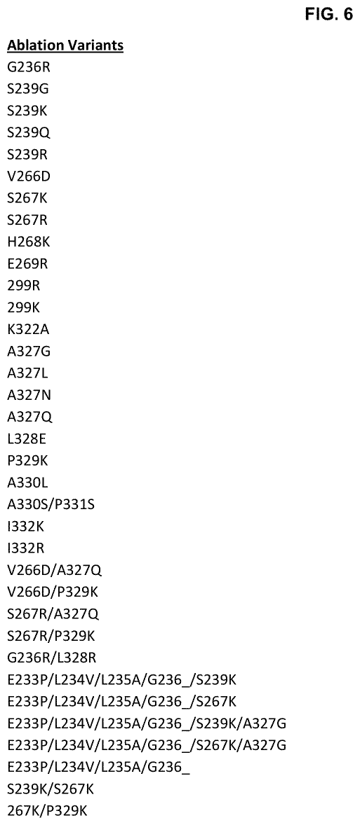

By “ablation” herein is meant a decrease or removal of binding and/or activity. Thus for example, “ablating FcγR binding” means the Fc region amino acid variant has less than 50% starting binding as compared to an Fc region not containing the specific variant, with more than 70-80-90-95-98% loss of binding being preferred, and in general, with the binding being below the level of detectable binding in a Biacore assay. Of particular use in the ablation of FcγR binding are those shown in FIG. 6. However, unless otherwise noted, the Fc monomers of the invention retain binding to the FcRn.

By “ADCC” or “antibody dependent cell-mediated cytotoxicity” as used herein is meant the cell-mediated reaction wherein nonspecific cytotoxic cells that express FcγRs recognize bound antibody on a target cell and subsequently cause lysis of the target cell. ADCC is correlated with binding to FcγRllla; increased binding to FcγRllla leads to an increase in ADCC activity. As is discussed herein, some embodiments ablate ADCC activity entirely.

By “modification” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y or 272Y refers to a variant polypeptide, in this case an Fc variant, in which the glutamic acid at position 272 is replaced with tyrosine. For clarity, a protein which has been engineered to change the nucleic acid coding sequence but not to change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid residue or sequence at a particular position in a parent polypeptide sequence. For example, -233E designates an insertion of glutamic acid after position 233 and before position 234. Additionally, -233ADE or A233ADE designates an insertion of AlaAspGlu after position 233 and before position 234.

By “amino acid deletion” or “deletion” as used herein is meant the removal of an amino acid residue or sequence at a particular position in a parent polypeptide sequence. For example, E233-, E233#, E2330, E233_, or E233del designates a deletion of glutamic acid at position 233. Additionally, EDA233- or EDA233# designates a deletion of the sequence GluAspAla that begins at position 233.

By “variant protein”, “protein variant”, or “variant” as used herein is meant a protein that differs from that of a parent protein by virtue of at least one modification. Protein variant may refer to the protein itself, a composition comprising the protein, the amino acid sequence that encodes it, or the DNA sequence that encodes it. Preferably, the protein variant has at least one amino acid modification compared to the parent protein, e.g. from about one to about seventy amino acid modifications, and preferably from about one to about five amino acid modifications compared to the parent. The modification can be an addition, deletion, or substitution. As described below, in some embodiments the parent protein, for example an Fc parent polypeptide, is a human wild type sequence, such as the Fc region from IgG1, IgG2, IgG3 or IgG4. The protein variant sequence herein will preferably possess at least about 80% identity with a parent protein sequence, and most preferably at least about 90% identity, more preferably at least about 95-98-99% identity. “Variant,” as used herein can also refer to particular amino acid modifications (e.g., substitutions, deletions, insertions) in a variant protein (e.g., a variant Fc domain), for example, heterodimerization variants, ablation variants, FcKO variants, etc., as disclosed in Section III below.

As used herein, by “protein” is meant at least two covalently attached amino acids, which includes proteins, polypeptides, oligopeptides and peptides. When a biologically functional molecule comprises two or more proteins, each protein may be referred to as a “monomer” or as a “subunit; and the biologically functional molecule may be referred to as a “complex.”

By “residue” as used herein is meant a position in a protein and its associated amino acid identity. For example, Asparagine 297 (also referred to as Asn297 or N297) is a residue at position 297 in the human antibody IgG1.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein with respect to an IgG domain is meant an amino acid modification that is not isotypic. For example, because none of the IgGs comprise a serine at position 434, the substitution 434S in IgG1, IgG2, IgG3, or IgG4 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “IgG Fc ligand” or “Fc ligand” as used herein is meant a molecule, preferably a polypeptide, from any organism that binds to the Fc region of an IgG antibody to form an Fc/Fc ligand complex. Fc ligands include but are not limited to FcγRIs, FeyRIIs, FeyRIIIs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190:123-136, entirely incorporated by reference). Fc ligands may include undiscovered molecules that bind Fc. Particular IgG Fc ligands are FcRn and Fc gamma receptors.

By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRlllb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “FcRn” or “neonatal Fc receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin (B2-microglobulin) and the heavy chain is encoded by the FcRn gene. Unless otherwise noted herein, FcRn or an FcRn protein refers to the complex of FcRn heavy chain with B2-microglobulin. A variety of Fc variants can be used to increase binding to the FcRn, and in some cases, to increase serum half-life. In general, unless otherwise noted, the Fc monomers of the invention retain binding to the FcRn (and, as noted below, can include amino acid variants to increase binding to the FcRn).

By “parent polypeptide” as used herein is meant a starting polypeptide that is subsequently modified to generate a variant. The parent polypeptide may be a naturally occurring polypeptide (i.e., a wildtype polypeptide), or a variant or engineered version of a naturally occurring polypeptide. Parent polypeptide may refer to the polypeptide itself, compositions that comprise the parent polypeptide, or the amino acid sequence that encodes it.

By “Fc” or “Fc region” or “Fc domain” as used herein is meant the polypeptide comprising the constant region of an antibody, in some instances, excluding all of the first constant region immunoglobulin domain (e.g., CH1) or a portion thereof, and in some cases, optionally including all or part of the hinge. For IgG, the Fc domain comprises immunoglobulin domains CH2 and CH3 (Cy2 and Cy3), and optionally all or a portion of the hinge region between CH1 (Cγ1) and CH2 (Cy2). Thus, in some cases, the Fc domain includes, from N- to C-terminus, CH2-CH3 and hinge-CH2-CH3. In some embodiments, the Fc domain is that from IgG1, IgG2, IgG3 or IgG4, with IgG1 hinge-CH2-CH3 and IgG4 hinge-CH2-CH3 finding particular use in many embodiments. Additionally, in certain embodiments, wherein the Fc domain is a human IgG1 Fc domain, the hinge includes a C220S amino acid substitution. Furthermore, in some embodiments where the Fc domain is a human IgG4 Fc domain, the hinge includes a S228P amino acid substitution. Although the boundaries of the Fc region may vary, the human IgG heavy chain Fc region is usually defined to include residues E216, C226, or A231 to its carboxyl-terminus, wherein the numbering is according to the EU index as in Kabat. In some embodiments, as is more fully described below, amino acid modifications are made to the Fc region, for example to alter binding to one or more FcγR or to the FcRn.

As will be appreciated by those in the art, the exact numbering and placement of the heavy constant region domains can be different among different numbering systems. A useful comparison of heavy constant region numbering according to EU and Kabat is as below, see Edelman et al., 1969, Proc Natl Acad Sci USA 63:78-85 and Kabat et al., 1991, Sequences of Proteins of Immunological Interest, 5th Ed., United States Public Health Service, National Institutes of Health, Bethesda, entirely incorporated by reference.

TABLE 1

| | EU Numbering | Kabat Numbering |
|---|---|---|
| CH1 | 118-215 | 114-223 |
| Hinge | 216-230 | 226-243 |
| CH2 | 231-340 | 244-360 |
| CH3 | 341-447 | 361-478 |

“Fc variant” or “variant Fc” as used herein is meant a protein comprising an amino acid modification in an Fc domain. The modification can be an addition, deletion, or substitution. The Fc variants of the present invention are defined according to the amino acid modifications that compose them. Thus, for example, N434S or 434S is an Fc variant with the substitution for serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as 434S/428L, and so on. For all positions discussed herein that relate to antibodies or derivatives and fragments thereof (e.g., Fc domains), unless otherwise noted, amino acid position numbering is according to the EU index. The “EU index” or “EU index as in Kabat” or “EU numbering” scheme refers to the numbering of the EU antibody (Edelman et al., 1969, Proc Natl Acad Sci USA 63:78-85, hereby entirely incorporated by reference). The modification can be an addition, deletion, or substitution.

By “fusion protein” as used herein is meant covalent joining of at least two proteins or protein domains. Fusion proteins may comprise artificial sequences, e.g. a domain linker, an Fc domain (e.g., a variant Fc domain), an IL-7 (e.g., a variant IL18), etc. as described herein. By “Fc fusion protein” or “immunoadhesin” herein is meant a protein comprising an Fc region, generally linked (optionally through a domain linker, as described herein) to one or more different protein domains. Accordingly, an “IL18-Fc fusion” includes an Fc domain linked (optionally through a domain linker) to an IL18, as described herein. In some instances, two Fc fusion proteins can form a homodimeric Fc fusion protein or a heterodimeric Fc fusion protein. In some embodiments, one monomer of the heterodimeric IL18-Fc fusion protein includes an Fc domain alone (e.g., an “empty Fc domain”) and the other monomer is an Fc fusion, comprising an IL18, as outlined herein. In other embodiments, both the first and second monomers are Fc fusion proteins that include an Fc domain and an IL18.

By “position” as used herein is meant a location in the sequence of a protein. Positions may be numbered sequentially, or according to an established format, for example the EU index for numbering of antibody domains (e.g., a CH1, CH2, CH3 or hinge domain).

By “strandedness” in the context of the monomers of the heterodimeric proteins of the invention herein is meant that, similar to the two strands of DNA that “match”, heterodimerization variants are incorporated into each monomer so as to preserve, create, and/or enhance the ability to “match” to form heterodimers. For example, if some pI variants are engineered into monomer A (e.g. making the pI higher), then steric variants that are “charge pairs” that can be utilized as well do not interfere with the pI variants, e.g. the charge variants that make a pI higher are put on the same “strand” or “monomer” to preserve both functionalities. Similarly, for “skew” variants that come in pairs of a set as more fully outlined below, the skilled artisan will consider pI in deciding into which strand or monomer that incorporates one set of the pair will go, such that pI separation is maximized using the pI of the skews as well.

By “wild type,” “wildtype” or WT″ herein is meant an amino acid sequence or a nucleotide sequence that is found in nature, including allelic variations. A WT protein has an amino acid sequence or a nucleotide sequence that has not been intentionally modified.

The IL18-Fc fusion proteins and variant IL18s provided herein are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated protein,” refers to a protein which is substantially free of other proteins from a cell culture such as host cell proteins. “Recombinant” means the proteins are generated using recombinant nucleic acid techniques in exogeneous host cells.

“Percent (%) amino acid sequence identity” with respect to a protein sequence is defined as the percentage of amino acid residues in a candidate sequence that are identical with the amino acid residues in the specific (parental) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. One particular program is the ALIGN-2 program outlined at paragraphs [0279] to [0280] of US Publ. App. No. 20160244525, hereby incorporated by reference.

The degree of identity between an amino acid sequence provided herein (“invention sequence”) and the parental amino acid sequence is calculated as the number of exact matches in an alignment of the two sequences, divided by the length of the “invention sequence,” or the length of the parental sequence, whichever is the shortest. The result is expressed in percent identity.

In some embodiments, two or more amino acid sequences are at least 50%, 60%, 70%, 80%, or 90% identical. In some embodiments, two or more amino acid sequences are at least 95%, 97%, 98%, 99%, or even 100% identical.

By “fused” or “covalently linked” is herein meant that the components (e.g., an IL18 and an Fc domain) are linked by peptide bonds, either directly or indirectly via domain linkers, outlined herein.

The strength, or affinity, of specific binding can be expressed in terms of dissociation constant (KD) of the interaction, wherein a smaller KD represents greater affinity and a larger KD represents lower affinity. Binding properties can be determined by methods well known in the art such as bio-layer interferometry and surface plasmon resonance based methods. One such method entails measuring the rates of antigen-binding site/antigen or receptor/ligand complex association and dissociation, wherein rates depend on the concentration of the complex partners, the affinity of the interaction, and geometric parameters that equally influence the rate in both directions. Thus, both the association rate (ka) and the dissociation rate (kd) can be determined, and the ratio of kd/ka is equal to the dissociation constant KD (See Nature 361:186-187 (1993) and Davies et al. (1990) Annual Rev Biochem 59:439-473).

Specific binding for a particular molecule or an epitope can be exhibited, for example, by a molecule (e.g., IL18) having a KD for its binding partner (e.g., IL18 receptor) of at least about 10-4 M, at least about 10-5 M, at least about 10-6 M, at least about 10-7 M, at least about 10-8 M, at least about 10-9 M, alternatively at least about 10-10 M, at least about 10-11 M, at least about 10-12 M, or greater. Typically, an antigen binding molecule that specifically binds an antigen will have a KD that is 20-, 50-, 100-, 500-, 1000-, 5,000-, 10,000- or more times greater for a control molecule relative to the antigen or epitope.

III. IL18 Fc Fusion Proteins

In some aspects, provided herein are IL18-Fc fusion proteins that include a first monomer that includes a IL18 protein (e.g., wildtype IL18 or a variant thereof) and a first Fc domain comprising pI variants as described below and a second monomer that includes a second Fc domain. The IL18-Fc fusion proteins are based on the self-assembling nature of the two Fc domains on each monomer leading to a IL18-Fc fusion proteins. Heterodimeric IL18-Fc fusion are made by altering the amino acid sequence of each monomer as more fully discussed below.

In one aspect, the IL18-Fc fusion protein is a heterodimeric Fc fusion protein. Such heterodimeric IL18-Fc fusion protein include a first monomer and a second monomer, each having an Fc domain with different amino acid sequences (e.g., a monovalent IL18-Fc fusion protein). As will be appreciated, discussion herein of components of the IL18-Fc fusion proteins encompassed by the present disclosure is applicable to both homodimeric and heterodimeric Fc fusion proteins as appropriate, unless otherwise specified.

In some embodiments, the IL18-Fc fusion protein is a monovalent IL18 fusion (i.e., includes only one IL18). In such embodiments, the first monomer includes an Fc domain and an IL18 and the second monomer includes an Fc domain alone (i.e., no IL18, an “empty Fc domain,”).

The Fc domains can be derived from IgG Fc domains, e.g., IgG1, IgG2, IgG3 or IgG4 Fc domains, with IgG1 Fc domains finding particular use in the invention. As described herein, IgG1 Fc domains may be used, often, but not always in conjunction with ablation variants to ablate effector function. Similarly, when low effector function is desired, IgG4 Fc domains may be used.

For any of the dimeric IL18-Fc fusion proteins described herein, the carboxy-terminal portion of each chain defines a constant region primarily responsible for effector function. Kabat et al. collected numerous primary sequences of the variable regions of heavy chains and light chains. Based on the degree of conservation of the sequences, they classified individual primary sequences into the CDRs and the framework and made a list thereof (see SEQUENCES OF IMMUNOLOGICAL INTEREST, 5th edition, NIH publication, No. 91-3242, E.A. Kabat et al., entirely incorporated by reference). Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

In the IgG subclass of immunoglobulins, there are several immunoglobulin domains in the heavy chain. By “immunoglobulin (Ig) domain” herein is meant a region of an immunoglobulin having a distinct tertiary structure. Of interest in the present IL18 Fc fusion proteins are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. As shown in Table 1, the exact numbering and placement of the heavy chain domains can be different among different numbering systems. As shown herein and described below, the pI variants can be in one or more of the CH regions, as well as the hinge region, discussed below.

By “hinge” or “hinge region” or “antibody hinge region” or “immunoglobulin hinge region” herein is meant the flexible polypeptide comprising the amino acids between the first and second heavy chain constant domains of an antibody. Structurally, the IgG CH1 domain ends at EU position 215, and the IgG CH2 domain begins at residue EU position 231. Thus for IgG the antibody hinge is herein defined to include positions 216 (E216 in IgG1) to 230 (P230 in IgG1), wherein the numbering is according to the EU index as in Kabat. In some embodiments, for example in the context of an Fc region, the hinge (full length or a fragment of the hinge) is included, generally referring to positions 216-230. As noted herein, pI variants can be made in the hinge region as well.

In some embodiments of the IL18 fusion proteins described herein, each of the first and second monomers include an Fc domain that has the formula hinge-CH2-CH3. In some embodiments of the IL18 fusion proteins described herein, each of the first and second monomers include an Fc domain that has the formula CH2-CH3.

In some embodiments, provided herein is an IL18-Fc fusion protein which is also referred to as “an IL18 x Fab-Fc fusion protein”. Such an IL18 x Fab-Fc fusion protein includes a first monomer containing a variable heavy chain and an Fc domain; a second monomer containing and an IL18 protein (e.g., a wildtype IL18 protein or a variant thereof) and an Fc domain comprising pI variants as described below, and a third monomer containing a variable light chain such that the variable heavy and light chains form a Fab. In certain embodiments, the IL18 is directly connected to the Fc domain. In some embodiments, the C-terminus of the IL18 is directly connected to the N-terminus of the Fc domain. In some embodiments described herein, the IL18 x Fab-Fc fusion protein also includes a second monomer that includes a second Fc domain and a Fab. In certain embodiments, the Fab is directly connected to the second Fc domain. In certain embodiments, the Fab is connected to the second Fc domain via a linker, such as but not limited to a domain linker. In some embodiments, the heavy chain of the Fab is directly connected to the second Fc domain. In some embodiments, the heavy chain of the Fab is connected to the second Fc domain via a linker, such as but not limited to a domain linker. In some embodiments, the C-terminus of the heavy chain of the Fab is directly connected to the N-terminus of the second Fc domain. In some embodiments, the light chain of the Fab is not directed connected to the second monomer. In certain embodiments, the light chain of the Fab is not directed connected to the first monomer.

In certain embodiments, the IL18 is connected to the Fc domain by a linker. In certain embodiments, the linker is a domain linker. Useful domain linker include, but are not limited to, those disclosed in FIG. 8. While any suitable linker can be used, many embodiments utilize a glycine-serine polymer, including for example (GS)n [SEQ ID NO:777], (GSGGS)n [SEQ ID NO:778], (GGGGS)n, and (GGGS)n [SEQ ID NO:779], where n is an integer of at least one (and generally from 0 to 1 to 2 to 3 to 4 to 5) as well as any peptide sequence that allows for recombinant attachment of the two domains with sufficient length and flexibility to allow each domain to retain its biological function. In some cases, and with attention being paid to “strandedness”, as outlined below, the linker is a charged domain linker.

In certain embodiments, the IL18 fusion protein includes a first monomer, wherein an IL18 is connected to the Fc domain by a domain linker. In some embodiments, the C-terminus of the IL18 is connected to the N-terminus of the Fc domain by a domain linker.

IV. Interleukin 18 Variants

The IL18-Fc fusion proteins provided herein include an IL18 protein. In some embodiments, the IL18-Fc fusion protein is a monovalent IL18-Fc fusion protein that includes one IL18. The IL18s that can be used with the IL18-Fc fusion proteins provided herein include wildtype IL18 (see FIGS. 1-3), functional fragments of such IL18s and variants that include 1, 2, 3, 4, 5, 6, 7, 8, 9, 10 amino acid modifications as compared to wildtype IL-18 (e.g., wildtype human IL18 and wildtype human IL18, mature form).

In some embodiments, the IL18 is a variant human IL18 that is at least 90, 91, 92, 93, 94, 95, 96, 97, 98, 99% identical to human IL18. In some embodiments, the IL18 is a variant human IL18 that is at least 90, 91, 92, 93, 94, 95, 96, 97, 98, 99% identical to human IL18, mature form. In particular embodiments, the IL18 includes 1, 2, 3, 4, 5, 6, 7, 8, 9, 10, 11, 12, 13, 14, 15, 16, 17, 18, 19 or 20 modifications as compared to wildtype human IL18. In particular embodiments, the IL18 includes 1, 2, 3, 4, 5, 6, 7, 8, 9, 10, 11, 12, 13, 14, 15, 16, 17, 18, 19 or 20 modifications as compared to wildtype human IL18.

Numbering of such IL18 modifications described herein are based on the human IL18 mature form sequence in FIG. 1, wherein the first amino acid of the sequence (“Y”) is amino acid position 1. Provided herein are compositions that include such a variant IL18 having one or more amino acid substitutions that reduce heterogeneity that may affect IL18-Fc fusion protein production and/or activity.

In certain embodiments, the IL18 variant includes one or more modifications to improve production. Such modifications are intended to improve yield and/or decrease molecular heterogeneity. Residues which may be modified to improve production include E31, C38, C68, C76, C127, D157. In some embodiments, the production variants include one or more amino acid substitutions selected from E31Q, C38S, C68S, C76S, C127S, D157del, D157S, and D157A.

In certain embodiments, the IL18 variant includes one or more modifications that are affinity variants which modulate binding affinity for IL18 receptors (such as IL18R1, IL18RAP, or the IL18R1:IL18RAP complex) and/or IL18BP. In some embodiments, the IL18 variant possesses one or more modifications that modulate IL18 potency and/or IL18 sink. Such modifications are believed to decrease the IL18BP sink and extend the half-life of the subject IL18-Fc fusion protein. Residues which may be modified to reduce binding affinity for IL18BP, IL18R1, IL18RAP, and/or the IL18R1:IL18RAP complex include Y1, E6, K8, D17, E31, D35, S36, D37, D40, N41, M51, K53, S55, Q56, P57, M60, Q103, H109, D110, N111, and D132. In some embodiments, the affinity variants include one or more amino acid substitutions selected from Y1F, Y1H, E6A, E6Q, K8Y, K8Q, K8E, D17N, E31Q, D35N, D35E, S36D, S36N, D37N, D40N, N41Q, M51K, M51Q, M51I, M51R, M51L, M51H, M51F, M51Y, K53A, K53S, K53G, K53T, K53I, K53L, K53N, K53D, K53R, K53H, K53M, K53E, K53Q, K53V, K53Y, K53F, S55N, S55Q, S55D, S55E, S55T, Q56L, Q56I, P57A, P57E, P57Q, P57D, P57Y, P57N, M60L, M60I, M60K, M60Y, M60F, Q103E, H109W, H109Y, D110N, D110Q, D110R, N111Q, N111S, N111T, N111E, D132Q, and D132E. In some embodiments, the amino acid substitution can include E6A/K53A, D35N/K53A, N41Q/K53A, D35N/N41Q/K53A, D35N/N41Q, D37N/K53A, E6Q/K53D, E6Q/M51K/K53D, M51K/K53D, M51K/K53E, E6Q/K53E, E6Q/M51K/K53E, E6Q/M51K/P57E, M51K/P57E, E6Q/P57E, K53G/P57E, K53T/P57E, K53A/P57E, M51L/K53D, K53D/D110R, K53D/N111T, K53D/S55T, K53D/S55T/D110R, M51L/K53D/S55T/D110R/N111T, M51L/K53D/S55T/D110R, K53D/S55T/D110R/N111T, K53D/S55T/N111T, D35N/D37N, E6Q/M51L/K53D/S55T/D110R/N111T, K53D/H109Y, D37N/K53D, D35N/K53D, K8E/K53D, N41Q/K53D, K53D/P57V, K53D/P57T, or E6Q/K53D/N111T. In certain embodiments, the IL18 variant includes one or more modifications that modulate binding to IL18R including K53V. In certain other embodiments, the IL18 variant includes one or more modification that modulate binding to IL18BP including M60K.

In certain embodiments, the IL18 variant includes one or more modifications to improve stability. In some embodiments, the IL18 variant possesses one or more modifications that improve stability in the context of cysteine engineering (such as by removing unpaired cysteines and/or introducing new disulfide bridges). Residues which may be modified to improve stability include S7, S10, V11, N14, L15, Q18, D23, R27, P28, L29, E31, M33, T34, C38, R39, R44, I46, I49, S50, K53, D54, Q56, P57, M60, A61, V62, T63, S65, K67, C68, E69, I71, C76, E77, I80, I81, N87, P88, D90, K93, T95, K96, S97, Q103, N111, M113, S119, A126, C127, L136, L138, K139, E141, L144, D146, R147, I149, M150, N155, E156, and D157. In some embodiments, the stability variants include one or more amino acid substitutions selected from S7C, S7P, S10C, V11I, N14C, N14W, L15C, Q18L, D23N, D23S, R27Q, P28C, L29V, E31Q, M33C, T34P, C38Q, C38R, C38E, C38L, C38I, C38V, C38K, C38D, C38S, R39T, R39S, R44Q, I46V, I49C, S50C, S50Y, K53N, D54C, Q56L, P57T, P57V, P57A, P57E, M60L, A61C, V62C, T63C, S65C, K67Q, C68I, C68F, C68Y, C68D, C68N, C68E, C68Q, C68K, C68S, E69K, I71M, C76E, C76K, C76S, E77K, 180T, I81V, I81L, N87S, P88C, D90E, K93D, K93N, T95E, K96G, K96Q, S97N, Q103C, Q103L, Q103I, N111D, M113I, S119L, A126C, C127W, C127Y, C127F, C127D, C127E, C127K, C127S, L136C, L138C, K139C, E141K, E141Q, L144N, D146Y, D146L, D146F, R147C, R147K, I149V, M150F, M150T, N155C, E156Q, and D157N. In some embodiments, the amino acid substitution can include Q56L/P57T, K93D/T95E, K93N/T95E, E156Q/D157N, D23N/R27Q, Q56L/T95E, K96Q/S119L, E141K/I149V, E141Q/I149V, S7P/S50Y, 180T/I81L, P57A/S119L, P57A/180T/I81L/S119L, P57A/K93D/T95E/S119L, I80T/S119L, 180T/I81L/K93D/T95E, P57A/180T/I81L/K93D/T95E/S119L, P57A/180T/S119L, N14C/S127C, M33C/S38C, S76C/L138C, S10C/I49C, Ll5C/R147C, P28C/L136C, S50C/P88C, T63C/P88C, V62C/Q103C, S10C/N155C, S65C/P88C, S7C/S50C, D54C/A61C, A126C/K139C, or C38R/C127W.

In some embodiments, one or more residues of IL18 selected from Y1, E6, S7, K8, S10, V11, N14, L15, D17, Q18, D23, R27, P28, L29, E31, M33, T34, D35, S36, D37, C38, R39, D40, N41, R44, I46, I49, S50, M51, K53, D54, S55, Q56, P57, M60, A61, V62, T63, S65, K67, C68, E69, I71, C76, E77, I80, I81, N87, P88, D90, K93, T95, K96, S97, Q103, H109, D110, N111, M113, S119, A126, C127, D132, L136, L138, K139, E141, L144, D146, R147, I149, M150, N155, E156, and D157 are modified. In some embodiments, the IL18 variant described herein includes one or more amino acid substitutions selected from Y1F, Y1H, E6A, E6Q, S7C, S7P, K8E, K8Q, K8Y, S10C, V11I, N14C, N14W, L15C, D17N, Q18L, D23N, D23S, R27Q, P28C, L29V, E31Q, M33C, T34P, D35N, D35E, S36D, S36N, D37N, C38S, C38Q, C38R, C38E, C38L, C38I, C38V, C38K, C38D, R39S, R39T, D40N, N41Q, R44Q, I46V, I49C, S50C, S50Y, M51I, M51K, M51Q, M51R, M51L, M51H, M51F, M51Y, K53A, K53D, K53E, K53G, K53H, K53I, K53L, K53M, K53N, K53Q, K53R, K53S, K53T, K53V, K53Y, K53F, D54C, S55N, S55Q, S55D, S55E, S55T, Q56I, Q56L, P57A, P57E, P57T, P57V, P57Q, P57D, P57Y, P57N, M60I, M60L, M60K, M60Y, M60F, A61C, V62C, T63C, S65C, K67Q, C68S, C68I, C68F, C68Y, C68D, C68N, C68E, C68Q, C68K, E69K, I71M, C76S, C76E, C76K, E77K, 180T, I81L, I81V, N87S, P88C, D90E, K93D, K93N, T95E, K96G, K96Q, S97N, Q103C, Q103E, Q103I, Q103L, H109W, H109Y, D110N, D110Q, D110R, N111D, N111Q, N111S, N111T, N111E, M113I, S119L, A126C, C127S, C127W, C127Y, C127F, C127D, C127E, C127K, D132Q, D132E, L136C, L138C, K139C, E141K, E141Q, L144N, D146F, D146L, D146Y, R147C, R147K, I149V, M150F, M150T, N155C, E156Q, D157A, D157S, D157N, and D157del. In some embodiments, the IL18 variant includes a 4CS substitution (C38S/C68S/C76S/C127S substitutions) and one or more of the additional substitutions including S38C, S38E, S38L, S38Q, S38R, S38V, S38K, S38D, S68C, S68D, S68E, S68F, S68I, S68N, S68Q, S68Y, S68K, S76C, S76E, S76K, S127C, S127D, S127F, S127W, S127K, and S127Y. In some embodiments, the amino acid substitution can include 4CS, 4CS/D193S, 4CS/D193A, 4CS/delD193, 4CS/S38E, 4CS/S68E, 4CS/S76E, 4CS/S127E, 4CS/S38K, 4CS/S68K, 4CS/S76K, 4CS/S127K, 4CS/S38D, 4CS/Y1F, 4CS/Y1H, 4CS/E6A, 4CS/E6Q, 4CS/D17N, 4CS/E31Q, 4CS/D35N, 4CS/D37N, 4CS/D40N, 4CS/N41Q, 4CS/K53R, 4CS/K53H, 4CS/K53M, 4CS/K53E, 4CS/K53Q, 4CS/K53A, 4CS/Q103E, 4CS/D110N, 4CS/N111Q, 4CS/E6A/K53A, 4CS/N14C/E31Q/S127C, 4CS/E31Q/K53A, 4CS/E31Q/D35N/K53A, 4C S/E31Q/N41Q/K53A, 4CS/E31Q/D35N/N41Q/K53A, 4CS/E31Q/D35N, 4CS/E31Q/N41Q, 4CS/E31Q/D35N/N41Q, 4CS/E31Q/D37N, 4CS/E31Q/D37N/K53A, 4C S/E31Q/M33C/S38C, 4CS/E31Q/S76C/L138C, 4CS/E31Q/S68I, 4CS/E31Q/S68F, 4CS/E31Q/S127W, 4C S/E31Q/S127Y, 4CS/E31Q/S127F, 4CS/S10C/E31Q/149C, 4C S/L15C/E31Q/R147C, 4C S/P28C/E31Q/L136C, 4CS/E31Q/S50C/P88C, 4CS/E31Q/T63C/P88C, 4C S/E31Q/V62C/Q103C, 4C5/510C/E31Q/N155C, 4CS/E31Q/S65C/P88C, 4CS/S7C/E31Q/S50C, 4CS/E31Q/D54C/A61C, 4C S/E31Q/A126C/K139C, 4CS/N14W/E31Q, 4CS/E31Q/D146Y, 4CS/E31Q/D146L, 4C S/E31Q/D146F, 4C S/E31Q/Q103L, 4CS/E31Q/Q103I, 4CS/E31Q/M150F, 4CS/Q18L/E31Q, 4CS/E31Q/S68Y, 4CS/E31Q/S38Q, 4C S/E31Q/S38R, 4CS/E31Q/S68D, 4CS/S7P/E31Q, 4CS/V11I/E31Q, 4CS/D23N/E31Q, 4CS/D23S/E31Q, 4CS/R27Q/E31Q, 4CS/L29V/E31Q, 4CS/E31Q/T34P, 4CS/E31Q/R39T, 4CS/E31Q/R39S, 4CS/E31Q/R44Q, 4CS/E31Q/I46V, 4CS/E31Q/S50Y, 4CS/E31Q/Q56L, 4CS/E31Q/Q56L/P57T, 4C S/E31Q/P57T, 4CS/E31Q/P57V, 4CS/E31Q/M60L, 4CS/E31Q/K67Q, 4CS/E31Q/E69K, 4CS/E31Q/I71M, 4CS/E31Q/E77K, 4CS/E31Q/I80T, 4CS/E31Q/I81V, 4CS/E31Q/I81L, 4C S/E31Q/N87S, 4C S/E31Q/D90E, 4CS/E31Q/K93D/T95E, 4CS/E31Q/K93N/T95E, 4CS/E31Q/T95E, 4CS/E31Q/K96G, 4CS/E31Q/S97N, 4CS/E31Q/N111D, 4CS/E31Q/M113I, 4CS/E31Q/S119L, 4CS/E31Q/L144N, 4CS/E31Q/R147K, 4CS/E31Q/I149V, 4CS/E31Q/M150T, 4CS/E31Q/E156Q/D157N, 4CS/K53S, 4CS/K53G, 4CS/K53T, 4CS/K53I, 4CS/K53L, 4CS/K53N, 4CS/K53D, 4CS/M51K, 4CS/M51Q, 4CS/M51I, 4CS/S55N, 4CS/S55Q, 4CS/Q56L, 4CS/Q56I, 4CS/P57A, 4CS/P57E, 4CS/M60L, 4CS/M60I, 4CS/K8Y, 4CS/K8Q, 4CS/K8E, 4CS/H109W, 4CS/H109Y, 4CS/E31Q/S38E, 4CS/E31Q/S38L, 4CS/E31Q/S38I, 4C S/E31Q/S38V, 4C S/E31Q/S68N, 4CS/E31Q/S68E, 4CS/E31Q/S68Q, 4C S/E31Q/S76C, 4C S/E31Q/S127D, 4CS/E31Q/S127E, 4CS/D23N/E31Q/R27Q, 4CS/E31Q/Q56L/T95E, 4CS/E31Q/K96Q/S119L, 4CS/E31Q/E141K/I149V, 4CS/E31Q/E141Q/I149V, 4CS/S7P/E31Q/S50Y, 4CS/E31Q/I80T/I81L/delD193, 4CS/E31Q/P57A/S119L/delD193, 4CS/E31Q/P57A/I80T/I81L/S119L/delD193, 4CS/E31Q/P57A/K93D/T95E/S119L/delD193, 4CS/E31Q/I80T/S119L/delD193, 4CS/E31Q/I80T/I81L/K93D/T95E/delD193, 4CS/E31Q/P57A/I80T/I81L/K93D/T95E/S119L/delD193, 4CS/S7C/E31Q/S50C/delD193, 4CS/S7C/E31Q/S50C/P57A/delD193, 4CS/S7C/E31Q/S50C/S119L/delD193, 4CS/S7C/E31Q/S50C/I80T/delD193, 4CS/S7C/E31Q/S50C/I80T/S119L/delD193, 4CS/S7C/E31Q/S50C/P57A/I80T/S119L/delD193, 4CS/S10C/E31Q/N155C/delD193, 4CS/S10C/E31Q/P57A/N155C/delD193, 4CS/S10C/E31Q/S119L/N155C/delD193, 4CS/S10C/E31Q/I80T/N155C/delD193, 4C S/S10C/E31Q/I80T/S119L/N155C/delD193, 4CS/S10C/E31Q/P57A/I80T/S119L/N155C/delD193, 4C S/S10C/E31Q/I49C/delD193, 4CS/L15C/E31Q/R147C/delD193, 4CS/E31Q/T63C/P88C/delD193, 4CS/N14C/E31Q/S127C/delD193, 4CS/E31Q/S38R/S127W/delD193, 4CS/S10C/D35E/N155C, 4CS/S10C/S36D/N155C, 4CS/S10C/S36N/N155C, 4CS/S10C/K53V/N155C, 4CS/S10C/K53Y/N155C, 4CS/S10C/K53F/N155C, 4CS/S10C/M51R/N155C, 4CS/S10C/M51L/N155C, 4C S/S10C/M51H/N155C, 4C S/S10C/M51F/N155C, 4CS/S10C/M51Y/N155C, 4CS/S10C/S55D/N155C, 4CS/S10C/S55E/N155C, 4CS/S10C/S55T/N155C, 4CS/S10C/P57Q/N155C, 4CS/S10C/P57D/N155C, 4CS/S10C/P57Y/N155C, 4CS/S10C/P57N/N155C, 4CS/S10C/M60Y/N155C, 4CS/S10C/M60F/N155C, 4CS/S10C/D110Q/N155C, 4CS/S10C/D110R/N155C, 4CS/S10C/N111D/N155C, 4CS/S10C/N111S/N155C, 4CS/S10C/N111T/N155C, 4CS/S10C/N111E/N155C, 4CS/S10C/D132Q/N155C, 4CS/S10C/D132EN155C, 4CS/E6Q/S10C/K53D/N155C, 4CS/E6Q/S10C/M51K/K53D/N155C, 4CS/S10C/E31Q/D35N/N41Q/K53A/N155C, 4CS/S10C/E31Q/N41Q/K53A/N155C, 4CS/S10C/E31Q/K53A/N155C, 4CS/S10C/K53T/N155C, 4CS/S10C/P57A/N155C, 4CS/S10C/N155C, 4CS/S10C/S76G/N155C, 4CS/S10C/S76A/N155C, 4CS/S10C/M51K/K53D/N155C, 4CS/S10C/M51K/K53E/N155C, 4CS/E6Q/S10C/K53E/N155C, 4CS/E6Q/S10C/M51K/K53E/N155C, 4CS/E6Q/S10C/M51K/P57E/N155C, 4CS/S10C/M51K/P57E/N155C, 4CS/E6Q/S10C/P57E/N155C, 4CS/S10C/E31Q/K53T/N155C, 4CS/S10C/K53G/P57E/N155C, 4CS/S10C/K53T/P57E/N155C, 4CS/S10C/K53A/P57E/N155C, 4CS/S10C/P57E/N155C, 4CS/S10C/K53D/N155C, 4CS/S10C/E31Q/N41Q/N155C, 4CS/S10C/K53A/N155C, 4CS/S10C/K53G/N155C, 4CS/S10C/K53E/N155C, 4CS/S10C/K53S/N155C, 4CS/S10C/M51L/K53D/N155C, 4CS/S10C/K53D/D110R/N155C, 4CS/S10C/K53D/N111T/N155C, 4CS/S10C/K53D/S55T/N155C, 4CS/S10C/K53D/S55T/D110R/N155C, 4CS/S10C/M51L/K53D/S55T/D110R/N111T/N155C, 4CS/S10C/M51L/K53D/S55T/D110R/N155C, 4CS/S10C/K53D/S55T/D110R/N111T/N155C, 4CS/S10C/K53D/S55T/N111T/N155C, 4CS/S10C/E31Q/D35N/N155C, 4CS/S10C/N41Q/N155C, 4CS/S10C/D35N/N155C, 4CS/S10C/D37N/N155C, 4CS/S10C/E31Q/D37N/N155C, 4CS/S10C/D35N/D37N/N155C, 4CS/E6Q/S10C/M51L/K53D/S55T/D110R/N111T/N155C, 4CS/S10C/K53D/H109Y/N155C, 4CS/S10C/D37N/K53D/N155C, 4CS/S10C/D35N/K53D/N155C, 4CS/K8E/S10C/K53D/N155C, 4CS/S10C/E31Q/K53D/N155C, 4CS/S10C/N41Q/K53D/N155C, 4CS/S10C/K53D/P57V/N155C, 4CS/S10C/K53D/P57T/N155C, 4CS/E6Q/S10C/K53D/N111T/N155C, E6A/K53A, D35N/K53A, N41Q/K53A, D35N/N41Q/K53A, D35N/N41Q, D37N/K53A, E6Q/K53D, E6Q/M51K/K53D, M51K/K53D, M51K/K53E, E6Q/K53E, E6Q/M51K/K53E, E6Q/M51K/P57E, M51K/P57E, E6Q/P57E, K53G/P57E, K53T/P57E, K53A/P57E, M51L/K53D, K53D/D110R, K53D/N111T, K53D/S55T, K53D/S55T/D110R, M51L/K53D/S55T/D110R/N111T, M51L/K53D/S55T/D110R, K53D/S55T/D110R/N111T, K53D/S55T/N111T, D35N/D37N, E6QN151L/K53D/S55T/D110R/N111T, K53D/H109Y, D37N/K53D, D35N/K53D, K8E/K53D, N41Q/K53D, K53D/P57V, K53D/P57T, E6Q/K53D/N111T, Q56L/P57T, K93D/T95E, K93N/T95E, E156Q/D157N, D23N/R27Q, Q56L/T95E, K96Q/S119L, E141K/I149V, E141Q/I149V, S7P/S50Y, 180T/I81L, P57A/S119L, P57A/180T/181L/S119L, P57A/K93D/T95E/S119L, I80T/S119L, 180T/I81L/K93D/T95E, P57A/180T/I81L/K93D/T95E/S119L, P57A/180T/S119L, N14C/S127C, M33C/S38C, S76C/L138C, S10C/I49C, L15C/R147C, P28C/L136C, S50C/P88C, T63C/P88C, V62C/Q103C, S10C/N155C, S65C/P88C, S7C/S50C, D54C/A61C, A126C/K139C, C38R/C127W, E31Q/K53A, E31Q/D35N/K53A, E31Q/N41Q/K53A, E31Q/D35N/N41Q/K53A, E31Q/D35N, E31Q/N41Q, E31Q/D35N/N41Q, E31Q/D37N, E31Q/D37N/K53A, S10C/E31Q/I49C, L15C/E31Q/R147C, P28C/E31Q/L136C, E31Q/S50C/P88C, E31Q/T63C/P88C, E31Q/V62C/Q103C, S10C/E31Q/N155C, E31Q/S65C/P88C, S7C/E31Q/S50C, E31Q/D54C/A61C, E31Q/A126C/K139C, N14W/E31Q, E31Q/D146Y, E31Q/D146L, E31Q/D146F, E31Q/Q103L, E31Q/Q103I, E31Q/M150F, Ql8L/E31Q, S7P/E31Q, V11I/E31Q, D23N/E31Q, D23 S/E31Q, R27Q/E31Q, L29V/E31Q, E31Q/T34P, E31Q/R39T, E31Q/R39S, E31Q/R44Q, E31Q/I46V, E31Q/S50Y, E31Q/Q56L, E31Q/Q56L/P57T, E31Q/P57T, E31Q/P57V, E31Q/M60L, E31Q/K67Q, E31Q/E69K, E31Q/I71M, E31Q/E77K, E31Q/I80T, E31Q/I81V, E31Q/I81L, E31Q/N87S, E31Q/D90E, E31Q/K93D/T95E, E31Q/K93N/T95E, E31Q/T95E, E31Q/K96G, E31Q/S97N, E31Q/N111D, E31Q/M1131, E31Q/S119L, E31Q/L144N, E31Q/R147K, E31Q/I149V, E31Q/M150T, E31Q/E156Q/D157N, D23N/E31Q/R27Q, E31Q/Q56L/T95E, E31Q/K96Q/S119L, E31Q/E141K/I149V, E31Q/E141Q/I149V, S7P/E31Q/S50Y, E31Q/180T/181L/delD193, E31Q/P57A/S119L/delD193, E31Q/P57A/180T/181L/S 11 9L/delD193, E31Q/P57A/K93D/T95E/S119L/delD193, E31Q/I80T/S119L/delD193, E31Q/I80T/I81L/K93D/T95E/delD193, E31Q/P57A/180T/181L/K93D/T95E/S119L/delD193, S7C/E31Q/S50C/delD193, S7C/E31Q/S50C/P57A/delD193, S7C/E31Q/S50C/S119L/delD193, S7C/E31Q/S50C/I80T/delD193, S7C/E31Q/S50C/I80T/S119L/delD193, S7C/E31Q/S50C/P57A/I80T/S119L/delD193, S10C/E31Q/N155C/delD193, S10C/E31Q/P57A/N155C/delD193, S10C/E31Q/S119L/N155C/delD193, S10C/E31Q/I80T/N155C/delD193, S10C/E31Q/I80T/S119L/N155C/delD193, S10C/E31Q/P57A/I80T/S119L/N155C/delD193, S10C/E31Q/I49C/delD193, L15C/E31Q/R147C/delD193, E31Q/T63C/P88C/delD193, S10C/D35E/N155C, S10C/S36D/N155C, S10C/S36N/N155C, S10C/K53V/N155C, S10C/K53Y/N155C, S10C/K53F/N155C, S10C/M51R/N155C, S10C/M51L/N155C, S10C/M51H/N155C, S10C/M51F/N155C, S10C/M51Y/N155C, S10C/S55D/N155C, S10C/S55E/N155C, S10C/S55T/N155C, S10C/P57Q/N155C, S10C/P57D/N155C, S10C/P57Y/N155C, S10C/P57N/N155C, S10C/M60Y/N155C, S10C/M60F/N155C, S10C/D110Q/N155C, S10C/D110R/N155C, S10C/N111D/N155C, S10C/N111S/N155C, S10C/N111T/N155C, S10C/N111E/N155C, S10C/D132Q/N155C, S10C/D132E/N155C, E6Q/S10C/K53D/N155C, E6Q/S10C/M51K/K53D/N155C, S10C/E31Q/D35N/N41Q/K53A/N155C, S10C/E31Q/N41Q/K53A/N155C, S10C/E31Q/K53A/N155C, S10C/K53T/N155C, S10C/P57A/N155C, S10C/M51K/K53D/N155C, S10C/M51K/K53E/N155C, E6Q/S10C/K53E/N155C, E6Q/S10C/M51K/K53E/N155C, E6Q/S10C/M51K/P57E/N155C, S10C/M51K/P57E/N155C, E6Q/S10C/P57E/N155C, S10C/E31Q/K53T/N155C, S10C/K53G/P57E/N155C, S10C/K53T/P57E/N155C, S10C/K53A/P57E/N155C, S10C/P57E/N155C, S10C/K53D/N155C, S10C/E31Q/N41Q/N155C, S10C/K53A/N155C, S10C/K53G/N155C, S10C/K53E/N155C, S10C/K53S/N155C, S10C/M51L/K53D/N155C, S10C/K53D/D110R/N155C, S10C/K53D/N111T/N155C, S10C/K53D/S55T/N155C, S10C/K53D/S55T/D110R/N155C, S10C/M51L/K53D/S55T/D110R/N111T/N155C, S10C/M51L/K53D/S55T/D110R/N155C, S10C/K53D/S55T/D110R/N111T/N155C, S10C/K53D/S55T/N111T/N155C, S10C/E31Q/D35N/N155C, S10C/N41Q/N155C, S10C/D35N/N155C, S10C/D37N/N155C, S10C/E31Q/D37N/N155C, S10C/D35N/D37N/N155C, E6Q/S10C/M51L/K53D/S55T/D110R/N111T/N155C, S10C/K53D/H109Y/N155C, S10C/D37N/K53D/N155C, S10C/D35N/K53D/N155C, K8E/S10C/K53D/N155C, S10C/E31Q/K53D/N155C, S10C/N41Q/K53D/N155C, S10C/K53D/P57V/N155C, S10C/K53D/P57T/N155C, or E6Q/S10C/K53D/N111T/N155C.

Additional IL-18 positions suitable for engineering substitutions, specific substitutions, and specific variants are known in the art which may find use in the IL18 variants and IL18-Fc fusions of the invention. Such positions, substitutions and variants are, for example, described in US 2021/0015891, US 2019/0070262, WO 2022/094473 and Zhou et al. 2020 (e.g., Y1, L5, K8, D17, E31, T34, D35, S36, D37, C38, D40, N41, M51, K53, S55, Q56, P57, G59, M60, C68, E77, Q103, 5105, H109, D110, N111, M113, R131, V153, and N155 as in Y1R, Y1H, Y1D, Y1L, Y1F, LSH, L51, L5Y, LSF, K8Q, K8R, D17G, D17R, D17H, D17A, E31K, E31A, E31T, E31G, E31R, T34E, T34K, T34A, D35A, D35S, D35Y, S36N, S36K, S36R, D37P, D37R, D37V, D37A, D37L, D37H, C38S, D40A, D4OS, D40Y, N41R, N41S, N41K, M51T, M51K, M51D, M51N, M51E, M51R, M51F, M51I, M51L, K53R, K53G, K53S, K53T, S55K, S55R, Q56E, Q56A, Q56R, Q56V, Q56G, Q56K, Q56L, Q56H, P57L, P57G, P57A, P57K, G59A, G59T, M60K, M60Q, M6OR, M60L, M60I, M60F, C68D, E77D, Q103E, Q103K, Q103P, Q103A, Q103R, Q103I, Q103L, S105D, S105N, S105R, S105K, S105A, H109A, H109P, H109D, D110K, D110H, D110N, D110Q, D110E, D110S, D110G,N111H, N111Y,N111D, N111R, N111S, N111G, M113V, M113R, M113T, M113K, M113F, M1131, M113L, R131S, V1531, V153T, V153A, N155K, N155H, M51T/M60K/S105D/D110K/N111H, M51T/S55K/G59A/M60K/S105D/D110K/N111H/V153I, Y1R/M51TNI60K/S105D/D110K/N111H, Y1R/M51T/K53R/M60K/S105N/D110K/N111Y, K8Q/M51T/S55K/G59TNI60K/S105R/D110H/N155K, K8R/M51K/S55K/G59A/M60Q/S105D/D110K/N111H/V153I, K8R/M51D/S55K/G59A/M60X/S105D/D110K/N111H/V153I, L5H/M51T/K53R/M60K/S105D/D110N/V153T, L5I/M51K/S55K/G59A/M60Q/S105K/D110Q/N111H/N155K, L51/M51T/S55R/M60K/Q103E/S105D/D110H/N111H/V153I, L51/M51T/S55K/M6OK/S105D/D110K/N111H/V153T/N155H, L51/M51T/S55K/G59A/M60K/S105R/D110H/N111H/V1531/N155K, L51/K8R/M51T/S55K/M6OK/S105D/N111Y/V1531/N155K, L5Y/K8R/M51T/K53R/M60K/S105D/D110E/N111H/N155K, Y1H/L5YN151T/K53R/M60K/S105D/D110H/N155K, Y1R/M51T/K53R/G59A/M60K/S105D/D110Q/N111H/V153A/N155K, Y1R/K8R/M51D/K53R/M6OR/Q103K/S105N/D110K/N111Y/N155H, Y1R/K8R/M51N/K53R/M60Q/Q103K/S105R/D110N/N111H/N155K, Y1R/K8R/M51T/M60K/S105D/D110K/N111H, Y1R/L5H/M51T/K53R/M60K/Q103E/S105N/D110K/N111Y, Y1R/K8R/M51T/K53R/G59A/M60K/Q103E/S105D/D110Q/N111H/V1531/N155X, Y1R/K8R/M51T/K53R/G59TNI60K/S105N/D110H/N111D/N155H, Y1R/K8R/M51T/G59A/M60K/Q103E/S105D/D110Q/N111H/V1531/N155K, Y1R/L5HNI51T/K53R/M60K/Q103E/S105N/D110K/N111Y, Y1R/L5YN151T/G59TNI60K/E77D/S105D/D110K/N111H, Y1R/K8R/M51T/K53R/G59TN160K/S105K/D110N/N111H/N155K, M51E/Q56E/P57L/M6OR/Q103P/S105A/D110N/N111R/M113V, M51K/Q56A/P57G/M60L/Q103E/S105D/D110S/M113V, M51K/K53G/Q56A/P57A/M60L/D110K/N111R, M51K/K53G/Q56R/P57G/M60L/Q103E/S105D/D110N/N111SN1113R, M51K/K53G/Q56VNI6OL/Q103A/S105A/D110S/N111R/M113T, M51K/K53S/Q56G/P57A/M60L/Q103A/S105A/D110G/N111R/M113T, M51K/K53S/Q56K/P57A/Q103A/S105D/D110S/N111SN1113R, M51K/K53S/Q56L/P57A/M60L/S105D/D110S/N111R, M51K/K53S/Q56R/P57A/M60L/S105N/D110G/N111R, M51K/K53S/Q56R/P57A/M60L/Q103A/D110G/N111R/M113T, M51K/K53S/Q56R/P57A/M60L/Q103A/S105D/D110S/N111GN1113R, M51K/K53T/Q56R/M60L/Q103E/S105D/D110S/N111SN1113K, M51K/K53T/Q56R/P57A/Q103E/S105D/D110N/N111D/M113R, M51R/Q56G/P57K/M60L/Q103R/D110S/N111R/M113V, M51K/K53G/Q56G/P57A/M60L/Q103E/S105D/D110S/N111G/M113V, M51K/K53G/Q56R/S105A/D110N/N111R, M51K/K53S/Q56L/P57A/M60L/S105D/D110S/N111R, M51K/K53S/Q56R/P57A/M60L/Q103A/D110G/N111R/M113T, M51K/K53S/Q56R/P57A/M60L/S105N/D110G/N111R, M51K/K53G/Q56VNI6OL/Q103A/S105A/D110S/N111R/M113T, M51K/K53S/Q56R/P57A/M60L/Q103A/D110G/N111R/M113T, M51K/K53S/Q56R/P57A/M60L/Q103A/S105D/D110S/N111GN1113R, Y1H/D17G/E31K/D35A/D40A/M51F/Q103I/H109A/M113F, Y1D/E31A/S36N/D37P/D40S/M151F/M60I/Q103L/H109P, Y1H/D17G/E31A/T34E/D35A/D37P/M51F/M60L/Q103L, Y1L/D17G/E31A/D35S/D37P/D40S/N41R/M51F/M6OF/Q103I/M113I, Y1H/E31T/T34E/D35S/S36K/D37P/N41S/M60I/Q103I/M113F, D17R/E31T/D35Y/S36K/M51F/M60F/Q103I/H109A/M113I, Y1H/D17G/E31G/T34E/D35A/D37P/M51F/Q103I/H109A/M113L, Y1H/D17G/E31T/D35S/N41K/M51F/M60L/Q103L/H109P/M113F, Y1H/D17G/E31A/T34E/D35S/S36N/D37R/N41S/M60F/Q103L/M113I, D17G/E31T/D35A/D37P/M51F/M60L/Q103L/M113I/D132S, Y1D/E31A/T34K/D35 S/S36N/D37V/D40A/Q103I/H109P/M113L, Y1H/D17G/E31G/T34E/D35A/D37P/D40A/N41K/M51I/M60F/Q103L/M113F/D132S, Y1H/D17G/E31A/T34A/D35S/S36N/D37R/D40A/M60F/Q103I/H109P/M113F, E31A/T34K/D35A/S36K/D37A/D40 S/M51F/M60F/Q103L/H109A/M113I, Y1H/D17H/E31T/T34A/S36N/D37A/M51F/M60L/Q103I/H109D, D17G/E31T/T34K/D35 S/S36N/D37L/D40 S/M51F/M60I/Q103I/H109D/M113I, Y1H/D17G/E31R/T34A/D35A/D37H/N41S/M51I/M60F/Q103L/M113I, E31A/T34A/D35S/S36N/D37P/D40Y/N41K/M60L/Q103L/H109A/M113L, Y1H/D17G/E31T/T34A/D35S/S36N/D37H/D40Y/M60L/Q103L/H109A/M113I, Y1H/D17G/E31A/S36N/D37P/D40 S/N41R/M51F/Q103I/M113F, D17G/E31T/D35A/D37H/D40 S/N41 S/M51L/Q103L/M113F, D17G/E31T/T34E/D35Y/S36R/D37L/D40A/M51F/Q56H/M60L/Q103L/H109D/M113L, D17G/E31T/T34E/D35S/D37L/M51L/Q103L/H109A/M113F/D132S, D17G/E31K/T34E/D35A/S36N/D40S/N41S/M51I/M60L/Q103I/H109A, Y1D/E31A/T34K/D35A/D37A/D40A/M60L/Q103L/M113F, D17G/E31G/D35A/S36K/D37H/D40A/N41R/M51F/Q103I/H109A/M113F, Y1F/L5F/D17G/E31T/T34E/D35S/S36K/N41R/M51F/M60L/Q103L/M113L, Y1F/D17G/E31A/T34A/D35S/S36N/D37H/D40A/M51L/M60L/Q103I, D17G/E31A/D35 S/S36K/D37R/M51L/M60F/Q103I/M113I, Y1L/L5H/D17G/E31G/T34E/D35S/D37P/D40Y/M51F/M60L/Q103I, D17A/E31T/D35A/D37P/D40 S/N41 S/M51L/M60L/Q103L/H109P/M113I, D17G/E31A/Q103L, D17G/E31A/D35S/M51F/Q103L, D17G/E31A/T34E/D35 S/M51F/M60L/Q103L, M51K/K53S/Q56L/D110N/N111R, M51K/K53S/Q56L/P57A/M60L/D110N/N111R, M51K/K53S/Q56L/P57A/M60L/S105D/D110N/N111R, C38 S/M51K/K53 S/Q56L/P57A/M60L/C68D/S105D/D110 S/N111R, M51G/K53A/Q56R/M60K, C38S/C68S, C38S/C68G, C38S/C68A, C38S/C68V, C38S/C68D, C38S/C68E, C38S/C68N, and Y1H/M51A/K53G/Q56R/P57A/M60K); WO2003057821 (SLOT, 112V, T45S, F47Y, Y52F, I64V, F101Y, LSV, L20V, L20I, F21Y, I22V, V66I, S72T, S148F, K4E, E6I, KBD, R131, L15R, D17K, R27K, F30A, D35K, D37F, C38E, R39A, D4OW, M51E, K53G, Q56I, R58A, V62K, D94K, T95F, R104L, G1081, N111K, K129F, R131D, D132L, L133E, F134A, M150T, and F151S); U.S. Pat. No. 6,800,479 (D35E and D40E); U.S. Pat. No. 7,524,488, U.S. Pat. No. 7,875,709, WO 2002/101049, and Kim et al. 2001 (e.g., E42, 185, K89, M96, D130, K132, P143, M149, and L189 as in E42A, K89A, and E42A/K89A); and Saetang et al. 2016 (e.g., E6K, M33Q, M60Q, and T63A). In some embodiments, one or more of the foregoing substitutions and variants may be used in combination with any of the other IL-18 substitutions and variants described herein (e.g. those that exhibit reduced binding affinity to the IL18 receptor 1 (IL18R1), IL18 receptor accessory protein (IL18RAP), IL18R1:IL18RAP complex and/or the IL18 binding protein (IL18BP), compared to wildtype human IL18; those that exhibit reduced heterogeneity; those that exhibit improved production yield; those that exhibit modulated potency; those that exhibit improved stability, and/or those that exhibit reduced IL18BP sink) to engineer IL18 variants of the invention. In some embodiments, each of these substitutions and variants may be used alone or in combination in the IL18-Fc fusion proteins of the invention. In some embodiments, each of these substitutions and variants may be used in combination with other IL-18 substitutions and variants described herein in the IL18-Fc fusion proteins of the invention. In some embodiments, the IL18 variants or IL18-Fc fusions include alternative substitutions at any of the foregoing positions.

In some embodiments, the IL18 variant of the IL18-Fc fusion protein includes one or more amino acid substitutions provided in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, and 62. In some embodiments, the IL18 variant of the IL18-Fc fusion protein is depicted in any one of FIGS. 13A-13B, 14, 15A-15D, 16A-16E, 17A-17B, 18, 19A-19P, 20A-20D, 41A-41C, 42A-42D, 43A-43B, and 44A-44C. In certain embodiments, the IL18 variant includes an amino acid sequence set forth in SEQ ID NOS:84-238 and 799-949.

V. Interleukin 18-Fusion Variants

In some embodiments, the IL18-Fc fusion proteins described include one or more modifications (including an amino acid addition, deletion and substitution) to reduce binding to IL18BP. In some embodiments, the IL18-Fc fusion proteins include one or more modifications to reduce binding to IL18 receptors. In some embodiments, the IL18-Fc fusion proteins include one or more modifications to improve stability of the fusion proteins compared to wildtype IL18. In some embodiments, the IL18-Fc fusion proteins include one or more modifications for improving the production (such as, but not limited to, reducing heterogeneity of such proteins during manufacturing) of such fusion proteins. In some embodiments, the IL18-Fc fusion proteins include one or more modifications to remove free cysteines.

In certain embodiments, the IL18 includes one or more modifications to reduce heterogeneity that may affect IL18-Fc fusion protein production and/or activity. In some embodiments, such IL18 variants include one or more modifications to remove one or more free cysteines. In some embodiments, a cysteine (C) of a wildtype IL18 is substituted with a serine (S). Exemplary residues that may be modified to reduce heterogeneity and improve production include one or more amino acid residues selected from the group consisting of C38, C68, C76 and C127.

Particular modifications to reduce heterogeneity include amino acid substitutions C38S, C68S, C76S, C127S, C38S/C68S, C38S/C76S, C38S/C127S, C68S/C76S, C68S/C127S, C76S/C127S, C38S/C68S/C76S, C38S/C68S/C127S, C38S/C76S/C127S, C68S/C76S/C127S, C38S/C68S/C76S/C127S, (also referred to as the “4CS” substitutions) and combinations thereof. In some embodiments, the amino acid substitutions C38S/C68S/C76S/C127S of IL18 is herein referred to as the “4CS” substitutions.

In certain embodiments, the IL18-Fc fusion proteins include one or more modifications to improve production of such fusion proteins. For instance, one or more of the modifications is directed to reduce the potential impact of post-translational modification related liabilities. Residues which may be modified include removing the C-terminal lysine on one or both Fc domains. In some embodiments, the modification includes a K447 deletion (K447 or K447del) in one or both Fc domains. Such modifications are depicted in the heterodimeric Fc backbones of FIGS. 9A-9D.

Exemplary IL18 variants that can be included in the IL18-Fc fusion proteins include, but are not limited to those in FIGS. 13A-20D and 41A-44C. In some embodiments, the IL18-Fc fusion protein includes one or more of the IL18 variants in FIGS. 13A-20D and 41A-44C. In certain embodiments, the IL18-Fc fusion protein includes an IL18 that includes 1, 2, 3, 4, 5, 6, 7, 8, 9, 10 or more additional modifications as compared to an IL18 variant in FIGS. 13A-20D and 41A-44C.

In some embodiments, the IL18 variant is engineered to remove a possible “DG” aspartic acid isomerization motif, such as those found when the C-terminus of IL18 is covalently attached to a G4S linker. In certain embodiments, the C-terminus of IL18 is covalently attached to the glutamic acid (E) residue of the hinge region of the Fc domain. In some embodiments, the IL18 variant of the fusion protein comprises a deletion or substitution at the C-terminal aspartic acid (D) residue (D). In certain embodiments, the IL18 variant includes a modification such as a D157del (D157_), D157S, or D157A modification. In some embodiments, the C-terminus of IL18 is covalently attached to a linker selected from the group consisting of AGGGG (SEQ ID NO: 39) or EAAAK (SEQ ID NO:40).

In some embodiments, the IL18-Fc fusion protein includes a format to improve the production of the fusion protein. In some instances, the IL18-Fc fusion protein includes a Fab-Fc chain occupying the empty-Fc chain. In some embodiments, the Fab arm is a silent Fv. In some embodiments, the silent Fv is based on SEQ ID NOS:23 and 24 as disclosed in the sequence listing of WO 2020/078905. In some embodiments, the format comprising a first monomer comprising a IL18 protein or variant thereof and a first Fc domain, a second monomer comprising the heavy chain of an Fab arm and a second Fc domain, and a third monomer comprising the light chain of the Fab arm.

In some embodiments, the empty-Fc chain of an IL18-Fc fusion protein includes the substitutions H435R/Y436F in the Fc domain. In some embodiments, the empty-Fc chain of the IL18-Fc fusion protein does not include the substitutions H435R/Y436F in the Fc domain. In some embodiments, the Fab-Fc chain of the IL18-Fc fusion protein includes the substitutions H435R/Y436F in the Fc domain. In some embodiments, the Fab-Fc chain of the IL18-Fc fusion protein does not include the substitutions H435R/Y436F in the Fc domain.

In some embodiments, the IL18 variant of the fusion protein includes a modification at position E31 of IL18. In some embodiments, the IL18 variant includes the amino acid substitution E31Q.

In some embodiments, the IL18 variant of the fusion protein includes a modification at position K53 of IL18. In some embodiments, the IL18 variant includes the amino acid substitution K53D.

In one aspect, provided herein are compositions that include any one of the IL18 variants described herein.

VI. Heterodimerization Variants

In some embodiments, the dimeric IL18-Fc fusion protein is a heterodimeric IL18-Fc fusion protein. Such heterodimeric proteins include two different Fc domains (one on each of the first and second monomers) that include modifications that facilitate the heterodimerization of the first and second monomers and/or allow for ease of purification of heterodimers over homodimers, collectively referred to herein as “heterodimerization variants.” As discussed below, heterodimerization variants can include skew variants (e.g., the “knobs and holes” and “charge pairs” variants described below) as well as “pI variants” that facilitates the separation of homodimers away from heterodimers. As is generally described in U.S. Pat. No. 9,605,084, hereby incorporated by reference in its entirety and specifically as below for the discussion of heterodimerization variants, useful mechanisms for heterodimerization include “knobs and holes” (“KIH”) as described in U.S. Pat. No. 9,605,084, “electrostatic steering” or “charge pairs” as described in U.S. Pat. No. 9,605,084, pI variants as described in U.S. Pat. No. 9,605,084, and general additional Fc variants as outlined in U.S. Pat. No. 9,605,084 and below.

1. Skew Variant

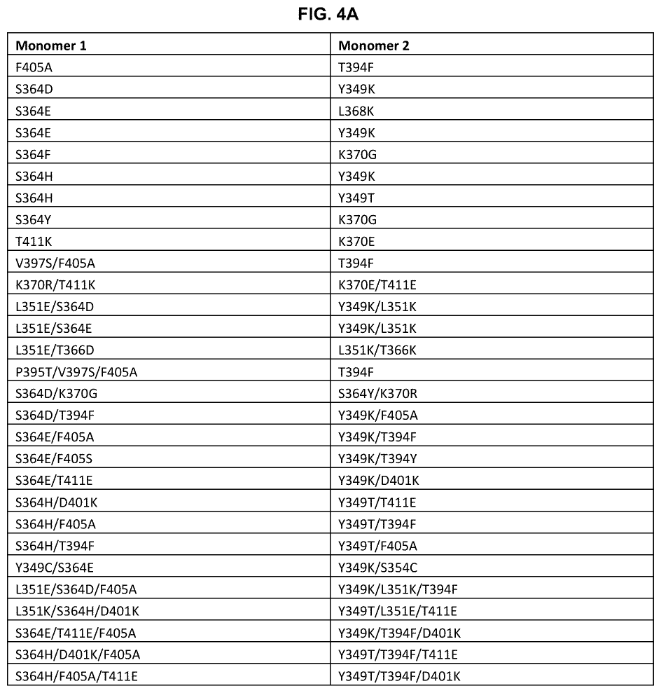

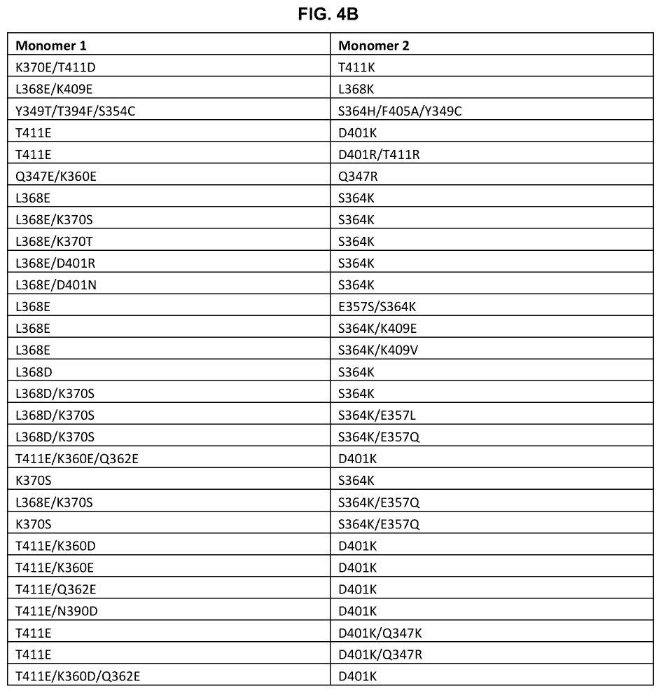

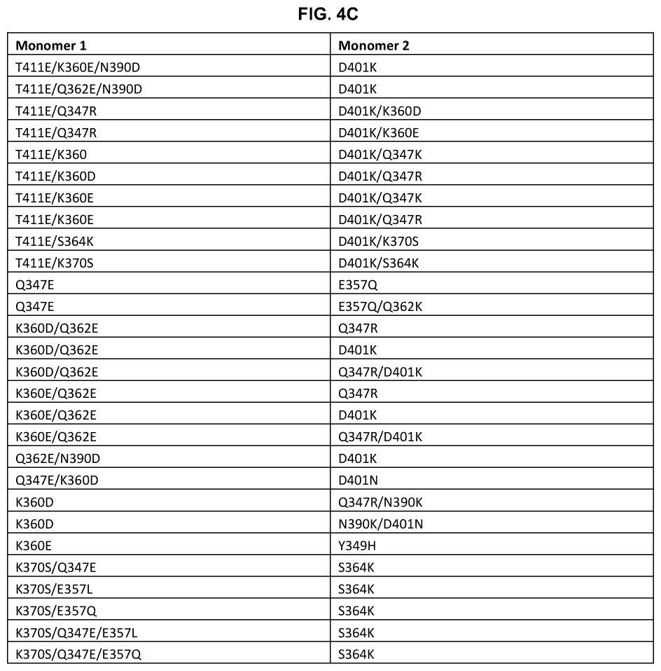

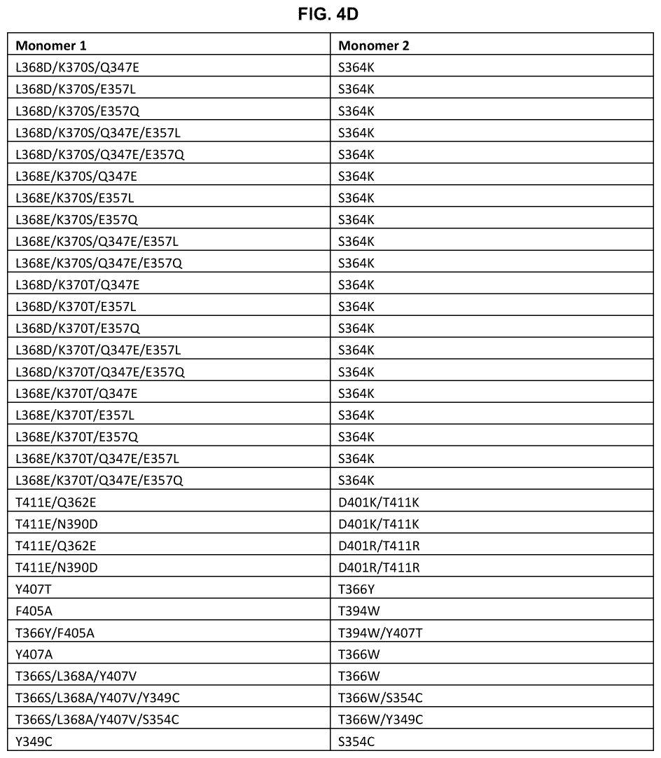

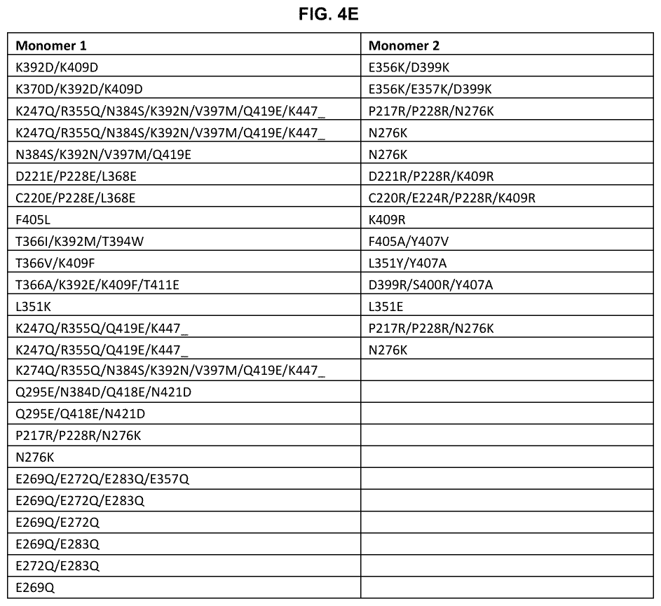

In some embodiments, the heterodimeric IL18-Fc fusion protein includes skew variants, which are one or more amino acid modifications in a first Fc domain (A) and/or a second Fc domain (B) that favor the formation of Fc heterodimers (Fc dimers that include the first and the second Fc domain; A-B) over Fc homodimers (Fc dimers that include two of the first Fc domain or two of the second Fc domain; A-A or B-B). Suitable skew variants are included in the FIG. 29 of US Publ. App. No. 2016/0355608, hereby incorporated by reference in its entirety and specifically for its disclosure of skew variants, as well as in FIG. 4.

One mechanism for skew variants is generally referred to in the art as “knobs and holes,” referring to amino acid engineering that creates steric influences to favor heterodimeric formation and disfavor homodimeric formation, as described in USSN 61/596,846, Ridgway et al., Protein Engineering 9(7):617 (1996); Atwell et al., J. Mol. Biol. 1997 270:26; U.S. Pat. No. 8,216,805, all of which are hereby incorporated by reference in their entirety and specifically for the disclosure of “knobs and holes” mutations. This is sometime referred to herein as “steric variants.” The figures identify a number of “monomer A — monomer B” pairs that rely on “knobs and holes”. In addition, as described in Merchant et al., Nature Biotech. 16:677 (1998), these “knobs and holes” mutations can be combined with disulfide bonds to further favor formation of Fc heterodimers.

An additional mechanism for skew variants that finds use in the generation of heterodimers is sometimes referred to as “electrostatic steering” as described in Gunasekaran et al., J. Biol. Chem. 285(25):19637 (2010), hereby incorporated by reference in its entirety. This is sometimes referred to herein as “charge pairs.” In this embodiment, electrostatics are used to skew the formation towards heterodimerization. As those in the art will appreciate, these may also have an effect on pI, and thus on purification, and thus could in some cases also be considered pI variants. However, as these were generated to force heterodimerization and were not used as purification tools, they are classified as “skew variants”. These include, but are not limited to, D221E/P228E/L368E paired with D221R/P228R/K409R (e.g., these are “monomer” corresponding sets) and C220E/P228E/368E paired with C220R/E224R/P228R/K409R.

In some embodiments, the skew variants advantageously and simultaneously favor heterodimerization based on both the “knobs and holes” mechanism as well as the “electrostatic steering” mechanism. In some embodiments, the heterodimeric IL18-Fc fusion proteins includes one or more sets of such heterodimerization skew variants. Exemplary skew variants that fall into this category include: S364K/E357Q: L368D/K370S; L368D/K370S: S364K; L368E/K370S: S364K; T411T/E360E/Q362E: D401K; L368D/K370S: S364K/E357L; K370S: S364K/E357Q; a T366S/L368A/Y407V: T366W (optionally including a bridging disulfide, T366S/L368A/Y407V/Y349C: T366W/S354C or T366S/L368A/Y407V/S354C: T366W/Y349C). These variants come in “pairs” of “sets.” That is, one set of the pair is incorporated into the first monomer and the other set of the pair is incorporated into the second monomer. In terms of nomenclature, the pair “S364K/E357Q: L368D/K370S” means that one of the monomers includes an Fc domain that includes the amino acid substitutions S364K and E357Q and the other monomer includes an Fc domain that includes the amino acid substitutions L368D and K370S; as above, the “strandedness” of these pairs depends on the starting pI. It should be noted that these sets do not necessarily behave as “knobs in holes” variants, with a one-to-one correspondence between a residue on one monomer and a residue on the other. That is, these pairs of sets may instead form an interface between the two monomers that encourages heterodimer formation and discourages homodimer formation, allowing the percentage of heterodimers that spontaneously form under biological conditions to be over 90%, rather than the expected 50% (25 homodimer A/A:50% heterodimer A/B:25% homodimer B/B). Exemplary heterodimerization “skew” variants are depicted in FIG. 4.

In exemplary embodiments, the heterodimeric IL18-Fc fusion protein includes a S364K/E357Q: L368D/K370S; L368D/K370S: S364K; L368E/K370S: S364K; T411T/E360E/Q362E: D401K; L368D/K370S: S364K/E357L; K370S: S364K/E357Q; or a T366S/L368A/Y407V: T366W (optionally including a bridging disulfide, T366S/L368A/Y407V/Y349C: T366W/S354C or T366S/L368A/Y407V/S354C: T366W/Y349C) “skew” variant amino acid substitution set. In an exemplary embodiment, the heterodimeric IL18-Fc fusion protein includes a “S364K/E357Q: L368D/K370S” amino acid substitution set.

In some embodiments, the skew variants provided herein can be optionally and independently incorporated with any other modifications, including, but not limited to, other skew variants (see, e.g., in FIG. 37 of US Publ. App. No. 2012/0149876, herein incorporated by reference, particularly for its disclosure of skew variants), pI variants, isotpypic variants, FcRn variants, ablation variants, etc. into one or both of the first and second Fc domains of the IL18-Fc fusion protein. Further, individual modifications can also independently and optionally be included or excluded from the subject IL18-Fc fusion proteins.

2. pI (Isoelectric point) Variants for Heterodimers

In some embodiments, the heterodimeric IL18-Fc fusion protein includes purification variants that advantageously allow for the separation of heterodimeric IL18-Fc fusion proteins from homodimeric proteins.

There are several basic mechanisms that can lead to ease of purifying heterodimeric proteins. One such mechanism relies on the use of pI variants which include one or more modifications that affect the isoelectric point of one or both of the monomers of the fusion protein, such that each monomer, and subsequently each dimeric species, has a different pI, thus allowing the isoelectric purification of A-A, A-B and B-B dimeric proteins. Alternatively, some formats also allow separation on the basis of size. As is further outlined below, it is also possible to “skew” the formation of heterodimers over homodimers using skew variants. Thus, a combination of heterodimerization skew variants and pI variants find particular use in the subject IL18 fusion proteins provided herein.

Additionally, as more fully outlined below, depending on the format of the heterodimeric Fc fusion protein, pI variants can be either contained within the constant region and/or Fc domains of a monomer, and/or domain linkers can be used. In some embodiments, the heterodimeric IL18-Fc fusion protein includes additional modifications for alternative functionalities can also create pI changes, such as Fc, FcRn and KO variants.

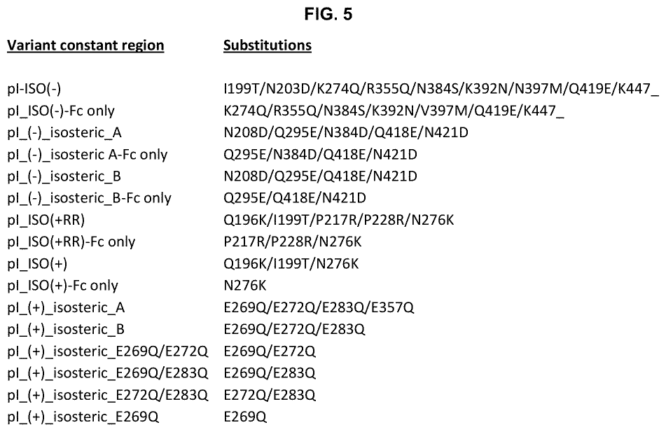

In the embodiments that utilizes pI as a separation mechanism to allow the purification of heterodimeric IL18-Fc fusion proteins, amino acid modifications can be introduced into one or both of the monomers of the heterodimeric IL18-Fc fusion protein. That is, the pI of one of the monomers (referred to herein for simplicity as “monomer A”) can be engineered away from monomer B, or both monomer A and B can be changed, with the pI of monomer A increasing and the pI of monomer B decreasing. As discussed, the pI changes of either or both monomers can be done by removing or adding a charged residue (e.g., a neutral amino acid is replaced by a positively or negatively charged amino acid residue, e.g., glutamine to glutamic acid), changing a charged residue from positive or negative to the opposite charge (e.g. aspartic acid to lysine) or changing a charged residue to a neutral residue (e.g., loss of a charge; lysine to serine.). A number of these variants are shown in the figures, including, FIGS. 4 and 5.

Creating a sufficient change in pI in at least one of the monomers such that heterodimers can be separated from homodimers can be done by using a “wild type” heavy chain constant region and a variant region that has been engineered to either increase or decrease its pI (wt A:B+ or wt A:B−), or by increasing one region and decreasing the other region (A+:B− or A−:B+).

Thus, in general, a component of some embodiments of the present subject fusion proteins are amino acid variants in the Fc domains or constant domain regions that are directed to altering the isoelectric point (p1) of at least one, if not both, of the monomers of a dimeric protein by incorporating amino acid substitutions (“pI variants” or “pI substitutions”) into one or both of the monomers. The separation of the heterodimers from the two homodimers can be accomplished if the pIs of the two monomers differ by as little as 0.1 pH unit, with 0.2, 0.3, 0.4 and 0.5 or greater all finding use in the present invention.

As will be appreciated by those in the art, the number of pI variants to be included on each or both monomer(s) of a heterodimeric IL18-Fc fusion protein to achieve good separation will depend in part on the starting pI of the components. That is, to determine which monomer to engineer or in which “direction” (e.g., more positive or more negative), the sequences of the Fc domains and any IL18 or linker included in each monomer are calculated and a decision is made from there based on the pIs of the monomers. As is known in the art, different Fc domains, linkers and IL18s will have different starting pIs. In general, as outlined herein, the pIs are engineered to result in a total pI difference of each monomer of at least about 0.1 logs, with 0.2 to 0.5 being preferred as outlined herein.

In general, as will be appreciated by those in the art, there are two general categories of amino acid modifications that affect p1: those that increase the pI of the protein (basic changes) and those that decrease the pI of the protein (acidic changes). As described herein, all combinations of these variants can be used: one monomer may include a wild type Fc domain, or a variant Fc domain that does not display a significantly different pI from wild-type, and the other monomer includes a Fc domain that is either more basic or more acidic. Alternatively, each monomer may be changed, one to more basic and one to more acidic.

In the case where pI variants are used to achieve heterodimerization, a more modular approach to designing and purifying heterodimeric IL18-Fc fusion proteins is provided. Thus, in some embodiments, heterodimerization variants (including skew and pI variants) must be engineered. In addition, in some embodiments, the possibility of immunogenicity resulting from the pI variants is significantly reduced by importing pI variants from different IgG isotypes such that pI is changed without introducing significant immunogenicity (see isotypic variants below). Thus, an additional problem to be solved is the elucidation of low pI constant domains with high human sequence content, e.g., the minimization or avoidance of non-human residues at any particular position. Alternatively, or in addition to isotypic substitutions, the possibility of immunogenicity resulting from the pI variants is significantly reduced by utilizing isosteric substitutions (e.g. Asn to Asp; and Gln to Glu).

A side benefit that can occur with this pI engineering is also the extension of serum half-life and increased FcRn binding. That is, as described in US Publ. App. No. US 2012/0028304 (incorporated by reference in its entirety and specifically for the disclosure of pI variants that provide additional function), lowering the pI of antibody constant domains (including those found in Fc fusions) can lead to longer serum retention in vivo. These pI variants for increased serum half-life also facilitate pI changes for purification.

In addition, it should be noted that the pI variants of the heterodimerization variants give an additional benefit for the analytics and quality control process of Fc fusion proteins, as the ability to either eliminate, minimize and distinguish when homodimers are present is significant. Similarly, the ability to reliably test the reproducibility of the heterodimeric Fc fusion protein production is important.

Exemplary combinations of pI variants are shown in FIGS. 4 and 5, and FIG. 30 of US Publ. App. No. 2016/0355608, all of which are herein incorporated by reference in its entirety and specifically for the disclosure of pI variants. As outlined herein and shown in the figures, these changes are shown relative to IgG1, but all isotypes can be altered this way, as well as isotype hybrids. In the case where the heavy chain constant domain is from IgG2-4, R133E and R133Q can also be used.

In one embodiment, the heterodimeric IL18-Fc fusion protein includes a monomer with a variant Fc domain having pI variant modifications 295E/384D/418E/421D (Q295E/N384D/Q418E/N421D when relative to human IgG1). In one embodiment, the heterodimeric IL18-Fc fusion protein includes a monomer with a variant Fc domain having pI variant modifications 217R/228R/276K (P217R/P228R/N276K when relative to human IgG1). Additional exemplary pI variant modification that can be incorporated into the Fc domain of a subject are depicted in FIG. 5.

In some embodiments, modifications are made in the hinge of the Fc domain, including positions 216, 217, 218, 219, 220, 221, 222, 223, 224, 225, 226, 227, 228, 229, and 230 based on EU numbering. Thus, pI mutations and particularly substitutions can be made in one or more of positions 216-230, with 1, 2, 3, 4 or 5 mutations finding use. Again, all possible combinations are contemplated, alone or with other pI variants in other domains.

Specific substitutions that find use in lowering the pI of hinge domains include, but are not limited to, a deletion at position 221, a non-native valine or threonine at position 222, a deletion at position 223, a non-native glutamic acid at position 224, a deletion at position 225, a deletion at position 235 and a deletion or a non-native alanine at position 236. In some cases, only pI substitutions are done in the hinge domain, and in others, these substitution(s) are added to other pI variants in other domains in any combination.

In some embodiments, mutations can be made in the CH2 region, including positions 233, 234, 235, 236, 274, 296, 300, 309, 320, 322, 326, 327, 334 and 339, based on EU numbering. It should be noted that changes in 233-236 can be made to increase effector function (along with 327A) in the IgG2 backbone. Again, all possible combinations of these 14 positions can be made; e.g., an IL18-Fc fusion protein may include a variant Fc domain with 1, 2, 3, 4, 5, 6, 7, 8, 9 or 10 CH2 pI substitutions.

Specific substitutions that find use in lowering the pI of CH2 domains include, but are not limited to, a non-native glutamine or glutamic acid at position274, a non-native phenylalanine at position 296, a non-native phenylalanine at position 300, a non-native valine at position 309, a non-native glutamic acid at position 320, a non-native glutamic acid at position 322, a non-native glutamic acid at position 326, a non-native glycine at position 327, a non-native glutamic acid at position 334, a non-native threonine at position 339, and all possible combinations within CH2 and with other domains.

In this embodiment, the modifications can be independently and optionally selected from position 355, 359, 362, 384, 389,392, 397, 418, 419, 444 and 447 (EU numbering) of the CH3 region. Specific substitutions that find use in lowering the pI of CH3 domains include, but are not limited to, a non-native glutamine or glutamic acid at position 355, a non-native serine at position 384, a non-native asparagine or glutamic acid at position 392, a non-native methionine at position 397, a non-native glutamic acid at position 419, a non-native glutamic acid at position 359, a non-native glutamic acid at position 362, a non-native glutamic acid at position 389, a non-native glutamic acid at position 418, a non-native glutamic acid at position 444, and a deletion or non-native aspartic acid at position 447.

3. Isotypic Variants

In addition, some embodiments of the IL18-Fc fusion proteins provided herein rely on the “importation” of pI amino acids at particular positions from one IgG isotype into another, thus reducing or eliminating the possibility of unwanted immunogenicity being introduced into the variants. A number of these are shown in FIG. 21 of US Publ. App. No. 2014/0370013, hereby incorporated by reference, particularly for its disclosure of isotypic variants. That is, IgG1 is a common isotype for therapeutic antibodies for a variety of reasons, including high effector function. However, the heavy constant region of IgG1 has a higher pI than that of IgG2 (8.10 versus 7.31). By introducing IgG2 residues at particular positions into the IgG1 backbone, the pI of the resulting monomer is lowered (or increased) and additionally exhibits longer serum half-life. For example, IgG1 has a glycine (pI 5.97) at position 137, and IgG2 has a glutamic acid (pI 3.22); importing the glutamic acid will affect the pI of the resulting protein. As is described below, a number of amino acid substitutions are generally required to significantly affect the pI of the variant Fc fusion protein. However, it should be noted as discussed below that even changes in IgG2 molecules allow for increased serum half-life.

In other embodiments, non-isotypic amino acid modifications are made, either to reduce the overall charge state of the resulting protein (e.g., by changing a higher pI amino acid to a lower pI amino acid), or to allow accommodations in structure for stability, etc. as is further described below.

In addition, by pI engineering both the heavy and light constant domains, significant modifications in each monomer of the heterodimer can be seen. As discussed herein, having the pIs of the two monomers differ by at least 0.5 can allow separation by ion exchange chromatography or isoelectric focusing, or other methods sensitive to isoelectric point.

4. Calculating pI

The pI of each monomer of the IL18-Fc fusion protein can depend on the pI of the variant Fc domain and the pI of the total monomer, including the variant Fc domain and any IL18 and/or domian linker included in the monomer. Thus, in some embodiments, the change in pI is calculated on the basis of the variant Fc domain, using the chart in the FIG. 19 of US Publ. App. No. 2014/0370013, hereby incorporated by reference, particularly for its disclosure of methods of calculating pI. As discussed herein, which monomer to engineer is generally decided by the inherent pI of each monomer.

5. pI Variants that also confer better FcRn in vivo binding

In the case where the pI variant(s) decreases the pI of the monomer, such modifications can have the added benefit of improving serum retention in vivo.

Fc regions are believed to have longer half-lives in vivo, because binding to FcRn at pH 6 in an endosome sequesters the Fc (Ghetie and Ward, 1997 Immunol Today. 18(12): 592-598, entirely incorporated by reference). The endosomal compartment then recycles the Fc to the cell surface. Once the compartment opens to the extracellular space, the higher pH, —7.4, induces the release of Fc back into the blood. In mice, Dall' Acqua et al. showed that Fc mutants with increased FcRn binding at pH 6 and pH 7.4 actually had reduced serum concentrations and the same half-life as wild-type Fc (Dall' Acqua et al. 2002, J. Immunol. 169:5171-5180, entirely incorporated by reference). The increased affinity of Fc for FcRn at pH 7.4 is thought to forbid the release of the Fc back into the blood. Therefore, the Fc modifications that will increase Fc's half-life in vivo will ideally increase FcRn binding at the lower pH while still allowing release of Fc at higher pH. The amino acid histidine changes its charge state in the pH range of 6.0 to 7.4. Thus, it is not surprising to find His residues at important positions in the Fc/FcRn complex.

VH. Other Fc Variants for Additional Functionality

In addition to heterodimerization variants, the subject heterodimeric IL18-Fc fusion proteins provided herein may independently include Fc modifications that affect functionality including, but not limited to, altering binding to one or more Fc receptors (e.g., FcγR and FcRn).

1. FcγR Variants

In one embodiment, the IL18-Fc fusion proteins includes one or more amino acid modifications that affect binding to one or more Fcγreceptors (i.e., “FcγR variants”). FcγR variants (e.g., amino acid substitutions) that result in increased binding as well as decreased binding can be useful. For example, it is known that increased binding to FcγRIIIa results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell). Similarly, decreased binding to FcγRIIb (an inhibitory receptor) can be beneficial as well in some circumstances. FcγR variants that find use in the IL18 fusion proteins include those listed in U.S. Pat. Nos. 8,188,321 (particularly FIGS. 41) and U.S. Pat. No. 8,084,582, and US Publ. App. Nos. 20060235208 and 20070148170, all of which are expressly incorporated herein by reference in their entirety and specifically for the variants disclosed therein that affect Fcγreceptor binding. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 243A, 243L, 264A, 264V and 299T.

In addition, amino acid substitutions that increase affinity for FcγRIIc can also be independently included in the Fc domain variants outlined herein. Useful substitutions that for FcγRIIc are described in, for example, U.S. Pat. Nos. 8,188,321 and 10,113,001, all of which are expressly incorporated herein by reference in their entirety and specifically for the variants disclosed therein that affect Fcγreceptor binding.

2. FcRn Variants

Further, IL18-Fc fusion proteins described herein can independently include Fc substitutions that confer increased binding to the FcRn and increased serum half-life. Such modifications are disclosed, for example, in U.S. Pat. No. 8,367,805, hereby incorporated by reference in its entirety, and specifically for Fc substitutions that increase binding to FcRn and increase half-life. Such modifications include, but are not limited to 434S, 434A, 428L, 308F, 259I, 428L/434S, 428L/434A, M252Y/S254T/T256E, 259I/308F, 436I/428L, 436I or V/434S, 436V/428L and 259I/308F/428L.

3. Ablation Variants

In some embodiments, the IL18-Fc fusion protein includes one or more modifications that reduce or remove the normal binding of the Fc domain to one or more or all of the Fcγ receptors (e.g., FcγR1, FcγRIIa, FcγRIIb, FcγRIIIa, etc.) to avoid additional mechanisms of action. Such modifications are referred to as “FcγR ablation variants” or “Fc knock out (FcKO or KO)” variants. In some embodiments, particularly in the use of immunomodulatory proteins, it is desirable to ablate FcγRIIIa binding to eliminate or significantly reduce ADCC activity such that one of the Fc domains comprises one or more Fcγreceptor ablation variants. These ablation variants are depicted in FIG. 31 of U.S. Pat. No. 10,259,887, which is herein incorporated by reference in its entirety, and each can be independently and optionally included or excluded, with preferred aspects utilizing ablation variants selected from the group consisting of G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del, according to the EU index. In addition, ablation variants of use in the subject IL18-Fc fusion proteins are also depicted in FIG. 6. It should be noted that the ablation variants referenced herein ablate FcγR binding but generally not FcRn binding.

VIII. Combination of Heterodimeric and Fc Variants

As will be appreciated by those in the art, the Fc modifications described herein can independently be combined. For example, all of the recited heterodimerization variants (including skew and/or pI variants) can be optionally and independently combined in any way, as long as they retain their “strandedness” or “monomer partition.”

In the case of pI variants, while embodiments finding particular use are shown in the figures, other combinations can be generated, following the basic rule of altering the pI difference between two monomers to facilitate purification.

In addition, any of the heterodimerization variants, may also be independently and optionally combined with other variants described herein including, but not limited to, Fc ablation variants, FcRn variants, and/or half/life extension variants as generally outlined herein.

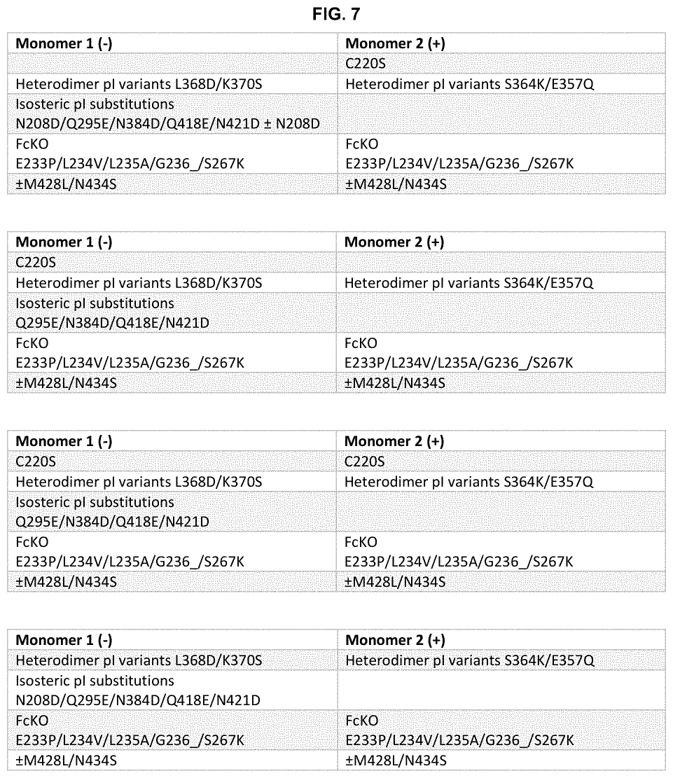

Exemplary combinations of modifications are shown in FIG. 7 and the backbone sequences in FIGS. 9 (heterodimeric backbones). In certain embodiments, the IL18-Fc fusion protein is heterodimeric and includes a combination of Fc domain modifications as depicted in FIG. 4. In some embodiments, the heterodimeric IL18-Fc fusion protein includes a first monomer having a first Fc domain with the backbone sequence of any one of the “monomer 1” backbones in FIG. 7 and a second Fc domain with the backbone sequence of a corresponding “monomer 2” backbone in FIG. 7. In certain embodiments, the homodimeric IL18-Fc fusion protein includes a first monomer with a first Fc domain and a second monomer with a second Fc domain, where the first and second Fc domains each have the sequence of any of the backbone sequences in FIGS. 9 and 10.

In some embodiments, wherein the IL18-Fc fusion protein is a monovalent (i.e., only one IL18), the first monomer includes a first Fc domain with heterodimer skew variants L368D/K370S, isosteric pI variants Q295E/N384D/Q418E/N421D, and FcKO variants E233P/L234V/L235A/G236del/S267K and the second monomer includes a second Fc domain with heterodimer skew variants S364K/E357Q and FcKO variants E233P/L234V/L235A/G236del/S267K, according to the EU index. In some embodiments, the first and second monomers each also include M428L/N434S half-life extension variants. In some embodiments, the first and second monomers each also include a C220S hinge amino acid substitution. In some embodiments, the first and second monomers each also include a N297A or N297S amino acid substitution that removes glycosylation. In some embodiments, the first monomer includes a first Fc domain with modifications C220 S/E233P/L234V/L235A/G236del/S267K/Q295E/L368D/K370 S/N384D/Q418E/N421D and optionally M428L/N434S and the second monomer includes a second Fc domain with modifications C220S/E233P/L234V/L235A/G236del/S267K/S364K/E357Q and optionally modifications M428L/N434S, according to the EU index.

In some embodiments, wherein the IL18-Fc fusion protein is a monovalent (i.e., only one IL18), the first monomer includes a first Fc domain with heterodimer skew variants L368D/K370S, isosteric pI variants Q295E/N384D/Q418E/N421D, and FcKO variants E233P/L234V/L235A/G236del/S267K and the second monomer includes a second Fc domain with heterodimer skew variants S364K and FcKO variants E233P/L234V/L235A/G236del/S267K, according to the EU index. In some embodiments, the first and second monomers each also include M428L/N434S half-life extension variants. In some embodiments, the first and second monomers each also include a C220S hinge amino acid substitution. In some embodiments, the first monomer includes a first Fc domain with modifications C220 S/E233P/L234V/L235A/G236del/S267K/Q295E/L368D/K370 S/N384D/Q418E/N421D and optionally M428L/N434S and the second monomer includes a second Fc domain with modifications C220S/E233P/L234V/L235A/G236del/S267K/S364K and optionally modifications M428L/N434S, according to the EU index.

In some embodiments, wherein the IL18-Fc fusion protein is a monovalent (i.e., only one IL18), the first monomer includes a first Fc domain with heterodimer skew variants L368E/K370S, isosteric pI variants Q295E/N384D/Q418E/N421D, and FcKO variants E233P/L234V/L235A/G236del/S267K and the second monomer includes a second Fc domain with heterodimer skew variants S364K and FcKO variants E233P/L234V/L235A/G236del/S267K, according to the EU index. In some embodiments, the first and second monomers each also include M428L/N434S half-life extension variants. In some embodiments, the first and second monomers each also include a C220S hinge amino acid substitution. In some embodiments, the first monomer includes a first Fc domain with modifications C220 S/E233P/L234V/L235A/G236del/S267K/Q295E/L368E/K370 S/N384D/Q418E/N421D and optionally M428L/N434S and the second monomer includes a second Fc domain with modifications C220S/E233P/L234V/L235A/G236del/S267K/S364K and optionally modifications M428L/N434S, according to the EU index.

In some embodiments, wherein the IL18-Fc fusion protein is a monovalent (i.e., only one IL18), the first monomer includes a first Fc domain with heterodimer skew variants K360E/Q362E/T411E, isosteric pI variants Q295E/N384D/Q418E/N421D, and FcKO variants E233P/L234V/L235A/G236del/S267K and the second monomer includes a second Fc domain with heterodimer skew variants D401K and FcKO variants E233P/L234V/L235A/G236del/S267K, according to the EU index. In some embodiments, the first and second monomers each also include M428L/N434S half-life extension variants. In some embodiments, the first and second monomers each also include a C220S hinge amino acid substitution. In some embodiments, the first monomer includes a first Fc domain with modifications C220S/E233P/L234V/L235A/G236del/S267K/Q295E/K360E/Q362E/384D/T411E/Q418E/N421D and optionally M428L/N434S and the second monomer includes a second Fc domain with modifications C220S/E233P/L234V/L235A/G236de1/D401K and optionally modifications M428L/N434S, according to the EU index.

In some embodiments, wherein the IL18-Fc fusion protein is a monovalent (i.e., only one IL18), the first monomer includes a first Fc domain with heterodimer skew variants L368D/K370S and a variant that ablates Fab arm exchange, S228P, and the second monomer includes a second Fc domain with heterodimeric pI variants S364K/E357Q and S228P to the EU index. In some embodiments, the first and second monomers each also include M428L/N434S half-life extension variants. In some embodiments, the first and second monomers each also include a C220S hinge amino acid substitution. In some embodiments, the first monomer includes a first Fc domain with modifications C220S/L368D/K370S and optionally M428L/N434S and the second monomer includes a second Fc domain with modifications C220S/S228P/S364K/E357Q and optionally modifications M428L/N434S, according to the EU index. In exemplary embodiments, the Fc domains are human IgG4 Fc domains.

In some embodiments, wherein the IL18-Fc fusion protein is a monovalent (i.e., only one IL18), the first monomer includes a first Fc domain with heterodimer skew variants L368D/K370S and isosteric pI variants Q295E/N384D/Q418E/N421D and the second monomer includes a second Fc domain with heterodimer skew variants S364K/E357Q, according to the EU index. In some embodiments, the first and second monomers each also include M428L/N434S half-life extension variants. In some embodiments, he first and second monomers each also include a C219S hinge modification. In some embodiments, the first monomer includes a first Fc domain with modifications Q295E/L368D/K370S/384D/Q418E/N421D and optionally M428L/N434S and the second monomer includes a second Fc domain with modifications S364K/E357Q and optionally modifications M428L/N434S, according to the EU index. In exemplary embodiments, the Fc domains are human IgG2 Fc domains.

In some embodiments, wherein the IL18-Fc fusion protein is a monovalent (i.e., only one IL18), the first monomer includes a first Fc domain with heterodimer skew variants L368D/K370S, isosteric pI variants Q295E/N384D/Q418E/N421D, and FcKO variant S267K and the second monomer includes a second Fc domain with heterodimer skew variants S364K/E357Q and FcKO variant S267K, according to the EU index. In some embodiments, the first and second monomers each also include M428L/N434S half-life extension variants. In some embodiments, he first and second monomers each also include a C219S hinge modification. In some embodiments, the first monomer includes a first Fc domain with modifications S267K/Q295E/L368D/K370S/384D/Q418E/N421D and optionally M428L/N434S and the second monomer includes a second Fc domain with modifications S267K/S364K/E357Q and optionally modifications M428L/N434S, according to the EU index. In exemplary embodiments, the Fc domains are human IgG2 Fc domains.

In some embodiments, wherein the IL18-Fc fusion protein is a monovalent (i.e., only one IL18), the first monomer includes a first Fc domain with heterodimer skew variants L368D/K370S and FcKO variants E233P/L234V/L235A/G236del/S267K and the second monomer includes a second Fc domain with heterodimer skew variants S364K/E357Q, isosteric pI variants P217R/P228R/N276K, and FcKO variants E233P/L234V/L235A/G236del/S267K, according to the EU index. In some embodiments, the first and second monomers each also include M428L/N434S half-life extension variants. In some embodiments, the first and second monomers each also include a C220S hinge amino acid substitution. In some embodiments, the first monomer includes a first Fc domain with modifications C220S/E233P/L234V/L235A/G236del/S267K/L368D/K370S and optionally M428L/N434S and the second monomer includes a second Fc domain with modifications P217R/C220S/P228R/E233P/L234V/L235A/G236del/S267K/N276K/S364K/E357Q and optionally modifications M428L/N434S, according to the EU index.

The variant Fc domains provided herein can also include 1, 2, 3, 4, 5, 6, 7, 8, 9, or 10 additional mutations in addition to the enumerated mutations.

IX. Domain Linkers

In some embodiments of the subject IL18-Fc fusion protein, an IL18 is covalently attached to an Fc domain by a linker (e.g., (IL18)i-L-Fc). In some embodiments, the linker is a “domain linker.” While any suitable linker can be used, many embodiments utilize a glycine-serine polymer, including for example (GS)ₙ, (GSGGS)ₙ, (GGGGS)ₙ, and (GGGS)ₙ, where n is an integer of at least 0 (and generally from 0 to 1 to 2 to 3 to 4 to 5), as well as any peptide sequence that allows for recombinant attachment of the two domains with sufficient length and flexibility to allow each domain to retain its biological function. In certain cases, useful linkers include (GGGGS)₁ or (GGGGS)₂. Illustrative domain linkers are depicted in FIG. 8. In some cases, and with attention being paid to “strandedness”, as outlined below, charged domain linkers can be used as discussed herein.

X. IL18-Fc Fusion Protein Formats

Useful IL18-Fc fusion protein formats are shown in FIG. 12. IL18-Fc fusion proteins provided herein include monovalent IL18-Fc fusion proteins as shown in FIGS. 12A and 21A-21M) and IL18xFab-Fc fusion proteins as shown in FIG. 12B and 22A-22CZ.

A. IL18-Fc Fusion Proteins

In some embodiments, the IL18-Fc fusion is monovalent IL18-Fc fusion protein that includes (a) a first monomer that includes an IL18 protein or a variant thereof covalently attached to a first Fc domain and (b) a second monomer that includes a second Fc domain alone (i.e., an “empty Fc”). In some embodiments, the IL18 fusion is monovalent IL18-Fc fusion protein includes (a) a monomer that includes an IL18 covalently attached to an Fc domain and (b) another monomer that includes another Fc domain alone (i.e., an “empty Fc”). See, the schematic diagram in FIG. 12A and the amino acid sequences of FIGS. 21A-21M.

Any of the IL18s described herein can be included in the monovalent IL18-Fc fusion protein. In some embodiments, the IL18 is wildtype mature human IL18 (FIG. 1A). In certain embodiments, the IL18 is a variant IL18 that includes one or more modifications as depicted in FIGS. 13A-13B, 14, 15A-15D, 16A-16E, 17A-B, 18, 19A-19P, 20A-20D, 31, 36, 37, 39A-39B, 40, 41A-41C, 42A-42D, 43A-13B, 44A-44C, 45, 46, 47, 48, 51, 54, and 62. In some embodiments, the IL18 of the monovalent IL18-Fc fusion proteins is a variant IL18 that includes a modification at one or more positions selected from Y1, E6, S7, K8, S10, V11, N14, L15, D17, Q18, D23, R27, P28, L29, E31, M33, T34, D35, S36, D37, C38, R39, D40, N41, R44, I46, I49, S50, M51, K53, D54, S55, Q56, P57, M60, A61, V62, T63, S65, K67, C68, E69, I71, C76, E77, 180, 181, N87, P88, D90, K93, T95, K96, S97, Q103, H109, D110, N111, M113, S119, A126, C127, D132, L136, L138, K139, E141, L144, D146, R147, I149, M150, N155, E156, and D157 are modified. In some embodiments, the IL18 of the monovalent IL18-Fc fusion proteins is a variant IL18 that includes one or more modifications (e.g., substitutions or deletions) selected from Y1F, Y1H, E6A, E6Q, S7C, S7P, K8E, K8Q, K8Y, S10C, V11I, N14C, N14W, L15C, D17N, Q18L, D23N, D23S, R27Q, P28C, L29V, E31Q, M33C, T34P, D35N, D35E, S36D, S36N, D37N, C38S, C38Q, C38R, C38E, C38L, C38I, C38V, C38K, C38D, R39S, R39T, D40N, N41Q, R44Q, I46V, I49C, S50C, S50Y, M51I, M51K, M51Q, M51R, M51L, M51H, M51F, M51Y, K53A, K53D, K53E, K53G, K53H, K53I, K53L, K53M, K53N, K53Q, K53R, K53S, K53T, K53V, K53Y, K53F, D54C, S55N, S55Q, S55D, S55E, S55T, Q56I, Q56L, P57A, P57E, P57T, P57V, P57Q, P57D, P57Y, P57N, M60I, M60L, M60K, M60Y, M60F, A61C, V62C, T63C, S65C, K67Q, C68S, C68I, C68F, C68Y, C68D, C68N, C68E, C68Q, C68K, E69K, I71M, C76S, C76E, C76K, E77K, 180T, I81L, I81V, N87S, P88C, D90E, K93D, K93N, T95E, K96G, K96Q, 597N, Q103C, Q103E, Q103I, Q103L, H109W, H109Y, D110N, N111D, D110Q, D110R, N111Q, N111S, N111T, N111E, M113I, S119L, A126C, C127S, C127W, C127Y, C127F, C127D, C127E, C127K, D132Q, D132E, L136C, L138C, K139C, E141K, E141Q, L144N, D146F, D146L, D146Y, R147C, R147K, I149V, M150F, M150T, N155C, E156Q, D157A, D157S, D157N, and D157del. In some embodiments, the IL18 variant includes a 4CS substitution (C38S/C68S/C76S/C127S substitutions) and one or more of additional substitutions including S38C, S38E, S38L, S38Q, S38R, S38V, S38K, S38D, S68C, S68D, S68E, S68F, S68I, S68N, S68Q, S68Y, S68K, S76C, S76E, S76K, S127C, S127D, S127F, S127W, S127K, and S127Y. In some embodiments, the amino acid substitution can include 4CS, 4CS/D193S, 4CS/D193A, 4CS/delD193, 4CS/S38E, 4CS/S68E, 4CS/S76E, 4CS/S127E, 4CS/S38K, 4CS/S68K, 4CS/S76K, 4CS/S127K, 4CS/S38D, 4CS/Y1F, 4CS/Y1H, 4CS/E6A, 4CS/E6Q, 4CS/D17N, 4CS/E31Q, 4CS/D35N, 4CS/D37N, 4CS/D40N, 4CS/N41Q, 4CS/K53R, 4CS/K53H, 4CS/K53M, 4CS/K53E, 4CS/K53Q, 4CS/K53A, 4CS/Q103E, 4CS/D110N, 4CS/N111Q, 4CS/E6A/K53A, 4CS/N14C/E31Q/S127C, 4CS/E31Q/K53A, 4CS/E31Q/D35N/K53A, 4CS/E31Q/N41Q/K53A, 4CS/E31Q/D35N/N41Q/K53A, 4CS/E31Q/D35N, 4CS/E31Q/N41Q, 4C S/E31Q/D35N/N41Q, 4C S/E31Q/D37N, 4CS/E31Q/D37N/K53A, 4CS/E31Q/M33C/S38C, 4C S/E31Q/S76C/L138C, 4CS/E31Q/568I, 4CS/E31Q/S68F, 4C S/E31Q/S127W, 4CS/E31Q/S127Y, 4C S/E31Q/S127F, 4CS/S10C/E31Q/149C, 4C S/L15C/E31Q/R147C, 4C S/P28C/E31Q/L136C, 4CS/E31Q/S50C/P88C, 4CS/E31Q/T63C/P88C, 4C S/E31Q/V62C/Q103C, 4CS/S10C/E31Q/N155C, 4CS/E31Q/S65C/P88C, 4CS/S7C/E31Q/S50C, 4CS/E31Q/D54C/A61C, 4C S/E31Q/A126C/K139C, 4CS/N14W/E31Q, 4CS/E31Q/D146Y, 4CS/E31Q/D146L, 4C S/E31Q/D146F, 4C S/E31Q/Q103L, 4CS/E31Q/Q103I, 4CS/E31Q/M150F, 4CS/Q18L/E31Q, 4CS/E31Q/S68Y, 4CS/E31Q/S38Q, 4C S/E31Q/S38R, 4CS/E31Q/S68D, 4CS/S7P/E31Q, 4CS/V11I/E31Q, 4CS/D23N/E31Q, 4CS/D23S/E31Q, 4CS/R27Q/E31Q, 4CS/L29V/E31Q, 4CS/E31Q/T34P, 4CS/E31Q/R39T, 4CS/E31Q/R39S, 4CS/E31Q/R44Q, 4CS/E31Q/I46V, 4CS/E31Q/S50Y, 4CS/E31Q/Q56L, 4CS/E31Q/Q56L/P57T, 4C S/E31Q/P57T, 4CS/E31Q/P57V, 4CS/E31Q/M60L, 4CS/E31Q/K67Q, 4CS/E31Q/E69K, 4CS/E31Q/I71M, 4CS/E31Q/E77K, 4CS/E31Q/I80T, 4CS/E31Q/I81V, 4CS/E31Q/I81L, 4C S/E31Q/N87S, 4CS/E31Q/D90E, 4CS/E31Q/K93D/T95E, 4CS/E31Q/K93N/T95E, 4CS/E31Q/T95E, 4CS/E31Q/K96G, 4CS/E31Q/S97N, 4CS/E31Q/N111D, 4CS/E31QN1113I, 4CS/E31Q/S119L, 4CS/E31Q/L144N, 4CS/E31Q/R147K, 4CS/E31Q/I149V, 4CS/E31Q/M150T, 4CS/E31Q/E156Q/D157N, 4CS/K53S, 4CS/K53G, 4CS/K53T, 4CS/K53I, 4CS/K53L, 4CS/K53N, 4CS/K53D, 4CS/M51K, 4CS/M51Q, 4CS/M51I, 4CS/S55N, 4CS/S55Q, 4CS/Q56L, 4CS/Q56I, 4CS/P57A, 4CS/P57E, 4CS/M60L, 4CS/M60I, 4CS/K8Y, 4CS/K8Q, 4CS/K8E, 4CS/H109W, 4CS/H109Y, 4CS/E31Q/S38E, 4CS/E31Q/S38L, 4CS/E31Q/S38I, 4C S/E31Q/S38V, 4C S/E31Q/S68N, 4CS/E31Q/S68E, 4CS/E31Q/S68Q, 4C S/E31Q/S76C, 4C S/E31Q/S127D, 4CS/E31Q/S127E, 4CS/D23N/E31Q/R27Q, 4CS/E31Q/Q56L/T95E, 4CS/E31Q/K96Q/S119L, 4CS/E31Q/E141K/I149V, 4CS/E31Q/E141Q/I149V, 4CS/S7P/E31Q/S50Y, 4CS/E31Q/I80T/I81L/delD193, 4CS/E31Q/P57A/S119L/delD193, 4CS/E31Q/P57A/I80T/I81L/S119L/delD193, 4CS/E31Q/P57A/K93D/T95E/S119L/delD193, 4CS/E31Q/I80T/S119L/delD193, 4CS/E31Q/I80T/I81L/K93D/T95E/delD193, 4CS/E31Q/P57A/I80T/I81L/K93D/T95E/S119L/delD193, 4CS/S7C/E31Q/S50C/delD193, 4CS/S7C/E31Q/S50C/P57A/delD193, 4CS/S7C/E31Q/S50C/S119L/delD193, 4CS/S7C/E31Q/S50C/I80T/delD193, 4CS/S7C/E31Q/S50C/I80T/S119L/delD193, 4CS/S7C/E31Q/S50C/P57A/I80T/S119L/delD193, 4CS/S10C/E31Q/N155C/delD193, 4CS/S10C/E31Q/P57A/N155C/delD193, 4CS/S10C/E31Q/S119L/N155C/delD193, 4CS/S10C/E31Q/I80T/N155C/delD193, 4C S/S10C/E31Q/I80T/S119L/N155C/delD193, 4CS/S10C/E31Q/P57A/I80T/S119L/N155C/delD193, 4C S/S10C/E31Q/I49C/delD193, 4CS/L15C/E31Q/R147C/delD193, 4CS/E31Q/T63C/P88C/delD193, 4CS/N14C/E31Q/S127C/delD193, 4CS/E31Q/S38R/S127W/delD193, 4CS/S10C/D35E/N155C, 4CS/S10C/S36D/N155C, 4CS/S10C/S36N/N155C, 4CS/S10C/K53V/N155C, 4CS/S10C/K53Y/N155C, 4CS/S10C/K53F/N155C, 4CS/S10C/M51R/N155C, 4CS/S10C/M51L/N155C, 4C S/S10C/M51H/N155C, 4C S/S10C/M51F/N155C, 4CS/S10C/M51Y/N155C, 4CS/S10C/S55D/N155C, 4CS/S10C/S55E/N155C, 4CS/S10C/S55T/N155C, 4CS/S10C/P57Q/N155C, 4CS/S10C/P57D/N155C, 4CS/S10C/P57Y/N155C, 4CS/S10C/P57N/N155C, 4CS/S10C/M60Y/N155C, 4CS/S10C/M60F/N155C, 4CS/S10C/D110Q/N155C, 4CS/S10C/D110R/N155C, 4CS/S10C/N111D/N155C, 4CS/S10C/N111S/N155C, 4CS/S10C/N111T/N155C, 4CS/S10C/N111E/N155C, 4CS/S10C/D132Q/N155C, 4CS/S10C/D132EN155C, 4CS/E6Q/S10C/K53D/N155C, 4C S/E6Q/S10C/M51K/K53D/N155C, 4CS/S10C/E31Q/D35N/N41Q/K53A/N155C, 4CS/S10C/E31Q/N41Q/K53A/N155C, 4CS/S10C/E31Q/K53A/N155C, 4CS/S10C/K53T/N155C, 4CS/S10C/P57A/N155C, 4CS/S10C/N155C, 4CS/S10C/S76G/N155C, 4CS/S10C/S76A/N155C, 4CS/S10C/M51K/K53D/N155C, 4CS/S10C/M51K/K53E/N155C, 4CS/E6Q/S10C/K53E/N155C, 4CS/E6Q/S10C/M51K/K53E/N155C, 4CS/E6Q/S10C/M51K/P57E/N155C, 4CS/S10C/M51K/P57E/N155C, 4CS/E6Q/S10C/P57E/N155C, 4CS/S10C/E31Q/K53T/N155C, 4CS/S10C/K53G/P57E/N155C, 4CS/S10C/K53T/P57E/N155C, 4CS/S10C/K53A/P57E/N155C, 4CS/S10C/P57E/N155C, 4CS/S10C/K53D/N155C, 4CS/S10C/E31Q/N41Q/N155C, 4CS/S10C/K53A/N155C, 4CS/S10C/K53G/N155C, 4CS/S10C/K53E/N155C, 4CS/S10C/K53S/N155C, 4CS/S10C/M51L/K53D/N155C, 4CS/S10C/K53D/D110R/N155C, 4CS/S10C/K53D/N111T/N155C, 4CS/S10C/K53D/S55T/N155C, 4CS/S10C/K53D/S55T/D110R/N155C, 4CS/S10C/M51L/K53D/S55T/D110R/N111T/N155C, 4CS/S10C/M51L/K53D/S55T/D110R/N155C, 4CS/S10C/K53D/S55T/D110R/N111T/N155C, 4CS/S10C/K53D/S55T/N111T/N155C, 4CS/S10C/E31Q/D35N/N155C, 4CS/S10C/N41Q/N155C, 4CS/S10C/D35N/N155C, 4CS/S10C/D37N/N155C, 4CS/S10C/E31Q/D37N/N155C, 4CS/S10C/D35N/D37N/N155C, 4CS/E6Q/S10C/M51L/K53D/S55T/D110R/N111T/N155C, 4CS/S10C/K53D/H109Y/N155C, 4CS/S10C/D37N/K53D/N155C, 4CS/S10C/D35N/K53D/N155C, 4CS/K8E/S10C/K53D/N155C, 4CS/S10C/E31Q/K53D/N155C, 4CS/S10C/N41Q/K53D/N155C, 4CS/S10C/K53D/P57V/N155C, 4CS/S10C/K53D/P57T/N155C, 4CS/E6Q/S10C/K53D/N111T/N155C, E6A/K53A, D35N/K53A, N41Q/K53A, D35N/N41Q/K53A, D35N/N41Q, D37N/K53A, E6Q/K53D, E6Q/M51K/K53D, M51K/K53D, M51K/K53E, E6Q/K53E, E6Q/M51K/K53E, E6Q/M51K/P57E, M51K/P57E, E6Q/P57E, K53G/P57E, K53T/P57E, K53A/P57E, M51L/K53D, K53D/D110R, K53D/N111T, K53D/S55T, K53D/S55T/D110R, M51L/K53D/S55T/D110R/N111T, M51L/K53D/S55T/D110R, K53D/S55T/D110R/N111T, K53D/S55T/N111T, D35N/D37N, E6Q/M51L/K53D/S55T/D110R/N111T, K53D/H109Y, D37N/K53D, D35N/K53D, K8E/K53D, N41Q/K53D, K53D/P57V, K53D/P57T, E6Q/K53D/N111T, Q56L/P57T, K93D/T95E, K93N/T95E, E156Q/D157N, D23N/R27Q, Q56L/T95E, K96Q/S119L, E141K/I149V, E141Q/I149V, S7P/S50Y, I80T/I81L, P57A/S119L, P57A/I80T/I81L/S119L, P57A/K93D/T95E/S119L, I80T/S119L, I80T/I81L/K93D/T95E, P57A/180T/I81L/K93D/T95E/S119L, P57A/180T/S119L, N14C/S127C, M33C/S38C, S76C/L138C, S10C/I49C, L15C/R147C, P28C/L136C, S50C/P88C, T63C/P88C, V62C/Q103C, S10C/N155C, S65C/P88C, S7C/S50C, D54C/A61C, A126C/K139C, C38R/C127W, E31Q/K53A, E31Q/D35N/K53A, E31Q/N41Q/K53A, E31Q/D35N/N41Q/K53A, E31Q/D35N, E31Q/N41Q, E31Q/D35N/N41Q, E31Q/D37N, E31Q/D37N/K53A, S10C/E31Q/I49C, L15C/E31Q/R147C, P28C/E31Q/L136C, E31Q/S50C/P88C, E31Q/T63C/P88C, E31Q/V62C/Q103C, S10C/E31Q/N155C, E31Q/S65C/P88C, S7C/E31Q/S50C, E31Q/D54C/A61C, E31Q/A126C/K139C, N14W/E31Q, E31Q/D146Y, E31Q/D146L, E31Q/D146F, E31Q/Q103L, E31Q/Q103I, E31Q/M150F, Ql8L/E31Q, S7P/E31Q, V11I/E31Q, D23N/E31Q, D23S/E31Q, R27Q/E31Q, L29V/E31Q, E31Q/T34P, E31Q/R39T, E31Q/R39S, E31Q/R44Q, E31Q/I46V, E31Q/S50Y, E31Q/Q56L, E31Q/Q56L/P57T, E31Q/P57T, E31Q/P57V, E31Q/M60L, E31Q/K67Q, E31Q/E69K, E31Q/I71M, E31Q/E77K, E31Q/I80T, E31Q/I81V, E31Q/I81L, E31Q/N87S, E31Q/D90E, E31Q/K93D/T95E, E31Q/K93N/T95E, E31Q/T95E, E31Q/K96G, E31Q/S97N, E31Q/N111D, E31Q/M1131, E31Q/S119L, E31Q/L144N, E31Q/R147K, E31Q/I149V, E31Q/M150T, E31Q/E156Q/D157N, D23N/E31Q/R27Q, E31Q/Q56L/T95E, E31Q/K96Q/S119L, E31Q/E141K/I149V, E31Q/E141Q/I149V, S7P/E31Q/S50Y, E31Q/180T/181L/delD193, E31Q/P57A/S119L/delD193, E31Q/P57A/180T/181L/S119L/delD193, E31Q/P57A/K93D/T95E/S119L/delD193, E31Q/I80T/S119L/delD193, E31Q/I80T/I81L/K93D/T95E/delD193, E31Q/P57A/180T/181L/K93D/T95E/S119L/delD193, S7C/E31Q/S50C/delD193, S7C/E31Q/S50C/P57A/delD193, S7C/E31Q/S50C/S119L/delD193, S7C/E31Q/S50C/I80T/delD193, S7C/E31Q/S50C/I80T/S119L/delD193, S7C/E31Q/S50C/P57A/I80T/S 119L/delD193, S10C/E31Q/N155C/delD193, S10C/E31Q/P57A/N155C/delD193, S10C/E31Q/S119L/N155C/delD193, S10C/E31Q/I80T/N155C/delD193, S10C/E31Q/I80T/S119L/N155C/delD193, S10C/E31Q/P57A/I80T/S119L/N155C/delD193, S10C/E31Q/I49C/delD193, L15C/E31Q/R147C/delD193, E31Q/T63C/P88C/delD193, S10C/D35E/N155C, S10C/S36D/N155C, S10C/S36N/N155C, S10C/K53V/N155C, S10C/K53Y/N155C, S10C/K53F/N155C, S10C/M51R/N155C, S10C/M51L/N155C, S10C/M51H/N155C, S10C/M51F/N155C, S10C/M51Y/N155C, S10C/S55D/N155C, S10C/S55E/N155C, S10C/S55T/N155C, S10C/P57Q/N155C, S10C/P57D/N155C, S10C/P57Y/N155C, S10C/P57N/N155C, S10C/M60Y/N155C, S10C/M60F/N155C, S10C/D110Q/N155C, S10C/D110R/N155C, S10C/N111D/N155C, S10C/N111S/N155C, S10CN111T/N155C, S10C/N111E/N155C, S10C/D132Q/N155C, S10C/D132E/N155C, E6Q/S10C/K53D/N155C, E6Q/S10C/M51K/K53D/N155C, S10C/E31Q/D35N/N41Q/K53A/N155C, S10C/E31Q/N41Q/K53A/N155C, S10C/E31Q/K53A/N155C, S10C/K53T/N155C, S10C/P57A/N155C, S10C/M51K/K53D/N155C, S10C/M51K/K53E/N155C, E6Q/S10C/K53E/N155C, E6Q/S10C/M51K/K53E/N155C, E6Q/S10C/M51K/P57E/N155C, S10C/M51K/P57E/N155C, E6Q/S10C/P57E/N155C, S10C/E31Q/K53T/N155C, S10C/K53G/P57E/N155C, S10C/K53T/P57E/N155C, S10C/K53A/P57E/N155C, S10C/P57E/N155C, S10C/K53D/N155C, S10C/E31Q/N41Q/N155C, S10C/K53A/N155C, S10C/K53G/N155C, S10C/K53E/N155C, S10C/K53S/N155C, S10C/M51L/K53D/N155C, S10C/K53D/D110R/N155C, S10C/K53D/N111TN155C, S10C/K53D/S55T/N155C, S10C/K53D/S55T/D110R/N155C, S10C/M51L/K53D/S55T/D110R/N111T/N155C, S10C/M51L/K53D/S55T/D110R/N155C, S10C/K53D/S55T/D110R/N111T/N155C, S10C/K53D/S55T/N111T/N155C, S10C/E31Q/D35N/N155C, S10C/N41Q/N155C, S10C/D35N/N155C, S10C/D37N/N155C, S10C/E31Q/D37N/N155C, S10C/D35N/D37N/N155C, E6Q/S10C/M51L/K53D/S55T/D110R/N111TN155C, S10C/K53D/H109Y/N155C, S10C/D37N/K53D/N155C, S10C/D35N/K53D/N155C, K8E/S10C/K53D/N155C, S10C/E31Q/K53D/N155C, S10C/N41Q/K53DN155C, S10C/K53D/P57V/N155C, S10C/K53D/P57T/N155C, or E6Q/S10C/K53D/N111T/N155C.

In some embodiments, the IL18 variant of the IL18-Fc fusion protein includes one or more amino acid substitutions provided in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-13B, 44A-44C, 45, 46, 47, 48, 51, 54, and 62. In some embodiments, the IL18 variant of the IL18-Fc fusion protein is depicted in any one of FIGS. 13A-13B, 14, 15A-15D, 16A-16E, 17A-B, 18, 19A-19P, 20A-20D, 41A-41C, 42A-42D, 43A-13B, and 44A-44C. In certain embodiments, the IL18 variant includes an amino acid sequence set forth in SEQ ID NOS:84-238 and 799-949.

Any Fc domains can be included in the monovalent IL18-Fc fusion protein, including the wildtype and variant Fc domains described herein. In some embodiments, each Fc domain includes a CH2 and CH3. In some embodiments, the first and second Fc domains include a hinge, CH2 and CH3. In one embodiment, the first and second Fc domains each have the formula, from N-terminus to C-terminus, hinge-CH2-CH3. In some embodiments, the first and second Fc domains of the monovalent IL18-Fc fusion protein are heterodimeric. Modifications for such Fc domains are described in Section III above.

In an exemplary embodiments, the monovalent IL18-Fc fusion protein is heterodimeric. In some embodiments, the first and second Fc domains include the amino acid substitution set L368D/K370S: S364K/E357Q. In some embodiments, the L368D/K370S modifications are in the first Fc domain and the S364K/E357Q modifications are in the second domain. In certain heterodimeric embodiments, the first Fc domain includes isosteric pI variants Q295E/N384D/Q418E/N421D. In some embodiments, the first Fc domain and the second Fc domain each include K447del modifications. In some embodiments, a IL18 protein or variant thereof is connected to the first Fc domain.

In certain embodiments, both the first and second Fc domains include FcKO variants: E233P/L234V/L235A/G236del/S267K, according to the EU numbering.

In some embodiments, the first monomer includes a first Fc domain with heterodimer skew variants L368D/K370S, isosteric pI variants Q295E/N384D/Q418E/N421D, and FcKO variants E233P/L234V/L235A/G236del/S267K and the second monomer includes a second Fc domain with heterodimer skew variants S364K/E357Q and FcKO variants E233P/L234V/L235A/G236del/S267K, according to the EU index. In some embodiments, the first and second monomers each also include M428L/N434S half-life extension variants. In some embodiments, a IL18 protein or variant thereof is connected to the first Fc domain.

In some embodiments, the first and second monomers each also include a C220S hinge amino acid substitution. In some embodiments, the first and second monomers each also include a N297A or N297S amino acid substitution that removes glycosylation. In some embodiments, the first monomer includes a first Fc domain with modifications C220 S/E233P/L234V/L235A/G236del/S267K/Q295E/L368D/K370 S/384D/Q418E/N421D and optionally M428L/N434S and the second monomer includes a second Fc domain with modifications C220S/E233P/L234V/L235A/G236del/S267K/S364K/E357Q and optionally modifications M428L/N434S, according to the EU numbering. In some embodiments, a IL18 protein or variant thereof is connected to the first Fc domain.

FIGS. 21A-21M depict amino acid modifications in the first and second monomers of a heterodimeric monovalent IL18-Fc fusion protein. Additional, exemplary Fc domain “backbone sequences” that find use in the subject monovalent IL18-Fc fusion proteins are depicted in FIGS. 9A-9D and 10.

In the formulas above, “IL18” is any IL18 provided herein (see, e.g., wildtype or variant IL18 depicted in FIGS. 13A-13B, 14, 15A-15D, 16A-16E, 17A-17B, 18, 19A-19P, 20A-20D, 41A-41C, 42A-42D, 43A-13B, and 44A-44C), “Fc domain” refers to any Fc domain provided herein (e.g., wildtype or variant Fc domains provided herein), and “linker” refers to any linker provided herein (see, e.g., FIG. 8). Further, “N” and “C” refer to the N-terminal and C-terminal orientation of each component in the second monomer. In such embodiments, the first monomer only includes an Fc domain (i.e., an “empty Fc domain”). In some embodiments, the each of the first and second Fc domains have the formula N-hinge-CH2-CH3-C. In certain embodiments, each of the first and second Fc domains have the formula N-CH2-CH3-C.

Exemplary monovalent IL18 fusion proteins include XENP30792, XENP31296, XENP31812, XENP31813, XENP31814, XENP34106, XENP34107, XENP34108, XENP34109, XENP34110, XENP34111, XENP34112, XENP34113, XENP34114, XENP34281, XENP37825, XENP37826, XENP38869, XENP39138, XENP39139, XENP39140, XENP39141, XENP39142, XENP39149, XENP39150, XENP39151, XENP39152, XENP39672, XENP39804, XENP39805, XENP40024, XENP40025, XENP40685, XENP40962, XENP40963, XENP40964, XENP40965, XENP40966, XENP40967, XENP40968, XENP41756, XENP71757, XENP41758, XENP41759, XENP41760, XENP41761, XENP41762, XENP41763, XENP41764, XENP41675, XENP41676, XENP41677, XENP41678, XENP41679, XENP41770, XENP41974, XENP41975, XENP42003, XENP42007, XENP42008, XENP42009, XENP42010, XENP42011, XENP42012, XENP42141, XENP42142, XENP42143, XENP42144, XENP42145, XENP42146, XENP42147, and XENP42148, as shown in FIGS. 21A-21M and the corresponding sequences (see, e.g., SEQ ID NOS:239-292 and 960-1039).

B. IL18 x Fab-Fc Fusion Proteins

In some embodiments, the IL18 fusion is IL18 x Fab-Fc fusion protein that includes (a) a first monomer comprising a variable heavy (VH) region covalently attached to the N-terminus of a first heterodimeric Fc chain, (b) a second monomer comprising an IL18 protein or variant thereof covalently attached to the N-terminus of a complementary second heterodimeric Fc chain (optionally via a domain linker), and (c) a third monomer that is a corresponding light chain that forms a Fab with the first monomer. The Fc chain of the first monomer and the Fc chain of the second monomer form a heterodimeric Fc complex. See, the schematic diagram in FIG. 12B and the amino acid sequences of FIGS. 22A-22CZ.

Any of the IL18s described herein can be included in the monovalent IL18-Fc fusion protein. In some embodiments, the IL18 is wildtype mature human IL18 (FIG. 1A). In certain embodiments, the IL18 is a variant IL18 that includes one or more modifications as depicted in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-13B, 44A-44C, 45, 46, 47, 48, 51, 54, and 62. In some embodiments, one or more residues of IL18 selected from Y1, E6, S7, K8, S10, V11, N14, L15, D17, Q18, D23, R27, P28, L29, E31, M33, T34, D35, S36, D37, C38, R39, D40, N41, R44, I46, I49, S50, M51, K53, D54, S55, Q56, P57, M60, A61, V62, T63, S65, K67, C68, E69, I71, C76, E77, I80, I81, N87, P88, D90, K93, T95, K96, S97, Q103, H109, D110, N111, M113, S119, A126, C127, D132, L136, L138, K139, E141, L144, D146, R147, I149, M150, N155, E156, and D157 are modified. In some embodiments, the IL18 of the IL18 x Fab-Fc fusion proteins is a variant IL18 that includes one or more modifications (e.g., substitutions or deletions) selected from Y1F, Y1H, E6A, E6Q, S7C, S7P, K8E, K8Q, K8Y, S10C, V11I, N14C, N14W, L15C, D17N, Q18L, D23N, D23S, R27Q, P28C, L29V, E31Q, M33C, T34P, D35N, D35E, S36D, S36N, D37N, C38S, C38Q, C38R, C38E, C38L, C38I, C38V, C38K, C38D, R39S, R39T, D40N, N41Q, R44Q, I46V, I49C, S50C, S50Y, M51I, M51K, M51Q, M51R, M51L, M51H, M51F, M51Y, K53A, K53D, K53E, K53G, K53H, K53I, K53L, K53M, K53N, K53Q, K53R, K53S, K53T, K53V, K53Y, K53F, D54C, S55N, S55Q, S55D, S55E, S55T, Q56I, Q56L, P57A, P57E, P57T, P57V, P57Q, P57D, P57Y, P57N, M60I, M60L, M60K, M60Y, M60F, A61C, V62C, T63C, S65C, K67Q, C68S, C68I, C68F, C68Y, C68D, C68N, C68E, C68Q, C68K, E69K, I71M, C76S, C76E, C76K, E77K, 180T, I81L, I81V, N87S, P88C, D90E, K93D, K93N, T95E, K96G, K96Q, S97N, Q103C, Q103E, Q103I, Q103L, H109W, H109Y, D110N, D110Q, D110R, N111D, N111Q, N111S, N111T, N111E, M113I, S119L, A126C, C127S, C127W, C127Y, C127F, C127D, C127E, C127K, D132Q, D132E, L136C, L138C, K139C, E141K, E141Q, L144N, D146F, D146L, D146Y, R147C, R147K, I149V, M150F, M150T, N155C, E156Q, D157A, D157S, D157N, and D157del. In some embodiments, the IL18 variant includes a 4CS substitution (C38S/C68S/C76S/C127S substitutions) and one or more of additional substitutions including S38C, S38E, S38L, S38Q, S38R, S38V, S38K, S38D, S68C, S68D, S68E, S68F, S68I, S68N, S68Q, S68Y, S68K, S76C, S76E, S76K, S127C, S127D, S127F, S127W, S127K, and S127Y. In some embodiments, the amino acid substitution can include 4CS, 4CS/D193S, 4CS/D193A, 4CS/delD193, 4CS/S38E, 4CS/S68E, 4CS/S76E, 4CS/S127E, 4CS/S38K, 4CS/S68K, 4CS/S76K, 4CS/S127K, 4CS/S38D, 4CS/Y1F, 4CS/Y1H, 4CS/E6A, 4CS/E6Q, 4CS/D17N, 4CS/E31Q, 4CS/D35N, 4CS/D37N, 4CS/D40N, 4CS/N41Q, 4CS/K53R, 4CS/K53H, 4CS/K53M, 4CS/K53E, 4CS/K53Q, 4CS/K53A, 4CS/Q103E, 4CS/D110N, 4CS/N111Q, 4C S/E6A/K53A, 4CS/N14C/E31Q/S127C, 4C S/E31Q/K53A, 4CS/E31Q/D35N/K53A, 4CS/E31Q/N41Q/K53A, 4CS/E31Q/D35N/N41Q/K53A, 4CS/E31Q/D35N, 4CS/E31Q/N41Q, 4C S/E31Q/D35N/N41Q, 4C S/E31Q/D37N, 4CS/E31Q/D37N/K53A, 4CS/E31Q/M33C/S38C, 4C S/E31Q/S76C/L138C, 4CS/E31Q/568I, 4CS/E31Q/S68F, 4CS/E31Q/S127W, 4CS/E31Q/S127Y, 4CS/E31Q/S127F, 4CS/S10C/E31Q/149C, 4C S/L15C/E31Q/R147C, 4C S/P28C/E31Q/L136C, 4CS/E31Q/S50C/P88C, 4CS/E31Q/T63C/P88C, 4C S/E31Q/V62C/Q103C, 4CS/S10C/E31Q/N155C, 4CS/E31Q/S65C/P88C, 4CS/S7C/E31Q/S50C, 4CS/E31Q/D54C/A61C, 4C S/E31Q/A126C/K139C, 4CS/N14W/E31Q, 4CS/E31Q/D146Y, 4CS/E31Q/D146L, 4C S/E31Q/D146F, 4C S/E31Q/Q103L, 4CS/E31Q/Q103I, 4CS/E31Q/M150F, 4C S/Q18L/E31Q, 4CS/E31Q/S68Y, 4CS/E31Q/S38Q, 4C S/E31Q/S38R, 4CS/E31Q/S68D, 4CS/S7P/E31Q, 4CS/V11I/E31Q, 4CS/D23N/E31Q, 4CS/D23S/E31Q, 4CS/R27Q/E31Q, 4CS/L29V/E31Q, 4CS/E31Q/T34P, 4CS/E31Q/R39T, 4CS/E31Q/R39S, 4CS/E31Q/R44Q, 4CS/E31Q/I46V, 4CS/E31Q/S50Y, 4CS/E31Q/Q56L, 4CS/E31Q/Q56L/P57T, 4C S/E31Q/P57T, 4CS/E31Q/P57V, 4CS/E31Q/M60L, 4CS/E31Q/K67Q, 4CS/E31Q/E69K, 4CS/E31Q/I71M, 4CS/E31Q/E77K, 4CS/E31Q/I80T, 4CS/E31Q/I81V, 4CS/E31Q/I81L, 4C S/E31Q/N87S, 4CS/E31Q/D90E, 4CS/E31Q/K93D/T95E, 4CS/E31Q/K93N/T95E, 4CS/E31Q/T95E, 4CS/E31Q/K96G, 4CS/E31Q/S97N, 4CS/E31Q/N111D, 4CS/E31QN1113I, 4CS/E31Q/S119L, 4CS/E31Q/L144N, 4CS/E31Q/R147K, 4CS/E31Q/I149V, 4CS/E31Q/M150T, 4CS/E31Q/E156Q/D157N, 4CS/K53S, 4CS/K53G, 4CS/K53T, 4CS/K53I, 4CS/K53L, 4CS/K53N, 4CS/K53D, 4CS/M51K, 4CS/M51Q, 4CS/M51I, 4CS/S55N, 4CS/S55Q, 4CS/Q56L, 4CS/Q56I, 4CS/P57A, 4CS/P57E, 4CS/M60L, 4CS/M60I, 4CS/K8Y, 4CS/K8Q, 4CS/K8E, 4CS/H109W, 4CS/H109Y, 4CS/E31Q/S38E, 4CS/E31Q/S38L, 4CS/E31Q/S38I, 4C S/E31Q/S38V, 4C S/E31Q/S68N, 4CS/E31Q/S68E, 4CS/E31Q/S68Q, 4C S/E31Q/S76C, 4C S/E31Q/S127D, 4CS/E31Q/S127E, 4CS/D23N/E31Q/R27Q, 4CS/E31Q/Q56L/T95E, 4CS/E31Q/K96Q/S119L, 4CS/E31Q/E141K/I149V, 4CS/E31Q/E141Q/I149V, 4CS/S7P/E31Q/S50Y, 4CS/E31Q/I80T/I81L/delD193, 4CS/E31Q/P57A/S119L/delD193, 4CS/E31Q/P57A/I80T/I81L/S119L/delD193, 4CS/E31Q/P57A/K93D/T95E/S119L/delD193, 4CS/E31Q/I80T/S119L/delD193, 4CS/E31Q/I80T/I81L/K93D/T95E/delD193, 4CS/E31Q/P57A/I80T/I81L/K93D/T95E/S119L/delD193, 4CS/S7C/E31Q/S50C/delD193, 4CS/S7C/E31Q/S50C/P57A/delD193, 4CS/S7C/E31Q/S50C/S119L/delD193, 4CS/S7C/E31Q/S50C/I80T/delD193, 4CS/S7C/E31Q/S50C/I80T/S119L/delD193, 4CS/S7C/E31Q/S50C/P57A/I80T/S119L/delD193, 4CS/S10C/E31Q/N155C/delD193, 4CS/S10C/E31Q/P57A/N155C/delD193, 4CS/S10C/E31Q/S119L/N155C/delD193, 4CS/S10C/E31Q/I80T/N155C/delD193, 4C S/S10C/E31Q/I80T/S119L/N155C/delD193, 4CS/S10C/E31Q/P57A/I80T/S119L/N155C/delD193, 4C S/S10C/E31Q/I49C/delD193, 4CS/L15C/E31Q/R147C/delD193, 4CS/E31Q/T63C/P88C/delD193, 4CS/N14C/E31Q/S127C/delD193, 4CS/E31Q/S38R/S127W/delD193, 4CS/S10C/D35E/N155C, 4CS/S10C/S36D/N155C, 4CS/S10C/S36N/N155C, 4CS/S10C/K53V/N155C, 4CS/S10C/K53Y/N155C, 4CS/S10C/K53F/N155C, 4CS/S10C/M51R/N155C, 4CS/S10C/M51L/N155C, 4C S/S10C/M51H/N155C, 4C S/S10C/M51F/N155C, 4CS/S10C/M51Y/N155C, 4CS/S10C/S55D/N155C, 4CS/S10C/S55E/N155C, 4CS/S10C/S55T/N155C, 4CS/S10C/P57Q/N155C, 4CS/S10C/P57D/N155C, 4CS/S10C/P57Y/N155C, 4CS/S10C/P57N/N155C, 4CS/S10C/M60Y/N155C, 4CS/S10C/M60F/N155C, 4CS/S10C/D110Q/N155C, 4CS/S10C/D110R/N155C, 4CS/S10C/N111D/N155C, 4CS/S10C/N111S/N155C, 4CS/S10C/N111T/N155C, 4CS/S10C/N111E/N155C, 4CS/S10C/D132Q/N155C, 4CS/S10C/D132EN155C, 4CS/E6Q/S10C/K53D/N155C, 4C S/E6Q/S10C/M51K/K53D/N155C, 4CS/S10C/E31Q/D35N/N41Q/K53A/N155C, 4CS/S10C/E31Q/N41Q/K53A/N155C, 4CS/S10C/E31Q/K53A/N155C, 4CS/S10C/K53T/N155C, 4CS/S10C/P57A/N155C, 4CS/S10C/N155C, 4CS/S10C/S76G/N155C, 4CS/S10C/S76A/N155C, 4CS/S10C/M51K/K53D/N155C, 4CS/S10C/M51K/K53E/N155C, 4CS/E6Q/S10C/K53E/N155C, 4CS/E6Q/S10C/M51K/K53E/N155C, 4CS/E6Q/S10C/M51K/P57E/N155C, 4CS/S10C/M51K/P57E/N155C, 4CS/E6Q/S10C/P57E/N155C, 4CS/S10C/E31Q/K53T/N155C, 4CS/S10C/K53G/P57E/N155C, 4CS/S10C/K53T/P57E/N155C, 4CS/S10C/K53A/P57E/N155C, 4CS/S10C/P57E/N155C, 4CS/S10C/K53D/N155C, 4CS/S10C/E31Q/N41Q/N155C, 4CS/S10C/K53A/N155C, 4CS/S10C/K53G/N155C, 4CS/S10C/K53E/N155C, 4CS/S10C/K53S/N155C, 4CS/S10C/M51L/K53D/N155C, 4CS/S10C/K53D/D110R/N155C, 4CS/S10C/K53D/N111T/N155C, 4CS/S10C/K53D/S55T/N155C, 4CS/S10C/K53D/S55T/D110R/N155C, 4CS/S10C/M51L/K53D/S55T/D110R/N111T/N155C, 4CS/S10C/M51L/K53D/S55T/D110R/N155C, 4CS/S10C/K53D/S55T/D110R/N111T/N155C, 4CS/S10C/K53D/S55T/N111T/N155C, 4CS/S10C/E31Q/D35N/N155C, 4CS/S10C/N41Q/N155C, 4CS/S10C/D35N/N155C, 4CS/S10C/D37N/N155C, 4CS/S10C/E31Q/D37N/N155C, 4CS/S10C/D35N/D37N/N155C, 4CS/E6Q/S10C/M51L/K53D/S55T/D110R/N111T/N155C, 4CS/S10C/K53D/H109Y/N155C, 4CS/S10C/D37N/K53D/N155C, 4CS/S10C/D35N/K53D/N155C, 4CS/K8E/S10C/K53D/N155C, 4CS/S10C/E31Q/K53D/N155C, 4CS/S10C/N41Q/K53D/N155C, 4CS/S10C/K53D/P57V/N155C, 4CS/S10C/K53D/P57T/N155C, 4CS/E6Q/S10C/K53D/N111T/N155C, E6A/K53A, D35N/K53A, N41Q/K53A, D35N/N41Q/K53A, D35N/N41Q, D37N/K53A, E6Q/K53D, E6Q/M51K/K53D, M51K/K53D, M51K/K53E, E6Q/K53E, E6Q/M51K/K53E, E6Q/M51K/P57E, M51K/P57E, E6Q/P57E, K53G/P57E, K53T/P57E, K53A/P57E, M51L/K53D, K53D/D110R, K53D/N111T, K53D/S55T, K53D/S55T/D110R, M51L/K53D/S55T/D110R/N111T, M51L/K53D/S55T/D110R, K53D/S55T/D110R/N111T, K53D/S55T/N111T, D35N/D37N, E6Q/M51L/K53D/S55T/D110R/N111T, K53D/H109Y, D37N/K53D, D35N/K53D, K8E/K53D, N41Q/K53D, K53D/P57V, K53D/P57T, E6Q/K53D/N111T, Q56L/P57T, K93D/T95E, K93N/T95E, E156Q/D157N, D23N/R27Q, Q56L/T95E, K96Q/S119L, E141K/I149V, E141Q/I149V, S7P/S50Y, I80T/I81L, P57A/S119L, P57A/I80T/I81L/S119L, P57A/K93D/T95E/S119L, I80T/S119L, I80T/I81L/K93D/T95E, P57A/I80T/I81L/K93D/T95E/S119L, P57A/I80T/S119L, N14C/S127C, M33C/S38C, S76C/L138C, S10C/I49C, L15C/R147C, P28C/L136C, S50C/P88C, T63C/P88C, V62C/Q103C, S10C/N155C, S65C/P88C, S7C/S50C, D54C/A61C, A126C/K139C, C38R/C127W, E31Q/K53A, E31Q/D35N/K53A, E31Q/N41Q/K53A, E31Q/D35N/N41Q/K53A, E31Q/D35N, E31Q/N41Q, E31Q/D35N/N41Q, E31Q/D37N, E31Q/D37N/K53A, S10C/E31Q/I49C, L15C/E31Q/R147C, P28C/E31Q/L136C, E31Q/S50C/P88C, E31Q/T63C/P88C, E31Q/V62C/Q103C, S10C/E31Q/N155C, E31Q/S65C/P88C, S7C/E31Q/S50C, E31Q/D54C/A61C, E31Q/A126C/K139C, N14W/E31Q, E31Q/D146Y, E31Q/D146L, E31Q/D146F, E31Q/Q103L, E31Q/Q103I, E31Q/M150F, Ql8L/E31Q, S7P/E31Q, V11I/E31Q, D23N/E31Q, D23 S/E31Q, R27Q/E31Q, L29V/E31Q, E31Q/T34P, E31Q/R39T, E31Q/R39S, E31Q/R44Q, E31Q/I46V, E31Q/S50Y, E31Q/Q56L, E31Q/Q56L/P57T, E31Q/P57T, E31Q/P57V, E31Q/M60L, E31Q/K67Q, E31Q/E69K, E31Q/I71M, E31Q/E77K, E31Q/I80T, E31Q/I81V, E31Q/I81L, E31Q/N87S, E31Q/D90E, E31Q/K93D/T95E, E31Q/K93N/T95E, E31Q/T95E, E31Q/K96G, E31Q/S97N, E31Q/N111D, E31Q/M1131, E31Q/S119L, E31Q/L144N, E31Q/R147K, E31Q/I149V, E31Q/M150T, E31Q/E156Q/D157N, D23N/E31Q/R27Q, E31Q/Q56L/T95E, E31Q/K96Q/S119L, E31Q/E141K/I149V, E31Q/E141Q/I149V, S7P/E31Q/S50Y, E31Q/180T/181L/delD193, E31Q/P57A/S119L/delD193, E31Q/P57A/180T/181L/S119L/delD193, E31Q/P57A/K93D/T95E/S119L/delD193, E31Q/I80T/S119L/delD193, E31Q/I80T/I81L/K93D/T95E/delD193, E31Q/P57A/180T/181L/K93D/T95E/S119L/delD193, S7C/E31Q/S50C/delD193, S7C/E31Q/S50C/P57A/delD193, S7C/E31Q/S50C/S119L/delD193, S7C/E31Q/S50C/I80T/delD193, S7C/E31Q/S50C/I80T/S119L/delD193, S7C/E31Q/S50C/P57A/I80T/S119L/delD193, S10C/E31Q/N155C/delD193, S10C/E31Q/P57A/N155C/delD193, S10C/E31Q/S119L/N155C/delD193, S10C/E31Q/I80T/N155C/delD193, S10C/E31Q/I80T/S119L/N155C/delD193, S10C/E31Q/P57A/I80T/S119L/N155C/delD193, S10C/E31Q/I49C/delD193, L15C/E31Q/R147C/delD193, E31Q/T63C/P88C/delD193, S10C/D35E/N155C, S10C/S36D/N155C, S10C/S36N/N155C, S10C/K53V/N155C, S10C/K53Y/N155C, S10C/K53F/N155C, S10C/M51R/N155C, S10C/M51L/N155C, S10C/M51H/N155C, S10C/M51F/N155C, S10C/M51Y/N155C, S10C/S55D/N155C, S10C/S55E/N155C, S10C/S55T/N155C, S10C/P57Q/N155C, S10C/P57D/N155C, S10C/P57Y/N155C, S10C/P57N/N155C, S10C/M60Y/N155C, S10C/M60F/N155C, S10C/D110Q/N155C, S10C/D110R/N155C, S10C/N111D/N155C, S10C/N111S/N155C, S10C/N111T/N155C, S10C/N111E/N155C, S10C/D132Q/N155C, S10C/D132E/N155C, E6Q/S10C/K53D/N155C, E6Q/S10C/M51K/K53D/N155C, S10C/E31Q/D35N/N41Q/K53A/N155C, S10C/E31Q/N41Q/K53A/N155C, S10C/E31Q/K53A/N155C, S10C/K53T/N155C, S 10C/P57A/N155C, S 10C/M51K/K53D/N155C, S 10C/M51K/K53E/N155C, E6Q/S10C/K53E/N155C, E6Q/S10C/M51K/K53E/N155C, E6Q/S10C/M51K/P57E/N155C, S 1 OC/M51K/P57E/N155C, E6Q/S10C/P57E/N155C, S 1 OC/E31Q/K53T/N155C, S10C/K53G/P57E/N155C, S10C/K53T/P57E/N155C, S10C/K53A/P57E/N155C, S10C/P57E/N155C, S10C/K53D/N155C, S10C/E31Q/N41Q/N155C, S10C/K53A/N155C, S10C/K53G/N155C, S10C/K53E/N155C, S10C/K53S/N155C, S10C/M51L/K53D/N155C, S10C/K53D/D110R/N155C, S10C/K53D/N111T/N155C, S10C/K53D/S55T/N155C, S10C/K53D/S55T/D110R/N155C, S10C/M51L/K53D/S55T/D110R/N111T/N155C, S10C/M51L/K53D/S55T/D110R/N155C, S10C/K53D/S55T/D110R/N111T/N155C, S10C/K53D/S55T/N111T/N155C, S10C/E31Q/D35N/N155C, S10C/N41Q/N155C, S10C/D35N/N155C, S10C/D37N/N155C, S10C/E31Q/D37N/N155C, S10C/D35N/D37N/N155C, E6Q/S10C/M51L/K53D/S55T/D110R/N111T/N155C, S10C/K53D/H109Y/N155C, S10C/D37N/K53D/N155C, S10C/D35N/K53D/N155C, K8E/S10C/K53D/N155C, S10C/E31Q/K53D/N155C, S10C/N41Q/K53D/N155C, S10C/K53D/P57V/N155C, S10C/K53D/P57T/N155C, or E6Q/S10C/K53D/N111T/N155C.

In some embodiments, the IL18 variant of the IL18 x Fab-Fc fusion protein includes one or more amino acid substitutions provided in FIGS. 13A-13B, 14, 15A-15D, 16A-16E, 17A-B, 19A-19P, 20A-20D, 31, 36, 37, 39A-39B, 40, 41A-41C, 42A-42D, 43A-13B, 44A-44C, 45, 46, 47, 48, 51, 54, and 62. In some embodiments, the IL18 variant of the IL18-Fc fusion protein is depicted in any one of FIGS. 13A-13B, 14, 15A-15D, 16A-16E, 17A-17B, 18, 19A-19P, 20A-20D, 41A-41C, 42A-42D, 43A-13B, and 44A-44C. In certain embodiments, the IL18 variant includes an amino acid sequence set forth in SEQ ID NOS:84-238 and 799-949.

Any Fc domains can be included in the IL18 x Fab-Fc fusion protein, including the wildtype and variant Fc domains described herein. In some embodiments, each Fc domain includes a CH2 and CH3. In certain embodiments, the first and second Fc domains include a hinge, CH2 and CH3. In one embodiment, the first and second Fc domains each have the formula, from N-terminus to C-terminus, hinge-CH2-CH3. In exemplary embodiments, the first and second Fc domains of the monovalent IL18-Fc fusion protein are heterodimeric. Modifications for such Fc domains are described in Sections VI-VIII above.

In exemplary embodiments, the IL18 x Fab-Fc fusion protein is a heterodimeric Fc fusion protein. In some heterodimeric embodiments, the first and second Fc domains include the amino acid substitution set L368D/K370S: S364K/E357Q. In some embodiments, the S364K/E357Q modifications are in the first Fc domain and the L368D/K370S modifications are in the second Fc domain. In certain heterodimeric embodiments, the second Fc domain includes isosteric pI variants Q295E/N384D/Q418E/N421D. In certain embodiments, both the first and second Fc domains include FcKO variants: E233P/L234V/L235A/G236del/S267K, according to the EU numbering. In some embodiments, the first Fc domain and the second Fc domain each include K447del modifications. In some embodiments, the IL18 protein or variant thereof is linked to the Fc domain that includes isosteric pI variants (e.g., the second Fc domain).

In exemplary embodiments, the IL18 x Fab-Fc fusion protein is a heterodimeric Fc fusion protein containing a first monomer, a second monomer and a third monomer. In some embodiments, the first monomer includes a variable heavy chain, the second monomer includes an IL18 protein or variant thereof, and the third monomer includes a variable light chain. In some embodiments, the first monomer includes a first Fc domain with heterodimer skew variants S364K/E357Q and FcKO variants E233P/L234V/L235A/G236del/S267K, according to the EU index. In some embodiments, the second monomer includes a second Fc domain with heterodimer skew variants L368D/K370S, isosteric pI variants Q295E/N384D/Q418E/N421D, and FcKO variants E233P/L234V/L235A/G236del/S267K.

In some embodiments, the first and second monomers each also include M428L/N434S half-life extension variants. In some embodiments, the first and second monomers each also include a C220S hinge amino acid substitution.

In some embodiments, the first monomer includes a second Fc domain with modifications C220S/E233P/L234V/L235A/G236del/S267K/S364K/E357Q and optionally modifications M428L/N434S, the second monomer includes a first Fc domain with modifications C220 S/E233P/L234V/L235A/G236del/S267K/Q295E/L368D/K370 S/384D/Q418E/N421D and optionally M428L/N434S, and according to the EU numbering and a third monomer that does not include an Fc domain. and according to the EU numbering. In some embodiments, the first Fc domain of the first monomer and the second Fc domain of the second monomer each include K447del modifications.

FIGS. 21A-21M depict amino acid modifications in the first and second monomers of a heterodimeric monovalent IL18-Fc fusion protein. Additional, exemplary Fc domain “backbone sequences” that find use in the subject monovalent IL18-Fc fusion proteins are depicted in FIGS. 9A-9D and 10.

In the formulas above, “IL18” is any IL18 provided herein (see, e.g., wildtype or variant IL18 depicted in FIGS. 13A-13B, 14, 15A-15E, 16A-16E, 17A-17B, 18, 19A-19P, 20A-20D, 41A-41C, 42A-42D, 43A-13B, and 44A-44C), “Fc domain” refers to any Fc domain provided herein (e.g., wildtype or variant Fc domains provided herein), and “linker” refers to any linker provided herein (see, e.g., FIG. 8). Further, “N” and “C” refer to the N-terminal and C-terminal orientation of each component in the second monomer. In such embodiments, the first monomer only includes an Fc domain (i.e., an “empty Fc domain”). In some embodiments, the each of the first and second Fc domains have the formula N-hinge-CH2-CH3-C. In certain embodiments, each of the first and second Fc domains have the formula N-CH2-CH3-C.

Exemplary IL18 x Fab-Fc fusion proteins include XENP37827, XENP37828, XENP38850, XENP38851, XENP38852, XENP38853, XENP38854, XENP38855, XENP38856, XENP38857, XENP38858, XENP38859, XENP38860, XENP38861, XENP38862, XENP38863, XENP38864, XENP38865, XENP38866, XENP38867, XENP38868, XENP38952, XENP38953, XENP38954, XENP38868, XENP38956, XENP38957, XENP39601, XENP39602, XENP39603, XENP39604, XENP40027, XENP40046, XENP40047, XENP40048, XENP40049, XENP40050, XENP40051, XENP40052, XENP40053, XENP40054, XENP40175, XENP40176, XENP40177, XENP40178, XENP40179, XENP40181, XENP40182, XENP40183, XENP40184, XENP40185, XENP40186, XENP40187, XENP40188, XENP40189, XENP40190, XENP40191, XENP40192, XENP40193, XENP40194, XENP40195, XENP40196, XENP40197, XENP40198, XENP40199, XENP40200, XENP40201, XENP40202, XENP40203, XENP40204, XENP40205, XENP40206, XENP40207, XENP40208, XENP40209, XENP40210, XENP40211, XENP40212, XENP40213, XENP40214, XENP40215, XENP40216, XENP40217, XENP40218, XENP40219, XENP40220, XENP40221, XENP40222, XENP40223, XENP40224, XENP40225, XENP40226, XENP40227, XENP40228, XENP40229, XENP40230, XENP40231, XENP40232, XENP40233, XENP40234, XENP40235, XENP40236, XENP40237, XENP40238, XENP40239, XENP40240, XENP40241, XENP40242, XENP40243, XENP40244, XENP40246, XENP40247, XENP40248, XENP40249, XENP40250, XENP40251, XENP40252, XENP40253, XENP40254, XENP40255, XENP40256, XENP40257, XENP40258, XENP40259, XENP40260, XENP40261, XENP40262, XENP40263, XENP40264, XENP40265, XENP40266, XENP40267, XENP40268, XENP40269, XENP40363, XENP40364, XENP40617, XENP40618, XENP40619, XENP40620, XENP40621, XENP40622, XENP40623, XENP40624, XENP40625, XENP40626, XENP40627, XENP40628, XENP40629, XENP40630, XENP40631, XENP40632, XENP40657, XENP40658, XENP40659, XENP40660, XENP40661, XENP40662, XENP40663, XENP40686, XENP40934, XENP40935, XENP40936, XENP40937, XENP40938, XENP40939, XENP40940, XENP40941, XENP40942, XENP40943, XENP40944, XENP40945, XENP40946, XENP40947, XENP40948, XENP40949, XENP40950, XENP40951, XENP40952, XENP40953, XENP40954, XENP40955, XENP40956, XENP40957, XENP40958, XENP40959, XENP40960, XENP40961, XENP41076, XENP41077, XENP41078, XENP41079, XENP41080, XENP41081, XENP41082, XENP41083, XENP41084, XENP41085, XENP41086, XENP41087, XENP41088, XENP41089, XENP41090, XENP41091, XENP41092, XENP41093, XENP41094, XENP41095, XENP41096, XENP41353, XENP41416, XENP41417, XENP41418, XENP41419, XENP41420, XENP41421, XENP41422, XENP41423, XENP41424, XENP41425, XENP41426, XENP41427, XENP41427, XENP41429, XENP41430, XENP41431, XENP41440, XENP41513, XENP41514, XENP41515, XENP41516, XENP41517, XENP41518, XENP41519, and XENP41520 as shown in FIGS. 22A-22CZ and the corresponding sequences (see, e.g., SEQ ID NOS:294-774 and 1040-1264).

XI. Nucleic Acids

In another aspect, provided herein are nucleic acid compositions encoding the subject IL18-Fc fusion proteins and IL18s (e.g., variant IL18s) described herein. As will be appreciated by those in the art, the nucleic acid compositions will depend on the format of the fusion protein. Thus, for example, when the format requires two amino acid sequences (e.g., heterodimeric

IL18-Fc fusions), two nucleic acid sequences can be incorporated into one or more expression vectors for expression.

As is known in the art, the nucleic acids encoding the monomer components of the IL18-Fc fusion proteins can be incorporated into expression vectors as is known in the art, and depending on the host cells used to produce the heterodimeric IL18-Fc fusion proteins. Generally, the nucleic acids are operably linked to any number of regulatory elements (promoters, origin of replication, selectable markers, ribosomal binding sites, inducers, etc.). The expression vectors can be extra-chromosomal or integrating vectors.

The nucleic acids and/or expression vectors are then transformed into any number of different types of host cells as is well known in the art, including, but not limited to, mammalian, bacterial, yeast, insect and/or fungal cells, with mammalian cells (e.g. CHO cells) being preferred.

In some embodiments, particularly heterodimeric IL18-Fc fusion proteins, nucleic acids encoding each monomer are each contained within a single expression vector, generally under different or the same promoter controls. In certain embodiments, each of the two nucleic acids are contained on a different expression vector.

The subject IL18-Fc fusion protein are made by culturing host cells comprising the expression vector(s) as is well known in the art. Once produced, traditional fusion protein or antibody purification steps are done, including an ion exchange chromatography step. As discussed herein, having the pIs of the two monomers differ by at least 0.5 can allow separation by ion exchange chromatography or isoelectric focusing, or other methods sensitive to isoelectric point. That is, the inclusion of pI variants that alter the isoelectric point (pI) of each monomer so that each monomer has a different pI and the resulting heterodimeric IL18-Fc fusion protein also has a distinct pI advantageously facilitates isoelectric purification of the heterodimer (e.g., anionic exchange chromatography, cationic exchange chromatography). These substitutions also aid in the determination and monitoring of any contaminating homodimers post-purification (e.g., IEF gels, cIEF, and analytical IEX columns).

XII. Biological and Biochemical Functionality of IL18 Fc Fusion Proteins

Biological activity of the subject IL18-Fc fusion proteins (including IL18 x Fab-Fc fusion) and variant IL18s can be assessed using any IL18 activity assay known in the art. In exemplary in vitro assays, the test IL18-Fc fusion proteins can be used to stimulate the human myelomonocytic cell line, KG-1, which produces IFNy and then upregulates PD-L1 which can be measured. IL18BP inhibition of the IL18-Fc fusion proteins can also be determined in such assays

The effects of subject IL18-Fc fusion protein and variant IL18s on the proliferation of various lymphocyte populations can be assessed using any method for lymphocyte proliferation, for example, but not limited to CFSE dilution method, Ki67 intracellular staining of immune effector cells, and ³H-thymidine incorporation method.

Biological activity of the subject IL18-Fc fusion proteins can also be tested in vivo in an animal model, such as a Graft-versus-Host Disease (GVHD) model conducted in immunodeficient mice with engraftment of foreign immune cells (e.g., human PBMCs).

Generally, the subject IL18-Fc fusion proteins are administered to patients in need thereof (e.g., a patient with a cancer) and efficacy is assessed, in a number of ways as described herein. Thus, while standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays.

For example, evaluation of changes in immune status (e.g., presence of ICOS+CD4+T cells following ipi treatment) along with traditional measurements such as tumor burden, size, invasiveness, LN involvement, metastasis, etc. can be done. Thus, any or all of the following can be evaluated: the inhibitory effects of PVRIG on CD4⁺ T cell activation or proliferation, CDS⁺ T (CTL) cell activation or proliferation, CDS⁺ T cell-mediated cytotoxic activity and/or CTL mediated cell depletion, NK cell activity and NK mediated cell depletion, the potentiating effects of PVRIG on Treg cell differentiation and proliferation and Treg- or myeloid derived suppressor cell (MDSC)-mediated immunosuppression or immune tolerance, and/or the effects of PVRIG on proinflammatory cytokine production by immune cells, e.g., IL-2, IFN-γ or TNF-α production by T or other immune cells.

In some embodiments, assessment of treatment is done by evaluating immune cell proliferation, using for example, CF SE dilution method, Ki67 intracellular staining of immune effector cells, and ³H-thymidine incorporation method.

In some embodiments, assessment of treatment is done by evaluating the increase in gene expression or increased protein levels of activation-associated markers, including one or more of: CD25, CD69, CD137, ICOS, PD1, GITR, OX40, and cell degranulation measured by surface expression of CD107A.

In general, gene expression assays are done as is known in the art.

In general, protein expression measurements are also similarly done as is known in the art.

In some embodiments, assessment of treatment is done by assessing cytotoxic activity measured by target cell viability detection via estimating numerous cell parameters such as enzyme activity (including protease activity), cell membrane permeability, cell adherence, ATP production, co-enzyme production, and nucleotide uptake activity. Specific examples of these assays include, but are not limited to, Trypan Blue or PI staining, ⁵¹Cr or ³⁵S release method, LDH activity, MTT and/or WST assays, Calcein-AM assay, Luminescent based assay, Annexin V staining, Zombie AquaTM staining and others.

In some embodiments, assessment of treatment is done by assessing T cell activity measured by cytokine production, measured either intracellularly or in culture supernatant using cytokines including, but not limited to, IFNy, TNFa, GM-CSF, IL2, IL6, IL4, ILS, IL10, IL13 using well known techniques.

Accordingly, assessment of treatment can be done using assays that evaluate one or more of the following: (i) increases in immune response, (ii) increases in activation of af3 and/or y6 T cells, (iii) increases in cytotoxic T cell activity, (iv) increases in NK and/or NKT cell activity, (v) alleviation of af3 and/or y6 T-cell suppression, (vi) increases in pro-inflammatory cytokine secretion, (vii) increases in IL-2 secretion; (viii) increases in interferon-y production, (ix) increases in Th1 response, (x) decreases in Th2 response, (xi) decreases or eliminates cell number and/or activity of at least one of regulatory T cells (Tregs).

XIII. Treatments

Once made, the subject IL18-Fc fusion proteins find use in a number of oncology applications, such as by promoting IL18-Fc related immune cell activation (e.g., T cells are no longer suppressed) and proliferation.

Accordingly, the subject IL18-Fc fusion proteins provided find use in the treatment of these cancers.

A. Fusion Protein Compositions for In Vivo Administration

Formulations of the IL18-Fc fusion proteins used in accordance with the present invention are prepared for storage by mixing a fusion protein having the desired degree of purity with optional pharmaceutically acceptable carriers, excipients or stabilizers (as generally outlined in Remington's Pharmaceutical Sciences 16th edition, Osol, A. Ed. [1980]), in the form of lyophilized formulations or aqueous solutions. Acceptable carriers, buffers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEENTM, PLURONICSTM or polyethylene glycol (PEG).

B. Administrative Modalities

The IL18-Fc fusion proteins and chemotherapeutic agents are administered to a subject, in accord with known methods, such as intravenous administration as a bolus or by continuous infusion over a period of time.

C. Treatment Modalities

In the methods of treatment provided herein, therapy is used to provide a positive therapeutic response with respect to a disease or condition (e.g., a cancer). By “positive therapeutic response” is intended an improvement in the disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased patient survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, bone scan imaging, endoscopy, and tumor biopsy sampling including bone marrow aspiration (BMA) and counting of tumor cells in the circulation.

In addition to these positive therapeutic responses, the subject undergoing therapy may experience the beneficial effect of an improvement in the symptoms associated with the disease.

Treatment according to the present invention includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the protein or protein portion are outweighed by the therapeutically beneficial effects.

A “therapeutically effective amount” for tumor therapy may also be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specification for the dosage unit forms of the present invention are dictated by and directly dependent on (a) the unique characteristics of the active compound and the particular therapeutic effect to be achieved, and (b) the limitations inherent in the art of compounding such an active compound for the treatment of sensitivity in individuals.

The efficient dosages and the dosage regimens for the heterodimeric proteins used in the present invention depend on the disease or condition to be treated and may be determined by the persons skilled in the art.

An exemplary, non-limiting range for a therapeutically effective amount of an heterodimeric proteins used in the present invention is about 0.1-100 mg/kg.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

### EXAMPLES

Examples are provided below to illustrate the present invention. These examples are not meant to constrain the present invention to any particular application or theory of operation. For all constant region positions discussed in the present invention, numbering is according to the EU index as in Kabat (Kabat et al., 1991, Sequences of Proteins of Immunological Interest, 5th Ed., United States Public Health Service, National Institutes of Health, Bethesda, entirely incorporated by reference). Those skilled in the art of antibodies will appreciate that this convention consists of nonsequential numbering in specific regions of an immunoglobulin sequence, enabling a normalized reference to conserved positions in immunoglobulin families. Accordingly, the positions of any given immunoglobulin as defined by the EU index will not necessarily correspond to its sequential sequence.

General and specific scientific techniques are outlined in US Publ. App. No. 2015/0307629, US Publ. App. No. 2014/0288275, US Patent No. US 9,605,084 and WO 2014/145806, all of which are expressly incorporated by reference in their entirety and particularly for the techniques outlined therein.

### Example 1

### Engineering and Production of IL18 Fusion Proteins

As depicted in FIG. 71, IL18R1 expression is biased towards NKs and memory T cell and can be targeted by IL18. As described above, cytokines such as IL18 have short half-life, and high dose treatment is required to achieve a concentration of cytokines at the target (e.g., tumor site) sufficient to induce an immune response. However, based on observations with other cytokines, high dose treatment with IL18 could potentially result in systemic toxicities. In order to address this issue, IL18 molecules were engineered as Fc fusions in various formats (collectively referred to hereon as IL18 fusion proteins or just IL18 fusions) with the aim to enhance serum half-life through FcRn-mediated recycling.

1A: IL18 Fusion Protein Formats

1A(a): IL18-Fc Fusions

Various IL18-Fc fusion formats were conceived. One such exemplary format of this category is the “monovIL18-Fc” format (cartoon schematic depicted in FIG. 12A) which comprises a first monomer comprising an IL18 monomer covalently attached to the N-terminus of a first heterodimeric Fc chain (optionally via a domain linker) and a second monomer comprising a complementary second heterodimeric Fc chain that is “Fc-only” or “empty-Fc”. Illustrative proteins of the monovIL18-Fc format are depicted in FIGS. 21A-21M.

1A(b): IL18 x Fab-Fc Fusions

For the reasons described in Example 2C, another category of IL18 fusions include a Fab arm. One such exemplary format of this category is the “IL18 x Fab-Fc” format (cartoon schematic depicted in FIG. 12B) which comprises a first monomer comprising an IL18 monomer covalently attached to the N-terminus of a first heterodimeric Fc chain (optionally via a domain linker), a second monomer comprising a variable heavy (VH) region covalently attached to the N-terminus of a complementary second heterodimeric Fc chain, and a third monomer that is a corresponding light chain that forms a Fab with the second monomer. Illustrative proteins of the IL18 x Fab-Fc format are depicted in FIGS. 22A-22CZ.

1B: Production and purification of IL18 Fusion Proteins

To produce IL18 fusions in the monovIL18-Fc or the IL18 x Fab-Fc formats, plasmida coding for IL18 (WT or variant) was constructed by standard gene synthesis, followed by subcloning into pTT5 expression vectors containing Fc fusion partners (e.g. domain linkers and heterodimeric Fc backbones as depicted in FIGS. 8 and 9A-9C). For IL18 fusions in the monovIL18-Fc format, an additional plasmid encoding a complementary heterodimeric empty-Fc was used. Additionally, in the case of IL18 fusions in the IL18 x Fab-Fc formats, plasmid coding for the variable heavy and variable light regions were constructed by standard gene synthesis, followed by subcloning into pTT5 expression vectors containing either Fc fusion partners (e.g. CH1 regions, domain linkers, and heterodimeric Fc backbones as depicted in FIGS. 8, 9A-9C, and 10) or constant light domain (e.g. Constant Light Domain—Kappa as depicted in FIG. 11). Proteins were produced by transient transfection of HEK293E or CHO cells with appropriate set of plasmids as described above.

As will be described in Example 2D, IL18 fusions may be engineered with Rapid Purification (RP) variants for ease of purification. IL18 fusions without the RP variants were purified via a two-step purification process comprising protein A chromatography (purification part 1) followed by ion exchange chromatography (purification part 2). IL18 fusions with the RP variants were purified via one-step purification comprising just protein A chromatography.

Post-purification material (from separation peaks) may be further characterized by analytical size-exclusion chromatography with multi-angle light scattering (aSEC-MALS) and analytical anion-exchange chromatography (aAEX) for identity, purity and homogeneity as generally described below.

For aSEC-MALS, the analysis was performed on an Agilent 1200 high-performance liquid chromatography (HPLC) system. Samples were injected onto a SuperdexTM 200 10/300 GL column (GE Healthcare Life Sciences) at 1.0 mL/min using 1× PBS, pH 7.4 as the mobile phase at 4oC for 25 minutes with UV detection wavelength at 280 nM. MALS was performed on a miniDAWN® TREOS® with an Optilab® T-rEX Refractive Index Detector (Wyatt Technology, Santa Barbara, CA). Analysis was performed using Agilent OpenLab Chromatography Data System (CDS) ChemStation Edition AIC version C.01.07 and ASTRA version 6.1.7.15.

For aAEX, The analysis was performed on an Agilent 1200 high-performance liquid chromatography (HPLC) system. Samples were injected onto a Proteomix SAX-NP5 5₁1.M non-porous column (Sepax Technologies, Inc., Newark, Del.) at 1.0 mL/min using 0-40% NaCl gradient in 20 mM IVIES, pH 8.5 buffer with UV detection wavelength at 280 nM. Analysis was performed using Agilent OpenLAB CDS ChemStation Edition AIC version C.01.07.

### Example 2

### Engineering IL18 Variants (and Additional Approaches) to Improve Production

In this section, engineering approaches to improve IL18 fusion production are described. Collectively, these include engineering approaches to improve yield as well as to decrease molecular heterogeneity and the variants are collectively referred to herein as production variants.

2A: Engineering IL18 to Remove Free Cysteines

Initially, IL18 fusion XENP30792 in the monovIL18-Fc format was produced using WT human IL18 (sequence as depicted in FIG. 1 for WT human IL18 mature form sequence; and in FIG. 21A for XENP30792). XENP30792 was produced and purified via the two-step purification process as generally described in Example 1B. FIG. 23A depicts the chromatogram showing purification part 2 of XENP30792 as a broad heterogeneous peak. The full pre-purified load and material from the broad peak were further characterized by aSEC-MALS which show that the material was predominantly heavy molecular weight species (FIG. 23B).

Based on the crystal structure of human IL18 as reported by Tsutsumi et al. (Nature Communications, 15 Dec. 2014, 5, 5340) [PDB code 3WO2] and modeling in Molecular Operating Environment (MOE; Chemical Computing Group, Montreal, Quebec, Canada), it was found that the four cysteine residues were spatially distant and unlikely to form intramolecular disulfide bridges. Accordingly, it was hypothesized that the heavy molecular weight species were aggregate material resulting from intermolecular mispairing of free cysteines (as would be consistent with Yamamoto 2004). This was likely not a problem for human IL18 recombinantly produced in E. coli as expression takes place at lower temperature and in a reduced environment. Therefore, an IL18 variant 4CS was engineered with C38S, C68S, C76S and C127S (sequence as depicted in FIG. 13). IL18 fusion XENP31296 with IL18-4CS in the monovIL18-Fc format (sequences as depicted in FIGS. 21A-21M) was produced and purified as generally described in Example 1B. FIG. 24A depicts the chromatogram showing purification part 2 of XENP31296 as 3 distinct peaks (P1, B, and A). The 3 peaks were further characterized by aSEC-MALS, and chromatograms are depicted in FIG. 24B along with MW of component species. The profiles show that peak B comprises a single species of —72.3 kD which is consistent with the calculated molecular weight of XENP31296 (based on amino acid sequence) of 70.4 kD. Hereon, IL18 variant including only the 4CS substitution is referred to as WT-4CS.

2B: Additional Engineering to Remove Liabilities

In view of the potential impact of post-translational modification related liabilities as observed in Example 2A, additional engineering approaches were utilized to remove other potential liabilities.

One such approach was removal of the C-terminal lysine on the Fc regions. Therefore, each of the backbones as depicted in FIGS. 9A-9C (and in fact each of the IL18 fusion sequences depicted herein) may include a K447 deletion (K447_ or K447del) in one or both Fc regions.

Another such approach was removal of a “DG” aspartic acid isomerization motif. Initial IL18 fusion constructs were produced with IL18 covalently attached to a G4S linker. The C-terminal D of IL18 and the glycine residue introduces such a DG motif. A first approach explored was removal of the G4S linker so that the IL18 is covalently attached to the N-terminal of E of the hinge region. This approach significantly reduced molecular heterogeneity; however, it drastically reduced yield. Additional approaches explored to remove the DG motif included deletion or substitution of the C-terminal D of IL18 (e.g., XENP31812, XENP31813, XENP31814, XENP38952 and XENP38953) or utilizing alternative linkers that do not introduce the DG motif (e.g., AG4 or EA3K as in XENP38954, XENP38955, XENP38956, and XENP38957).

2C: Engineering IL18-Fc with a Silent Fab Arm

Another approach conceived for improving the production of IL18 fusions was to produce the molecules with a Fab arm occupying the empty-Fc side. To avoid non-specific activity of the IL18 fusions, the Fab arm used was a silent Fv based on SEQ ID NOs:23 and 24 as disclosed in the sequence listing of WO 2020/078905.

FIG. 25A depicts the chromatogram showing purification part 2 of XENP37827 as 2 distinct peaks (B and C) which is an improvement over the purification profile of XENP31296 as 3 distinct peaks. Peak B was from purification part 2 was further characterized by aSEC-MALS, and chromatograms are depicted in FIG. 25B along with MW of component species. The profiles show that peak B comprises a single species of —119.7 kD which is consistent with the calculated molecular weight of XENP37827 (based on amino acid sequence) of 116.2 kD.

Surprisingly, this format also enabled enhanced yield (data not shown). Although the E31Q variant was later identified as a variant which enhanced yield, this format was advantageous for further screening of variants so that substitutions which may be detrimental to yield but otherwise advantageous for other properties (e.g. improving therapeutic index) could be recovered.

Additionally, alternative silent Fvs (e.g. germline sequences) may also be employed. An illustrative IL18 fusions in the IL18 x Fab-Fc format utilizing the DP47GS germline sequence is depicted in FIG. 22CA as XENP40686.

2D: Engineering IL18 Fusions for Rapid Purification (RP)

To simplify purification to enable higher throughput screening of IL18 variants, IL18 fusions were engineered with Rapid Purification or RP variants. Specifically, the empty-Fc or Fab-Fc chain was engineered with the H435R/Y436F variants to ablate Protein A binding. Combined with DNA ratio optimization which reduced formation of IL18-Fc homodimers, this approach enabled a single-step Protein A purification which removed empty-Fc homodimers (in the case of monovIL18-Fc) or Fab-Fc homodimers (in the case of IL18 x Fab-Fc). Although many of the IL18 fusion sequences described herein include the RP variants, the IL18 fusions may be engineered and produced without the RP variants.

2E: E31Q Variant Enhances Yield

In engineering IL18 affinity variants as described in Example 3, the E31Q substitution was unexpectedly found to improve production yield. In combination with DNA ratio optimization, the E31Q substitution removed the necessity for engineering IL18 fusions in the IL18 x Fab-Fc format.

### Example 3

### Engineering IL18 Variants to Improve Therapeutic Index

In vivo, IL18's proinflammatory effect is attenuated by IFNy-dependent expression of IL18BP (sequence depicted in FIGS. 1-3, respectively for human, mouse, and F). IL18BP binds free IL18 and prevents IL18 binding to IL18R1. In order to overcome the IL18BP sink, it would be necessary to dose IL18 fusions at high concentrations; however, this may lead to systemic toxicity.

It was reasoned that decreasing the affinity of IL18 for IL18 receptors (and by extension, decreasing their potency) would allow for higher dosing to overcome the IL18BP sink and extend the half-life of the IL18 fusion proteins. Alternatively, or in addition to this approach, it was reasoned that decreasing the affinity of IL18 for IL18BP would overcome this sink. Accordingly, IL18 variants were engineered with the aim to achieve one or both of these targets, and these variants are collectively referred to herein as affinity variants. It should be noted that variants with improved binding to IL18BP and/or the IL18 receptors were also identified and useful in certain contexts, and these variants are also considered as affinity variants.

3A: Kinetic Binding Assay

Binding of variant IL18 to IL18 receptors or IL18BP were investigated using Octet, a BioLayer Interferometry (BLI)-based method. Experimental steps for Octet generally include the following: Immobilization (capture of ligand to a biosensor); Association (dipping of ligand-coated biosensors into wells containing serial dilutions of the analyte); and Dissociation (returning of biosensors to well containing buffer) in order to determine the affinity of the test articles. A reference well containing buffer alone was also included in the method for background correction during data processing. In particular, ligands used were IL18R1, IL18R1xIL18RAP heterodimer, or IL18BP, and the analytes were IL18 fusions. Illustrative sequences for these antigens are depicted in FIGS. 1-3 (respectively for human, mouse, and cynomolgus monkey).

3B: In Vitro Assay

To screen for IL18 variants engineered for reduced activation via IL18 receptors or reduced neutralization by IL18BP, in vitro assays were also used. For these assays, human myelomonocytic cell line, KG-1, which produces IFNy and upregulates PD-L1 in response to human IL18 was utilized.

In one assay format, dose dependent activation by IL18 fusions was investigated by stimulating KG-1 cells with increasing concentrations of IL18 fusions alone (to investigate reduced activation potency) or in the presence of fixed amount of IL18BP (to investigate reduced neutralization by IL18BP). IL18BP levels in NSCLC patient serum has been reported to be in the range of 10-50 ng/ ml (or 0.5-3 nM). Accordingly in this assay format, 100 ng/ml (5.6 nM) IL18BP was added to simulate higher end of in vivo IL18BP levels.

In another assay format, the dose dependent neutralization effect of IL18BP was investigated by stimulated KG-1 cells with fixed amount of IL18 fusions in the presence of increasing concentrations of IL18BP.

Illustrative examples of these two assay formats utilizing recombinantly produced IL18 (MBL, Cat: #B001-5) and IL18BP (Acro, Cat. ILP-H5222) are depicted in FIGS. 26A-26B.

3C: Affinity Variants (Library 1)

Several approaches were used in a first round of engineering. Crystal structure of the IL18 signaling ternary complex as reported by Tsutsumi et al., supra [PDB code 3W04] and crystal structure of human IL18 in complex with Ectromelia virus IL18 binding protein as reported by Krumm et al. (Proc Natl Acad Sci USA, 30 Dec 2008, 105(52):20711-20715) [PDB code 3F62] were used to identify residues on IL18 which interacted with any part of the IL18 receptors or IL18BP. In particular, MOE software's free energy calculations (AMBER force field parameters) was used to calculate energy between amino acid residues at the IL18:IL18R1 interface or the IL18:IL18BP interface. This round primarily focused on residues such as D, E, N, and Q at which isosteric substitutions could be introduced (reasoning that isosteric substitutions have less potential for immunogenicity) and identified E6, D17, E31, D35, D37, D40, N41, Q103, D110, and N111 as suitable residues for engineering. However, Y1 and K53 were also identified by the free energy calculations. In particular, K53 was calculated to be a residue very important for IL18:IL18BP binding. Based on this analysis, a number of IL18 variants were engineered (sequences for which are depicted in FIGS. 15A-15D) and formatted as IL18 x Fab-Fc fusions (sequences for which are depicted in FIGS. 22A-22CZ as noted above in Example 2A, these variants were engineered on the 4CS background).

The variant IL18 x Fab-Fc fusions were investigated in vitro as generally described above in Example 3B. In a first experiment, KG-1 cells were stimulated with increasing concentrations of IL18 fusions for 48 hours after which IL18 activity was evaluated by staining for PD-L1 expression as an indication of KG-1 cell activation. In a second set of experiments, KG-1 cells were stimulated with increasing concentrations of IL18 fusions for 48 hours in the absence or presence of fixed concentration of IL18BP. The data as depicted in FIG. 27 show that a number of the variants including E31Q, D35N, D37N, N41Q, K53M, and K53A decreased potency in KG-1 activation relative to WT-4CS IL18. The data as depicted in FIGS. 28A-28S show that for most of the variants, incubation with IL18BP shifts the activation potency; however, XENP38865 having the K53A appear minimally impacted by IL18BP (FIG. 28P) indicating that the K53A substitution reduces binding affinity and sink by IL18BP. It should be noted that, while not shown, molar equivalent (relative to XENP37827) of recombinant human IL-18 induces PD-L1 expression with similar potency as XENP37827.

In another set of experiments, KG-1 cells were stimulated with fixed concentration of IL18 fusions (10 nM) or molar equivalent of recombinant IL18 (5.6 nM) for 48 hours in the presence of increasing concentrations of IL18BP. IL18 activity was evaluated by staining for PD-L1 expression as an indication of KG-1 cell activation, data for which are depicted in FIGS. 29-30. Notably as depicted in FIG. 29 and consistent with the above, the specific substitution at K53A IL18's ability to escape IL18BP neutralization. For example, K53R substitution has no impact relative to WT. At this fixed concentration of IL18 fusion proteins, the K53M, K53Q and K53E variants exhibit slightly reduced inhibition potency relative to WT-4CS (as depicted in FIG. 29), but also exhibits reduced activation efficacy. The K53A variant exhibits reduced inhibition potency only.

Interestingly as seen in FIGS. 28C and 28D, the E6A and E6Q variants were found to improve binding to IL18R1. While this is opposite to the engineering target, these substitutions could be useful for introducing into very weak IL18R1-binding variants which may have otherwise advantageous properties (e.g., reduced IL18BP binding) to restore IL18R1 binding. Additionally as this residue is clearly important for binding between IL18 and IL18R1, other substitutions at this position may conversely reduce binding to IL18R1.

3D: Affinity Variants (Library 2)

Following the success from the first library of affinity variants, a further library was made to identify additional residues for introducing substitutions as well as to investigate combinations of substitutions. This library focused on positions along the IL18:IL18BP interface to identify variants with decreased binding to IL18BP. As this interface overlaps with the IL18:IL18R1 interface, mutations may have an effect on interactions with both IL18BP and IL18R1. Accordingly, this library further focused on variants that drastically reduced IL18BP binding while minimally impacted IL18R1 binding; improved binding to IL18R1 while minimally impacting binding to IL18BP; or decreased binding to both IL18R1 and IL18RAP. Additional residues identified include K8, M51, S55, Q56, P57, M60, and H109. Additional IL18 variants were engineered (sequences for which are depicted in FIGS. 16A-16E and 17A-17B) and formatted as IL18 x Fab-Fc fusions (sequences for which are depicted in FIGS. 22A-22CZ; as above, these variants were engineered on the 4CS background).

Kinetic binding experiments as generally described in Example 3A were performed to investigate the binding of the variant IL18 x Fab-Fc fusions to IL18R1 antigen, ILBP antigen, and IL18R1xIL18RAP heterodimer antigen, data for which are summarized in FIG. 31. Notably, a K53D single substitution variant (XENP40253) was found to have a weaker binding response to IL18R1 and IL18R1xRAP but advantageously also no binding response to IL18BP. The K53N single substitution variant (XENP40252) also abrogated binding to IL18BP, but also resulted in much weaker IL18R1xRAP binding. Additional observations made were as follows: several other substitutions at K53 also enabled weakened binding for both IL18R1 and IL18BP, albeit not to the same level of weaked IL18BP binding as K53D and K53N; S55N and S55Q enabled weakened binding to IL18R1 and IL18BP, although at the expense of stability (as depicted in FIGS. 36); and M41K enabled enhanced binding for IL18R1.

Next, in vitro assays as generally described in Example 3B were performed to investigate activation potency of the variant IL18 x Fab-Fc fusions. KG-1 cells stimulated with indicated concentrations of the IL18 x Fab-Fc fusions for 48 hours after which cells were stained with anti-PDL1 mAb to evaluate IL18 activity. Data are depicted in FIGS. 32-35.

Notably, K53T may be a promising variant as it demonstrated good induction activity with very quick off-rate (as determined by Octet binding). Additionally, K53E demonstrated good induction of activity at higher concentration. P57E demonstrated similar induction of activity in comparison to K53E, but as will be described in Example 4C, the P57E additionally improves stability.The K53D and K53N variants demonstrated activity which is promising, and in addition, the K53N variant improves stability (as depicted in Example 4C) possibly by introducing an N-linked glycoylation.

### Example 4

### Engineering IL18 Variants to Improve Stability

4A: Stability Assay

Stability of the IL18 fusions were assessed using Differential Scanning Fluorimetry (DSF). DSF experiments were performed using a Bio-Rad CFX Connect Real-Time PCR Detection System. Proteins were mixed with SYPRO Orange fluorescent dye and diluted to 0.2 mg/mL in PBS. The final concentration of SYPRO Orange was 10X. After an initial 10 minute incubation period at 25° C., proteins were heated from 25 to 95° C. using a heating rate of1° C/min. A fluorescence measurement was taken every 30 sec. Melting temperatures (Tm) were calculated using the instrument software. IL18 x Fab-Fc fusions had a first unfolding event (Tm1) of ˜40° C. Generally, the stability of IgG1 is limited by the Tm1 of the Fc region at 66° C. Therefore, a IgG1-based Fc fusion with a Tm1 of —40° C. is suboptimal. Accordingly, IL18 variants were engineered with the aim to improve stability, and these variants are referred to herein as stability variants.

4B: Stepwise Reversion of C4S Substitutions

An initial avenue explored for improving stability was stepwise reversion of the substituted cysteines (from Example 2A) in the event that they were important for stability. Sequences of such IL18 variants are depicted in FIGS. 13A-13B and IL18 x Fab-Fc fusions based on these variants are depicted in FIGS. 22A-22CZ. In data not shown, restoration of cysteine at position 76 (i.e., C38S/C68S/C127S/D157_) improved Tm by 1.5° C. relative to 4CS/D157 in the context of IL18-G4S-His molecules.

4C: Certain Affinity Variants Improves Stability

In engineering IL18 affinity variants as described in Example 3C, the E31Q substitution was unexpectedly found to improve stability. Stability of the IL18 x Fab-Fc fusions comprising the variants in Example 3C were investigated as described in Example 4A. It was found that while most of the IL18 x Fab-Fc fusions had a Tm1 ranging from 35.5-42.5° C., XENP38855 having the E31Q IL18 variant had a Tm1 of 45° C. (FIG. 36). Furthermore, the improved stability conferred by the E31Q substitution is retained when combined with other variants, such as the combination variants as described in Example 3D (data not shown). Additionally in engineering the additional single substitution affinity variants in Example 3D, it was surprisingly found that K53N, P57A, and P57E advantageously increased Tm1 respectively by 5, 9.5, and 12° C.; notably, several substitutions, including alternative substitutions as the aforementioned positions, decreased Tm1 by as much as 11.5° C. (FIG. 37).

4D: Stability Variants (Library 1)

Several approaches were used in a first round of engineering. As in Example 3, MOE software's free energy calculations were used. The engineering focused on introducing disulfide bridges using existing cysteine residues, introducing disulfide bridges at novel sites, and mutating existing cysteine residues or core positions to hydrophobic residues. Additionally based on a sequence alignment of IL18 from various species such as Equus caballus, Bos taurus, and others (see FIG. 38), mutations to commonly observed residues were made. Based on these approaches, a number of IL18 variants were engineered (sequences for which are depicted in FIGS. 19A-19P; It should be noted that each of these stability variants include the 4CS substitutions, unless reversion to cysteine is explicitly denoted i.e., S38C, S68C, S76C, and/or S127C) and formatted as IL18 x Fab-Fc fusions (sequences for which are depicted in FIGS. 22A-22CZ). It should be noted that in the context of the IL18 x Fab-Fc fusions, the variants all include E31Q to improve production (and the 4CS substitutions, unless reversion to cysteine explicitly called out i.e. S38C, S68C, S76C, and/or S127C). The data as depicted in FIG. 39 show a number of the variants improved Tm1 by up to 20° C. (relative to the E31Q variant having Tm1 of 45° C.) such as the disulfide bridge introducing S10C/N155C variant. Melting curves for illustrative His-tagged IL18 variants are shown in FIG. 72. Notably, these stabilizing disulfide bridges were able to overcome the requirement for E31Q.

4E: Stability Variants (Library 2)

Following the success from the first library of stability variants, a further library was made. The emphasis of this second library was to further explore native unpaired cysteines and mutate them to residues other than serine. Additionally, potentially interacting variants from the sequence alignment were mutated. Based on these approaches, a number of additional IL18 stability variants were engineered (sequences for which are depicted in FIGS. 20A-20D; It should be noted that each of these stability variants include the 4CS substitutions, unless alternative substitution at residues 38, 68, 76, and/or 127 is explicitly denoted e.g., S38E) and formatted as IL18 x Fab-Fc fusions (sequences for which are depicted in FIGS. 22A-22CZ). Stability was assayed as described in Example 4A, data for which are depicted in FIG. 40. As above, in the context of the IL18 x Fab-Fc fusions, the variants all include E31Q to improve production (and the 4CS substitutions, unless alternative substitution at residues 38, 68, 76, and/or 127 is explicitly denoted e.g., S38E). Notably, substitution of S38 for other residues such as L, I, or V improved stability. Restoration of cysteine at residue 76 improved stability which is consistent with findings as described in Example 4B. E141K/I149V and E141Q/I149V also enabled minor improvement in stability.

### Example 5

### Further IL18 Variants

Further IL18 variants were engineered based on the foregoing efforts. For example in Further Variants (Library 1), favorite stability variants identified in Example 4 were combined (sequences for which are depicted in FIG. 41).

In Further Variants (Library 2), favorite positions identified in Affinity Variants (Libraries 1 and 2) (see Example 3C and 3D) were revisited and alternative amino acids at these positions were explored. Additionally, further structural modeling identified S36 and D132 as additional potential positions for engineering affinity modulating variants. Sequences for Further Variants (Library 2) are depicted in FIG. 42.

As identified Stability Variants (Library 1) (see Example 4D), disulfide bridge introducing S10C/N155C variant improved Tm by 20° C. and is suitable for combining with other variants; thus in Further Variants (Library 3), favorite affinity variants were combined with S10C/N155C (sequences for which are depicted in FIG. 43).

As noted in Example 3D, K53D abrogated IL18BP binding, but also reduced IL18R1 and IL18R1xRAP binding. Additionally as described in Example 3D, certain variants found to improve IL18R1 binding could be used to restore IL18R1 binding (and, by extension, activity). Accordingly in Further Variants (Library 4), combination variants were explored combining affinity variants (those that modulate affinity for IL18 receptors and/or those that modulate affinity for IL18BP e.g. K53D) (sequences for which are depicted in FIG. 44). In particular, IL18R1 enhancing mutations combined with K53D included E6Q, S55T, and/or N111T. As depicted in FIG. 62, K53D as in XENP41762 resulted in >1000 fold reduction in potency relative to WT XENP41756. Incorporating E6Q and N111T as in XENP42006 resulted in —300 fold reduction in potency relative to WT XENP41756.

The new variants were formatted as IL18 x Fab-Fc or monovIL18-Fc fusions (sequences for which are depicted in FIGS. 21-22) and assayed as described in Example 3A and 3B, data for which are depicted in FIGS. 48-62 and 74. It should be noted that while 100 ng/mL IL18BP were previously utilized, 1 pg/mL IL18BP were utilized in some of these additional experiments. As will be described in Example 7A, it was found that IL18BP was upregulated by the IL18 fusion proteins of the invention, and therefore, IL18 variants that can tolerate higher concentrations of IL18BP would be preferred.

It should be noted that variants across the various libraries were often stepwise combined with previous favorites. For example, while Further Variants (Library 4) was exploring combinations of affinity variants, each of the variants depicted in FIG. 44 further includes 4CS and S10C/N155C. Accordingly, FIGS. 45-47 summarizes positions and substitutions that were explored for each purpose (e.g. Affinity Variants vs. Stability Variants).

### Example 6

### IL18 x Fab-Fc vs. monovIL18-Fc

As with the incorporation off E31Q and S10C/N155C, it is possible to express stable IL18 fusions as either IL18 x Fab-Fc or monovIL18-Fc, the activity of equivalent variants in either format was further investigated. The data depicted in FIG. 63 show that the IL18 x Fab-Fc fusions have lower Ymax (lower efficacy) than their monovIL18-Fc counterpart. This both suggests that it is difficult to compare variants between formats and that it is important that engineering efforts to “fix” monovIL18-Fc fusions (e.g. introducing novel disulfide bridges) were successful.

### Example 7

### In Vivo Characterization of Potency Reduced IL18 Fusions

To determine if the in vitro potency reduction in the IL18 fusions translated to activity in vivo, a graft-versus-host disease (GvHD) model conducted in NSG (NOD-SCID-gamma) immunodeficient mice was used. When the NSG mice are injected with human PBMCs, the human PBMCs develop an autoimmune response against mouse cells. Treatment of huPBMC-engrafted mice with IL18 fusions should activate and expand the engrafted human T cells and exacerbate disease.

7A: GvHD Study #1

In a first study, the aim was to examine biological activities of potency reduced IL18 fusions and explore how potency can be reduced while still maintaining activity. 10 x 10⁶ were engrafted into NSG mice on Day 0 along with 5 mg/kg or 0.5 mg/kg XENP40967 (3-fold reduction from WT), XENP40685 (17-fold reduction from WT), XENP40966 (219-fold reduction from WT), XENP40962 (210-fold reduction from WT), and XENP40965 (9220-fold reduction from WT). Mice were further dosed on Days 7, 14, and 21. Mice were weighed twice a week (change in body weight as an indicator of GvHD), data for which are depicted in FIG. 64. Blood was drawn on Day 7, 14, and 21 to investigate cytokine secretion and lymphocyte expansion and activation, data for which are shown in FIGS. 65-69. The data show that change in body weight, human T cell expansion, NK cell expansion, and cytokine secretion generally correlated with IL18 potency and was dose dependent. Additionally, the body weights and T cell counts correlated with the variants that were engineered to avoid IL18BP inhibition. The effect is less pronounced at higher 5 mg/kg doses, but at lower 0.5 mg/kg doses, the K53T and K53D variants as in XENP40967 and XENP40962 noticeably enhanced GVHD. While K53T likely enhances GVHD due to its higher potency, K53D is lower potency but enhances GVHD in comparison to variants with similar potency but which do not avoid IL18BP inhibition. Notably as depicted in FIG. 65, the IL18 fusions induced IL18BP. This further highlights the importance of detuning IL18BP binding.

7B: PK Study #1

To characterize the in vivo stability of the IL18 fusion proteins, PK experiments were performed in C57/B16 mice. Mice (n=4) were intravenously dosed on Day 0 with 2 mg/kg XENP39804 (monovIL18-Fc with 4CS/E31Q variant), XENP40685 (monovIL18-Fc with 4CS/E31Q variant and further including S 10C/N155C), and XENP40686 (IL18 x Fab-Fc with 4CS/E31Q variant and further including S10C/N155C). Blood was drawn 1 hour post dose and on Days 2, 5, 8, 13, and 16, and serum concentration of the IL18 fusion proteins was determined by anti-human IL18 detection antibody. PK interpretative analysis was performed using Phoenix WinNonlin software (Version 6.4.0.768) with PK parameters for non-compartmental analysis of free drug serum concentration versus time, data for which are shown in FIG. 70. The data show dramatic improvement in serum levels upon introduction of the S10C/N155C disulfide variant into the E31Q base and enabled antibody-like PK with an estimated half-life of 3 weeks.In addition, the IL18 x Fab-Fc format did not appear to confer any further improvements.

7C: GvHD Study #2

In another study, dose dependent in vivo activity of XENP41770, XENP42006, and XENP41762 was investigated. 10 x 106 were engrafted into NSG mice on Day 0 along with indicated concentrations of XENP41770, XENP42006, and XENP41762. Mice were further dosed on Days 7, 14, 21, and 28. Mice were weighed twice a week (change in body weight as an indicator of GvHD), data for which are depicted in FIG. 75. Notably by Day 8, 3 mg/kg XENP42006 enabled earliest enhanced GvHD (i.e. significantly enhanced in comparison to PBS control). Over the course of the study, XENP42006 continued to outperform XENP41770 and XENP41762 even at lower doses. Blood was drawn on to investigate cytokine secretion and lymphocyte expansion, data for which are shown in FIGS. 76-77.

7C: PK Study #2

Next, PK experiments were performed in cynomolgus monkeys. Monkeys were dosed on Day 0 and Day 14 with either XENP41974 (stabilized, WT affinity) and XENP42007 (stabilized, affinity-optimized which is XENP42006+M428L/N434S half-life extension Fc variant+Rapid Purification variant; note that XENP42143 is 42007 without the Rapid Purification variant). Data depicting total IL18-Fc vs. active IL18-Fc concentration over time are depicted in FIGS. 78-79. Total IL18-Fc includes IL18-Fc with and without bound IL18BP, while active IL18-Fc is unbound. The data show that active XENP41974 is rapidly cleared, while XENP42007 exhibits greatly improved PK (both slow receptor-mediated clearance and avoiding IL18BP sink). Additionally, the IL18-Fc was well tolerated and the cynomolgus monkeys exhibited no clinical observations.

### Example 8

### Further Characterization of Fc Variants

A majority of the data depicted herein were in the context of IL18 x Fab-Fc fusions or in the context of Fc fusions having Rapid Purification variants. Accordingly, it is important to confirm that the IL18 variants demonstrated their expected effect in the context of other Fc fusions havin alternative variants (e.g. having M428L/N434S half-life extension Fc variant and excluding the Rapid Purification variant). Accordingly, the in vitro activity of XENP42141, XENP42143, and XENP42145 were investigated in a cytokine release assay. Human PBMC was stimulated with the IL18-Fc fusions plus human IL-12 and secretion of cytokine IP-10 was assessed. As expected, the data as depicted in FIG. 81 show that XENP42141 having WT potency most potently induced IP10 secretion while XENP42145 least potently induced IP10 secretion with XENP42143 (having the potency restoring E6Q/N111T variants) falling in between.

### Example 9

### Identifying Additional IL18 Variants By Investigating Murine IL18

As depicted in Example 4D, engineering IL18 variants based on sequence alignment of IL18 from other species may be useful. Accordingly, additional murine IL18 variants were engineered (sequences as depicted in FIG. 80A-80P in the context of murine IL18-Fc fusions).

To screen these variants, mouse splenocytes were stimulated with murine IL18-Fc fusions plus murine IL-12 and activation of NK1.1 cells (indicated by % intracellular IFNγ+) was investigated, data for which are depicted in FIG. 82. Additionally, binding to murine IL18BP was also investigated as generally described herein, data for which are depicted in FIGS. 83. Collectively, the following observations were made: L59K dramatically lowers affinity to IL18BP with beneficial effect on receptor potency; L59K in combination with K52X knocks out IL18BP binding; E55R and M5OG are silent, even when combined with each other or with L59K; K52 variants modulates receptor affinity like corresponding K53 in human IL18 with K52A >K52V >K52D; E30 and D34 variants also modulate receptor affinity like corresponding E31 and D3 5 varinats in human IL18 (although does not modulate IL18BP binding). As K52 variants in murine IL18 demonstrated similar effect as corresponding K53 variants in human IL18 (*note: corresponding based on alignment in FIG. 38), it is expected that other murine variants (e.g. at L59, and in particular, L59K) convey similar effect in human IL18. Accordingly, for example, it is expected that M6OK in human IL18 (which corresponds to murine L59K based on alignment in FIG. 38) may reduce or knockout IL18BP binding and may be combined with K53 variants to further abrogate IL18BP binding. Similarly, it is expected that K53V in human IL18 may be useful in modulating receptor affinity.

1. A composition comprising a variant human IL18 protein, wherein the variant IL18 protein comprises a modification at one or more amino acid positions selected from the group consisting of Y1, E6, S7, K8, S10, V11, N14, L15, D17, Q18, D23, R27, P28, L29, E31, M33, T34, D35, S36, D37, C38, R39, D40, N41, R44, I46, I49, S50, M51, K53, D54, S55, Q56, P57, M60, A61, V62, T63, S65, K67, C68, E69, I71, C76, E77, I80, I81, N87, P88, D90, K93, T95, K96, S97, Q103, H109, D110, N111, M113, S119, A126, C127, D132, L136, L138, K139, E141, L144, D146, R147, I149, M150, N155, E156, and D157, as compared to wildtype human IL18.

2.-15. (canceled)

16. A monovalent Fc fusion protein comprising:
(a) a first monomer comprising from N-terminus to C-terminus: a wildtype or variant IL-18 protein and a first Fc domain; and
(b) a second monomer comprising a second Fc domain.

17.-33. (canceled)

34. A monovalent Fc fusion protein comprising:
(a) a first monomer comprising from N-terminus to C-terminus: a variant IL-18 protein and a first Fc domain, wherein the variant human IL-18 protein comprises amino acid substitutions 4CS/E6Q/S10C/K53D/N111T/N155C, and wherein the variant IL-18 protein is covalently attached to the N-terminus of the first Fc domain; and
(b) a second monomer comprising a second Fc domain, wherein the first Fc domain comprises amino acid substitutions C220S/PVA JS267K/L368D/K370S/M428LJN434S, and the second Fc domain comprises amino acid substitutions PVA_/S267K/S364K/E357Q/M428L/N434S, according to EU numbering.

35.-38. (canceled)

39. A Fab-Fc fusion protein comprising:
(a) a first monomer comprising from N-terminus to C-terminus: a variable heavy (VH) chain and a first Fc domain;
(b) a second monomer comprising from N-terminus to C-terminus: a wildtype or variant IL-18 protein and a second Fc domain; and
(c) a third monomer comprising a variable light (VL) chain, wherein the VH and VL form an antigen binding fragment (Fab).

40.-56. (canceled)

57. A Fab-Fc fusion protein comprising:
(a) a first monomer comprising from N-terminus to C-terminus: a variable heavy (VH) chain and a first Fc domain;
(b) a second monomer comprising from N-terminus to C-terminus: a variant IL-18 protein and a second Fc domain, wherein the variant human IL-18 protein comprises amino acid substitutions 4CS/E6Q/S10C/K53D/N111T/N155C; and
(c) a third monomer comprising a variable light (VL) chain, wherein the VH and VL form an antigen binding fragment (Fab),
wherein the first Fc domain comprises amino acid substitutions C220S/PVA JS267K/L368D/K370S/M428LJN434S, and the second Fc domain comprises amino acid substitutions PVA JS267K/S364K/E357Q/M428L/N434S, according to EU numbering.

58.-65. (canceled)
